(19) United States
(12) Patent Application Publication
(10) Pub. No.: US 2024/0309398 A1
(43) Pub. Date: Sep. 19, 2024

(54) ENHANCERS DRIVING EXPRESSION IN MOTOR NEURONS

(71) Applicant: President and Fellows of Harvard College, Cambridge, MA (US)

(72) Inventors: Sinisa Hrvatin, Cambridge, MA (US); Michael E. Greenberg, Cambridge, MA (US); Mark Aurel Nagy, Cambridge, MA (US); Eric C. Griffith, Cambridge, MA (US)

(73) Assignee: President and Fellows of Harvard College, Cambridge, MA (US)

(21) Appl. No.: 18/410,249

(22) Filed: Jan. 11, 2024

Related U.S. Application Data
(63) Continuation of application No. PCT/US2022/037340, filed on Jul. 15, 2022.
(60) Provisional application No. 63/222,864, filed on Jul. 16, 2021.

(51) Int. Cl. C12N 15/86

(52) U.S. Cl. CPC C12N 15/86; C12N 2750/14143; C12N 2830/008

(57) ABSTRACT

The technology described herein is directed to a gene regulatory element, e.g., enhancer, vectors comprising the same, adeno-associated vectors comprising the same and cells comprising said vectors. In another aspect, described herein are methods of treating a motor neuron disease or disorder comprising administration of said vectors, e.g., AAV vectors. In another aspect, described herein are nucleic acid compositions comprising the gene regulatory element as described herein.

### RELATED APPLICATIONS

The instant application is a continuation of International Application No. PCT/US2022/037340, filed Jul. 15, 2022, which claims priority to U.S. Provisional Application No. 63/222,864, filed Jul. 16, 2021, the entire contents of which are expressly incorporated by reference herein in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 9, 2024, is named “117823-32102_SL” and is 354,902 bytes in size. The sequence listing contained in this .XML file is part of the specification and is hereby incorporated by reference herein in its entirety.

### TECHNICAL FIELD

Described herein are compositions related to regulatory elements, such as elements directing cell type specific expression.

### BACKGROUND OF THE INVENTION

Spinal muscular atrophy (SMA) and amyotrophic lateral sclerosis (ALS) are highly debilitating diseases affecting spinal motor neurons (MNs). SMA, resulting from loss-of-function mutations in the SMN1 gene, represents a particularly appealing candidate for gene therapy-based interventions, and an adeno-associated virus (AAV)-based treatment to restore SMN1 expression was recently reported to improve motor function in an early-stage single-site clinical trial. Despite this progress, the current generation of gene therapy vectors employs ubiquitously active gene regulatory elements (GREs) to drive strong payload expression in all transduced cells, and poorly restricted payload delivery represents a potentially serious source of clinical toxicity. Indeed, recent findings from primate models showed non-immune-based toxicity with systemic delivery of high dosage AAVs for which payload expression is not restricted to the target organ. Thus, MN-restricted viral expression might result in increased safety and an expanded therapeutic window for SMA and ALS treatment.

To address these issues, the present disclosure provides methods and compositions for generating cell-type-specific AAV drivers, to generate novel AAVs capable of driving restricted gene expression within spinal cord MNs. The resulting viral constructs will represent promising candidates for the basis of next-generation motor neuron disease or disorder (e.g., SMA and ALS) gene therapeutics.

### SUMMARY OF THE INVENTION

Accordingly, in one aspect, the present invention provides a nucleic acid comprising a regulatory element sequence that has at least 85% identity to SEQ ID NOs: 1-14 or 60-71 or a fragment thereof.

In some embodiments, the regulatory element sequence has at least 90%, 95%, 98%, 99%, or 100% identity to SEQ ID NOs: 1-14 or 60-71. In some embodiments, the sequence comprises at least one modification relative to SEQ ID NO: 1-14 or 60-71, optionally a substitute modification. In some embodiments, the nucleic acid comprises at least one additional regulatory element sequence. In some embodiments, the nucleic acid comprises at least two, three, four, five or six additional regulatory element sequences.

In some embodiments, the at least one additional regulatory element sequence has at least 85% identity to SEQ ID NO: 1-14 and 60-71.

In some embodiments, the at least one additional regulatory element sequence has at least 90%, 95%, 98%, 99%, or 100% identity to SEQ ID NOs: 1-14 or 60-71. In some embodiments, the at least one additional regulatory sequence comprises at least one modification relative to SEQ ID NO: 1-14 or 60-71, optionally a substitute modification.

In some embodiments, the nucleic acid comprises two or more identical copies of a regulatory element sequence selected from the group consisting of SEQ ID NO: 1-14 or 60-71.

In some embodiments, the nucleic acid comprises two, three, four, five or six identical copies of the regulatory element sequence. In some embodiments, the nucleic acid comprises at least two or more versions of a regulatory element sequence having at least 85%, 90%, 95%, 98%, 99%, or 100% identity to SEQ ID NO: 1-14 or 60-71, wherein the first version of the regulatory element sequence differs from the second version of the regulatory element sequence.

In some embodiments, the nucleic acid further comprises a promoter.

In some embodiments, the nucleic acid further comprises a heterologous gene.

In some embodiments, the regulatory element comprises SEQ ID NO: 1-14 or 60-71. In some embodiments, the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides.

In some embodiments, the regulatory element comprises one or more transcription factor binding sites. In some embodiments, the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA) or a combination thereof.

In some embodiments, the heterologous gene is naturally expressed in a neuron. In some embodiments, the neuron is selected from the group consisting of a motor neuron, a sensory neuron, and an interneuron. In some embodiments, the neuron is a motor neuron. In some embodiments, the heterologous gene is selectively expressed in a motor neuron. In some embodiments, the heterologous gene is selectively expressed in a motor neuron, but is not expressed in dorsal root ganglia.

In some embodiments, the heterologous gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPI1 (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPT1C (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kincsin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), and a combination thereof.

In some embodiments, the heterologous gene is SMN1. In some embodiments, the SMN1 gene comprises the sequence set forth in SEQ ID NO: 25-28. In some embodiments, the SMN1 gene comprises the sequence set forth in SEQ ID NO: 28.

In some embodiments, the heterologous gene is an inhibitory nucleic acid. In some embodiments, the inhibitory nucleic acid is a microRNA (miRNA), artificial microRNA (amiRNA), or short hairpin RNA (shRNA).

In some embodiments, the inhibitory nucleic acid is a microRNA, wherein the microRNA binds to mRNA of a target gene. In some embodiments, the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPI1 (Triosephosphate Isomerase 1), ATL1 (Atlastin GTPase 1), SPAST (Spastin), NIPA1 (Non-imprinted in Prader-Willi/Angelman syndrome region protein 1), KIAA 1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2). SOD1 (superoxide dismutase 1), C9orf72 (C9orf72-SMCR8 Complex Subunit), or a combination thereof.

In some embodiments, the target gene is SOD1. In some embodiments, the SOD1 gene comprises the sequence set forth in SEQ ID NO: 33.

In some embodiments, the target gene is C9orf72. In some embodiments, the C9orf72 gene comprises the sequence set forth in SEQ ID NO: 35, 36, or 37.

In some embodiments, the promoter is a promoter from a gene expressed in motor neurons, optionally wherein the gene expressed in motor neurons is selected from the group consisting of a Dnajc22, Sycp1, Slit3, Hrasls5, Otop3, Conb3, Nlrp3, Hormad1, Chat, Anxa4, Tnfsf4, Myo3b, Cdh15, Nr2e1, I117f, Apela, Gnb3, Pappa, Tmprss15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, I122ra1, Galp, Meil, Aox1, Prph, Slc25a54, Cdhr1. Tgm6, Ppm1j. Esrp1, Gem, Is11, Itpr3, Sec16b, Pde6b, Haol, Oaslf, I121r. Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd, Terb2, Ins15, Card11, Platr7, Miat, Slc5a7, Iqcg. Topaz1. Tex 14, Slc5a10, Map4k1, Calcb, Got111, Slc46a2, Nanos2, Plekhg4, Themis3, Cpa3, Aknad1, Cdcp2, Uts2, Slc44a4, Popdc3, Tbata, Pkp1, Dysf, Pkp2, Sds, Nipsnap3a, Apo17e, Tex22, Mapk11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNC1H1, PLEKHG5, SIGMAR1, DNAJB2, SMAX3, TPI1, ATL1, SPAST, NIPA1, KIAA1096, KIFSA, RTN2, Hsp60, SPG37, SPG41, SLC33A1. REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC. ANG, TARDBP, FIG. 4, OPTN, ATXN2, VCP, CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, and C9orf72.

In some embodiments, the gene expressed in motor neurons is ChAT, Slc5a7, Is11, Mnx1, Lhx3, or Lhx4. In some embodiments, the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx1 promoter, Lhx3 promoter, Lhx4 promoter, and variants thereof.

In some embodiments, the nucleic acid is associated with an adeno-associated virus comprising a capsid that crosses the blood brain barrier and/or the blood spinal cord barrier. In some embodiments, the adeno-associated virus comprising a capsid is selected from the group consisting of AAV1. AAV2, AAV3, AAV4, AAV5, AAV6, AAV7, AAV8, AAV9, AAV10, AAV11, AAVrh. 10, AAV1 R6, AAV1 R7, rAAVrh.8, AAV-BR1, AAV-PHP.S, AAV-PHP.B, AAV-PPS, and AAV-PHP.eB.

Accordingly, in another aspect, the present invention provides a vector comprising the nucleic acid of the above aspects or any other aspect of the invention delineated herein.

In some embodiments, the vector is a viral vector. In some embodiments, the viral vector is a recombinant adeno-associated viral (AAV) vector.

Accordingly, in another aspect, the present invention provides a recombinant adeno-associated viral (rAAV) vector comprising a regulatory element sequence that has at least 85% identity to SEQ ID NOs: 1-14 or 60-71 or a fragment thereof.

In some embodiments, the regulatory element sequence has at least 90%, 95%, 98%, 99%, or 100% identity to SEQ ID NO: 1-14 or 60-71.

In some embodiments, the sequence comprises at least one modification relative to SEQ ID NO: 1-14 or 60-71, optionally a substitute modification.

In some embodiments, the nucleic acid comprises at least one additional regulatory element sequence. In some embodiments, the nucleic acid comprises at least two, three, four, five or six additional regulatory element sequences. In some embodiments, the at least one additional regulatory element sequence has at least 85% identity to SEQ ID NO: 1-14 and 60-71.

In some embodiments, the at least one additional regulatory element sequence has at least 90%, 95%, 98%, 99%, or 100% identity to SEQ ID NOs: 1-14 or 60-71. In some embodiments, the at least one additional regulatory sequence comprises at least one modification relative to SEQ ID NO: 1-14 or 60-71, optionally a substitute modification.

In some embodiments, the nucleic acid comprises two or more identical copies of a regulatory element sequence selected from the group consisting of SEQ ID NO: 1-14 or 60-71. In some embodiments, the nucleic acid comprises two, three, four, five or six identical copies of the regulatory element sequence.

In some embodiments, the nucleic acid comprises at least two or more versions of a regulatory element sequence having at least 85%, 90%, 95%, 98%, 99%, or 100% identity to SEQ ID NO: 1-14 or 60-71, wherein the first version of the regulatory element sequence differs from the second version of the regulatory element sequence.

In some embodiments, the nucleic acid further comprises a heterologous gene.

In some embodiments, the regulatory element comprises SEQ ID NOs: 1-14 or 60-71.

In some embodiments, the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides.

In some embodiments, the regulatory element comprises one or more transcription factor binding sites. In some embodiments, the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA), or a combination thereof.

In some embodiments, the heterologous gene is naturally expressed in a neuron. In some embodiments, the neuron is selected from the group consisting of a motor neuron, a sensory neuron, and an interneuron. In some embodiments, the neuron is a motor neuron. In some embodiments, the heterologous gene is selectively expressed in a motor neuron. In some embodiments, the heterologous gene is selectively expressed in a motor neuron, but is not expressed in dorsal root ganglia.

In some embodiments, the heterologous gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPI1 (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinasc 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinasc), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), and a combination thereof.

In some embodiments, the heterologous gene is SMN1. In some embodiments, the SMN1 gene comprises the sequence set forth in SEQ ID NO: 28.

In some embodiments, the heterologous gene is an inhibitory nucleic acid. In some embodiments, the inhibitory nucleic acid is a microRNA (miRNA), artificial microRNA (amiRNA), or short hairpin RNA (shRNA).

In some embodiments, the inhibitory nucleic acid is a microRNA, wherein the microRNA binds to mRNA of a target gene. In some embodiments, the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPI1 (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEK1 (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), SOD1 (superoxide dismutase 1), C9orf72 (C9orf72-SMCR8 Complex Subunit), or a combination thereof.

In some embodiments, the target gene is SOD1. In some embodiments, the SOD1 gene comprises the sequence set forth in SEQ ID NO: 33.

In some embodiments, the target gene is C9orf72. In some embodiments, the C9orf72 gene comprises the sequence set forth in SEQ ID NO: 35, 36, or 37.

In some embodiments, the rAAV further comprises a promoter. In some embodiments, the promoter is a promoter from a gene expressed in motor neurons, optionally wherein the gene expressed in motor neurons is selected from the group consisting of a Dnajc22, Sycp1, Slit3, Hrasls5, Otop3, Conb3. Nlrp3, Hormad1, Chat, Anxa4, Tnfsf4, Myo3b, Cdh15, Nr2e1, I117f, Apela, Gnb3, Pappa. Tmprss15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, 1122ra1, Galp, Meil, Aox1, Prph, Slc25a54, Cdhr1. Tgm6, Ppm1j, Esrp1, Gem, Is11. Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd, Terb2, Ins15, Card11, Platr7, Miat, Slc5a7, Iqcg, Topaz1, Tex14, Slc5a10, Map4k1, Calcb, Got111, Slc46a2, Nanos2, Plekhg4, Themis3, Cpa3, Aknad1, Cdcp2, Uts2, Slc44a4, Popdc3, Tbata, Pkp1. Dysf. Pkp2, Sds, Nipsnap3a, Apo17e. Tex22, Mapk11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNC1H1, PLEKHG5, SIGMAR1, DNAJB2, SMAX3, TPI1, ATL1, SPAST, NIPA1, KIAA1096, KIFSA, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP, CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, and C9orf72.

In some embodiments, the gene expressed in motor neurons is ChAT, Slc5a7, Is11, Mnx1. Lhx3, or Lhx4. In some embodiments, the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx1 promoter, Lhx3 promoter, Lhx4 promoter, and variants thereof.

In some embodiments, the rAAV vector is replication-competent.

Accordingly, in another aspect, the present invention provides a transgenic cell comprising the nucleic acid of the above aspects or any other aspect of the invention delineated herein and/or a vector of the above aspects or any other aspect of the invention delineated herein. In some embodiments, the transgenic cell is a neuron. In some embodiments, the transgenic cell is selected from the group consisting of a motor neuron, a sensory neuron, and an interneuron. In some embodiments, the transgenic cell is a motor neuron. In some embodiments, the transgenic cell is murine, human, or non-human primate.

Accordingly, in another aspect, the present invention provides a composition comprising the nucleic acid of the above aspects or any other aspect of the invention delineated herein, the vector of the above aspects or any other aspect of the invention delineated herein, the rAAV vector of the above aspects or any other aspect of the invention delineated herein, or the transgenic cell of the above aspects or any other aspect of the invention delineated herein; and a pharmaceutically acceptable excipient.

Accordingly, in another aspect, the present invention provides a method for selectively expressing a heterologous gene within a population of neural cells in vivo or in vitro, the method comprising providing the composition of the above aspects or any other aspect of the invention delineated herein in a sufficient dosage and for a sufficient time to a sample or a subject comprising the population of neural cells thereby selectively expressing the gene within the population of neural cells.

Accordingly, in another aspect, the present invention provides a method for selectively expressing a heterologous gene within a population of neural cells in vivo or in vitro, the method comprising providing a composition comprising a nucleic acid of the above aspects or any other aspect of the invention delineated herein and a pharmaceutically acceptable excipient in a therapeutically effective dosage to a sample or a subject comprising the population of neural cells thereby selectively expressing the gene within the population of neural cells.

In some embodiments, the composition is a lipid formulation, n some embodiments, the lipid formulation comprises one or more cationic lipids, non-cationic lipids, and/or PEG-modified lipids, or a combination thereof. In some embodiments, the pharmaceutical composition comprises a lipid nanoparticle.

In some embodiments, the providing comprises administering to a living subject. In some embodiments, the living subject is a human, non-human primate, or a mouse.

In some embodiments, the administering to a living subject is through injection. In some embodiments, the injection comprises intracerebroventricular (ICV) or intravenous (IV) injection, optionally into the cisterna magna (ICM).

Accordingly, in another aspect, the present invention provides a method for the treatment of a motor neuron disease or disorder in a subject in need thereof, the method comprising administering a recombinant adeno-associated virus (rAAV) comprising a regulatory element sequence that has at least 85% identity to SEQ ID NOs: 1-14 or 60-71 or a fragment thereof to the subject, wherein one or more symptoms associated with the motor neuron disease or disorder are inhibited or prevented.

In some embodiments, the regulatory element sequence has at least 90%, 95%, 98%, 99%, or 100% identity to SEQ ID NOs: 1-14 or 60-71. In some embodiments, the sequence comprises at least one modification relative to SEQ ID NOs: 1-14 or 60-71, optionally a substitute modification.

In some embodiments, the nucleic acid comprises at least one additional regulatory element sequence. In some embodiments, the nucleic acid comprises at least two, three, four, five or six additional regulatory element sequences. In some embodiments, the at least one additional regulatory element sequence has at least 85% identity to SEQ ID NO: 1-14 and 60-71. In some embodiments, the at least one additional regulatory element sequence has at least 90%, 95%, 98%, 99%, or 100% identity to SEQ ID NOs: 1-14 or 60-71. In some embodiments, the at least one additional regulatory sequence comprises at least one modification relative to SEQ ID NO: 1-14 or 60-71, optionally a substitute modification.

In some embodiments, the nucleic acid comprises two or more identical copies of a regulatory element sequence selected from the group consisting of SEQ ID NO: 1-14 or 60-71. In some embodiments, the nucleic acid comprises two, three, four, five or six identical copies of the regulatory element sequence.

In some embodiments, the nucleic acid comprises at least two or more versions of a regulatory element sequence having at least 85%, 90%, 95%, 98%, 99%, or 100% identity to SEQ ID NO: 1-14 or 60-71, wherein the first version of the regulatory element sequence differs from the second version of the regulatory element sequence.

In some embodiments, the nucleic acid further comprises a heterologous gene.

In some embodiments, the regulatory element comprises SEQ ID NOs: 1-14 or 60-71. In some embodiments, the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides.

In some embodiments, the regulatory element comprises one or more transcription factor binding sites. In some embodiments, the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA), or a combination thereof.

In some embodiments, the rAAV further comprises a promoter. In some embodiments, the promoter is a promoter from a gene expressed in motor neurons, optionally wherein the gene expressed in motor neurons is selected from the group consisting of a Dnajc22. Sycp1, Slit3, Hrasls5, Otop3, Ccnb3, Nlrp3, Hormad1, Chat, Anxa4, Tnfsf4, Myo3b, Cdh15, Nr2e1, I117f, Apela, Gnb3, Pappa. Tmprss15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, 1122ra1, Galp, Meil, Aox1, Prph, Slc25a54, Cdhr1, Tgm6, Ppm1j. Esrp1, Gem, Is11. Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd. Terb2, Ins15, Card11, Platr7, Miat, Slc5a7, Iqcg. Topaz1, Tex14, Slc5a10, Map4k1, Calcb, Got111, Slc46a2, Nanos2, Plekhg4, Themis3, Cpa3, Aknad1, Cdcp2, Uts2, Slc44a4, Popdc3, Tbata, Pkp1. Dysf, Pkp2, Sds, Nipsnap3a, Apo17c. Tex22, Mapk11, Fndc3c1, Axdnd1, Olah, Arhgap9, Cd4, Cpnc6, Etnk2. Slc38a8, Sapcd1, Esp11, Glyat, Htr2a, Slc10a4, Retn, Abcb11, Fam71f1, Is12, Lcp1, Usp50, Echdc2. Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNC1H1, PLEKHG5, SIGMAR1, DNAJB2, SMAX3, TPII, ATLI, SPAST, NIPA1, KIAA1096, KIFSA, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP, CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, and C9orf72.

In some embodiments, the gene expressed in motor neurons is ChAT, Slc5a7, Is11, Mnx1, Lhx3, or Lhx4. In some embodiments, the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx1 promoter, Lhx3 promoter, Lhx4 promoter, and variants thereof.

In some embodiments, the heterologous gene is naturally expressed in a motor neuron. In some embodiments, the heterologous gene is selectively expressed in a motor neuron. In some embodiments, the heterologous gene is selectively expressed in a motor neuron, but is not expressed in dorsal root ganglia.

In some embodiments, the heterologous gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPI1 (Triosephosphate Isomerase 1), ATL1 (Atlastin GTPase 1), SPAST (Spastin), NIPA1 (Non-imprinted in Prader-Willi/Angelman syndrome region protein 1), KIAA 1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEK1 (NIMA Related Kinase 1), DAO (D-Amino Acid Oxidasc), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), or a combination thereof.

In some embodiments, the heterologous gene is SMN1. In some embodiments, the SMN1 gene comprises the sequence set forth in SEQ ID NO: 28.

In some embodiments, the heterologous gene is an inhibitory nucleic acid. In some embodiments, the inhibitory nucleic acid is a microRNA (miRNA), artificial microRNA (amiRNA), or short hairpin RNA (shRNA). In some embodiments, the inhibitory nucleic acid is a microRNA, wherein the microRNA binds to mRNA of a target gene. In some embodiments, the target gene is selected from the group consisting of survival of motor neuron 1 (SMN1). AR (androgen receptor). BICD2 (BICD Cargo Adaptor 2), TRIP4 (Thyroid Hormone Receptor Interactor 4), HSPB1 (Heat Shock Protein Family B (Small) Member 1). HSPB8 (Heat Shock Protein Family B (Small) Member 8). HSPB3 (Heat Shock Protein Family B (Small) Member 3), FBXO38 (F-Box Protein 38), REEP1 (Receptor Accessory Protein 1). BSCL2 (BSCL2 Lipid Droplet Biogenesis Associated. Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2. SETX (Senataxin). DCTN1 (Dynactin Subunit 1). DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1). DNAJB2 (DnaJ Heat Shock Protein Family (Hsp40) Member B2), SMAX3 (spinal muscular atrophy-3). TPII (Triosephosphate Isomerase 1), ATL1 (Atlastin GTPase 1). SPAST (Spastin), NIPA1 (Non-imprinted in Prader-Willi/Angelman syndrome region protein 1). KIAA1096. KIF5A (Kinesin Family Member 5A), RTN2 (Reticulon 2), Heat Shock Protein Family D (Hsp60). SPG37 (Spastic Paraplegia 37). SPG41 (Spastic Paraplegia 41). SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1). ALDH18A1 (Aldehyde Dehydrogenase 18 Family Member A1), SPG11 (SPG11 Vesicle Trafficking Associated. Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26). SPG20 (Spastic paraplegia 20, autosomal recessive). SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1). KIFIA (Kinesin Family Member 1A), AP5Z1 (Adaptor Related Protein Complex 5 Subunit Zeta 1). FARS2 (Phenylalanyl-TRNA Synthetase 2. Mitochondrial), L1CAM (L1 Cell Adhesion Molecule). PLP1 (Proteolipid Protein 1). ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein). VapBC (virulence associated proteins B and C), ANG (Angiogenin). TARDBP (TAR DNA Binding Protein), FIG4 (FIG4 Phosphoinositide 5-Phosphatase), OPTN (Optineurin). ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEK1 (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), SOD1 (superoxide dismutase 1), C9orf72 (C9orf72-SMCR8 Complex Subunit), or a combination thereof.

In some embodiments, the target gene is SOD1. In some embodiments, the SOD1 gene comprises the sequence set forth SEQ ID NO: 33.

In some embodiments, the target gene is C9orf72. In some embodiments, the C9orf72 gene comprises the sequence set forth SEQ ID NO: 35, 36, or 37.

In some embodiments, the target gene is silenced. In some embodiments, the motor neuron disease or disorder is Spinal-bulbar muscular atrophy (SBMA), Spinal muscular atrophy (SMA) (e.g., non-5q SMA. Spinal muscular atrophy with congenital bone fractures, Autosomal dominant spinal muscular atrophy, and lower extremity-predominant 2 (SMALED2)), Distal hereditary motor neuropathy (dHMN) (e.g., autosomal dominant, autosomal recessive, type 1. 2, 4, 5, 6, 7, 7b), Triosephosphate isomerase deficiency (TIP), Hereditary spastic paraplegia (HSP) (also known as familial spastic paraparesis (FSP))(e.g., autosomal dominant or autosomal recessive or X-linked), amyotrophic lateral sclerosis (ALS), ALS with frontotemporal dementia (FTD), Adrenomyeloneuropathy (AMN), Allgrove syndrome (also known as triple A (3A) syndrome), Friedreich ataxia (FA), Spinocerebellar ataxia (SCA), Pontocerebellar hypoplasias (PCH), Neurodegeneration with brain iron accumulation (NBIA), mitochondrial complex I deficiency nuclear type 21 (MC1DN21), GM2 gangliosidosis (also known as Tay-Sachs disease or HexA deficiency), Charcot-Marie-Tooth disease (CMT), or a combination thereof.

In some embodiments, the one or more symptoms associated with the motor neuron disease or disorder are muscle weakness and decreased muscle tone, limited mobility, breathing problems, problems eating and swallowing, delayed gross motor skills, congenital hemolytic anemia, progressive neuromuscular dysfunction, spontaneous tongue movements, behavioral/cognitive symptoms, cerebellar degeneration, or scoliosis.

Accordingly, in another aspect, the present invention provides a method of silencing the expression of a target gene in a motor neuron, the method comprising contacting a recombinant adeno-associated virus (rAAV) comprising a regulatory element sequence that has at least 85% identity to SEQ ID NOs: 1-14 or 60-71 or a fragment thereof to the motor neuron.

In some embodiments, the regulatory element sequence has at least 90%, 95%, 98%, 99%, or 100% identity to SEQ ID NOs: 1-14 or 60-71. In some embodiments, the sequence comprises at least one modification relative to SEQ ID NOs: 1-14 or 60-71, optionally a substitute modification.

In some embodiments, the nucleic acid comprises at least one additional regulatory element sequence. In some embodiments, the nucleic acid comprises at least two, three, four, five or six additional regulatory element sequences.

In some embodiments, the at least one additional regulatory element sequence has at least 85% identity to SEQ ID NO: 1-14 and 60-71. In some embodiments, the at least one additional regulatory element sequence has at least 90%, 95%, 98%, 99%, or 100% identity to SEQ ID NOs: 1-14 or 60-71. In some embodiments, the at least one additional regulatory sequence comprises at least one modification relative to SEQ ID NO: 1-14 or 60-71, optionally a substitute modification.

In some embodiments, the nucleic acid comprises two or more identical copies of a regulatory element sequence selected from the group consisting of SEQ ID NO: 1-14 or 60-71. In some embodiments, the nucleic acid comprises two, three, four, five or six identical copies of the regulatory element sequence.

In some embodiments, the nucleic acid comprises at least two or more versions of a regulatory element sequence having at least 85%, 90%, 95%, 98%, 99%, or 100% identity to SEQ ID NO: 1-14 or 60-71, wherein the first version of the regulatory element sequence differs from the second version of the regulatory element sequence.

In some embodiments, the nucleic acid further comprising a heterologous gene.

In some embodiments, the regulatory element comprises SEQ ID NOs: 1-14 or 60-71.

In some embodiments, the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides.

In some embodiments, the regulatory element comprises one or more transcription factor binding sites. In some embodiments, the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA), or a combination thereof.

In some embodiments, the rAAV further comprises a promoter. In some embodiments, the promoter is a promoter from a gene expressed in motor neurons, optionally wherein the gene expressed in motor neurons is selected from the group consisting of a Dnajc22, Sycp1, Slit3, Hrasls5, Otop3, Ccnb3. Nlrp3, Hormad1, Chat, Anxa4, Tnfsf4, Myo3b, Cdh15, Nr2e1, I117f, Apela, Gnb3, Pappa. Tmprss15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, 1122ra1, Galp, Meil, Aox1, Prph, Slc25a54, Cdhr1. Tgm6, Ppm1j, Esrp1, Gem, Is11. Itpr3, Scc16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd. Terb2, Ins15, Card11, Platr7, Miat, Slc5a7, Iqcg, Topaz1, Tex 14, Slc5a10, Map4k1, Calcb, Got111, Slc46a2, Nanos2, Plekhg4, Themis3, Cpa3, Aknad1, Cdcp2. Uts2, Slc44a4, Popdc3, Tbata, Pkp1. Dysf, Pkp2, Sds, Nipsnap3a, Apo17e, Tex22, Mapk11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1. Usp50, Echdc2, Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNC1H1, PLEKHG5, SIGMAR1, DNAJB2, SMAX3, TPII, ATL1, SPAST, NIPA1, KIAA1096, KIFSA, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP, CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, and C9orf72. In some embodiments, the gene expressed in motor neurons is ChAT, Slc5a7, Is11, Mnx1, Lhx3, or Lhx4.

In some embodiments, the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx1 promoter, Lhx3 promoter, Lhx4 promoter, and variants thereof.

In some embodiments, the heterologous gene is an inhibitory nucleic acid. In some embodiments, the inhibitory nucleic acid is a microRNA (miRNA), artificial microRNA (amiRNA), or short hairpin RNA (shRNA).

In some embodiments, the inhibitory nucleic acid is a microRNA, wherein the microRNA binds to mRNA of a target gene. In some embodiments, the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1). BSCL2 (BSCL2 Lipid Droplet Biogenesis Associated. Seipin), GARS1 (Glycyl-TRNA Synthetase 1). SLC5A7 (Solute Carrier Family 5 Member 7). TRPV4 (Transient Receptor Potential Cation Channel Subfamily V Member 4), ATP7A (ATPase Copper Transporting Alpha), IGHMBP2 (Immunoglobulin Mu DNA Binding Protein 2. SETX (Senataxin), DCTN1 (Dynactin Subunit 1). DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2). SMAX3 (spinal muscular atrophy-3), TPI1 (Triosephosphate Isomerase 1). ATL1 (Atlastin GTPase 1), SPAST (Spastin), NIPA1 (Non-imprinted in Prader-Willi/Angelman syndrome region protein 1). KIAA1096. KIF5A (Kinesin Family Member 5A). RTN2 (Reticulon 2). Heat Shock Protein Family D (Hsp60). SPG37 (Spastic Paraplegia 37). SPG41 (Spastic Paraplegia 41), SLC33A1 (Solute Carrier Family 33 Member 1). REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated. Spatacsin), CYP7B1 (Cytochrome P450 Family 7 Subfamily B Member 1). SPG7 (SPG7 Matrix AAA Peptidase Subunit. Paraplegin). ZFYVE26 (Zinc Finger FYVE-Type Containing 26). SPG20 (Spastic paraplegia 20, autosomal recessive). SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A). AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7). TBK1 (TANK-binding kinase 1), ABCD1 (ATP Binding Cassette Subfamily D Member 1). ALADIN (ALacrima Achalasia aDrenal Insufficiency Neurologic disorder), FXN (Frataxin), NOP56 (NOP56 Ribonucleoprotein), ANO10 (Anoctamin 10). EXOSC3 (Exosome Component 3), C19orf12 (Chromosome 19 Open Reading Frame 12). NUBPL (Nucleotide Binding Protein Like), FUS (FUS RNA Binding Protein). VapBC (virulence associated proteins B and C). ANG (Angiogenin). TARDBP (TAR DNA Binding Protein), FIG4 (FIG4 Phosphoinositide 5-Phosphatase). OPTN (Optineurin), ATXN2 (Ataxin 2). VCP (Valosin Containing Protein), CHMP2B (Charged Multivesicular Body Protein 2B), PFN1 (Profilin 1). ERBB4 (Erb-B2 Receptor Tyrosine Kinase 4). HNRNPA2B1 (Heterogeneous Nuclear Ribonucleoprotein A2/B1), MATR3 (Matrin 3). TUBA4A (Tubulin Alpha 4a). ANXA11 (Annexin A11), NEK1 (NIMA Related Kinase 1), DAO (D-Amino Acid Oxidase), NEFH (Neurofilament Heavy Chain). SQSTM1 (Sequestosome 1), CYLD (CYLD Lysine 63 Deubiquitinase), CHCHD10 (Coiled-Coil-Helix-Coiled-Coil-Helix Domain Containing 10). UBQLN2 (Ubiquilin 2), HEXA (Hexosaminidase Subunit Alpha). MFN2 (Mitofusin 2), RAB7A (RAB7A. Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2). SOD1 (superoxide dismutase 1), C9orf72 (C9orf72-SMCR8 Complex Subunit), or a combination thereof.

In some embodiments, the target gene is SOD1. In some embodiments, the SOD1 gene comprises the sequence set forth SEQ ID NO: 33.

In some embodiments, the target gene is C9orf72. In some embodiments, the C9orf72 gene comprises the sequence set forth SEQ ID NO: 35, 36, or 37.

In some embodiments, the neuron is from a subject. In some embodiments, the subject is mammalian. In some embodiments, the subject is human.

In some embodiments, the subject has been diagnosed or is suspected of having a motor neuron disease or disorder. In some embodiments, the motor neuron disease or disorder is Spinal-bulbar muscular atrophy (SBMA), Spinal muscular atrophy (SMA) (e.g., non-5q SMA, Spinal muscular atrophy with congenital bone fractures, Autosomal dominant spinal muscular atrophy, and lower extremity-predominant 2 (SMALED2)), Distal hereditary motor neuropathy (dHMN) (e.g., autosomal dominant, autosomal recessive, type 1, 2, 4, 5, 6, 7, 7b), Triosephosphate isomerase deficiency (TIP), Hereditary spastic paraplegia (HSP) (also known as familial spastic paraparesis (FSP))(e.g., autosomal dominant or autosomal recessive or X-linked), amyotrophic lateral sclerosis (ALS), ALS with frontotemporal dementia (FTD), Adrenomyeloneuropathy (AMN), Allgrove syndrome (also known as triple A (3A) syndrome), Friedreich ataxia (FA), Spinocerebellar ataxia (SCA), Pontocerebellar hypoplasias (PCH), Neurodegeneration with brain iron accumulation (NBIA), mitochondrial complex I deficiency nuclear type 21 (MC1DN21), GM2 gangliosidosis (also known as Tay-Sachs disease or HexA deficiency), Charcot-Marie-Tooth disease (CMT), or a combination thereof.

In some embodiments, the promoter is pBG (optionally comprising SEQ ID NO: 55). In some embodiments, the nucleic acid further comprises a pBG intron (optionally comprising SEQ ID NO: 56), optionally wherein there is a linker between the pBG and pBG intron of zero to 500 nucleotides. In some embodiments, the promoter is pBG (optionally comprising SEQ ID NO: 55). In some embodiments, the rAAV vector, further comprises a pBG intron (optionally comprising SEQ ID NO: 56), optionally wherein there is a linker between the pBG and pBG intron of zero to 500 nucleotides.

In some embodiments, the promoter is pBG (optionally comprising SEQ ID NO: 55). In some embodiments, the rAAV further comprises a pBG intron (optionally comprising SEQ ID NO: 56), optionally wherein there is a linker between the pBG and pBG intron of zero to 500 nucleotides.

In some embodiments, the promoter is pBG (optionally comprising SEQ ID NO: 55). In some embodiments, the rAAV further comprises a pBG intron (optionally comprising SEQ ID NO: 56), optionally wherein there is a linker between the pBG and pBG intron of zero to 500 nucleotides.

### BRIEF DESCRIPTION OF THE DRAWINGS

FIG. 1A depicts expression of GFP in the spinal cord under the control of the Enh98 enhancer and beta globin promoter (pBG).

FIG. 1B depicts expression of GFP in the spinal cord under the control of only the beta globin promoter (pBG).

FIG. 2 depicts a graph quantifying the expression of GFP in the spinal cord under the control of the Enh57 and Enh98 enhancer compared to no enhancer and a saline control. Expression was compared across dorsal cells, the ventral horn, and dorsal root ganglion (DRG).

FIG. 3 depicts expression of GFP in the spinal cord under the control of CAG promoter. 3E+13 gc/ml, n=2 animals.

FIG. 4 depicts expression of GFP in dorsal root ganglion cells under the control of CAG promoter. 3E+13 gc/ml, n=2 animals.

FIG. 5 depicts expression of GFP in the spinal cord under the control of CAG promoter. 3E+13 gc/ml, n=2 animals.

FIG. 6 depicts expression of GFP in dorsal root ganglion cells under the control of pBG and Enh98 (mouse), n=2 animals.

FIG. 7 depicts expression of GFP in spinal cord under the control of pChAT and Enh98 (mouse), n=2 animals.

FIG. 8 depicts expression of GFP in dorsal root ganglion cells under the control of pChAT and Enh98 (mouse), n=2 animals.

FIGS. 9A-9G are related to motor neuron cis-regulatory element identification. FIG. 9A depicts the experimental design. FIG. 9B depicts an immunohistochemistry example of Chat-Sun1 cross labeling motor neuron nuclear envelope. FIG. 9C depicts an example of IP-specific and nonspecific cis-regulatory element ATAC-seq data. FIG. 9D depicts a genome-wide fixed-line-plot of ATAC-seq signal for all spinal cord peaks. FIG. 9E depicts summary plots showing average ATAC-seq signal intensity (left) and conservation (right) across spinal cord peaks. FIG. 9F depicts an MA plot of Enh MN-enrichment as a function of mean ATAC signal for each peak. FIG. 9G depicts a subselection of putative MN-selective Enhs by conservation.

FIGS. 10A-10E are related to preliminary Enhancer screening by confocal microscopy. FIG. 10A depicts a volcano plot (top) and plot of conservation (bottom) demonstrating candidate element selection thresholds. FIG. 10B depicts a table of selected elements. FIG. 10C depicts vector maps of screen AAV genomes. FIG. 10C depicts representative images from screen for all constructs evaluated by confocal microscopy. FIG. 10D depicts quantification of native GFP signal intensity in ventral and dorsal horns for all constructs evaluated.

FIGS. 11A-11G are related to immunohistochemistry quantification of hit specificity. FIG. 11A depicts representative images for all conditions assayed by IHC. FIG. 11B depicts percentage of GFP positivity quantification for NeuN+Chat+ and NeuN+Chat-neurons of spinal cord. FIG. 11C depicts mean GFP signal intensity quantification for NeuN+Chat+ and NeuN+Chat-neurons of spinal cord. FIG. 11D depicts relative GFP signal intensity of Enh98 compared to CAG in NeuN+Chat+ and NeuN+Chat-neurons of spinal cord. FIG. 11E depicts representative images for off-target GFP expression in DRG. FIG. 11F depicts percentage of GFP positivity quantification for neurons of the DRG. FIG. 11G depicts mean GFP signal intensity quantification for neurons of the DRG.

FIGS. 12A-12F are related to the identification of core functional components of Enh98. FIG. 12A depicts a scatter plot of TF motif significance as a function of enrichment for expression of that TF in motor neurons (left) and associated position-weight matrix (PWM) representation for significantly enriched motifs (denoted in green, right). FIG. 12B depicts a genomic map of TFBS position and truncated Enh98 construct design. FIG. 12C depicts a percentage of GFP positivity quantification for NeuN+Chat+ and Neun+Chat-neurons of spinal cord. FIG. 12D depicts a mean GFP signal intensity quantification for NeuN+Chat+ and Neun+Chat-neurons of spinal cord. FIG. 12E depicts distributions of GFP intensity of Enh98-pBG and Enh98-pCHAT promoter in the ventral horn of spinal cord and DRG. FIG. 12F depicts distributions of GFP intensity for all truncated constructs compared to CAG in the DRG.

FIG. 13A depicts heat map showing gene expression of specific markers in various cell types. FIG. 13B depicts a volcano plot of the fold change of gene expression of the markers shown in FIG. 13A. FIG. 13C depicts IP-specific and nonspecific Enh Fragment distribution. FIG. 13D depicts ATAC-seq principal component analysis (PCA), FIG. 13E depicts ATAC-seq correlation.

FIG. 14A depicts percent positive GFP cells comparing NeuN+/Chat-, NeuN+/Chat+interneurons, NeuN+/Chat+visceral motor neurons, and NeuN+/Chat+skeletal motor neurons when different Enhancers were used. Enhancers: Enh57, Enh98, and Enh119. Controls: Saline, ΔEnh, and CAG promoter. FIG. 14B depicts mean GFP intensity in cells from FIG. 14A.

### DETAILED DESCRIPTION

The present disclosure provides compositions and methods for cell-type specific expression of a heterologous gene. Also described herein are compositions and methods for expression of a heterologous gene comprising one or more regulatory elements which, when operably linked to a heterologous gene, can facilitate the expression of the heterologous in one or more target cell types or tissues. In some embodiments, the one or more regulatory elements disclosed herein drive expression of a heterologous gene in a cell or in vivo, in vitro, and/or ex vivo.

The present disclosure also provides a viral vector comprising a heterologous gene operably linked to a regulatory element, which induces expression of the heterologous gene in a cell-type specific manner. In some embodiments, the regulatory element is SEQ ID NOs: 1-14. In some embodiments, the heterologous gene is survival of motor neuron 1 (SMN1). The viral vector is a recombinant adeno-associated vector (rAAV). In some embodiments, a recombinant AAV viral particle comprises the rAAV comprising the heterologous gene operably linked to the regulatory element.

In some embodiments, the heterologous gene is expressed in a neuron. In some embodiments, the heterologous gene is expressed preferentially in a motor neuron and little to no expression in a non-motor neuron cell, for example, a dorsal root ganglia cell.

In another aspect, the present disclosure provides for a method of treating a subject having a motor neuron disease or disorder, comprising administering a recombinant adeno-associated virus (rAAV) which comprises a heterologous gene operably linked to a regulatory element, wherein one or more symptoms associated with the motor neuron disease or disorder are inhibited or prevented. In some embodiments, the heterologous gene is preferentially expressed in motor neuron and little to no expression in a non-motor neuron cell, for example, a dorsal root ganglia cell. In some embodiments, the regulatory element is SEQ ID NOs: 1-14 or 60-71, or a variant or fragment thereof. In some embodiments, the heterologous gene is survival of motor neuron 1 (SMN1).

### Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “encode” or “encoding” refers to a property of sequences of nucleic acids, such as a vector, a plasmid, a gene, cDNA, mRNA, to serve as templates for synthesis of other molecules such as proteins.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

It should be noted that whenever a value or range of values of a parameter are recited, it is intended that values and ranges intermediate to the recited values are also intended to be part of this invention.

### Regulatory Elements

As used herein, the term “regulatory elements” refers to elements that can function to modulate gene expression selectivity in a cell type of interest at a DNA and/or RNA level. Regulatory elements can function to modulate gene expression at the transcriptional phase, post-transcriptional phase, or at the translational phase of gene expression. Regulatory elements include, but are not limited to, promoter, enhancer, intronic, or other non-coding sequences. At the RNA level, regulation can occur at the level of translation (e.g., stability elements that stabilize mRNA for translation), RNA cleavage, RNA splicing, and/or transcriptional termination. In some cases, regulatory elements can recruit transcriptional factors to a coding region that increase gene expression selectivity in a cell type of interest. In some cases, regulatory elements can increase the rate at which RNA transcripts are produced, increase the stability of RNA produced, and/or increase the rate of protein synthesis from RNA transcripts.

Regulatory elements are nucleic acid sequences or genetic elements which are capable of influencing (e.g., increasing) expression of a gene (e.g., a reporter gene such as EGFP or luciferase; a transgene; or a therapeutic gene) in one or more cell types or tissues. In some cases, a regulatory element can be a transgene, an intron, a promoter, an enhancer, UTR, an inverted terminal repeat (ITR) sequence, a long terminal repeat sequence (LTR), stability element, posttranslational response element, or a polyA sequence, or a combination thereof. In some cases, the regulatory element is derived from a human sequence (e.g., SEQ ID NOs: 1-14 or 60-71). In some embodiments, the regulatory element is a variant of SEQ ID NO: 1-14 or 60-71, for example, containing a substitute mutation. In some embodiments, the regulatory element includes a fragment or fragments of SEQ ID NO: 1-14 or 60-71, which serves to modulate gene expression. In some embodiments, the regulatory element sequences used to induce cell-type specific expression accordingly to methods and compositions disclosed herein include SEQ ID NOs: 1-14 or 60-71.

As provided herein, the nucleic acid can comprise one or more regulatory element sequences. For example, in one embodiment, the nucleic acid comprises one regulatory element sequence. In some embodiments, the nucleic acid comprises at least one additional regulatory element sequence, for example, two, three, four, five, six, or more regulatory element sequences. In one embodiment, the at least one additional regulatory element sequence has at least 85% identity to SEQ ID NO: 1-14 and 60-71. In one embodiment, the at least one additional regulatory element sequence has at least 90%, 95%, 98%, 99%, or 100% identity to SEQ ID NOs: 1-14 or 60-71. In one embodiment, the at least one additional regulatory sequence comprises at least one modification relative to SEQ ID NO: 1-14 or 60-71, optionally a substitute modification.

In one embodiment, the nucleic acid sequence comprises two or more identical copies, for example, three, four, five or six copies, of a regulatory element selected from the group consisting of SEQ ID NO: 1-14 or 60-71.

In another embodiment, the nucleic acid comprises at least two or more versions of a regulatory element sequence having at least 85%, 90%, 95%, 98%, 99%, or 100% identity to SEQ ID NO: 1-14 or 60-71, wherein the first version of the regulatory element sequence differs from the second version of the regulatory element sequence. For example, the nucleic acid may include a first version of SEQ ID NO: 1 having 95% identity to SEQ ID NO: 1, and a second version of SEQ ID NO: 1 having 100% identity to SEQ ID NO: 1. Further by way of example, the nucleic acid may have a third and fourth versions of SEQ ID NO: 1, having 90% and 98% identity to SEQ ID NO: 1.

As provided herein, “enhancers” or “enhancer elements” induce expression of a gene, e.g., heterologous gene. In some embodiments, enhancers can induce expression of a heterologous gene in a cell-type specific manner. As used herein, “cell-type specific” or “cell-type specific induced expression” refer to expression being induced in certain cell types and not all cell types. In some embodiments, cell-type specific expression is induced in a specific cell type, e.g., neuron cell, but not other cell types, e.g., a non-neural cell. In some embodiments, the cell-type specific expression is induced in a specific cell type, e.g., motor neuron, and little to no expression in other cell types, e.g. . , dorsal cells. Cell-type specific induced expression does not eliminate the possibility that expression can occur in other cell-types at a low level. In some embodiments, cell-type specific induced expression results in expression of a heterologous gene in a specific cell-type at a higher level when compared to a control cell-type.

The specific enhancers described herein sometimes are referred to with the prefix “Enh”, or alternatively may be referred to as cis-regulatory elements (“CREs”) or gene regulatory elements (“GREs”). These terms and prefixes as used herein are interchangeable.

In some embodiments, the present disclosure provides for cell-type specific regulatory elements that induce expression of a heterologous gene in a specific cell-type. In some embodiments, the regulatory element is SEQ ID NOs: 1-14 or 60-71, a variant thereof or a fragment thereof. Accordingly, in one embodiment, the regulatory element has at least about 80% identity with the entire sequence of SEQ ID NOs: 1-14 or 60-71. Accordingly, in one embodiment, the regulatory element has at least about 90% identity with the entire sequence of SEQ ID NOs: 1-14 or 60-71. Accordingly, in one embodiment, the regulatory element has at least about 95% identity with the entire sequence of SEQ ID NOs: 1-14 or 60-71. Accordingly, in one embodiment, the regulatory element has at least about 85%, 86%, 87%, 88%, 89%, 90%, 91%, 92%, 93%, 94%, 95%, 96%, 97%, 98%, 99%, or 100% identity with the entire sequence of SEQ ID NOs: 1-14 or 60-71. In another embodiment, the regulatory element comprises the sequence of SEQ ID NOs: 1-14 or 60-71. In yet another embodiment the regulatory element consists of the sequence of SEQ ID NOs: 1-14 or 60-71.

In one embodiment, the regulatory element comprises at least about 500 nucleotides, at least about 450 nucleotides, at least about 400 nucleotides, at least about 350 nucleotides, at least about 300 nucleotides, at least about 250 nucleotides, at least about 200 nucleotides, at least about 150 nucleotides, at least about 100 nucleotides, at least about 50 nucleotides, or at least 25 nucleotides of SEQ ID NOs: 1-14 or 60-71. In one embodiment, the regulatory element comprises about 25 nucleotides to about 500 nucleotides, about 25 nucleotides to about 400 nucleotides, about 25 nucleotides to about 300 nucleotides, about 25 nucleotides to about 200 nucleotides, about 25 nucleotides to about 100 nucleotides, about 25 nucleotides to about 50 nucleotides, about 50 nucleotides to about 500 nucleotides, about 50 nucleotides to about 400 nucleotides, about 50 nucleotides to about 300 nucleotides, about 50 nucleotides to about 200 nucleotides, about 50 nucleotides to about 100 nucleotides, about 100 nucleotides to about 500 nucleotides, about 100 nucleotides to about 400 nucleotides, about 100 nucleotides to about 300 nucleotides, about 100 nucleotides to about 200 nucleotides, about 200 nucleotides to about 500 nucleotides, about 200 nucleotides to about 400 nucleotides, about 200 nucleotides to about 300 nucleotides, about 300 nucleotides to about 500 nucleotides, about 300 nucleotides to about 400 nucleotides, or about 400 to about 500 nucleotides of SEQ ID NOs: 1-14 or 60-71. In one embodiment, the regulatory element comprises no more than 500 nucleotides, no more than 400 nucleotides, no more than 300 nucleotides, no more than 200 nucleotides, no more than 100 nucleotides, no more than 50 nucleotides, or no more than 25 nucleotides of SEQ ID NOs: 1-14 or 60-71.

In some embodiments, the regulatory element comprises at least about 500 nucleotides and has at least about 85%, 86%, 87%, 88%, 89%, 90%, 91%, 92%, 93%, 94%, 95%, 96%, 97%, 98%, 99%, or 100% identity with the equivalent 500 nucleotide sequence of SEQ ID NOs: 1-14 or 60-71. In some embodiments, the regulatory element comprises at least about 400 nucleotides and has at least about 85%, 86%, 87%, 88%, 89%, 90%, 91%, 92%, 93%, 94%, 95%, 96%, 97%, 98%, 99%, or 100% identity with the equivalent 400 nucleotide sequence of SEQ ID NOs: 1-14 or 60-71. In some embodiments, the regulatory element comprises at least about 300 nucleotides and has at least about 85%, 86%, 87%, 88%, 89%, 90%, 91%, 92%, 93%, 94%, 95%, 96%, 97%, 98%, 99%, or 100% identity with the equivalent 300 nucleotide sequence of SEQ ID NOs: 1-14 or 60-71. In some embodiments, the regulatory element comprises at least about 200 nucleotides and has at least about 85%, 86%, 87%, 88%, 89%, 90%, 91%, 92%, 93%, 94%, 95%, 96%, 97%, 98%, 99%, or 100% identity with the equivalent 200 nucleotide sequence of SEQ ID NOs: 1-14 or 60-71. In some embodiments, the regulatory element comprises at least about 100 nucleotides and has at least about 85%, 86%, 87%, 88%, 89%, 90%, 91%, 92%, 93%, 94%, 95%, 96%, 97%, 98%, 99%, or 100% identity with the equivalent 100 nucleotide sequence of SEQ ID NOs: 1-14 or 60-71. In some embodiments, the regulatory element comprises at least about 50 nucleotides and has at least about 85%, 86%, 87%, 88%, 89%, 90%, 91%, 92%, 93%, 94%, 95%, 96%, 97%, 98%, 99%, or 100% identity with the equivalent 50 nucleotide sequence of SEQ ID NOs: 1-14 or 60-71.

In some embodiments, the present disclosure provides for cell-type specific regulatory elements that induce expression of a heterologous gene in a specific cell-type. In some embodiments, the regulatory element is SEQ ID NOs: 7-14 or 60-65. Accordingly, in one embodiment, the regulatory element has at least about 80% identity with the entire sequence of SEQ ID NOs: 7-14 or 60-65. Accordingly, in one embodiment, the regulatory element has at least about 90% identity with the entire sequence of SEQ ID NOs: 7-14 or 60-65. Accordingly, in one embodiment, the regulatory element has at least about 95% identity with the entire sequence of SEQ ID NOs: 7-14 or 60-65. Accordingly, in one embodiment, the regulatory element has at least about 85%, 86%, 87%, 88%, 89%, 90%, 91%, 92%, 93%, 94%, 95%, 96%, 97%, 98%, 99%, or 100% identity with the entire sequence of SEQ ID NOs: 7-14 or 60-65. In another embodiment, the regulatory element comprises the sequence of SEQ ID NOs: 7-14 or 60-65. In yet another embodiment the regulatory element consists of the sequence of SEQ ID NOs: 7-14 or 60-65.

In one embodiment, the regulatory element comprises at least about 500 nucleotides, at least about 450 nucleotides, at least about 400 nucleotides, at least about 350 nucleotides, at least about 300 nucleotides, at least about 250 nucleotides, at least about 200 nucleotides, at least about 150 nucleotides, at least about 100 nucleotides, at least about 50 nucleotides, or at least 25 nucleotides of SEQ ID NOs: 7-14 or 60-65. In one embodiment, the regulatory element comprises about 25 nucleotides to about 500 nucleotides, about 25 nucleotides to about 400 nucleotides, about 25 nucleotides to about 300 nucleotides, about 25 nucleotides to about 200 nucleotides, about 25 nucleotides to about 100 nucleotides, about 25 nucleotides to about 50 nucleotides, about 50 nucleotides to about 500 nucleotides, about 50 nucleotides to about 400 nucleotides, about 50 nucleotides to about 300 nucleotides, about 50 nucleotides to about 200 nucleotides, about 50 nucleotides to about 100 nucleotides, about 100 nucleotides to about 500 nucleotides, about 100 nucleotides to about 400 nucleotides, about 100 nucleotides to about 300 nucleotides, about 100 nucleotides to about 200 nucleotides, about 200 nucleotides to about 500 nucleotides, about 200 nucleotides to about 400 nucleotides, about 200 nucleotides to about 300 nucleotides, about 300 nucleotides to about 500 nucleotides, about 300 nucleotides to about 400 nucleotides, or about 400 to about 500 nucleotides of SEQ ID NOs: 7-14 or 60-65. In one embodiment, the regulatory element comprises no more than 500 nucleotides, no more than 400 nucleotides, no more than 300 nucleotides, no more than 200 nucleotides, no more than 100 nucleotides, no more than 50 nucleotides, or no more than 25 nucleotides of SEQ ID NOs: 7-14 or 60-65.

In some embodiments, the regulatory element comprises at least about 500 nucleotides and has at least about 85%, 86%, 87%, 88%, 89%, 90%, 91%, 92%, 93%, 94%, 95%, 96%, 97%, 98%. 99%, or 100% identity with the equivalent 500 nucleotide sequence of SEQ ID NOs: 7-14 or 60-65. In some embodiments, the regulatory element comprises at least about 400 nucleotides and has at least about 85%, 86%, 87%, 88%, 89%, 90%, 91%, 92%, 93%, 94%, 95%, 96%, 97%, 98%, 99%, or 100% identity with the equivalent 400 nucleotide sequence of SEQ ID NOs: 7-14 or 60-65. In some embodiments, the regulatory element comprises at least about 300 nucleotides and has at least about 85%, 86%, 87%, 88%, 89%, 90%, 91%, 92%, 93%, 94%, 95%, 96%, 97%, 98%, 99%, or 100% identity with the equivalent 300 nucleotide sequence of SEQ ID NOs: 7-14 or 60-65. In some embodiments, the regulatory element comprises at least about 200 nucleotides and has at least about 85%, 86%, 87%, 88%, 89%, 90%, 91%, 92%, 93%, 94%, 95%, 96%, 97%, 98%, 99%, or 100% identity with the equivalent 200 nucleotide sequence of SEQ ID NOs: 7-14 or 60-65. In some embodiments, the regulatory element comprises at least about 100 nucleotides and has at least about 85%, 86%, 87%, 88%, 89%, 90%, 91%, 92%, 93%, 94%, 95%, 96%, 97%, 98%, 99%, or 100% identity with the equivalent 100 nucleotide sequence of SEQ ID NOs: 7-14 or 60-65. In some embodiments, the regulatory element comprises at least about 50 nucleotides and has at least about 85%, 86%, 87%, 88%, 89%, 90%, 91%, 92%, 93%, 94%, 95%, 96%, 97%, 98%, 99%, or 100% identity with the equivalent 50 nucleotide sequence of SEQ ID NOs: 7-14 or 60-65.

In one embodiment, the regulatory element of SEQ ID NOs: 1-14 or 60-71 comprise sequences that are transcription factor binding sites. In some embodiments, the transcription factor binding sites are, but not limited to, LIM Homeobox 3 (Lhx3) (TTAATTAG), LIM Homeobox 4 (Lhx4) (TAATTAATTAAGT (SEQ ID NO: 16)), Motor Neuron and Pancreas Homeobox 1 (Mnx1) (TTAATTAA), Insulin gene enhancer protein ISL-2 (Is12) (GCACTTAA), Ras Responsive Element Binding Protein 1 (RREB1) (GCACTGGGGATGGGGGTGGG (SEQ ID NO: 19)), Signal Transducer And Activator Of Transcription 4 (STAT4) (TTTCCGGGAATGGC (SEQ ID NO: 20), Estrogen Related Receptor Beta (Esrrb) (TGGCCAAGGGCA (SEQ ID NO: 21)), and Myb (AACTGCCA). In some embodiments, the enhancer contains transcription factor binding sites LIM Homeobox 3 (Lhx3), LIM Homeobox 4 (Lhx4), Motor Neuron and Pancreas Homeobox 1 (Mnx1), Insulin gene enhancer protein ISL-2 (Is12), Ras Responsive Element Binding Protein 1 (RREB1), Signal Transducer And Activator Of Transcription 4 (STAT4), and Estrogen Related Receptor Beta (Esrrb), or a combination thereof.

In some embodiments, the transcription factor binding site for Lhx3 has 90% identity with the entire sequence of TTAATTAG. In one embodiment, the transcription factor binding site for Lhx3 has at least about 95% identity with the entire sequence of TTAATTAG. In a further embodiment, the transcription factor binding site for Lhx3 has at least about 85%, 86%, 87%, 88%, 89%, 90%, 91%, 92%, 93%, 94%, 95%, 96%, 97%, 98%, or 99% identity with the entire sequence of TTAATTAG. In another embodiment, the transcription factor binding site for Lhx3 comprises the sequence of TTAATTAG. In yet another embodiment, the transcription factor binding site for Lhx3 consists of the sequence of TTAATTAG.

In some embodiments, the transcription factor binding site for Lhx4 has 90% identity with the entire sequence of SEQ ID NO: 16. In one embodiment, the transcription factor binding site for Lhx4 has at least about 95% identity with the entire sequence of SEQ ID NO: 16. In a further embodiment, the transcription factor binding site for Lhx4 has at least about 85%, 86%, 87%, 88%, 89%, 90%, 91%, 92%, 93%, 94%, 95%, 96%, 97%, 98%, or 99% identity with the entire sequence of SEQ ID NO: 16. In another embodiment, the transcription factor binding site for Lhx4 comprises the sequence of SEQ ID NO: 16. In yet another embodiment the transcription factor binding site for Lhx4 consists of the sequence of SEQ ID NO: 16.

In some embodiments, the transcription factor binding site for Mnx1 has 90% identity with the entire sequence of TTAATTAA. In one embodiment, the transcription factor binding site for Mnx1 has at least about 95% identity with the entire sequence of TTAATTAA. In a further embodiment, the transcription factor binding site for Mnx 1 has at least about 85%, 86%, 87%, 88%, 89%, 90%, 91%, 92%, 93%, 94%, 95%, 96%, 97%, 98%, or 99% identity with the entire sequence of TTAATTAA. In another embodiment, the transcription factor binding site for Mnx1 comprises the sequence of TTAATTAA. In yet another embodiment, the transcription factor binding site for Mnx1 consists of the sequence of TTAATTAA.

In some embodiments, the transcription factor binding site for Is12 has 90% identity with the entire sequence of GCACTTAA. In one embodiment, the transcription factor binding site for Is12 has at least about 95% identity with the entire sequence of GCACTTAA. In a further embodiment, the transcription factor binding site for Is12 has at least about 85%, 86%, 87%, 88%, 89%, 90%, 91%, 92%, 93%, 94%, 95%, 96%, 97%, 98%, or 99% identity with the entire sequence of GCACTTAA. In another embodiment, the transcription factor binding site for Is12 comprises the sequence of GCACTTAA. In yet another embodiment, the transcription factor binding site for Is12 consists of the sequence of GCACTTAA.

In some embodiments, the transcription factor binding site for RREB1 has 90% identity with the entire sequence of SEQ ID NO: 19. In one embodiment, the transcription factor binding site for RREB1 has at least about 95% identity with the entire sequence of SEQ ID NO: 19. In a further embodiment, the transcription factor binding site for RREB1 has at least about 85%, 86%, 87%, 88%, 89%, 90%, 91%, 92%, 93%, 94%, 95%, 96%, 97%, 98%, or 99% identity with the entire sequence of SEQ ID NO: 19. In another embodiment, the transcription factor binding site for RREB1 comprises the sequence of SEQ ID NO: 19. In yet another embodiment, the transcription factor binding site for RREB1 consists of the sequence of SEQ ID NO: 19.

In some embodiments, the transcription factor binding site for STAT4 has 90% identity with the entire sequence of SEQ ID NO: 20. In one embodiment, the transcription factor binding site for STAT4 has at least about 95% identity with the entire sequence of SEQ ID NO: 20. In a further embodiment, the transcription factor binding site for STAT4 has at least about 85%, 86%, 87%, 88%, 89%, 90%, 91%, 92%, 93%, 94%, 95%, 96%, 97%, 98%, or 99% identity with the entire sequence of SEQ ID NO: 20. In another embodiment, the transcription factor binding site for STAT4 comprises the sequence of SEQ ID NO: 20. In yet another embodiment, the transcription factor binding site for STAT4 consists of the sequence of SEQ ID NO: 20.

In some embodiments, the transcription factor binding site for Esrrb has 90% identity with the entire sequence of SEQ ID NO: 21. In one embodiment, the transcription factor binding site for Esrrb has at least about 95% identity with the entire sequence of SEQ ID NO: 21. In a further embodiment, the transcription factor binding site for Esrrb has at least about 85%, 86%, 87%, 88%, 89%, 90%, 91%, 92%, 93%, 94%, 95%, 96%, 97%, 98%, or 99% identity with the entire sequence of SEQ ID NO: 21. In another embodiment, the transcription factor binding site for Esrrb comprises the sequence of SEQ ID NO: 21. In yet another embodiment, the transcription factor binding site for Esrrb consists of the sequence of SEQ ID NO: 21.

In some embodiments, the transcription factor binding site for Myb has 90% identity with the entire sequence of AACTGCCA. In one embodiment, the transcription factor binding site for Myb has at least about 95% identity with the entire sequence of AACTGCCA. In a further embodiment, the transcription factor binding site for Myb has at least about 85%, 86%, 87%, 88%, 89%, 90%, 91%, 92%, 93%, 94%, 95%, 96%, 97%, 98%, or 99% identity with the entire sequence of AACTGCCA. In another embodiment, the transcription factor binding site for Myb comprises the sequence of AACTGCCA. In yet another embodiment, the transcription factor binding site for Myb consists of the sequence of AACTGCCA.

### Promoters

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell-or tissue-specific manner, in response to different environmental or physiological conditions, or in response to specific compounds.

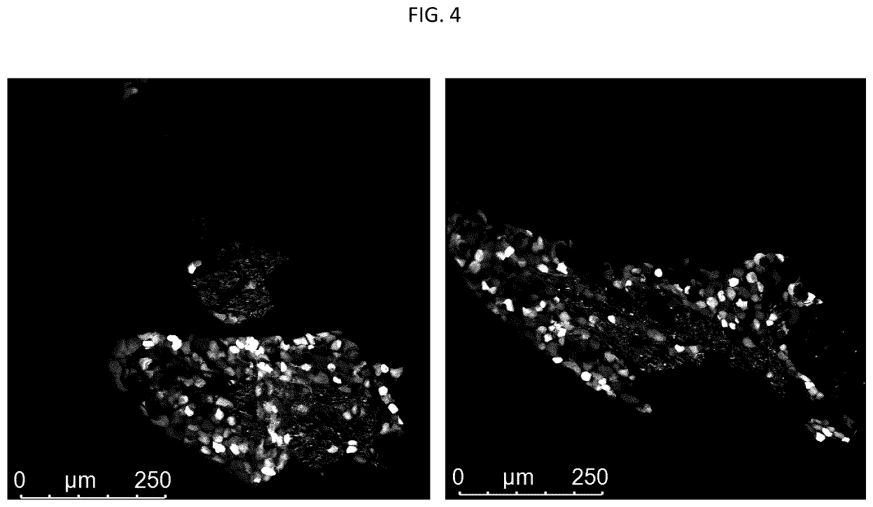

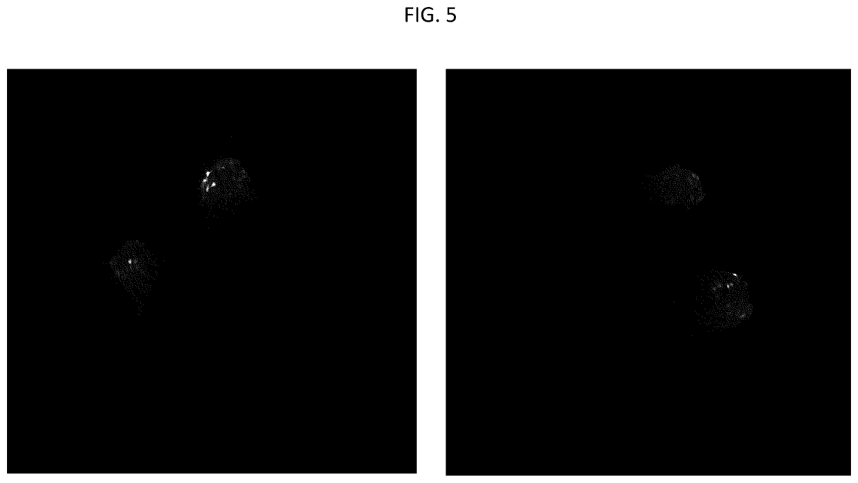

Promoters, as described herein, are promoters of genes expressed in motor neurons. Motor neuron enriched genes include, but are not limited to, Dnajc22, Sycp1, Slit3, Hrasls5, Otop3, Conb3, Nlrp3, Hormad1, Chat, Anxa4, Tnfsf4, Myo3b, Cdh15, Nr2e1, I117f, Apela, Gnb3, Pappa, Tmprss15, Crp. Nxpe5, Tex21, Ttc24, Ttc231, Ahnak2, Vipr2, Gstt4, Aox3, Plac811, Grin3b, Adam2, Co15a1, Clca3a1. Serpinb7, Edn2, Mgarp, Atp12a, Lhx4, Pip5k11, Slc25a48, Tfcp211, Clec18a, Spint2, I122ra1, Galp, Meil, Aox1, Prph, Slc25a54, Cdhr1, Tgm6, Ppm1j. Esrp1, Gem, Is11, Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd. Terb2, Ins15, Card11, Platr7, Miat, Slc5a7, Iqcg, Topaz1. Tex14, Slc5a10, Map4k1, Calcb, Got111, Slc46a2, Nanos2, Plekhg4, Themis3, Cpa3, Aknad1, Cdcp2, Uts2, Slc44a4, Popdc3, Tbata, Pkp1. Dysf, Pkp2, Sds, Nipsnap3a, Apo17e, Tex22, Mapk11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, TRPV4, ATP7A, IGHMBP2, DCTN1, DYNC1H1, PLEKHG5, SIGMAR1, DNAJB2, SMAX3, TPI1, ATLI, SPAST, NIPA1, KIAA1096, KIFSA, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1. ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP. CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, and C9orf72.

Promoters include, but not limited to, beta globin promoter (pBG) (for example, comprising SEQ ID NO: 55) and choline acetyltransferase promoter (pChAT) (for example, comprising SEQ ID NO: 23), CAG promoter (pCAG) (for example, comprising SEQ ID NO: 24 or 57), minimal CMV promoter, human synapsin promoter, chicken beta actin, PGK promoter, Efla promoter, ubiquitin promoter, TATA-box containing promoters, or fragments thereof. In some embodiments, the promoter is of genes expressed selectively in motor neurons (e.g., Chat, Slc5a7, Is11, Mnx1, Lhx3, Lhx4, and other genes listed above).

In some embodiments, the promoter is a beta globin promoter (pBG). In some embodiments, the pBG promoter comprises the pBG promoter alone (for example, comprising SEQ ID NO: 55). In some embodiments, the pBG promoter is attached to a pBG intron (for example, SEQ ID NO: 56). In some embodiments, the pBG promoter and the pBG intron are connected by Xₙ, where “X” can be nucleotides C. G. T, or A, and “n” can be zero nucleotides up to and including 500 nucleotides. In some embodiments, the nucleic acid sequence, vector or virus comprises pBG-X₍₀₋₅₀₀₎-pBG intron (SEQ ID NO: 22).

### Exemplary Promoters and Introns

| Description/ | |
|---|---|
| SEQ ID NO | Sequence |
| pBG promoter | CTGGGCATAAAAGTCAGGGCAGAGCCATCT |
| (pBG) | ATTGCTTACATTTGCTTCT |
| SEQ ID NO: 55 | |
| pBG intron | GTAAGTATCAAGGTTACAAGACAGGTTTAA |
| SEQ ID NO: 56 | GGAGACCAATAGAAACTGGGCTTGTCGAGA |
| | CAGAGAAGACTCTTGCGTTTCTGATAGGCA |
| | CCTATTGGTCTTACTGACATCCACTTTGCC |
| | TTTCTCTCCACAG |
| pBG-X₍₀₋₅₀₀₎- | CTGGGCATAAAAGTCAGGGCAGAGCCATCT |
| pBG intron | ATTGCTTACATTTGCTTCT X₍₀₋₅₀₀₎GT |
| promoter* | AAGTATCAAGGTTACAAGACAGGTTTAAGG |
| SEQ ID NO: 22 | AGACCAATAGAAACTGGGCTTGTCGAGACA |
| | GAGAAGACTCTTGCGTTTCTGATAGGCACC |
| | TATTGGTCTTACTGACATCCACTTTGCCTT |
| | TCTCTCCACAG |
| pChAT promoter | TCTCTTGTCCAATGGGGCTTGGAGCACCGA |
| SEQ ID NO: 23 | GGCCAGCGAAGCCATCGCGCTCCTTGCGGA |
| | GGTGAAGAGGACCCTGAGTCCCCACCTGCG |
| | GCTCCCCTGTGTAGAGCCTGCATCTGTCTG |
| | TCCTTCCTTCCATTGCTCCCAGTGCCAAAC |
| | TTGGGCCGCTGCACCGCGGCGCCTCCGCCC |
| | AAATCAATAAACTGTGTCTGTCCCAGGAGG |
| | CCGAGTCTCTTTACTGGTGGGGGGTGCGTG |
| | GAGGCGCGCAGGGCCAGAGCAGAGGGGAGG |
| | GTGAACTGGGTCTCCAAGTCCCAATCCAGA |
| | CCTAAGCCAAACTAACACGTAGGCACCTGT |
| | AGCTGTTTTTCTACCTGGAAAAGGGGATAG |
| | GAAGGAAGCAAACCCAACAAAGGCTGTCAC |
| | CCACGGTCACCAAGGAGCACCATGCTCCCC |
| | TCAGCCCAGGATAGACCCTCTTTTCCAGGC |
| | CTAGCGCAGAGCCCGGGGATGCCGCCCGGG |
| | GGAGCCTGAGGACCCGCTCCAGCTAGGCAC |
| | GCCAGGCCCCGCCCTTTGAGGACACGCCCC |
| | ACACCAGCCTCAGAGCTCTGAGGTGCCTGG |
| | GCTGAGCTTCCCTTCAGACCAGAATCCCGC |
| | CCCGTTGAGGCTTTGAGAAAGGAGTAGGAG |
| | CCGAGCATTCCGGCAGAGGAAGAAAAACGG |
| | CCC |
| pCAG promoter | GCGTTACATAACTTACGGTAAATGGCCCGC |
| SEQ ID NO: 24 | CTGGCTGACCGCCCAACGACCCCCGCCCAT |
| | TGACGTCAATAATGACGTATGTTCCCATAG |
| | TAACGCCAATAGGGACTTTCCATTGACGTC |
| | AATGGGTGGAGTATTTACGGTAAACTGCCC |
| | ACTTGGCAGTACATCAAGTGTATCATATGC |
| | CAAGTACGCCCCCTATTGACGTCAATGACG |
| | GTAAATGGCCCGCCTGGCATTATGCCCAGT |
| | ACATGACCTTATGGGACTTTCCTACTTGGC |
| | AGTACATCTACGTATTAGTCATCGCTATTA |
| | CCATGGTCGAGGTGAGCCCCACGTTCTGCT |
| | TCACTCTCCCCATCTCCCCCCCCTCCCCAC |
| | CCCCAATTTTGTATTTATTTATTTTTTAAT |
| | TATTTTGTGCAGCGATGGGGGCGGGGGGGG |
| | GGGGGGGGCGCGCGCCAGGCGGGGCGGGGC |
| | GGGGCGAGGGGCGGGGCGGGGCGAGGCGGA |
| | GAGGTGCGGCGGCAGCCAATCAGAGCGGCG |
| | CGCTCCGAAAGTTTCCTTTTATGGCGAGGC |
| | GGCGGCGGCGGCGGCCCTATAAAAAGCGAA |
| | GCGCGCGGCGGGCG |
| pCAG promoter | CGTTACATAACTTACGGTAAATGGCCCGCC |
| (long) | TGGCTGACCGCCCAACGACCCCCGCCCATT |
| SEQ ID NO: 57 | GACGTCAATAATGACGTATGTTCCCATAGT |
| | AACGCCAATAGGGACTTTCCATTGACGTCA |
| | ATGGGTGGAGTATTTACGGTAAACTGCCCA |
| | CTTGGCAGTACATCAAGTGTATCATATGCC |
| | AAGTACGCCCCCTATTGACGTCAATGACGG |
| | TAAATGGCCCGCCTGGCATTATGCCCAGTA |
| | CATGACCTTATGGGACTTTCCTACTTGGCA |
| | GTACATCTACGTATTAGTCATCGCTATTAC |
| | CATGGTCGAGGTGAGCCCCACGTTCTGCTT |
| | CACTCTCCCCATCTCCCCCCCCTCCCCACC |
| | CCCAATTTTGTATTTATTTATTTTTTAATT |
| | ATTTTGTGCAGCGATGGGGGCGGGGGGGGG |
| | GGGGGGGCGCGCGCCAGGCGGGGCGGGGCG |
| | GGGCGAGGGGCGGGGCGGGGCGAGGCGGAG |
| | AGGTGCGGCGGCAGCCAATCAGAGCGGCGC |
| | GCTCCGAAAGTTTCCTTTTATGGCGAGGCG |
| | GCGGCGGCGGCGGCCCTATAAAAAGCGAAG |
| | CGCGCGGCGGGCGGGAGTCGCTGCGCGCTG |
| | CCTTCGCCCCGTGCCCCGCTCCGCCGCCGC |
| | CTCGCGCCGCCCGCCCCGGCTCTGACTGAC |
| | CGCGTTACTCCCACAGGTGAGCGGGCGGGA |
| | CGGCCCTTCTCCTCCGGGCTGTAATTAGCG |
| | CTTGGTTTAATGACGGCTTGTTTCTTTTCT |
| | GTGGCTGCGTGAAAGCCTTGAGGGGCTCCG |
| | GGAGGGCCCTTTGTGCGGGGGGAGCGGCTC |
| | GGGGGGTGCGTGCGTGTGTGTGTGCGTGGG |
| | GAGCGCCGCGTGCGGCTCCGCGCTGCCCGG |
| | CGGCTGTGAGCGCTGCGGGCGCGGCGCGGG |
| | GCTTTGTGCGCTCCGCAGTGTGCGCGAGGG |
| | GAGCGCGGCCGGGGGCGGTGCCCCGCGGTG |
| | CGGGGGGGGCTGCGAGGGGAACAAAGGCTG |
| | CGTGCGGGGTGTGTGCGTGGGGGGGTGAGC |
| | AGGGGGTGTGGGCGCGTCGGTCGGGCTGCA |
| | ACCCCCCCTGCACCCCCCTCCCCGAGTTGC |
| | TGAGCACGGCCCGGCTTCGGGTGCGGGGCT |
| | CCGTACGGGGCGTGGCGCGGGGCTCGCCGT |
| | GCCGGGCGGGGGGTGGCGGCAGGTGGGGGT |
| | GCCGGGCGGGGCGGGGCCGCCTCGGGCCGG |
| | GGAGGGCTCGGGGGAGGGGCGCGGCGGCCC |
| | CCGGAGCGCCGGCGGCTGTCGAGGCGCGGC |
| | GAGCCGCAGCCATTGCCTTTTATGGTAATC |
| | GTGCGAGAGGGCGCAGGGACTTCCTTTGTC |
| | CCAAATCTGTGCGGAGCCGAAATCTGGGAG |
| | GCGCCGCCGCACCCCCTCTAGCGGGCGCGG |
| | GGCGAAGCGGTGCGGCGCCGGCAGGAAGGA |
| | AATGGGCGGGGAGGGCCTTCGTGCGTCGCC |
| | GCGCCGCCGTCCCCTTCTCCCTCTCCAGCC |
| | TCGGGGCTGTCCGCGGGGGGACGGCTGCCT |
| | TCGGGGGGGACGGGGCAGGGCGGGGTTCGG |
| | CTTCTGGCGTGTGACCGGCGGCTCTAGAGC |
| | CTCTGCTAACCATGTTCATGCCTTCTTCTT |
| | TTTCCTACAG |

*“X” refers to nucleotides C, G, T, or A.

### Heterologous Gene

As used herein, the term “gene” may include not only coding sequences but also regulatory regions such as promoters, enhancers, and termination regions. The term further can include all introns and other DNA sequences spliced from the mRNA transcript, along with variants resulting from alternative splice sites. The term further refers to a coding sequence for a desired expression product of a polynucleotide sequence such as a polypeptide, peptide, protein or interfering RNA including short interfering RNA (siRNA), miRNA or small hairpin RNA (shRNA). The sequences can also include degenerate codons of a reference sequence or sequences that may be introduced to provide codon preference in a specific organism or cell type. As used herein, the term “heterologous gene” refers gene provided to the target cell by an exogenous source, such as a viral vector, e.g., rAAV. In some embodiments, the gene encodes a polypeptide or a nucleic acid molecule, such as microRNA (miRNA), artificial microRNA (amiRNA), and short hairpin RNA (shRNA).

In some embodiments, the heterologous gene is selected from the group consisting of survival of motor neuron 1 (SMN1). AR (androgen receptor). BICD2 (BICD Cargo Adaptor 2). TRIP4 (Thyroid Hormone Receptor Interactor 4), HSPB1 (Heat Shock Protein Family B (Small) Member 1). HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated. Scipin), GARS1 (Glycyl-TRNA Synthetase 1), SLC5A7 (Solute Carrier Family 5 Member 7). TRPV4 (Transient Receptor Potential Cation Channel Subfamily V Member 4). ATP7A (ATPase Copper Transporting Alpha), IGHMBP2 (Immunoglobulin Mu DNA Binding Protein 2. SETX (Senataxin), DCTN1 (Dynactin Subunit 1). DYNC1H1 (Dynein Cytoplasmic 1 Heavy Chain 1), PLEKHG5 (Pleckstrin Homology And RhoGEF Domain Containing G5). SIGMAR1 (Sigma Non-Opioid Intracellular Receptor 1), DNAJB2 (DnaJ Heat Shock Protein Family (Hsp40) Member B2). SMAX3 (spinal muscular atrophy-3), TPI1 (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2). Heat Shock Protein Family D (Hsp60). SPG37 (Spastic Paraplegia 37). SPG41 (Spastic Paraplegia 41), SLC33A1 (Solute Carrier Family 33 Member 1). REEP2 (Receptor Accessory Protein 2), CPTIC (Carnitine Palmitoyltransferase 1C), UBAP1 (Ubiquitin Associated Protein 1). ALDH18A1 (Aldehyde Dehydrogenase 18 Family Member A1), SPG11 (SPG11 Vesicle Trafficking Associated. Spatacsin), CYP7B1 (Cytochrome P450 Family 7 Subfamily B Member 1). SPG7 (SPG7 Matrix AAA Peptidase Subunit, Paraplegin). ZFYVE26 (Zinc Finger FYVE-Type Containing 26). SPG20 (Spastic paraplegia 20, autosomal recessive). SPG21(ACP33) (Spastic paraplegia 21, autosomal recessive), GJC2 (Gap Junction Protein Gamma 2). SPG24 (Spastic Paraplegia 24 (Autosomal Recessive)), DDHD1 (DDHD Domain Containing 1), KIFIA (Kinesin Family Member 1A). AP5Z1 (Adaptor Related Protein Complex 5 Subunit Zeta 1), FARS2 (Phenylalanyl-TRNA Synthetase 2. Mitochondrial), L1CAM (L1 Cell Adhesion Molecule), PLP1 (Proteolipid Protein 1). ALS2 (Alsin Rho Guanine Nucleotide Exchange Factor ALS2). WDR7 (WD Repeat Domain 7). TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein). ANO10 (Anoctamin 10). EXOSC3 (Exosome Component 3). C19orf12 (Chromosome 19 Open Reading Frame 12). NUBPL (Nucleotide Binding Protein Like), FUS (FUS RNA Binding Protein), VapBC (virulence associated proteins B and C). ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1). ERBB4 (Erb-B2 Receptor Tyrosine Kinase 4), HNRNPA2B1 (Heterogeneous Nuclear Ribonucleoprotein A2/B1), MATR3 (Matrin 3). TUBA4A (Tubulin Alpha 4a), ANXA11 (Annexin A11), NEK1 (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), or a combination thereof.

In some embodiments, the heterologous gene is SMN1.

In some embodiments, the heterologous gene has 90% identity with the entire sequence of SEQ ID NO: 25. Accordingly, in one embodiment, the heterologous gene has at least about 95% identity with the entire sequence of SEQ ID NO: 25. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 25. In another embodiment, the heterologous gene comprises the sequence of SEQ ID NO: 25. In yet another embodiment the heterologous gene consists of the sequence of SEQ ID NO: 25.

In some embodiments, the heterologous gene has 90% identity with the entire sequence of SEQ ID NO: 26. Accordingly, in one embodiment, the heterologous gene has at least about 95% identity with the entire sequence of SEQ ID NO: 26. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 26. In another embodiment, the heterologous gene comprises the sequence of SEQ ID NO: 26. In yet another embodiment the heterologous gene consists of the sequence of SEQ ID NO: 26.

In some embodiments, the heterologous gene has 90% identity with the entire sequence of SEQ ID NO: 27. Accordingly, in one embodiment, the heterologous gene has at least about 95% identity with the entire sequence of SEQ ID NO: 27. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 27. In another embodiment, the heterologous gene comprises the sequence of SEQ ID NO: 27. In yet another embodiment the heterologous gene consists of the sequence of SEQ ID NO: 27.

In some embodiments, the heterologous gene has 90% identity with the entire sequence of SEQ ID NO: 28. Accordingly, in one embodiment, the heterologous gene has at least about 95% identity with the entire sequence of SEQ ID NO: 28. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 28. In another embodiment, the heterologous gene comprises the sequence of SEQ ID NO: 28. In yet another embodiment the heterologous gene consists of the sequence of SEQ ID NO: 28.

In some embodiments, the heterologous gene encodes a transcriptional regulator (e.g., represses expression of a gene or enhances expression of a target gene). In some embodiments, the transcription regulator is an engineered zinc finger polypeptide, Transcription activator-like effector nucleases (TALEN), or Cas9 (CRISPR associated protein 9, formerly called Cas5, Csn1, or Csx12) or dCas9 (nuclease deficient Cas9), rtTA (reverse tetracycline-controlled transactivator), tetracycline transactivator (tTA), ribozymes, RNA-editing proteins, other DNA editing enzymes (e.g., DNA base editing proteins, prime editing proteins, CRISPR family proteins, etc.).

In some embodiments, the transcriptional regulator regulates expression of one or more target genes. In some embodiments, the one or more target gene is SMN1, AR, BICD2, TRIP4, HSPB1. HSPB8, HSPB3, FBXO38, REEP1, BSCL2, GARS1, SLC5A7, TRPV4, ATP7A, IGHMBP2, DCTN1, DYNC1H1, PLEKHG5, SIGMAR1, DNAJB2, SMAX3, TPII, ATLI, SPAST, NIPA1, KIAA1096, KIFSA, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP, CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, C9orf72, or a combination thereof.

In some embodiments, the heterologous gene encodes a microRNA. In some embodiments, the microRNA inhibits expression of one or more target genes. In some embodiments, the target gene is SMN1, AR, BICD2, TRIP4, HSPB1, HSPB8, HSPB3, FBXO38, REEP1, BSCL2, GARS1, SLC5A7. TRPV4, ATP7A, IGHMBP2, DCTN1, DYNC1H1, PLEKHG5, SIGMAR1, DNAJB2, SMAX3, TPI1, ATLI, SPAST, NIPA1, KIAA1096, KIFSA, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS. VapBC, ANG, TARDBP, FIG. 4, OPTN, ATXN2, VCP, CHMP2B, PFN1, ERBB4, HNRNPA2B1, MATR3, TUBA4A, ANXA11, NEK1, DAO, NEFH, SQSTM1, CYLD, CHCHD10, UBQLN2, HEXA, MEN2, RAB7A, NEFL, GDAP1, LRSAM1, MORC2, SOD1, C9orf72, or a combination thereof.

In some embodiments, the target gene is SOD1.

In some embodiments, the heterologous gene has 90% identity with the entire sequence of SEQ ID NO: 33. Accordingly, in one embodiment, the heterologous gene has at least about 95% identity with the entire sequence of SEQ ID NO: 33. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 33. In another embodiment, the heterologous gene comprises the sequence of SEQ ID NO: 33. In yet another embodiment the heterologous gene consists of the sequence of SEQ ID NO: 33.

In some embodiments, the target gene is C9orf72.

In some embodiments, the heterologous gene has 90% identity with the entire sequence of SEQ ID NO: 35. Accordingly, in one embodiment, the heterologous gene has at least about 95% identity with the entire sequence of SEQ ID NO: 35. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 35. In another embodiment, the heterologous gene comprises the sequence of SEQ ID NO: 35. In yet another embodiment the heterologous gene consists of the sequence of SEQ ID NO: 35.

In some embodiments, the heterologous gene has 90% identity with the entire sequence of SEQ ID NO: 36. Accordingly, in one embodiment, the heterologous gene has at least about 95% identity with the entire sequence of SEQ ID NO: 36. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 36. In another embodiment, the heterologous gene comprises the sequence of SEQ ID NO: 36. In yet another embodiment the heterologous gene consists of the sequence of SEQ ID NO: 36.

In some embodiments, the heterologous gene has 90% identity with the entire sequence of SEQ ID NO: 37. Accordingly, in one embodiment, the heterologous gene has at least about 95% identity with the entire sequence of SEQ ID NO: 37. Accordingly, in one embodiment, the heterologous gene has at least about 85%, 86%, 87%, 88%, 89%, 90%, 91%, 92%, 93%, 94%, 95%, 96%, 97%, 98%, or 99% identity with the entire sequence of SEQ ID NO: 37. In another embodiment, the heterologous gene comprises the sequence of SEQ ID NO: 37. In yet another embodiment the heterologous gene consists of the sequence of SEQ ID NO: 37.

### Exemplary Survival of Motor Neuron 1 (SMN1) Nucleic Acid Sequence

| Accession No. | Sequences |
|---|---|
| NM_022875 | GCACCCGCGGGTTTGCTATGGCGAT |
| SMN1 Isoform a | GAGCAGCGGCGGCAGTGGTGGCGGC |
| (SEQ ID NO: 25) | GTCCCGGAGCAGGAGGATTCCGTGC |
| | TGTTCCGGCGCGGCACAGGCCAGAG |
| | CGATGATTCTGACATTTGGGATGAT |
| | ACAGCACTGATAAAAGCATATGATA |
| | AAGCTGTGGCTTCATTTAAGCATGC |
| | TCTAAAGAATGGTGACATTTGTGAA |
| | ACTTCGGGTAAACCAAAAACCACAC |
| | CTAAAAGAAAACCTGCTAAGAAGAA |
| | TAAAAGCCAAAAGAAGAATACTGCA |
| | GCTTCCTTACAACAGTGGAAAGTTG |
| | GGGACAAATGTTCTGCCATTTGGTC |
| | AGAAGACGGTTGCATTTACCCAGCT |
| | ACCATTGCTTCAATTGATTTTAAGA |
| | GAGAAACCTGTGTTGTGGTTTACAC |
| | TGGATATGGAAATAGAGAGGAGCAA |
| | AATCTGTCCGATCTACTTTCCCCAA |
| | TCTGTGAAGTAGCTAATAATATAGA |
| | ACAAAATGCTCAAGAGAATGAAAAT |
| | GAAAGCCAAGTTTCAACAGATGAAA |
| | GTGAGAACTCCAGGTCTCCTGGAAA |
| | TAAATCAGATAACATCAAGCCCAAA |
| | TCTGCTCCATGGAACTCTTTTCTCC |
| | CTCCACCACCCCCCATGCCAGGGCC |
| | AAGACTGGGACCAGGAAAGCCAGGT |
| | CTAAAATTCAATGGCCCACCACCGC |
| | CACCGCCACCACCACCACCCCACTT |
| | ACTATCATGCTGGCTGCCTCCATTT |
| | CCTTCTGGACCACCAATAATTCCCC |
| | CACCACCTCCCATATGTCCAGATTC |
| | TCTTGATGATGCTGATGCTTTGGGA |
| | AGTATGTTAATTTCATGGTACATGA |
| | GTGGCTATCATACTGGCTATTATAT |
| | GGAAATGCTGGCATAGAGCAGCACT |
| | AAATGACACCACTAAAGAAACGATC |
| | AGACAGATCTGGAATGTGAAGCGTT |
| | ATAGAAGATAACTGGCCTCATTTCT |
| | TCAAAATATCAAGTGTTGGGAAAGA |
| | AAAAAGGAAGTGGAATGGGTAACTC |
| | TTCTTGATTAAAAGTTATGTAATAA |
| | CCAAATGCAATGTGAAATATTTTAC |
| | TGGACTCTATTTTGAAAAACCATCT |
| | GTAAAAGACTGAGGTGGGGGTGGGA |
| | GGCCAGCACGGTGGTGAGGCAGTTG |
| | AGAAAATTTGAATGTGGATTAGATT |
| | TTGAATGATATTGGATAATTATTGG |
| | TAATTTTATGAGCTGTGAGAAGGGT |
| | GTTGTAGTTTATAAAAGACTGTCTT |
| | AATTTGCATACTTAAGCATTTAGGA |
| | ATGAAGTGTTAGAGTGTCTTAAAAT |
| | GTTTCAAATGGTTTAACAAAATGTA |
| | TGTGAGGCGTATGTGGCAAAATGTT |
| | ACAGAATCTAACTGGTGGACATGGC |
| | TGTTCATTGTACTGTTTTTTTCTAT |
| | CTTCTATATGTTTAAAAGTATATAA |
| | TAAAAATATTTAATTTTTTTTTAAA |
| | TTA |
| NM_022876 | CCACAAATGTGGGAGGGCGATAACC |
| SMN1 isoform b | ACTCGTAGAAAGCGTGAGAAGTTAC |
| (SEQ ID NO: 26) | TACAAGCGGTCCTCCCGGCCACCGT |
| | ACTGTTCCGCTCCCAGAAGCCCCGG |
| | GCGGCGGAAGTCGTCACTCTTAAGA |
| | AGGGACGGGGCCCCACGCTGCGCAC |
| | CCGCGGGTTTGCTATGGCGATGAGC |
| | AGCGGCGGCAGTGGTGGCGGCGTCC |
| | CGGAGCAGGAGGATTCCGTGCTGTT |
| | CCGGCGCGGCACAGGCCAGAGCGAT |
| | GATTCTGACATTTGGGATGATACAG |
| | CACTGATAAAAGCATATGATAAAGC |
| | TGTGGCTTCATTTAAGCATGCTCTA |
| | AAGAATGGTGACATTTGTGAAACTT |
| | CGGGTAAACCAAAAACCACACCTAA |
| | AAGAAAACCTGCTAAGAAGAATAAA |
| | AGCCAAAAGAAGAATACTGCAGCTT |
| | CCTTACAACAGTGGAAAGTTGGGGA |
| | CAAATGTTCTGCCATTTGGTCAGAA |
| | GACGGTTGCATTTACCCAGCTACCA |
| | TTGCTTCAATTGATTTTAAGAGAGA |
| | AACCTGTGTTGTGGTTTACACTGGA |
| | TATGGAAATAGAGAGGAGCAAAATC |
| | TGTCCGATCTACTTTCCCCAATCTG |
| | TGAAGTAGCTAATAATATAGAACAA |
| | AATGCTCAAGAGAATGAAAATGAAA |
| | GCCAAGTTTCAACAGATGAAAGTGA |
| | GAACTCCAGGTCTCCTGGAAATAAA |
| | TCAGATAACATCAAGCCCAAATCTG |
| | CTCCATGGAACTCTTTTCTCCCTCC |
| | ACCACCCCCCATGCCAGGGCCAAGA |
| | CTGGGACCAGGAAAGATAATTCCCC |
| | CACCACCTCCCATATGTCCAGATTC |
| | TCTTGATGATGCTGATGCTTTGGGA |
| | AGTATGTTAATTTCATGGTACATGA |
| | GTGGCTATCATACTGGCTATTATAT |
| | GGGTTTTAGACAAAATCAAAAAGAA |
| | GGAAGGTGCTCACATTCCTTAAATT |
| | AAGGAGAAATGCTGGCATAGAGCAG |
| | CACTAAATGACACCACTAAAGAAAC |
| | GATCAGACAGATCTGGAATGTGAAG |
| | CGTTATAGAAGATAACTGGCCTCAT |
| | TTCTTCAAAATATCAAGTGTTGGGA |
| | AAGAAAAAAGGAAGTGGAATGGGTA |
| | ACTCTTCTTGATTAAAAGTTATGTA |
| | ATAACCAAATGCAATGTGAAATATT |
| | TTACTGGACTCTATTTTGAAAAACC |
| | ATCTGTAAAAGACTGAGGTGGGGGT |
| | GGGAGGCCAGCACGGTGGTGAGGCA |
| | GTTGAGAAAATTTGAATGTGGATTA |
| | GATTTTGAATGATATTGGATAATTA |
| | TTGGTAATTTTATGAGCTGTGAGAA |
| | GGGTGTTGTAGTTTATAAAAGACTG |
| | TCTTAATTTGCATACTTAAGCATTT |
| | AGGAATGAAGTGTTAGAGTGTCTTA |
| | AAATGTTTCAAATGGTTTAACAAAA |
| | TGTATGTGAGGCGTATGTGGCAAAA |
| | TGTTACAGAATCTAACTGGTGGACA |
| | TGGCTGTTCATTGTACTGTTTTTTT |
| | CTATCTTCTATATGTTTAAAAGTAT |
| | ATAATAAAAATATTTAATTTTTTTT |
| | TAAATTAAAAAAA |
| NM_022877 | CCACAAATGTGGGAGGGCGATAACC |
| SMN1 isoform c | ACTCGTAGAAAGCGTGAGAAGTTAC |
| (SEQ ID NO: 27) | TACAAGCGGTCCTCCCGGCCACCGT |
| | ACTGTTCCGCTCCCAGAAGCCCCGG |
| | GCGGCGGAAGTCGTCACTCTTAAGA |
| | AGGGACGGGGCCCCACGCTGCGCAC |
| | CCGCGGGTTTGCTATGGCGATGAGC |
| | AGCGGCGGCAGTGGTGGCGGCGTCC |
| | CGGAGCAGGAGGATTCCGTGCTGTT |
| | CCGGCGCGGCACAGGCCAGAGCGAT |
| | GATTCTGACATTTGGGATGATACAG |
| | CACTGATAAAAGCATATGATAAAGC |
| | TGTGGCTTCATTTAAGCATGCTCTA |
| | AAGAATGGTGACATTTGTGAAACTT |
| | CGGGTAAACCAAAAACCACACCTAA |
| | AAGAAAACCTGCTAAGAAGAATAAA |
| | AGCCAAAAGAAGAATACTGCAGCTT |
| | CCTTACAACAGTGGAAAGTTGGGGA |
| | CAAATGTTCTGCCATTTGGTCAGAA |
| | GACGGTTGCATTTACCCAGCTACCA |
| | TTGCTTCAATTGATTTTAAGAGAGA |
| | AACCTGTGTTGTGGTTTACACTGGA |
| | TATGGAAATAGAGAGGAGCAAAATC |
| | TGTCCGATCTACTTTCCCCAATCTG |
| | TGAAGTAGCTAATAATATAGAACAA |
| | AATGCTCAAGAGAATGAAAATGAAA |
| | GCCAAGTTTCAACAGATGAAAGTGA |
| | GAACTCCAGGTCTCCTGGAAATAAA |
| | TCAGATAACATCAAGCCCAAATCTG |
| | CTCCATGGAACTCTTTTCTCCCTCC |
| | ACCACCCCCCATGCCAGGGCCAAGA |
| | CTGGGACCAGGAAAGATAATTCCCC |
| | CACCACCTCCCATATGTCCAGATTC |
| | TCTTGATGATGCTGATGCTTTGGGA |
| | AGTATGTTAATTTCATGGTACATGA |
| | GTGGCTATCATACTGGCTATTATAT |
| | GGAAATGCTGGCATAGAGCAGCACT |
| | AAATGACACCACTAAAGAAACGATC |
| | AGACAGATCTGGAATGTGAAGCGTT |
| | ATAGAAGATAACTGGCCTCATTTCT |
| | TCAAAATATCAAGTGTTGGGAAAGA |
| | AAAAAGGAAGTGGAATGGGTAACTC |
| | TTCTTGATTAAAAGTTATGTAATAA |
| | CCAAATGCAATGTGAAATATTTTAC |
| | TGGACTCTATTTTGAAAAACCATCT |
| | GTAAAAGACTGAGGTGGGGGTGGGA |
| | GGCCAGCACGGTGGTGAGGCAGTTG |
| | AGAAAATTTGAATGTGGATTAGATT |
| | TTGAATGATATTGGATAATTATTGG |
| | TAATTTTATGAGCTGTGAGAAGGGT |
| | GTTGTAGTTTATAAAAGACTGTCTT |
| | AATTTGCATACTTAAGCATTTAGGA |
| | ATGAAGTGTTAGAGTGTCTTAAAAT |
| | GTTTCAAATGGTTTAACAAAATGTA |
| | TGTGAGGCGTATGTGGCAAAATGTT |
| | ACAGAATCTAACTGGTGGACATGGC |
| | TGTTCATTGTACTGTTTTTTTCTAT |
| | CTTCTATATGTTTAAAAGTATATAA |
| | TAAAAATATTTAATTTTTTTTTAAA |
| | TTAAAAAAA |
| NM_000344.4 | GCACCCGCGGGTTTGCTATGGCGAT |
| SMN1 isoform d | GAGCAGCGGCGGCAGTGGTGGCGGC |
| (SEQ ID NO: 28) | GTCCCGGAGCAGGAGGATTCCGTGC |
| | TGTTCCGGCGCGGCACAGGCCAGAG |
| | CGATGATTCTGACATTTGGGATGAT |
| | ACAGCACTGATAAAAGCATATGATA |
| | AAGCTGTGGCTTCATTTAAGCATGC |
| | TCTAAAGAATGGTGACATTTGTGAA |
| | ACTTCGGGTAAACCAAAAACCACAC |
| | CTAAAAGAAAACCTGCTAAGAAGAA |
| | TAAAAGCCAAAAGAAGAATACTGCA |
| | GCTTCCTTACAACAGTGGAAAGTTG |
| | GGGACAAATGTTCTGCCATTTGGTC |
| | AGAAGACGGTTGCATTTACCCAGCT |
| | ACCATTGCTTCAATTGATTTTAAGA |
| | GAGAAACCTGTGTTGTGGTTTACAC |
| | TGGATATGGAAATAGAGAGGAGCAA |
| | AATCTGTCCGATCTACTTTCCCCAA |
| | TCTGTGAAGTAGCTAATAATATAGA |
| | ACAAAATGCTCAAGAGAATGAAAAT |
| | GAAAGCCAAGTTTCAACAGATGAAA |
| | GTGAGAACTCCAGGTCTCCTGGAAA |
| | TAAATCAGATAACATCAAGCCCAAA |
| | TCTGCTCCATGGAACTCTTTTCTCC |
| | CTCCACCACCCCCCATGCCAGGGCC |
| | AAGACTGGGACCAGGAAAGCCAGGT |
| | CTAAAATTCAATGGCCCACCACCGC |
| | CACCGCCACCACCACCACCCCACTT |
| | ACTATCATGCTGGCTGCCTCCATTT |
| | CCTTCTGGACCACCAATAATTCCCC |
| | CACCACCTCCCATATGTCCAGATTC |
| | TCTTGATGATGCTGATGCTTTGGGA |
| | AGTATGTTAATTTCATGGTACATGA |
| | GTGGCTATCATACTGGCTATTATAT |
| | GGGTTTCAGACAAAATCAAAAAGAA |
| | GGAAGGTGCTCACATTCCTTAAATT |
| | AAGGAGAAATGCTGGCATAGAGCAG |
| | CACTAAATGACACCACTAAAGAAAC |
| | GATCAGACAGATCTGGAATGTGAAG |
| | CGTTATAGAAGATAACTGGCCTCAT |
| | TTCTTCAAAATATCAAGTGTTGGGA |
| | AAGAAAAAAGGAAGTGGAATGGGTA |
| | ACTCTTCTTGATTAAAAGTTATGTA |
| | ATAACCAAATGCAATGTGAAATATT |
| | TTACTGGACTCTATTTTGAAAAACC |
| | ATCTGTAAAAGACTGGGGTGGGGGT |
| | GGGAGGCCAGCACGGTGGTGAGGCA |
| | GTTGAGAAAATTTGAATGTGGATTA |
| | GATTTTGAATGATATTGGATAATTA |
| | TTGGTAATTTTATGAGCTGTGAGAA |
| | GGGTGTTGTAGTTTATAAAAGACTG |
| | TCTTAATTTGCATACTTAAGCATTT |
| | AGGAATGAAGTGTTAGAGTGTCTTA |
| | AAATGTTTCAAATGGTTTAACAAAA |
| | TGTATGTGAGGCGTATGTGGCAAAA |
| | TGTTACAGAATCTAACTGGTGGACA |
| | TGGCTGTTCATTGTACTGTTTTTTT |
| | CTATCTTCTATATGTTTAAAAGTAT |
| | ATAATAAAAATATTTAATTTTTTTT |
| | TAAATTA |

### Exemplary Survival of Motor Neuron 1 (SMN 1) Amino Acid Sequences

| Accession No. | Sequences |
|---|---|
| NP_001284644 | MAMSSGGSGGGVPEQEDSVLFRRGT |
| SMN1 Isoform a | GQSDDSDIWDDTALIKAYDKAVASF |
| (SEQ ID NO: 29) | KHALKNGDICETSGKPKTTPKRKPA |
| | KKNKSQKKNTAASLQQWKVGDKCSA |
| | IWSEDGCIYPATIASIDFKRETCVV |
| | VYTGYGNREEQNLSDLLSPICEVAN |
| | NIEQNAQENENESQVSTDESENSRS |
| | PGNKSDNIKPKSAPWNSFLPPPPPM |
| | PGPRLGPGKPGLKFNGPPPPPPPPP |
| | PHLLSCWLPPFPSGPPIIPPPPPIC |
| | PDSLDDADALGSMLISWYMSGYHTG |
| | YYMEMLA |
| NP_075012.1 | MAMSSGGSGGGVPEQEDSVLFRRGT |
| SMN1 isoform b | GQSDDSDIWDDTALIKAYDKAVASF |
| (SEQ ID NO: 30) | KHALKNGDICETSGKPKTTPKRKPA |
| | KKNKSQKKNTAASLQQWKVGDKCSA |
| | IWSEDGCIYPATIASIDFKRETCVV |
| | VYTGYGNREEQNLSDLLSPICEVAN |
| | NIEQNAQENENESQVSTDESENSRS |
| | PGNKSDNIKPKSAPWNSFLPPPPPM |
| | PGPRLGPGKIIPPPPPICPDSLDDA |
| | DALGSMLISWYMSGYHTGYYMGFRQ |
| | NQKEGRCSHSLN |
| NP_075015 | MAMSSGGSGGGVPEQEDSVLFRRGT |
| SMN1 isoform c | GQSDDSDIWDDTALIKAYDKAVASF |
| (SEQ ID NO: 31) | KHALKNGDICETSGKPKTTPKRKPA |
| | KKNKSQKKNTAASLQQWKVGDKCSA |
| | IWSEDGCIYPATIASIDFKRETCVV |
| | VYTGYGNREEQNLSDLLSPICEVAN |
| | NIEQNAQENENESQVSTDESENSRS |
| | PGNKSDNIKPKSAPWNSFLPPPPPM |
| | PGPRLGPGKIIPPPPPICPDSLDDA |
| | DALGSMLISWYMSGYHTGYYMEMLA |
| NP_000335 | MAMSSGGSGGGVPEQEDSVLFRRGT |
| SMN1 isoform d | GQSDDSDIWDDTALIKAYDKAVASF |
| (SEQ ID NO: 32) | KHALKNGDICETSGKPKTTPKRKPA |
| | KKNKSQKKNTAASLQQWKVGDKCSA |
| | IWSEDGCIYPATIASIDFKRETCVV |
| | VYTGYGNREEQNLSDLLSPICEVAN |
| | NIEQNAQENENESQVSTDESENSRS |
| | PGNKSDNIKPKSAPWNSFLPPPPPM |
| | PGPRLGPGKPGLKFNGPPPPPPPPP |
| | PHLLSCWLPPFPSGPPIIPPPPPIC |
| | PDSLDDADALGSMLISWYMSGYHTG |
| | YYMGFRQNQKEGRCSHSLN |

### Exemplary Superoxide Dismutase 1 (SOD 1) Nucleotide Sequence

| Accession No. | Sequences |
|---|---|
| NM_000454.5 | GCGTCGTAGTCTCCTGCAGCGTCTG |
| (SEQ ID NO: 33) | GGGTTTCCGTTGCAGTCCTCGGAAC |
| | CAGGACCTCGGCGTGGCCTAGCGAG |
| | TTATGGCGACGAAGGCCGTGTGCGT |
| | GCTGAAGGGCGACGGCCCAGTGCAG |
| | GGCATCATCAATTTCGAGCAGAAGG |
| | AAAGTAATGGACCAGTGAAGGTGTG |
| | GGGAAGCATTAAAGGACTGACTGAA |
| | GGCCTGCATGGATTCCATGTTCATG |
| | AGTTTGGAGATAATACAGCAGGCTG |
| | TACCAGTGCAGGTCCTCACTTTAAT |
| | CCTCTATCCAGAAAACACGGTGGGC |
| | CAAAGGATGAAGAGAGGCATGTTGG |
| | AGACTTGGGCAATGTGACTGCTGAC |
| | AAAGATGGTGTGGCCGATGTGTCTA |
| | TTGAAGATTCTGTGATCTCACTCTC |
| | AGGAGACCATTGCATCATTGGCCGC |
| | ACACTGGTGGTCCATGAAAAAGCAG |
| | ATGACTTGGGCAAAGGTGGAAATGA |
| | AGAAAGTACAAAGACAGGAAACGCT |
| | GGAAGTCGTTTGGCTTGTGGTGTAA |
| | TTGGGATCGCCCAATAAACATTCCC |
| | TTGGATGTAGTCTGAGGCCCCTTAA |
| | CTCATCTGTTATCCTGCTAGCTGTA |
| | GAAATGTATCCTGATAAACATTAAA |
| | CACTGTAATCTTAAAAGTGTAATTG |
| | TGTGACTTTTTCAGAGTTGCTTTAA |
| | AGTACCTGTAGTGAGAAACTGATTT |
| | ATGATCACTTGGAAGATTTGTATAG |
| | TTTTATAAAACTCAGTTAAAATGTC |
| | TGTTTCAATGACCTGTATTTTGCCA |
| | GACTTAAATCACAGATGGGTATTAA |
| | ACTTGTCAGAATTTCTTTGTCATTC |
| | AAGCCTGTGAATAAAAACCCTGTAT |
| | GGCACTTATTATGAGGCTATTAAAA |
| | GAATCCAAATTCAAACTAAA |

### Exemplary Superoxide Dismutase 1 (SOD 1) Amino Acid Sequence

| Accession No. | Sequences |
|---|---|
| NP_000445.1 | MATKAVCVLKGDGPVQGIINFE |
| (SEQ ID NO: 34) | QKESNGPVKVWGSIKGLTEGLH |
| | GFHVHEFGDNTAGCTSAGPHFN |
| | PLSRKHGGPKDEERHVGDLGNV |
| | TADKDGVADVSIEDSVISLSGD |
| | HCIIGRTLVVHEKADDLGKGGN |
| | EESTKTGNAGSRLACGVIGIAQ |

| Accession No. | Sequences |
|---|---|
| NM_001256054.3 | ACGTAACCTACGGTGTCCCGCTAGG |
| C9orf72 | AAAGAGAGGTGCGTCAAACAGCGAC |
| transcript | AAGTTCCGCCCACGTAAAAGATGAC |
| variant 3 | GCTTGGTGTGTCAGCCGTCCCTGCT |
| (SEQ ID NO: 35) | GCCCGGTTGCTTCTCTTTTGGGGGC |
| | GGGGTCTAGCAAGAGCAGGTGTGGG |
| | TTTAGGAGATATCTCCGGAGCATTT |
| | GGATAATGTGACAGTTGGAATGCAG |
| | TGATGTCGACTCTTTGCCCACCGCC |
| | ATCTCCAGCTGTTGCCAAGACAGAG |
| | ATTGCTTTAAGTGGCAAATCACCTT |
| | TATTAGCAGCTACTTTTGCTTACTG |
| | GGACAATATTCTTGGTCCTAGAGTA |
| | AGGCACATTTGGGCTCCAAAGACAG |
| | AACAGGTACTTCTCAGTGATGGAGA |
| | AATAACTTTTCTTGCCAACCACACT |
| | CTAAATGGAGAAATCCTTCGAAATG |
| | CAGAGAGTGGTGCTATAGATGTAAA |
| | GTTTTTTGTCTTGTCTGAAAAGGGA |
| | GTGATTATTGTTTCATTAATCTTTG |
| | ATGGAAACTGGAATGGGGATCGCAG |
| | CACATATGGACTATCAATTATACTT |
| | CCACAGACAGAACTTAGTTTCTACC |
| | TCCCACTTCATAGAGTGTGTGTTGA |
| | TAGATTAACACATATAATCCGGAAA |
| | GGAAGAATATGGATGCATAAGGAAA |
| | GACAAGAAAATGTCCAGAAGATTAT |
| | CTTAGAAGGCACAGAGAGAATGGAA |
| | GATCAGGGTCAGAGTATTATTCCAA |
| | TGCTTACTGGAGAAGTGATTCCTGT |
| | AATGGAACTGCTTTCATCTATGAAA |
| | TCACACAGTGTTCCTGAAGAAATAG |
| | ATATAGCTGATACAGTACTCAATGA |
| | TGATGATATTGGTGACAGCTGTCAT |
| | GAAGGCTTTCTTCTCAATGCCATCA |
| | GCTCACACTTGCAAACCTGTGGCTG |
| | TTCCGTTGTAGTAGGTAGCAGTGCA |
| | GAGAAAGTAAATAAGATAGTCAGAA |
| | CATTATGCCTTTTTCTGACTCCAGC |
| | AGAGAGAAAATGCTCCAGGTTATGT |
| | GAAGCAGAATCATCATTTAAATATG |
| | AGTCAGGGCTCTTTGTACAAGGCCT |
| | GCTAAAGGATTCAACTGGAAGCTTT |
| | GTGCTGCCTTTCCGGCAAGTCATGT |
| | ATGCTCCATATCCCACCACACACAT |
| | AGATGTGGATGTCAATACTGTGAAG |
| | CAGATGCCACCCTGTCATGAACATA |
| | TTTATAATCAGCGTAGATACATGAG |
| | ATCCGAGCTGACAGCCTTCTGGAGA |
| | GCCACTTCAGAAGAAGACATGGCTC |
| | AGGATACGATCATCTACACTGACGA |
| | AAGCTTTACTCCTGATTTGAATATT |
| | TTTCAAGATGTCTTACACAGAGACA |
| | CTCTAGTGAAAGCCTTCCTGGATCA |
| | GGTCTTTCAGCTGAAACCTGGCTTA |
| | TCTCTCAGAAGTACTTTCCTTGCAC |
| | AGTTTCTACTTGTCCTTCACAGAAA |
| | AGCCTTGACACTAATAAAATATATA |
| | GAAGACGATACGCAGAAGGGAAAAA |
| | AGCCCTTTAAATCTCTTCGGAACCT |
| | GAAGATAGACCTTGATTTAACAGCA |
| | GAGGGCGATCTTAACATAATAATGG |
| | CTCTGGCTGAGAAAATTAAACCAGG |
| | CCTACACTCTTTTATCTTTGGAAGA |
| | CCTTTCTACACTAGTGTGCAAGAAC |
| | GAGATGTTCTAATGACTTTTTAAAT |
| | GTGTAACTTAATAAGCCTATTCCAT |
| | CACAATCATGATCGCTGGTAAAGTA |
| | GCTCAGTGGTGTGGGGAAACGTTCC |
| | CCTGGATCATACTCCAGAATTCTGC |
| | TCTCAGCAATTGCAGTTAAGTAAGT |
| | TACACTACAGTTCTCACAAGAGCCT |
| | GTGAGGGGATGTCAGGTGCATCATT |
| | ACATTGGGTGTCTCTTTTCCTAGAT |
| | TTATGCTTTTGGGATACAGACCTAT |
| | GTTTACAATATAATAAATATTATTG |
| | CTATCTTTTAAAGATATAATAATAG |
| | GATGTAAACTTGACCACAACTACTG |
| | TTTTTTTGAAATACATGATTCATGG |
| | TTTACATGTGTCAAGGTGAAATCTG |
| | AGTTGGCTTTTACAGATAGTTGACT |
| | TTCTATCTTTTGGCATTCTTTGGTG |
| | TGTAGAATTACTGTAATACTTCTGC |
| | AATCAACTGAAAACTAGAGCCTTTA |
| | AATGATTTCAATTCCACAGAAAGAA |
| | AGTGAGCTTGAACATAGGATGAGCT |
| | TTAGAAAGAAAATTGATCAAGCAGA |
| | TGTTTAATTGGAATTGATTATTAGA |
| | TCCTACTTTGTGGATTTAGTCCCTG |
| | GGATTCAGTCTGTAGAAATGTCTAA |
| | TAGTTCTCTATAGTCCTTGTTCCTG |
| | GTGAACCACAGTTAGGGTGTTTTGT |
| | TTATTTTATTGTTCTTGCTATTGTT |
| | GATATTCTATGTAGTTGAGCTCTGT |
| | AAAAGGAAATTGTATTTTATGTTTT |
| | AGTAATTGTTGCCAACTTTTTAAAT |
| | TAATTTTCATTATTTTTGAGCCAAA |
| | TTGAAATGTGCACCTCCTGTGCCTT |
| | TTTTCTCCTTAGAAAATCTAATTAC |
| | TTGGAACAAGTTCAGATTTCACTGG |
| | TCAGTCATTTTCATCTTGTTTTCTT |
| | CTTGCTAAGTCTTACCATGTACCTG |
| | CTTTGGCAATCATTGCAACTCTGAG |
| | ATTATAAAATGCCTTAGAGAATATA |
| | CTAACTAATAAGATCTTTTTTTCAG |
| | AAACAGAAAATAGTTCCTTGAGTAC |
| | TTCCTTCTTGCATTTCTGCCTATGT |
| | TTTTGAAGTTGTTGCTGTTTGCCTG |
| | CAATAGGCTATAAGGAATAGCAGGA |
| | GAAATTTTACTGAAGTGCTGTTTTC |
| | CTAGGTGCTACTTTGGCAGAGCTAA |
| | GTTATCTTTTGTTTTCTTAATGCGT |
| | TTGGACCATTTTGCTGGCTATAAAA |
| | TAACTGATTAATATAATTCTAACAC |
| | AATGTTGACATTGTAGTTACACAAA |
| | CACAAATAAATATTTTATTTAAAAT |
| | TCTGGAAGTAATATAAAAGGGAAAA |
| | TATATTTATAAGAAAGGGATAAAGG |
| | TAATAGAGCCCTTCTGCCCCCCACC |
| | CACCAAATTTACACAACAAAATGAC |
| | ATGTTCGAATGTGAAAGGTCATAAT |
| | AGCTTTCCCATCATGAATCAGAAAG |
| | ATGTGGACAGCTTGATGTTTTAGAC |
| | AACCACTGAACTAGATGACTGTTGT |
| | ACTGTAGCTCAGTCATTTAAAAAAT |
| | ATATAAATACTACCTTGTAGTGTCC |
| | CATACTGTGTTTTTTACATGGTAGA |
| | TTCTTATTTAAGTGCTAACTGGTTA |
| | TTTTCTTTGGCTGGTTTATTGTACT |
| | GTTATACAGAATGTAAGTTGTACAG |
| | TGAAATAAGTTATTAAAGCATGTGT |
| | AAACATTGTTATATATCTTTTCTCC |
| | TAAATGGAGAATTTTGAATAAAATA |
| | TATTTGAAATTTT |
| NM_018325.5 | GGTTGCGGTGCCTGCGCCCGCGGCG |
| C9orf72 | GCGGAGGCGCAGGCGGTGGCGAGTG |
| transcript | GATATCTCCGGAGCATTTGGATAAT |
| variant 2 | GTGACAGTTGGAATGCAGTGATGTC |
| (SEQ ID NO: 36) | GACTCTTTGCCCACCGCCATCTCCA |
| | GCTGTTGCCAAGACAGAGATTGCTT |
| | TAAGTGGCAAATCACCTTTATTAGC |
| | AGCTACTTTTGCTTACTGGGACAAT |
| | ATTCTTGGTCCTAGAGTAAGGCACA |
| | TTTGGGCTCCAAAGACAGAACAGGT |
| | ACTTCTCAGTGATGGAGAAATAACT |
| | TTTCTTGCCAACCACACTCTAAATG |
| | GAGAAATCCTTCGAAATGCAGAGAG |
| | TGGTGCTATAGATGTAAAGTTTTTT |
| | GTCTTGTCTGAAAAGGGAGTGATTA |
| | TTGTTTCATTAATCTTTGATGGAAA |
| | CTGGAATGGGGATCGCAGCACATAT |
| | GGACTATCAATTATACTTCCACAGA |
| | CAGAACTTAGTTTCTACCTCCCACT |
| | TCATAGAGTGTGTGTTGATAGATTA |
| | ACACATATAATCCGGAAAGGAAGAA |
| | TATGGATGCATAAGGAAAGACAAGA |
| | AAATGTCCAGAAGATTATCTTAGAA |
| | GGCACAGAGAGAATGGAAGATCAGG |
| | GTCAGAGTATTATTCCAATGCTTAC |
| | TGGAGAAGTGATTCCTGTAATGGAA |
| | CTGCTTTCATCTATGAAATCACACA |
| | GTGTTCCTGAAGAAATAGATATAGC |
| | TGATACAGTACTCAATGATGATGAT |
| | ATTGGTGACAGCTGTCATGAAGGCT |
| | TTCTTCTCAATGCCATCAGCTCACA |
| | CTTGCAAACCTGTGGCTGTTCCGTT |
| | GTAGTAGGTAGCAGTGCAGAGAAAG |
| | TAAATAAGATAGTCAGAACATTATG |
| | CCTTTTTCTGACTCCAGCAGAGAGA |
| | AAATGCTCCAGGTTATGTGAAGCAG |
| | AATCATCATTTAAATATGAGTCAGG |
| | GCTCTTTGTACAAGGCCTGCTAAAG |
| | GATTCAACTGGAAGCTTTGTGCTGC |
| | CTTTCCGGCAAGTCATGTATGCTCC |
| | ATATCCCACCACACACATAGATGTG |
| | GATGTCAATACTGTGAAGCAGATGC |
| | CACCCTGTCATGAACATATTTATAA |
| | TCAGCGTAGATACATGAGATCCGAG |
| | CTGACAGCCTTCTGGAGAGCCACTT |
| | CAGAAGAAGACATGGCTCAGGATAC |
| | GATCATCTACACTGACGAAAGCTTT |
| | ACTCCTGATTTGAATATTTTTCAAG |
| | ATGTCTTACACAGAGACACTCTAGT |
| | GAAAGCCTTCCTGGATCAGGTCTTT |
| | CAGCTGAAACCTGGCTTATCTCTCA |
| | GAAGTACTTTCCTTGCACAGTTTCT |
| | ACTTGTCCTTCACAGAAAAGCCTTG |
| | ACACTAATAAAATATATAGAAGACG |
| | ATACGCAGAAGGGAAAAAAGCCCTT |
| | TAAATCTCTTCGGAACCTGAAGATA |
| | GACCTTGATTTAACAGCAGAGGGCG |
| | ATCTTAACATAATAATGGCTCTGGC |
| | TGAGAAAATTAAACCAGGCCTACAC |
| | TCTTTTATCTTTGGAAGACCTTTCT |
| | ACACTAGTGTGCAAGAACGAGATGT |
| | TCTAATGACTTTTTAAATGTGTAAC |
| | TTAATAAGCCTATTCCATCACAATC |
| | ATGATCGCTGGTAAAGTAGCTCAGT |
| | GGTGTGGGGAAACGTTCCCCTGGAT |
| | CATACTCCAGAATTCTGCTCTCAGC |
| | AATTGCAGTTAAGTAAGTTACACTA |
| | CAGTTCTCACAAGAGCCTGTGAGGG |
| | GATGTCAGGTGCATCATTACATTGG |
| | GTGTCTCTTTTCCTAGATTTATGCT |
| | TTTGGGATACAGACCTATGTTTACA |
| | ATATAATAAATATTATTGCTATCTT |
| | TTAAAGATATAATAATAGGATGTAA |
| | ACTTGACCACAACTACTGTTTTTTT |
| | GAAATACATGATTCATGGTTTACAT |
| | GTGTCAAGGTGAAATCTGAGTTGGC |
| | TTTTACAGATAGTTGACTTTCTATC |
| | TTTTGGCATTCTTTGGTGTGTAGAA |
| | TTACTGTAATACTTCTGCAATCAAC |
| | TGAAAACTAGAGCCTTTAAATGATT |
| | TCAATTCCACAGAAAGAAAGTGAGC |
| | TTGAACATAGGATGAGCTTTAGAAA |
| | GAAAATTGATCAAGCAGATGTTTAA |
| | TTGGAATTGATTATTAGATCCTACT |
| | TTGTGGATTTAGTCCCTGGGATTCA |
| | GTCTGTAGAAATGTCTAATAGTTCT |
| | CTATAGTCCTTGTTCCTGGTGAACC |
| | ACAGTTAGGGTGTTTTGTTTATTTT |
| | ATTGTTCTTGCTATTGTTGATATTC |
| | TATGTAGTTGAGCTCTGTAAAAGGA |
| | AATTGTATTTTATGTTTTAGTAATT |
| | GTTGCCAACTTTTTAAATTAATTTT |
| | CATTATTTTTGAGCCAAATTGAAAT |
| | GTGCACCTCCTGTGCCTTTTTTCTC |
| | CTTAGAAAATCTAATTACTTGGAAC |
| | AAGTTCAGATTTCACTGGTCAGTCA |
| | TTTTCATCTTGTTTTCTTCTTGCTA |
| | AGTCTTACCATGTACCTGCTTTGGC |
| | AATCATTGCAACTCTGAGATTATAA |
| | AATGCCTTAGAGAATATACTAACTA |
| | ATAAGATCTTTTTTTCAGAAACAGA |
| | AAATAGTTCCTTGAGTACTTCCTTC |
| | TTGCATTTCTGCCTATGTTTTTGAA |
| | GTTGTTGCTGTTTGCCTGCAATAGG |
| | CTATAAGGAATAGCAGGAGAAATTT |
| | TACTGAAGTGCTGTTTTCCTAGGTG |
| | CTACTTTGGCAGAGCTAAGTTATCT |
| | TTTGTTTTCTTAATGCGTTTGGACC |
| | ATTTTGCTGGCTATAAAATAACTGA |
| | TTAATATAATTCTAACACAATGTTG |
| | ACATTGTAGTTACACAAACACAAAT |
| | AAATATTTTATTTAAAATTCTGGAA |
| | GTAATATAAAAGGGAAAATATATTT |
| | ATAAGAAAGGGATAAAGGTAATAGA |
| | GCCCTTCTGCCCCCCACCCACCAAA |
| | TTTACACAACAAAATGACATGTTCG |
| | AATGTGAAAGGTCATAATAGCTTTC |
| | CCATCATGAATCAGAAAGATGTGGA |
| | CAGCTTGATGTTTTAGACAACCACT |
| | GAACTAGATGACTGTTGTACTGTAG |
| | CTCAGTCATTTAAAAAATATATAAA |
| | TACTACCTTGTAGTGTCCCATACTG |
| | TGTTTTTTACATGGTAGATTCTTAT |
| | TTAAGTGCTAACTGGTTATTTTCTT |
| | TGGCTGGTTTATTGTACTGTTATAC |
| | AGAATGTAAGTTGTACAGTGAAATA |
| | AGTTATTAAAGCATGTGTAAACATT |
| | GTTATATATCTTTTCTCCTAAATGG |
| | AGAATTTTGAATAAAATATATTTGA |
| | AATTTT |
| NM_145005.7 | ACGTAACCTACGGTGTCCCGCTAGG |
| C9orf72 | AAAGAGAGGTGCGTCAAACAGCGAC |
| transcript | AAGTTCCGCCCACGTAAAAGATGAC |
| variant 1 | GCTTGATATCTCCGGAGCATTTGGA |
| (SEQ ID NO: 37) | TAATGTGACAGTTGGAATGCAGTGA |
| | TGTCGACTCTTTGCCCACCGCCATC |
| | TCCAGCTGTTGCCAAGACAGAGATT |
| | GCTTTAAGTGGCAAATCACCTTTAT |
| | TAGCAGCTACTTTTGCTTACTGGGA |
| | CAATATTCTTGGTCCTAGAGTAAGG |
| | CACATTTGGGCTCCAAAGACAGAAC |
| | AGGTACTTCTCAGTGATGGAGAAAT |
| | AACTTTTCTTGCCAACCACACTCTA |
| | AATGGAGAAATCCTTCGAAATGCAG |
| | AGAGTGGTGCTATAGATGTAAAGTT |
| | TTTTGTCTTGTCTGAAAAGGGAGTG |
| | ATTATTGTTTCATTAATCTTTGATG |
| | GAAACTGGAATGGGGATCGCAGCAC |
| | ATATGGACTATCAATTATACTTCCA |
| | CAGACAGAACTTAGTTTCTACCTCC |
| | CACTTCATAGAGTGTGTGTTGATAG |
| | ATTAACACATATAATCCGGAAAGGA |
| | AGAATATGGATGCATAAGGAAAGAC |
| | AAGAAAATGTCCAGAAGATTATCTT |
| | AGAAGGCACAGAGAGAATGGAAGAT |
| | CAGGGTCAGAGTATTATTCCAATGC |
| | TTACTGGAGAAGTGATTCCTGTAAT |
| | GGAACTGCTTTCATCTATGAAATCA |
| | CACAGTGTTCCTGAAGAAATAGATA |
| | TAGCTGATACAGTACTCAATGATGA |
| | TGATATTGGTGACAGCTGTCATGAA |
| | GGCTTTCTTCTCAAGTAAGAATTTT |
| | TCTTTTCATAAAAGCTGGATGAAGC |
| | AGATACCATCTTATGCTCACCTATG |
| | ACAAGATTTGGAAGAAAGAAAATAA |
| | CAGACTGTCTACTTAGATTGTTCTA |
| | GGGACATTACGTATTTGAACTGTTG |
| | CTTAAATTTGTGTTATTTTTCACTC |
| | ATTATATTTCTATATATATTTGGTG |
| | TTATTCCATTTGCTATTTAAAGAAA |
| | CCGAGTTTCCATCCCAGACAAGAAA |
| | TCATGGCCCCTTGCTTGATTCTGGT |
| | TTCTTGTTTTACTTCTCATTAAAGC |
| | TAACAGAATCCTTTCATATTAAGTT |
| | GTACTGTAGATGAACTTAAGTTATT |
| | TAGGCGTAGAACAAAATTATTCATA |
| | TTTATACTGATCTTTTTCCATCCAG |
| | CAGTGGAGTTTAGTACTTAAGAGTT |
| | TGTGCCCTTAAACCAGACTCCCTGG |
| | ATTAATGCTGTGTACCCGTGGGCAA |
| | GGTGCCTGAATTCTCTATACACCTA |
| | TTTCCTCATCTGTAAAATGGCAATA |
| | ATAGTAATAGTACCTAATGTGTAGG |
| | GTTGTTATAAGCATTGAGTAAGATA |
| | AATAATATAAAGCACTTAGAACAGT |
| | GCCTGGAACATAAAAACACTTAATA |
| | ATAGCTCATAGCTAACATTTCCTAT |
| | TTACATTTCTTCTAGAAATAGCCAG |
| | TATTTGTTGAGTGCCTACATGTTAG |
| | TTCCTTTACTAGTTGCTTTACATGT |
| | ATTATCTTATATTCTGTTTTAAAGT |
| | TTCTTCACAGTTACAGATTTTCATG |
| | AAATTTTACTTTTAATAAAAGAGAA |
| | GTAAAAGTATAAAGTATTCACTTTT |
| | ATGTTCACAGTCTTTTCCTTTAGGC |
| | TCATGATGGAGTATCAGAGGCATGA |
| | GTGTGTTTAACCTAAGAGCCTTAAT |
| | GGCTTGAATCAGAAGCACTTTAGTC |
| | CTGTATCTGTTCAGTGTCAGCCTTT |
| | CATACATCATTTTAAATCCCATTTG |
| | ACTTTAAGTAAGTCACTTAATCTCT |
| | CTACATGTCAATTTCTTCAGCTATA |
| | AAATGATGGTATTTCAATAAATAAA |
| | TACATTAATTAAATGATATTATACT |
| | GACTAATTGGGCTGTTTTAAGGCTC |
| | AATAAGAAAATTTCTGTGAAAGGTC |
| | TCTAGAAAATGTAGGTTCCTATACA |
| | AATAAAAGATAACATTGTGCTTATA |

### Exemplary (9Orf72 Nucleotide Sequence

### Exemplary (9Orf72 Amino Acid Sequence

| Accession No. | Sequences |
|---|---|
| NP_001242983.1 | MSTLCPPPSPAVAKTEIALSGKSPL |
| C9orf72 | LAATFAYWDNILGPRVRHIWAPKTE |
| isoform a | QVLLSDGEITFLANHTLNGEILRNA |
| (variant 3) | ESGAIDVKFFVLSEKGVIIVSLIFD |
| (SEQ ID NO: 38) | GNWNGDRSTYGLSIILPQTELSFYL |
| | PLHRVCVDRLTHIIRKGRIWMHKER |
| | QENVQKIILEGTERMEDQGQSIIPM |
| | LTGEVIPVMELLSSMKSHSVPEEID |
| | IADTVLNDDDIGDSCHEGFLLNAIS |
| | SHLQTCGCSVVVGSSAEKVNKIVRT |
| | LCLFLTPAERKCSRLCEAESSFKYE |
| | SGLFVQGLLKDSTGSFVLPFRQVMY |
| | APYPTTHIDVDVNTVKQMPPCHEHI |
| | YNQRRYMRSELTAFWRATSEEDMAQ |
| | DTIIYTDESFTPDLNIFQDVLHRDT |
| | LVKAFLDQVFQLKPGLSLRSTFLAQ |
| | FLLVLHRKALTLIKYIEDDTQKGKK |
| | PFKSLRNLKIDLDLTAEGDLNIIMA |
| | LAEKIKPGLHSFIFGRPFYTSVQER |
| | DVLMTF |
| NP_060795.1 | MSTLCPPPSPAVAKTEIALSGKSPL |
| C9orf72 | LAATFAYWDNILGPRVRHIWAPKTE |
| isoform a | QVLLSDGEITFLANHTLNGEILRNA |
| (variant 2) | ESGAIDVKFFVLSEKGVIIVSLIFD |
| (SEQ ID NO: 39) | GNWNGDRSTYGLSIILPQTELSFYL |
| | PLHRVCVDRLTHIIRKGRIWMHKER |
| | QENVQKIILEGTERMEDQGQSIIPM |
| | LTGEVIPVMELLSSMKSHSVPEEID |
| | IADTVLNDDDIGDSCHEGFLLNAIS |
| | SHLQTCGCSVVVGSSAEKVNKIVRT |
| | LCLFLTPAERKCSRLCEAESSFKYE |
| | SGLFVQGLLKDSTGSFVLPFRQVMY |
| | APYPTTHIDVDVNTVKQMPPCHEHI |
| | YNQRRYMRSELTAFWRATSEEDMAQ |
| | DTIIYTDESFTPDLNIFQDVLHRDT |
| | LVKAFLDQVFQLKPGLSLRSTFLAQ |
| | FLLVLHRKALTLIKYIEDDTQKGKK |
| | PFKSLRNLKIDLDLTAEGDLNIIMA |
| | LAEKIKPGLHSFIFGRPFYTSVQER |
| | DVLMTF |
| NP_659442.2 | MSTLCPPPSPAVAKTEIALSGKSPL |
| C9orf72 | LAATFAYWDNILGPRVRHIWAPKTE |
| isoform b | QVLLSDGEITFLANHTLNGEILRNA |
| (variant 1) | ESGAIDVKFFVLSEKGVIIVSLIFD |
| (SEQ ID NO: 40) | GNWNGDRSTYGLSIILPQTELSFYL |
| | PLHRVCVDRLTHIIRKGRIWMHKER |
| | QENVQKIILEGTERMEDQGQSIIPM |
| | LTGEVIPVMELLSSMKSHSVPEEID |
| | IADTVLNDDDIGDSCHEGFLLK |

### Viral Vector

Viral vector is widely used to refer to a nucleic acid molecule that includes virus-derived nucleic acid elements that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

As used herein, the term “adenovirus vector” refers to those constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. As used herein, the term “AAV vector” in the context of the present invention includes without limitation AAV type 1. AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. Sec, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388 and Table 1), which are also encompassed by the term “AAV.” Adenovirus is particularly suitable for use as a gene transfer vector because of its mid-sized genome, case of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1 B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In some embodiments, adenovirus type 5 of subgroup C is the preferred starting material in order to obtain a conditional replication-defective adenovirus vector for use in some embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in selected cell populations, scAAV refers to a self-complementary AAV, pAAV refers to a plasmid adeno-associated virus, rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retrovirus. Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in some embodiments include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In some embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In some embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and US regions. The R region plays a role during reverse transcription in permitting the transfer of nascent DNA from one end of the genome to the other.

In some embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al, 1999, J. Virol., 73:2886); the posttranscriptional regulatory element present in hepatitis B virus (HPRE) (Smith et al., Nucleic Acids Res. 26(21):4818-4827, 1998); and the like (Liu et al., 1995, Genes Dev., 9: 1766). In some embodiments, vectors include a posttranscriptional regulatory element such as a WPRE or HPRE. In some embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In some embodiments, vectors include a polyadenylation sequence 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40 pA. In some embodiments, a preferred embodiment of an expression construct includes a terminator element. These elements can serve to enhance transcript levels and to minimize read through from the construct into other plasmid sequences.

In some embodiments, a viral vector further includes one or more insulator elements. Insulator elements may contribute to protecting viral vector-expressed sequences, e.g., effector elements or expressible elements, from integration site effects, which may be mediated by as—acting elements present in genomic DNA and lead to deregulated expression of transferred sequences (i.e., position effect; see, e.g., Burgess-Beusse et al, PNAS., USA, 99: 16433, 2002; and Zhan et al., Hum. Genet., 109:471, 2001). In some embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken b-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33: 198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In some embodiments, suitable expression vectors include any plasmid, cosmid or phage construct that is capable of supporting expression of encoded genes in mammalian cell, such as pUC or Bluescript plasmid series.

TABLE 1
Particular embodiments of vectors disclosed herein

| Nucleic Acid Constructs | Description |
|---|---|
| Enh98-pBG-GFP | ITR - mEnh98 - beta globin promoter - GFP - WPRE - pA - ITR |
| (SEQ ID NO: 46) | |
| Enh57-pBG-GFP | ITR - mEnh57 - beta globin promoter - GFP - WPRE - pA - ITR |
| (SEQ ID NO: 47) | |
| Enh98-pChat-GFP | ITR - mEnh98 - choline acetyltransferase promoter - GFP - WPRE - |
| (SEQ ID NO: 48) | pA - ITR |
| Enh57-pChat-GFP | ITR - mEnh57 - choline acetyltransferase promoter - GFP - WPRE - |
| (SEQ ID NO: 49) | pA - ITR |

In some embodiments, vectors (e.g., AAV) with capsids that cross the blood-brain barrier (BBB) or blood-spinal cord barrier (BSCB) are selected. In some embodiments, vectors are modified to include capsids that cross the BBB or BSCB. Examples of AAV with viral capsids that cross the blood brain barrier include AAV9 (Gombash et al., Front Mol Neurosci. 2014; 7:81), AAVrh.10 (Yang, et al., Mol Ther. 2014; 22(7): 1299-1309), AAV1 R6, AAV1 R7 (Albright et al., Mol Ther. 2018; 26(2): 510), rAAVrh.8 (Yang, et al., supra), AAV-BR1 (Marchio et al., EMBO Mol Med. 2016; 8(6): 592), AAV-PHP.S (Chan et al., Nat Neurosci. 2017; 20(8): 1 172), AAV-PHP.B (Deverman et al., Nat Biotechnol. 2016; 34(2): 204), and AAV-PPS (Chen et al., Nat Med. 2009; 15: 1215). The PHP.eB capsid differs from AAV9 such that, using AAV9 as a reference, the sequence DGTLA VPFK (SEQ ID NO: 41) is inserted between amino acids residues 586 and 587 of AAV9.

In some embodiments, AAV comprises AAV type 1 (AAV1), AAV type 2 (AAV2), AAV type 3 (including types AAV3A and AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), and AAV type 11 (AAV11) and any other AAV now known or later discovered.

In some embodiments, the AAV genome comprises AAV1 (GenBank Accession No. NC_002077, AF063497) Adeno-associated NC_002077, AAV2 (GenBank Accession No. NC_001401), AAV3 (GenBank Accession No. NC_001729), AAV3B (GenBank Accession No. NC_001863), AAV4 (GenBank Accession No. NC_001829), AAV5 (GenBank Accession No. Y18065, AF085716), or AAV6 (GenBank Accession No. NC_001862).

In some embodiments, the AAV comprises a capsid protein VPI gene of Hu.48 (GenBank Accession No. AY530611), Hu 43 (GenBank Accession No. AY530606), Hu 44 (GenBank Accession No. AY530607), Hu 46 (GenBank Accession No. AY530609), Hu. 19 (GenBank Accession No. AY530584), Hu. 20 (GenBank Accession No. AY530586), Hu 23 (GenBank Accession No. AY530589), Hu22 (GenBank Accession No. AY530588), Hu24 (GenBank Accession No. AY530590), Hu21 (GenBank Accession No. AY530587), Hu27 (GenBank Accession No. AY530592), Hu28 (GenBank Accession No. AY530593), Hu 29 (GenBank Accession No. AY530594), Hu63 (GenBank Accession No. AY530624), Hu64 (GenBank Accession No. AY530625), Hu13 (GenBank Accession No. AY530578), Hu56 (GenBank Accession No. AY530618), Hu57 (GenBank Accession No. AY530619), Hu49 (GenBank Accession No. AY530612), Hu58 (GenBank Accession No. AY530620), Hu34 (GenBank Accession No. AY530598), Hu35 (GenBank Accession No. AY53059), Hu45 (GenBank Accession No. AY530608), Hu47 (GenBank Accession No. AY530610), Hu51 (GenBank Accession No. AY530613), Hu52 (GenBank Accession No. AY53061), Hu T41 (GenBank Accession No. AY695378), Hu S17 (GenBank Accession No. AY695376), Hu T88 (GenBank Accession No. AY695375), Hu T71 (GenBank Accession No. AY695374), Hu T70 (GenBank Accession No. AY695373), Hu T40 (GenBank Accession No. AY695372), Hu T32 (GenBank Accession No. AY695371), Hu T17 (GenBank Accession No. AY695370), Hu LG15 (GenBank Accession No. AY695377), Hu9 (GenBank Accession No. AY530629), Hu10 (GenBank Accession No. AY530576), Hull (GenBank Accession No. AY530577), Hu53 (GenBank Accession No. AY530615), Hu55 (GenBank Accession No. AY530617), Hu54 (GenBank Accession No. AY530616), Hu7 (GenBank Accession No. AY530628), Hu18 (GenBank Accession No. AY530583), Hu15 (GenBank Accession No. AY530580), Hu16 (GenBank Accession No. AY530581), Hu25 (GenBank Accession No. AY530591), Hu60 (GenBank Accession No. AY530622) Hu3 (GenBank Accession No. AY530595), Hu1 (GenBank Accession No. AY530575), Hu4 (GenBank Accession No. AY530602), Hu2 (GenBank Accession No. AY530585), Hu61 (GenBank Accession No. AY530623), Rh62 (GenBank Accession No. AY530573), Rh48 (GenBank Accession No. AY530561), Rh54 (GenBank Accession No. AY530567), Rh55 (GenBank Accession No. AY530568), Rh35 (GenBank Accession No. AY243000), Rh38 (GenBank Accession No. AY530558), Hu66 (GenBank Accession No. AY530626), Hu42 (GenBank Accession No. AY530605), Hu67 (GenBank Accession No. AY530627), Hu40 (GenBank Accession No. AY530603), Hu41 (GenBank Accession No. AY530604), Hu37 (GenBank Accession No. AY530600), Rh40 (GenBank Accession No. AY530559), Hu17 (GenBank Accession No. AY530582), Hu6 (GenBank Accession No. AY530621), Rh25 (GenBank Accession No. AY530557), Pi2 (GenBank Accession No. AY530554), Pil (GenBank Accession No. AY530553), Pi3 (GenBank Accession No. AY530555), Rh57 (GenBank Accession No. AY530569), Rh50 (GenBank Accession No. AY530563), Rh49 (GenBank Accession No. AY530562), Hu39 (GenBank Accession No. AY530601), Rh58 (GenBank Accession No. AY530570), Rh61 (GenBank Accession No. AY530572), Rh52 (GenBank Accession No. AY530565), Rh53 (GenBank Accession No. AY530566), Rh51 (GenBank Accession No. AY530564), Rh64 (GenBank Accession No. AY530574), Rh43 (GenBank Accession No. AY530560), Rh1 (GenBank Accession No. AY530556), Hu14 (GenBank Accession No. AY530579), Hu31 (GenBank Accession No. AY530596), or Hu32 (GenBank Accession No. AY530597).

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31 (4): 317), for example, as described in relation to clinical trials for the treatment of superior mesenteric artery (SMA) syndrome by AveXis (AVXS-101, NCT03505099) and the treatment of CLN3 gene-Related Neuronal Ceroid-Lipofuscinosis (NCT03770572).

AAVrh.10, was originally isolated from rhesus macaques and shows low seropositivity in humans when compared with other common serotypes used for gene delivery applications (Selot et al., Front Pharmacol. 2017; 8: 441) and has been evaluated in clinical trials LYS-SAF302, LYSOGENE, and NCT03612869.

AAV1 R6 and AAV1 R7, two variants isolated from a library of chimeric AAV vectors (AAV1 capsid domains swapped into AAVrh. 10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 42) epitope that was isolated during in vivo screening of a random AAV display peptide library. It shows high specificity accompanied by high transgene expression in the brain with minimal off-target affinity (including for the liver) (Korbelin et al., EMBO Mol Med. 2016; 8(6): 609). AAV-PHP.S (Addgene, Watertown, MA) is a variant of AAV9 generated with the CREATE method that encodes the 7-mer sequence QAVRTSL (SEQ ID NO: 43), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 44). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 45) epitope into the capsid of AAV2, shows a dramatically improved brain tropism relative to AAV2.

### Formulations

Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in some embodiments, when administered intravenously (e.g., at the retro-orbital plexus).

In some embodiments, compositions can be formulated for intravenous, intraocular, intravitreal, parenteral, subcutaneous, intracerebro-ventricular, intramuscular, intracerebroventricular, intravenous injection into the cisterna magna (ICM), intrathecal, intraspinal, oral, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

As used herein, the term “lipid nanoparticle” refers to a vesicle formed by one or more lipid components. Lipid nanoparticles are typically used as carriers for nucleic acid delivery in the context of pharmaceutical development. They work by fusing with a cellular membrane and repositioning its lipid structure to deliver a drug or active pharmaceutical ingredient (API). Generally, lipid nanoparticle compositions for such delivery are composed of synthetic ionizable or cationic lipids, phospholipids (especially compounds having a phosphatidylcholine group), cholesterol, and a polyethylene glycol (PEG) lipid; however, these compositions may also include other lipids. The sum composition of lipids typically dictates the surface characteristics and thus the protein (opsonization) content in biological systems thus driving biodistribution and cell uptake properties.

As used herein, the “liposome” refers to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “ionizable lipid” refers to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. Ionizable lipids are also referred to as cationic lipids herein.

As used herein, the term “non-cationic lipid” refers to any amphipathic lipid as well as any other neutral lipid or anionic lipid. Accordingly, the non-cationic lipid can be a neutral uncharged, zwitterionic, or anionic lipid.

As used herein, the term “conjugated lipid” refers to a lipid molecule conjugated with a non-lipid molecule, such as a PEG, polyoxazoline, polyamide, or polymer (e.g., cationic polymer).

As used herein, the term “excipient” refers to pharmacologically inactive ingredients that are included in a formulation with the AP1, e.g., ceDNA and/or lipid nanoparticles to bulk up and/or stabilize the formulation when producing a dosage form. General categories of excipients include, for example, bulking agents, fillers, diluents, antiadherents, binders, coatings, disintegrants, flavours, colors, lubricants, glidants, sorbents, preservatives, sweeteners, and products used for facilitating drug absorption or solubility or for other pharmacokinetic considerations.

The formation and use of liposomes is generally known to those of skill in the art. Liposomes have been developed with improved serum stability and circulation half-times (see, for instance, U.S. Pat. No. 5,741 0.516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24(12): 11 13-1 128, 1998; Quintanar-Guerrero et al, Pharm Res. 15(7): 1056-1062, 1998; Quintanar-Guerrero et al., J. Microencapsul. 15(1): 107-1 19, 1998; Douglas et al, Crit Rev Ther Drug Carrier Syst 3(3):233-261. 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69(2): 199-202, 1980; Couvreur et al., Crit Rev Ther Drug Carrier Syst. 5(1)1-20, 1988; zur Muhlen et al., EurJ Pharm Biopharm, 45(2): 149-155, 1998; Zambau x et al., J Control Release 50(1-3):31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In some embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al, Prog Retin Eye Res, 17(1):33-58, 1998), transdermal matrices (U.S. Pat. Nos. 5,770,219; 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In some embodiments, for administration to humans, compositions should meet sterility, pyrogenicity, and the general safety and purity standards as required by United States Food and Drug Administration (FDA) or other applicable regulatory agencies in other countries.

### Cell Lines

The present disclosure includes cells including an artificial expression construct described herein. A cell that has been transformed with an artificial expression construct can be used for many purposes, including in neuroanatomical studies, assessments of functioning and/or non-functioning proteins, and drug screens that assess the regulatory properties of enhancers.

WO 91/13150 describes a variety of cell lines, including neuronal cell lines, and methods of producing them. Similarly, WO 97/39117 describes a neuronal cell line and methods of producing such cell lines. The neuronal cell lines disclosed in these patent applications are applicable for use in the present disclosure.

In some embodiments, a “neural cell” refers to a cell or cells located within the central nervous system, and includes neurons and glia, and cells derived from neurons and glia, including neoplastic and tumor cells derived from neurons or glia. A “cell derived from a neural cell” refers to a cell which is derived from or originates or is differentiated from a neural cell.

In some embodiments, “neuronal” describes something that is of, related to, or includes, neuronal cells. Neuronal cells are defined by the presence of an axon and dendrites. The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

Methods to differentiate stem cells into neuronal cells include replacing a stem cell culture media with a media including basic fibroblast growth factor (bFGF) heparin, an N2 supplement (e.g., transferrin, insulin, progesterone, putrescine, and selenite), laminin and polyornithine. A process to produce myelinating oligodendrocytes from stem cells is described in Hu, et al., 2009, Nat. Protoc. 4: 1614-22. Bihel, et al., 2007, Nat. Protoc. 2:1034-43 describes a protocol to produce glutamatergic neurons from stem cells while Chatzi, et at., 2009, Exp. Neurol. 217:407-16 describes a procedure to produce GABAergic neurons. This procedure includes exposing stem cells to all-trans-RA for three days. After subsequent culture in serum-free neuronal induction medium including IMeurobasai medium supplemented with B27, bFGF and EGF, 95% GABA neurons develop.

U.S. Publication No. 2012/0329714 describes use of prolactin to increase neural stem cell numbers while U.S. Publication No. 2012/0308530 describes a culture surface with amino groups that promotes neuronal differentiation into neurons, astrocytes and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No. 5,766,948; FGF-1, FGF-2); Neurotrophin-3 (NT-3) and Neurotrophin-4 (NT-4); Caldwell, et al., 2001, Nat. Biotechnol. 1; 19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-a and TGF-b (U.S. Pat. Nos. 5,851,832 and 5,753,506).

Transgenic animals are described below. Cell lines may also be derived from such transgenic animals. For example, primary tissue culture from transgenic mice (e.g., also as described below) can provide cell lines with the expression construct already integrated into the genome (for an example see Mackenzie & Quinn, Proc Natl Acad Sci USA 96: 15251-15255, 1999).

### Transgenic Animals

Another aspect of the disclosure includes transgenic animals, the genome of which contains an artificial expression construct including regulatory elements (e.g., SEQ ID NOs: 7-14 or 60-65) operatively linked to a heterologous gene. In some embodiments, the genome of a transgenic animal includes the Enh98-pBG-GFP, Enh57-pBG-GFP, Enh98-pChat-GFP, or Enh57-pChat-GFP. In some embodiments, when a non-integrating vector is utilized, a transgenic animal includes an artificial expression construct including regulatory elements (e.g., SEQ ID NO: 7-14 or 60-65) and/or Enh98-pBG-GFP. Enh57-pBG-GFP, Enh98-pChat-GFP, or Enh57-pChat-GFP within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In some embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to heterologous expression of the construct. A disadvantage of this approach is that the expression of the introduced DNA may be affected by the specific genetic background of the host animal.

As indicated above in relation to cell lines, the artificial expression constructs of this disclosure can be used to genetically modify mouse embryonic stem cells using techniques known in the art. Typically, the artificial expression construct is introduced into cultured murine embryonic stem cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually, the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice be crossed with an appropriate strain to produce offspring that will carry the transgene.

In addition to the methods of delivery described above, the following techniques are also contemplated as alternative methods of delivering artificial expression constructs to target cells or selected tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17(1):33-58, 1998); transdermal matrices (U.S. Pat. Nos. 5,770,219; 5,783,208); and feedback-controlled delivery (U.S. Pat. No. 5,697,899).

### Methods of Use

In some embodiments, a composition including a physiologically active component described herein is administered to a subject that has a motor neuron disease or disorder.

As used herein, the term “motor neuron disease or disorder” refers to a disease or disorder involving the abnormal function of motor neurons resulting from abnormal protein expression, e.g., loss-of-function SMN1 protein.

In some embodiments, the disease or disorder is Spinal-bulbar muscular atrophy (SBMA), Spinal muscular atrophy (SMA) (e.g., non-5q SMA, Spinal muscular atrophy with congenital bone fractures, Autosomal dominant spinal muscular atrophy, and lower extremity-predominant 2 (SMALED2)), Distal hereditary motor neuropathy (dHMN) (e.g., autosomal dominant, autosomal recessive, type 1, 2, 4, 5, 6, 7, 7b), Triosephosphate isomerase deficiency (TIP), Hereditary spastic paraplegia (HSP) (also known as familial spastic paraparesis (FSP))(e.g., autosomal dominant or autosomal recessive or X-linked), amyotrophic lateral sclerosis (ALS), ALS with frontotemporal dementia (FTD), Adrenomyeloneuropathy (AMN), Allgrove syndrome (also known as triple A (3A) syndrome), Friedreich ataxia (FA), Spinocerebellar ataxia (SCA), Pontocerebellar hypoplasias (PCH), Neurodegeneration with brain iron accumulation (NBIA), mitochondrial complex I deficiency nuclear type 21 (MC1DN21), GM2 gangliosidosis (also known as Tay-Sachs disease or HexA deficiency), Charcot-Marie-Tooth disease (CMT), or a combination thereof.

In some embodiments, symptoms associated with the motor neuron disease or disorder are muscle weakness and decreased muscle tone, limited mobility, breathing problems, problems eating and swallowing, delayed gross motor skills, congenital hemolytic anemia, progressive neuromuscular dysfunction, spontaneous tongue movements, behavioral/cognitive symptoms, cerebellar degeneration, or scoliosis.

In some embodiments, the disclosure includes the use of the artificial expression constructs described herein to modulate expression of a heterologous gene which is either partially or wholly encoded in a location downstream to that enhancer in an engineered sequence. Thus, there are provided herein methods of use of the disclosed artificial expression constructs in the research, study, and potential development of medicaments for preventing, treating or ameliorating the symptoms of a disease, dysfunction, or disorder.

In some embodiments include methods of administering to a subject an artificial expression construct that includes SEQ ID NOs: 1-14 or 60-71, as described herein to drive selective expression of a gene in a selected neural cell type.

In some embodiments include methods of administering to a subject an artificial expression construct that includes Enh98-pBG-GFP, Enh57-pBG-GFP, Enh98-pChat-GFP, or Enh57-pChat-GFP, as described herein to drive selective expression of a gene in a selected neural cell type wherein the subject can be an isolated cell, a network of cells, a tissue slice, an experimental animal, a veterinary animal, or a human.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in some embodiments, a dose could be from 10⁵ to 10100 copies of an artificial expression construct of the disclosure. In some embodiments, a patient receiving intravenous, intraspinal, retro-orbital, or intrathecal administration can be infused with from 10⁶ to 10²² copies of the artificial expression construct.

An “effective amount” is the amount of a composition necessary to result in a desired physiological change in the subject. Effective amounts are often administered for research purposes. Effective amounts disclosed herein can cause a statistically-significant effect in an animal model or in vitro assay.

In some embodiments, constructs disclosed herein can be utilized to treat spinal muscular atrophy (SMA). In some embodiments, the methods reduce or prevent muscle weakness, or symptoms thereof in a patient in need thereof. In some embodiments, the methods provided may reduce or prevent one or more symptoms associated with SMA, e.g., muscle weakness and decreased muscle tone, limited mobility, breathing problems, problems eating and swallowing, delayed gross motor skills, spontaneous tongue movements, or scoliosis.

The amount of expression constructs and time of administration of such compositions will be within the purview of the skilled artisan having benefit of the present teachings. It is likely, however, that the administration of effective amounts of the disclosed compositions may be achieved by a single administration, such as for example, a single injection of sufficient numbers of infectious particles to provide an effect in the subject. Alternatively, in some circumstances, it may be desirable to provide multiple, or successive administrations of the artificial expression construct compositions or other genetic constructs, either over a relatively short, or a relatively prolonged period of time, as may be determined by the individual overseeing the administration of such compositions. For example, the number of infectious particles administered to a mammal may be 10⁷, 10⁸, 10⁹, 10¹⁰, 10¹¹, 10¹², 10¹³, or even higher, infectious particles/ml given either as a single dose or divided into two or more administrations as may be required to achieve an intended effect. In fact, in certain embodiments, it may be desirable to administer two or more different expression constructs in combination to achieve a desired effect.

In certain circumstances it will be desirable to deliver the artificial expression construct in suitably formulated compositions disclosed herein either by pipette, retro-orbital injection, subcutaneously, intraocularly, intravitreally, parenterally, subcutaneously, intravenously, intracerebroventricular (ICV), intravenous injection into the cisterna magna (ICM), intracerebro-ventricularly, intramuscularly, intrathecally, intraspinally, orally, intraperitoneally, by oral or nasal inhalation, or by direct application or injection to one or more cells, tissues, or organs.

### Kits

Kits and commercial packages contain an artificial expression construct described herein. The expression construct can be isolated. In some embodiments, the components of an expression product can be isolated from each other. In some embodiments, the expression product can be within a vector, within a viral vector, within a cell, within a tissue slice or sample, and/or within a transgenic animal. Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition.

### EXAMPLES

### Example 1: Cell-Type Specific Expression of GFP with Enhancer 98

Regulatory elements Enh57 and Enh98 were cloned in front of the beta globin minimal promoter driving GFP. The constructs were packaged into adeno-associated virus AAV PhP-eB.

The AAVs were injected into the cerebral ventricle of ChAT-Cre; Sun1-GFP B6/C57 newborn mice in which nuclei of Chat+spinal cord motor-neurons are labeled enabling isolation by FACS. Individual rAAV-GRE constructs were injected into the lateral ventricle of newborn mice at a titer of 3×10¹³ genome copies/mL (2-4 μL).

Two weeks following transduction, animals were sacrificed, the spinal cord and dorsal root ganglia (DRG) dissected. Mice were sacrificed and perfused with 4% PFA followed by PBS. The brain was dissected out of the skull and post-fixed with 4% PFA for 1-3 days at 4° C. The brain was mounted on the vibratome (Leica™ VT1000S) and coronally sectioned into 100 μm slices. Sections containing VI were arrayed on glass slides and mounted using DAPI Fluoromount-G (Southern Biotech™). Sections containing VI were imaged on a Leica™ SPE confocal microscope using an ACS APO 10x/0.30 CS objective. Tiled VI cortical areas of −1.2 mm by −0.5 mm were imaged at a single optical section to avoid counting the same cell across multiple optical sections. Channels were imaged sequentially to avoid any optical crosstalk.

Spinal cord motor neuron nuclei were isolated by FACS. RNA-sequencing of spinal cord motor neurons, spinal cord non-motor neurons and DRG cells were used to measure the expression of enhancer-driven AAV vectors across these tissues. Immunostaining and/or fluorescent in situ hybridization was used to identify the cell types in which the GFP expression was observed.

Across all images, coordinates were registered for each GFP+ cell that could be visually discerned. An automated ImageJ script was developed to quantify the intensity of each acquired channel for a given GFP+ cell. The Inventors created a circular mask (radius=5.7 μm) at each coordinate representing a GFP positive cell, background subtracted (rolling ball, radius=72 μm) each channel, and quantified the mean signal of the masked area. To identify the threshold intensity used to classify each GFP+ cell as either SST+, VIP+ or PV+, the Inventors first determined the background signal in the channel representing SST, VIP or PV by selecting multiple points throughout the area visually identified as background. These background points were masked as small circular areas (radius=5.7 μm), over which the mean background signal was quantified. The highest mean background signal for SST, VIP and PV was conservatively chosen as the threshold for classifying GFP+ cells as SST+, VIP+ or PV+, respectively.

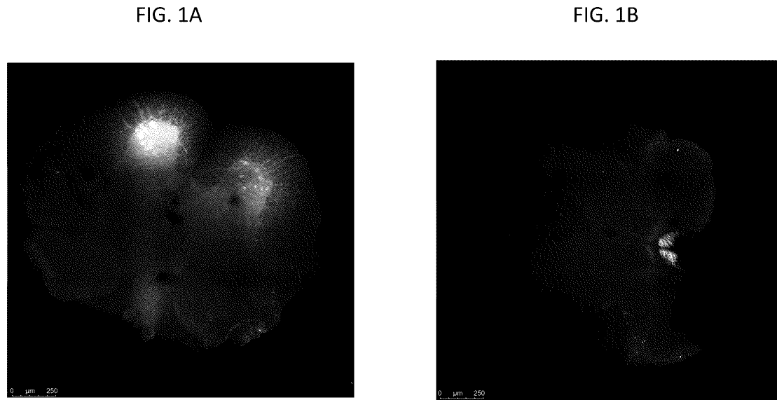

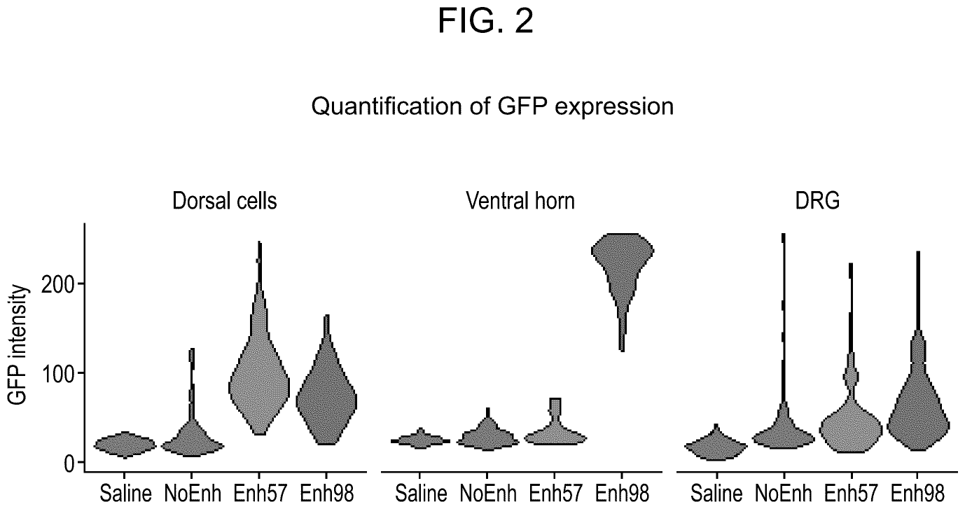

GFP expression was observed via immunostaining and fluorescent in situ hybridization in spinal cord after transduction with Enh98-pBG-GFP (FIG. 1A) and no Enh-pBG-GFP (FIG. 1B). Intensity of expression of GFP under control of Enh98 suggests Enh98 is specific for motor neuron in the ventral horn and less so for dorsal cells and DRG cells. Quantification of GFP expression comparing Enh98-pBG-GFP. Enh57-pBG-GFP, and no Enh-pBG-GFP shows that Enh98 induced strong expression in the ventral horn and less expression in the dorsal cells and DRG cells. Expression of GFP in the ventral horn induced by Enh57 was similar to expression without an enhancer.

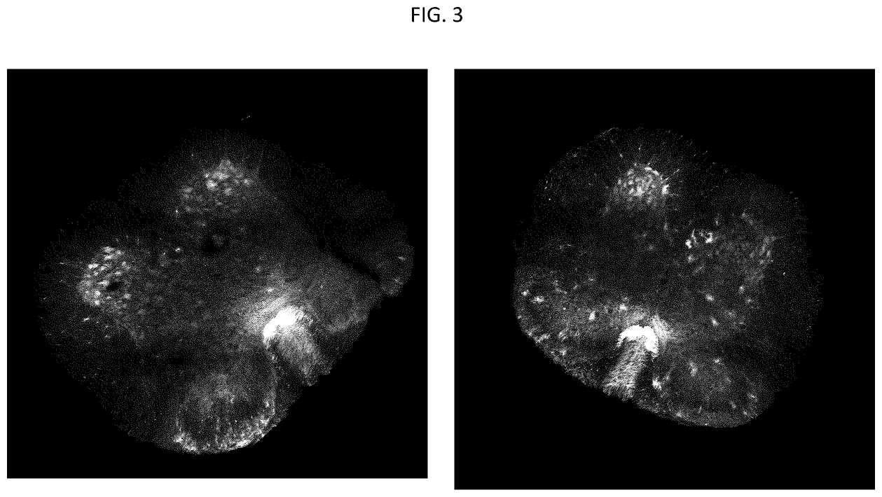

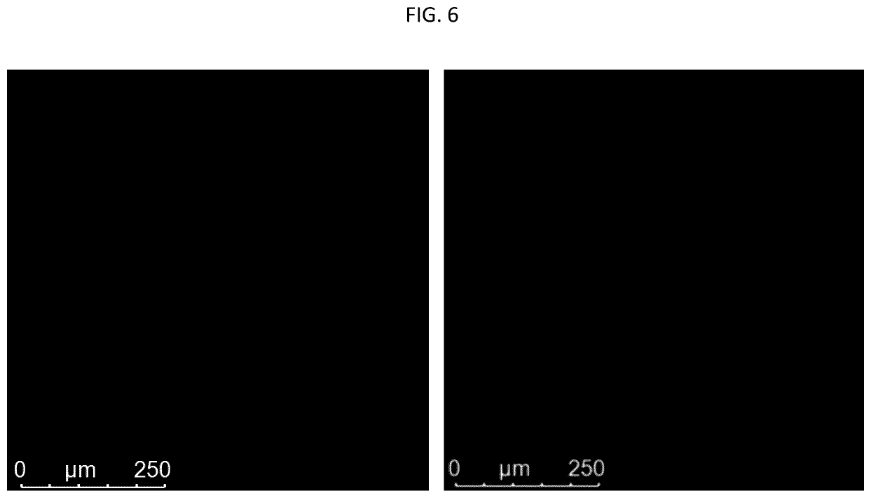

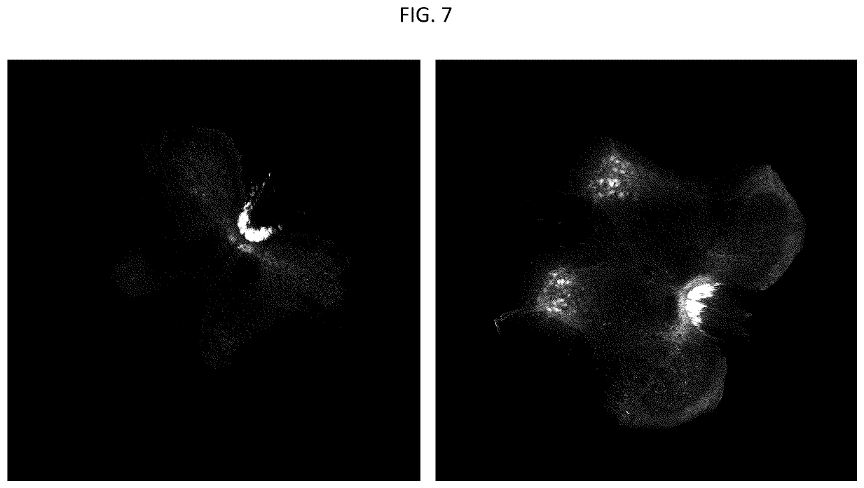

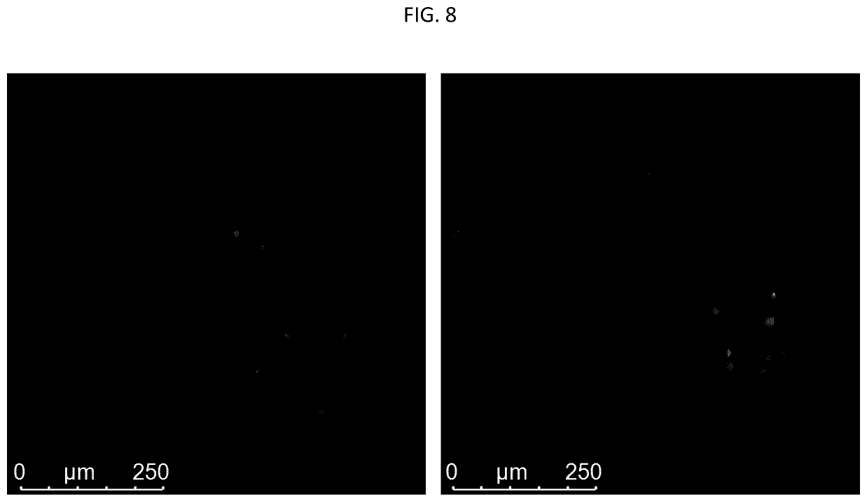

GFP expression was observed in spinal cord under the control of pCAG/no enhancer (FIG. 3), pBG/Enh98 (FIG. 5), pChAT/Enh98 (FIG. 7). GFP expression was observed in DRG cells under the control of pCAG/no enhancer (FIG. 4), pBG/Enh98 (FIG. 6), pChAT/Enh98 (FIG. 8).

### Example 2: Regulatory Elements for Spinal Cord Motor Neuron-Specific Viral Vectors

### SUMMARY

RNA-sequencing (RNA-seq) and the assay for transposase-accessible chromatin using sequencing (ATAC-seq) (Buenrostro et al., 2015) were used to generate a quantitative, genome-wide dataset of chromatin accessibility in lower motor neurons of the spinal cord in adult mouse. A subset of these gene regulatory elements (GREs) was selected and functionally evaluated by immunohistochemistry (IHC) for a GRE-driven reporter gene to identify two novel GREs with increased motor neuron specificity and substantial detargeting of liver and DRG compared to the industry standard CAG promoter. The molecular mechanisms by which these elements confer motor neuron-specific expression were investigated and a core sequence of transcription factor binding sites capable of reproducing the selectivity of the full-length sequence with reduced packaging size was identified.

### Results

### Candidate Cis-Regulatory Element Identification and Selection

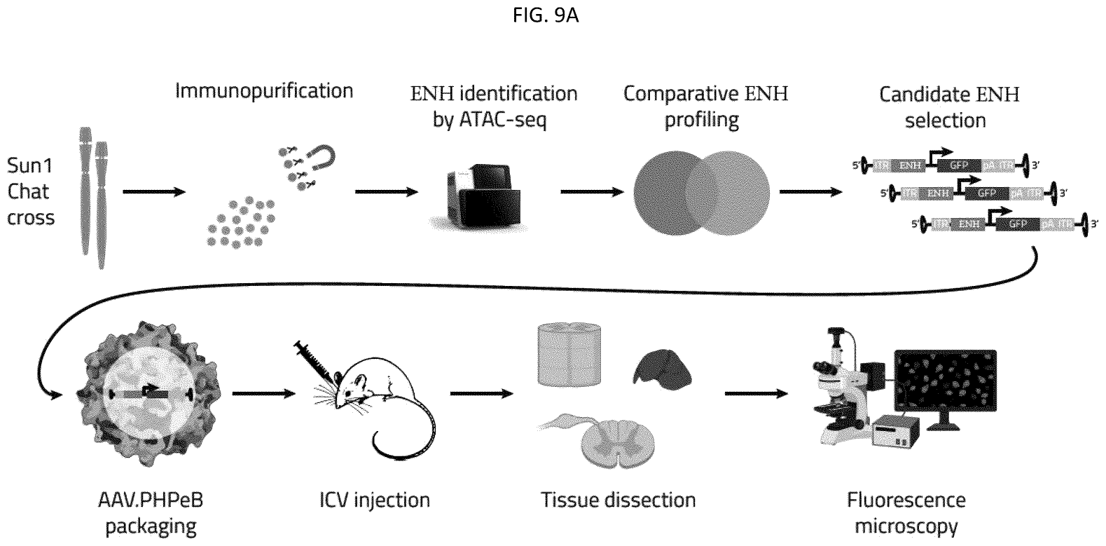

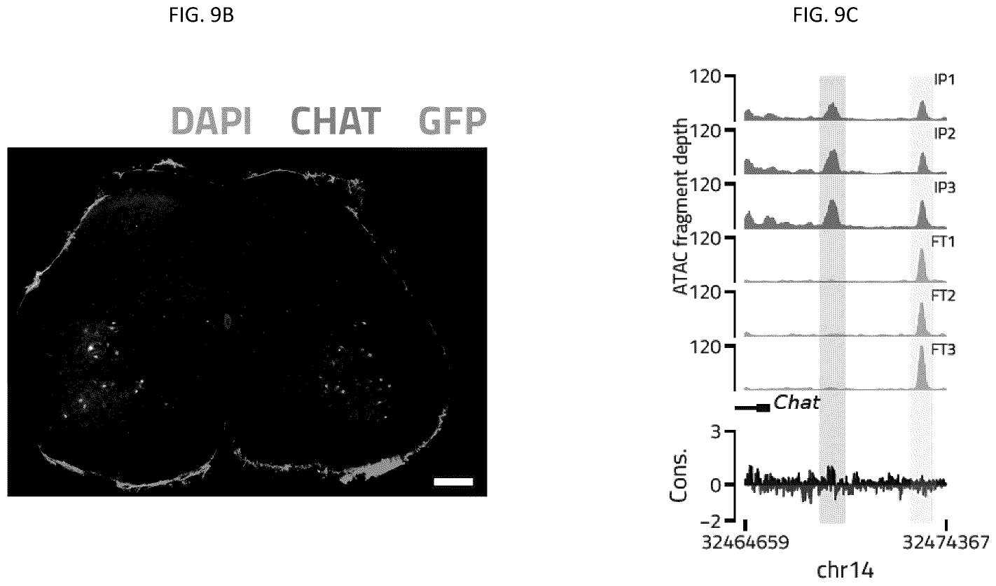

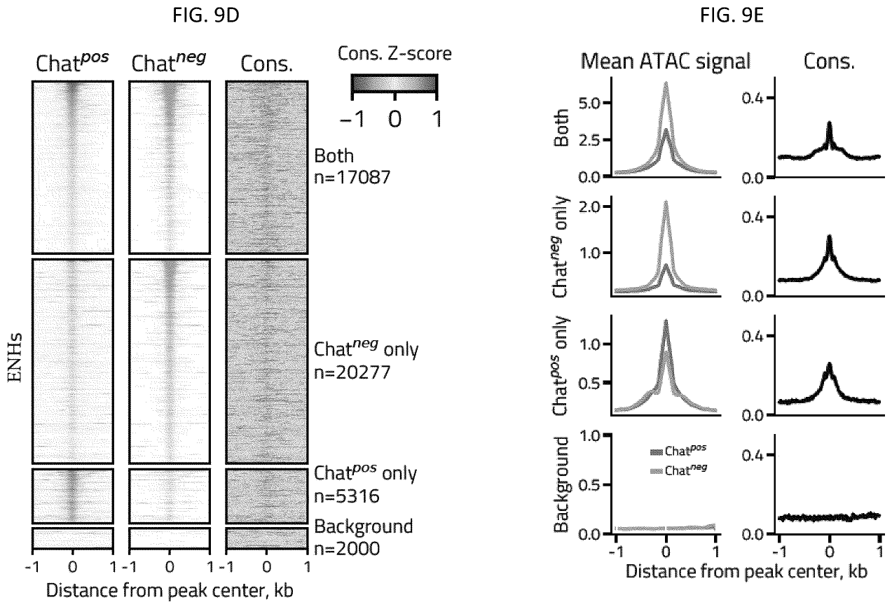

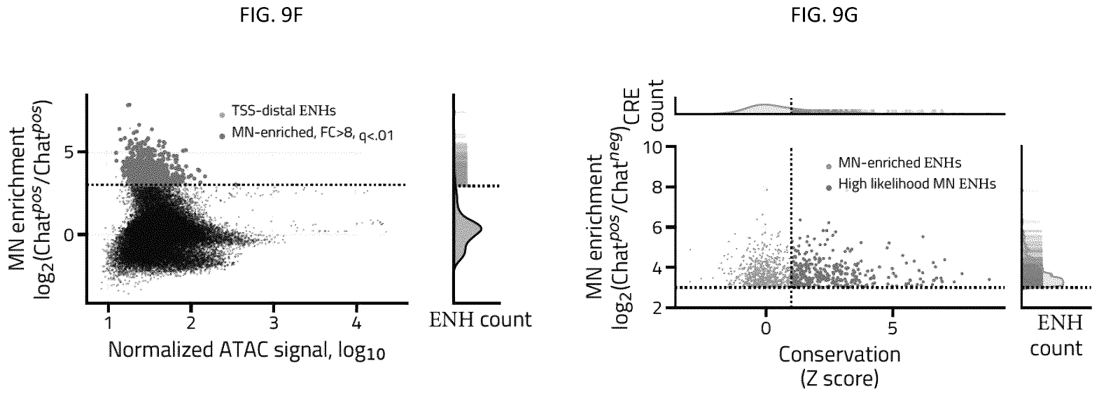

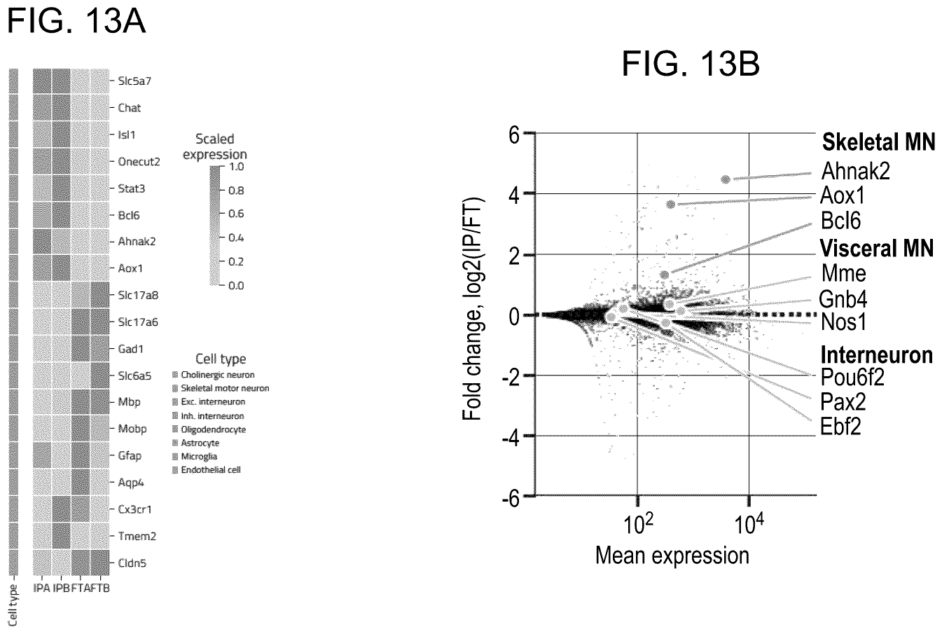

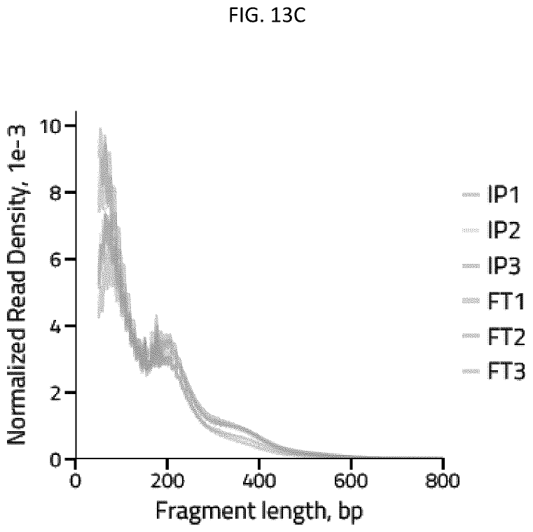

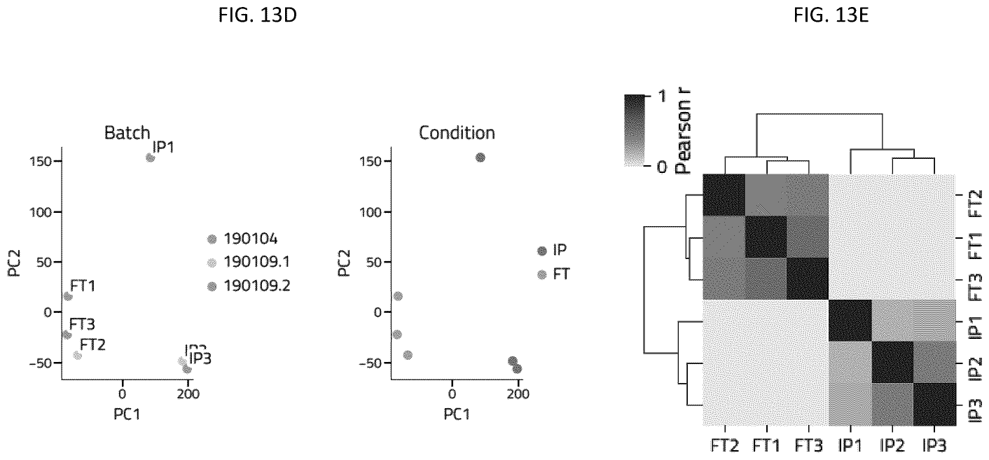

To identify motor neuron specific enhancers (Enh), also termed as cis-regulatory elements (CREs) or gene regulatory elements (GREs), spinal motor neuron nuclei were tagged and immunopurified using the Chat-Cre; Sun1-sfGFP-6xMyc mouse line cross (Chat-Sun1, Mo et al., 2015), which stably marks the nuclear envelope of Chat-expressing cells in animals of age E12.5 or older (Rossi et al. 2011; Patel et al. 2021). In the spinal cord, this population comprises skeletal motor neurons (target) and the off-target visceral motor neuron and cholinergic interneuron populations (Sathyamurthy et al., 2018). The composition of the immunolabeled population (Chatpos) by two complementary approaches was investigated. Confocal microscopy of immunohistochemically labeled Chat and GFP confirmed restriction of GFP in Sun1-Chat animals to skeletal motor neurons, identified by their distinctive large somata, positive ChAT co-staining, and anatomic localization in the ventral horn (FIG. 9B), as opposed to pericanalicular (interneuron) or in the lateral horn (visceral motor neuron). Next, bulk RNA-seq of Chatpos and putatively motor neuron-depleted flow through (Chatneg) nuclei was performed to identify differentially expressed genes across these two populations. Expression of the cholinergic marker genes Slc5a8 and Chat was enriched in the Chatpos population relative to Chatneg, while excitatory (Slc17a8, Slc17a6) and inhibitory (Gad1, Slc6a5) interneuron, oligodendrocyte (Mbp, Mobp), astrocyte (Gfap, Aqp4), microglia (Cx3cr1, Tmem2), and endothelial (Cldn5) marker genes (Sathyamurthy et al. 2018; Alkaslasi et al. 2021; Rhee et al. 2016; Patel et al. 2021) showed no such enrichment, confirming successful purification of Chat-expressing populations relative to the other major cell types of the spinal cord (FIG. 9C). To further distinguish between Chat-expressing subpopulations, the relative enrichment of skeletal motor neuron (Bc16, Ahnak2, and Aox1), visceral motor neuron (Mme, Gnb4, Nos1), and cholinergic interneuron (Pou6f2, Pax2, Ebf2) marker genes was assessed across the Chatpos and Chatneg populations. Only skeletal motor neuron markers demonstrated significant enrichment in the Chatpos population (q-value <. 01, FC>2. DESeq2) over Chatneg, confirming that skeletal motor neurons comprised the majority of purified nuclei (FIG. 13A).

The relative chromatin accessibility of a Enh has proven to be a useful tool to identify potential functional regulators of gene expression. Having verified the predominantly skeletal motor neuron identity of our Chatpos population, bulk ATAC-seq (Buenrostro et al., 2015) was employed to identify high-confidence chromatin accessible regions (i.e. peaks) in Chatpos and Chatneg nuclei (n=22,403 and 37,365 peaks, respectively) (FIG. 9D-FLP. FIG. 9E-summary of FLP, FIG. 9F—example tracks). The dataset passed several standardized quality control metrics, including nucleosomal ATAC-seq fragment size distribution, high irreproducible discovery rate (IDR), and appropriately higher correlation among than across conditions (FIG. 13C-Fragment distribution, FIG. 13D-ATAC-scq PCA, FIG. 13E ATAC-seq correlation)(Landt et al. 2012).

To facilitate selection of promising candidates for eventual screening from this pool of Enhs, candidate Enhs were ranked by their selective, local chromatin accessibility across the Chatpos/Chatneg comparison. Accessibility was quantified across the union of all peaks in both conditions (n=42.680 peaks) and differential accessibility analysis was performed with the DeSEQ2 algorithm to obtain relative enrichment (Chatpos/Chatneg) for each peak in the unioned set (Love et al., 2014). After filtering differentially accessible peaks within 250 bp of known transcriptional start sites (TSS) to remove accessible sequences of likely tied to promoter activity at actively transcribed genes, peaks of at least 32-fold enrichment were identified at false discovery rate (FDR)-adjusted significance q<. 01 (FIG. 9G). To increase the likelihood of functionality, the most evolutionarily conserved elements were subselected from this population to obtain a final set of high-likelihood motor neuron-enriched ENHs for potential downstream functional evaluation (FIG. 9H).

### Functional GRE Evaluation by AAV Reporter Imaging

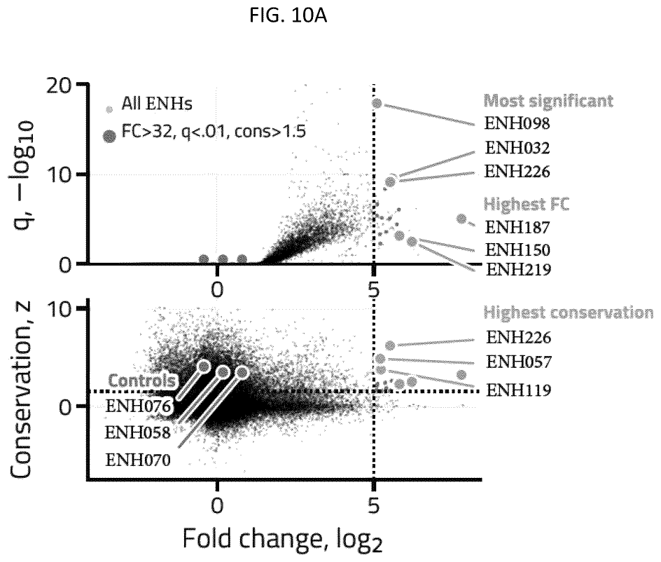

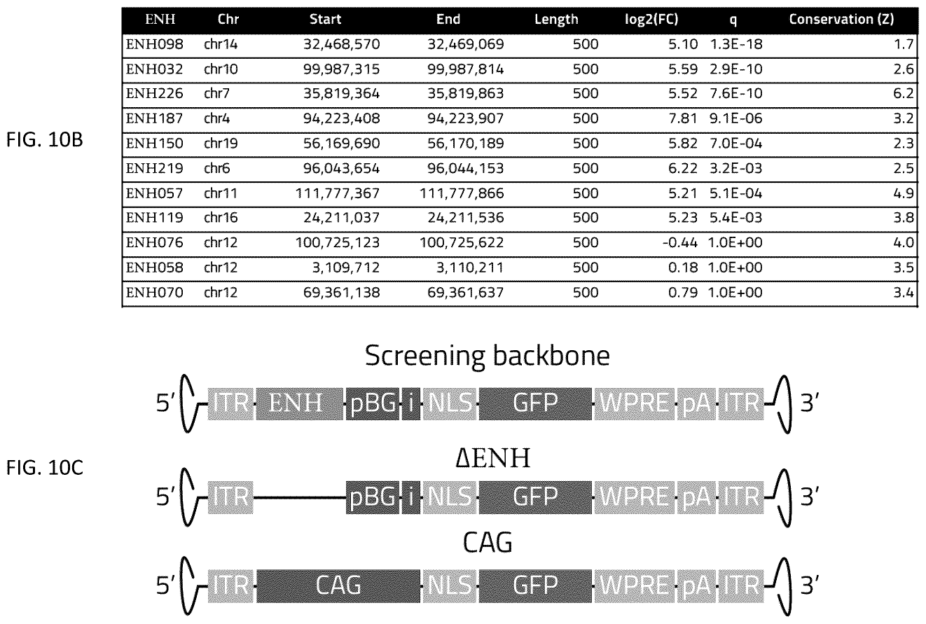

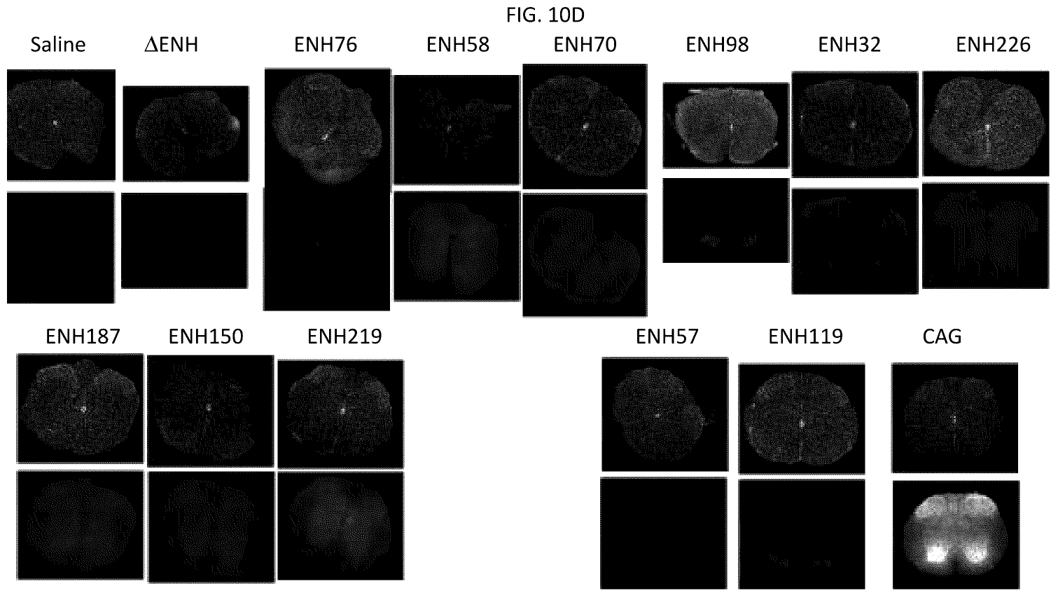

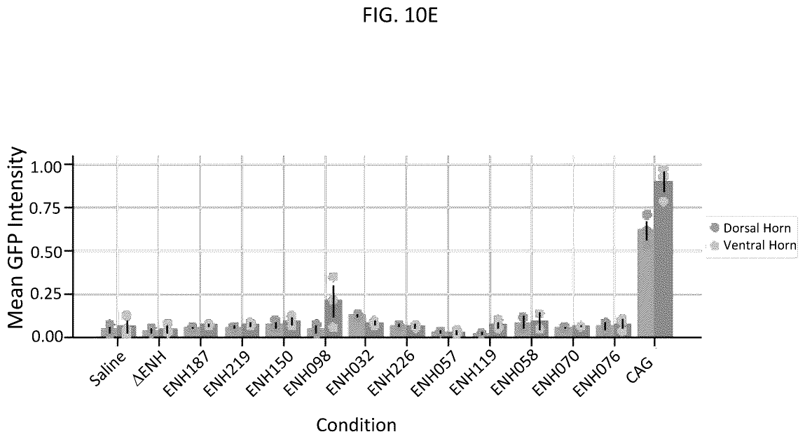

A small number of promising candidates were evaluated for motor neuron-selective expression of a GFP payload via fluorescence microscopy. To this end, three elements exhibiting the greatest magnitude of motor neuron enrichment (Enh187, Enh219, Enh150), the greatest statistical significance for motor neuron enrichment (Enh98, Enh32, Enh226), and the greatest mammalian conservation (Enh226, Enh057, Enh119) were selected from the list of high-confidence motor neuron-enriched Enhs. Inclusion of three additional Enhs that performed poorly across these metrics as negative controls (Enh58, Enh70, Enh76) yielded a total of 11 elements to be cloned for screening (Enh226 appeared in two categories, FIG. 10A, FIG. 10B).

The chosen Enhs were amplified from wild-type mouse genomic DNA and incorporated into a GFP reporter AAV2 vector backbone as described previously: 5′-ITR-ENH-pBG-GFP-barcode-WPRE-polyA-ITR-3′ (Hrvatin et al., 2019) (FIG. 10C-vector map, administration route). Each vector, as well as a negative control construct lacking an introduced Enh (\ENH) and a positive control, enhancerless CAG promoter, was then packaged into the PHP.eB AAV capsid, which efficiently penetrates the mouse blood-brain-barrier and demonstrates neuronal tropism in the spinal cord after intracerebroventricular (ICV) administration (Armbruster et al., 2016; Chan et al., 2017).

To characterize the patterns of GFP expression driven by each of the candidate Enhs, wild-type postnatal day 0 (PO) mice (n=3 per condition) were singly dosed with 1.2×10¹¹ viral genome copies/mL (4 μL) of AAV or saline. Two weeks post-injection, thoracic spinal cords were then dissected, transversely sectioned, and imaged for DAPI and native GFP expression (n=3 sections per animal). Of the 14 evaluated conditions, three (Enh98, Enh119, and CAG) demonstrated increased GFP signal in the ventral horn (FIGS. 10D and 10E) relative to the dorsal horn. Only Enh98 and Enh119 achieved this expression while maintaining skeletal motor neuron-specific expression, with significantly reduced off-target GFP expression in DAPI-stained nuclei of the dorsal horn compared to that of \ENH and other elements (FIG. 10F).

Validation and Further Characterization of GRE-Driven Viral Transgene Expression in the spinal cord

Native GFP fluorescence was measured broadly across the grossly defined ventral and dorsal horns and identified Enh98 and Enh119 as putatively skeletal motor neuron-selective elements by anatomical localization. To more rigorously validate these findings and to quantify the relative specificity and strength of expression conferred by each Enh, transgene expression was measured via immunohistochemistry (IHC) and confocal microscopy in confirmed skeletal motor neurons of the ventral horn, identified by positive co-staining for ChAT and the neuronal marker NeuN. Six conditions were assessed by IHC: saline, the enhancerless (\Enh) and inactive (Enh57) negative controls, the two putative hits (Enh98, Enh119), and the enhancerless CAG positive control construct (Day et al., 2022). To this end, experimental animals (n=3 per condition) were injected with 4 μL (by right 1CV) of either saline or 1.2×10¹¹ viral genome copies/mL of a nuclear GFP-expressing AAV vector driven by CAG or [Enh57, Enh98, Enh119]-pBG regulatory element combinations. As ChAT staining is densest in the soma, a nuclear localization sequence (NLS) was incorporated into all AAV vector constructs to increase GFP overlap with ChAT and facilitate signal quantification. Thoracic and lumbar spinal cord and dorsal root ganglia (DRG), liver, and brain were then dissected two weeks after injection for processing and analysis.

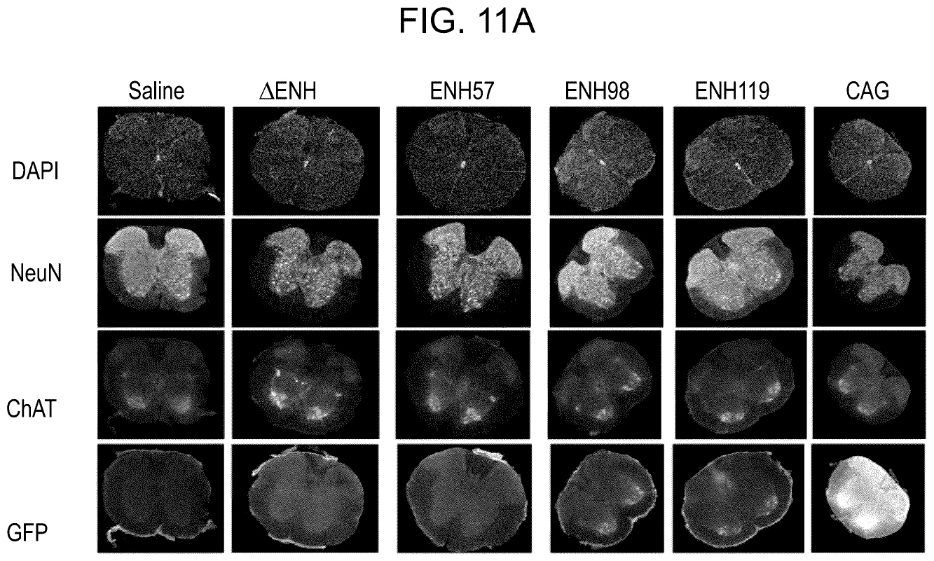

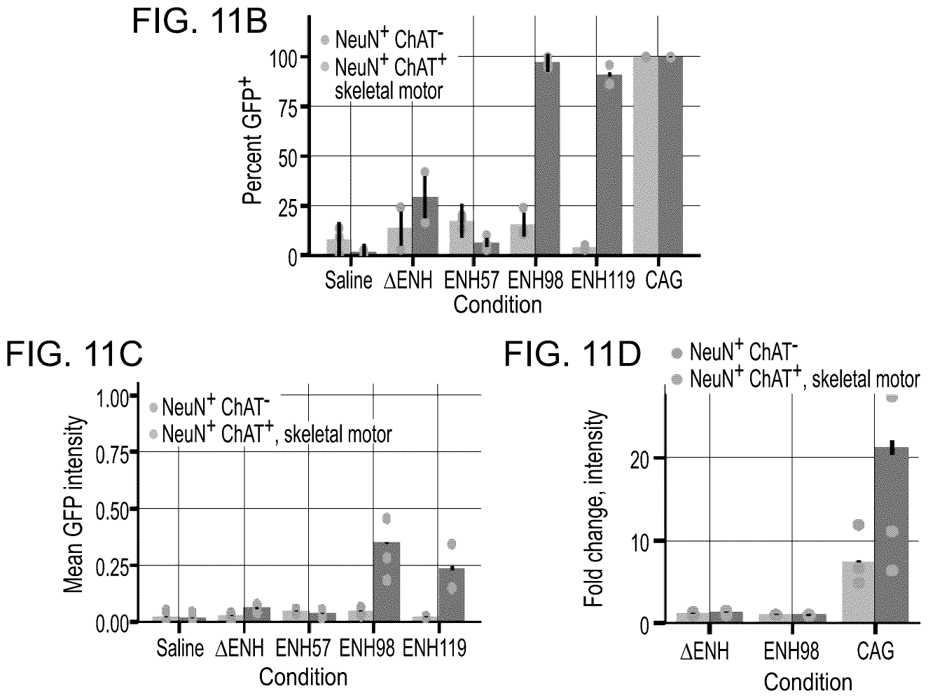

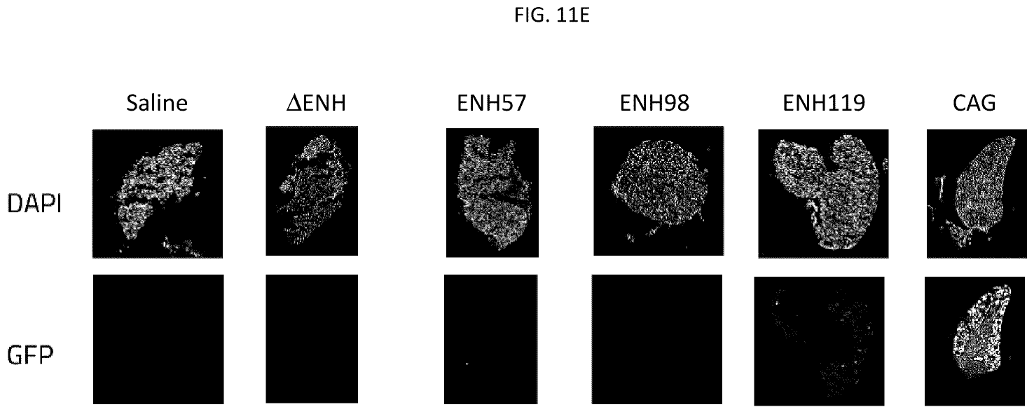

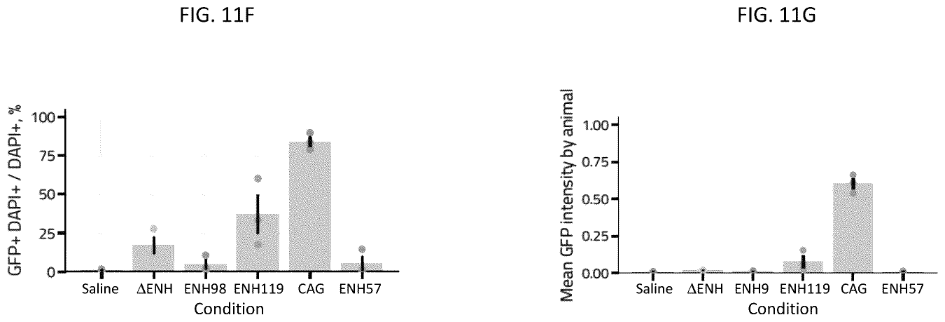

In the spinal cord, the Enh98 and Enh119 constructs drove reporter expression in 97.0% and 91.1% in the on-target NeuN+ChAT+skeletal motor neuron population of the ventral horn, with off-target rates of 15.6 and 3.9% in NeuN+ChAT-neurons (FIG. 11A-representative images, FIG. 11B —motor neuron fraction). The JEnh and Enh57 constructs drove weak reporter expression in the spinal cord (29.2% and 6.2% on-target respectively, 13.5% and 17.2% off-target). By contrast, CAG positivity rate was comparable to Enh98 (100%), but was totally non-specific with an equally high mean off-target rate (100%). These findings reinforce the previous findings, providing more formal quantification of specificity of the Enh98 and Enh119 constructs.

In addition to specificity, the strength of expression is an essential determining factor for therapeutic utility/function. Image intensity was therefore quantified and compared across conditions to determine the relative on-target strength of expression of the tested constructs. On-target signal intensity in the Enh98 and Enh119 conditions (0.33 and 0.24) was significantly greater than off-target populations (0.05 and 0.02), and greater than on-target saline or JEnh (0.03 and 0.09) as well. (FIG. 11C—motor neuron intensity Enhs/Motor neuron intensity CAG). In the previous analysis, image window parameters were selected to emphasize intensity differences across the Enh constructs, which led to truncation of CAG signal. To compare the elements against CAG directly, alternate parameters that captured the full dynamic range of CAG signal intensity were used to evaluate the CAG, Enh98, and JENH conditions (FIG. 11C-CAG windowed images). Using these altered image acquisition settings reveals a relative intensity difference of 21.2-fold increased intensity in CAG compared to Enh98 (FIG. 11D).

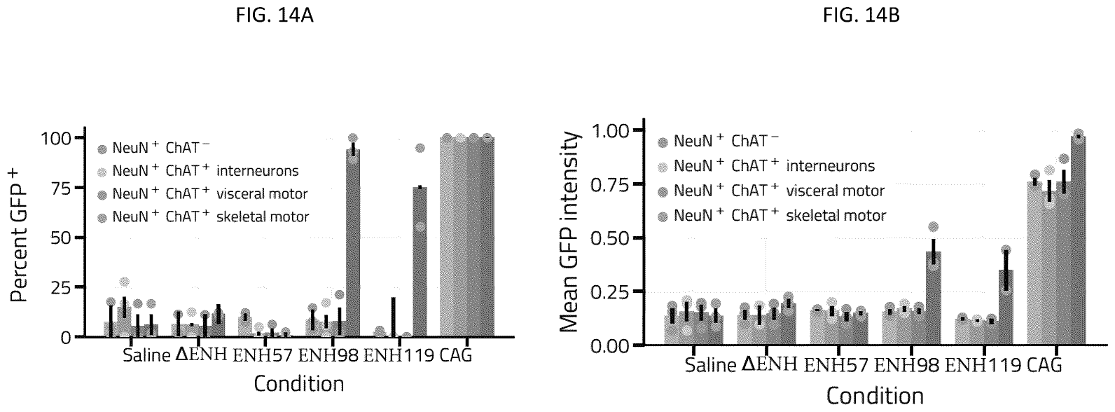

To confirm Enh98 and Enh119 driven expression was restricted to skeletal motor neurons as opposed to all cholinergic neurons of the spinal cord, fluorescence intensity was quantified in subcategorized skeletal motor, visceral motor, and interneuronal cholinergic neurons defined by their anatomic localization to ventral horn, lateral horn, and pericanalicular regions. 89.0% and 75.1% enrichment for Enh98 and Enh119 were observed specifically for ventral Chat+NeuN+neurons compared to Chat+NeuN+neurons outside of the ventral horn (4.2%, 10.3%, 2.2%, and 100.0% for saline, ΔENH, Enh57, and CAG respectively; FIGS. 14A-14B).

### ENH-Driven Viral Transgene Expression Outside the Spinal Cord

In clinical contexts, off-target AAV transduction and payload expression in DRG and liver can introduce safety concerns that impede the therapeutic efficacy of viral vectors. To explore the extent to which Enh98 and Enh119 restrict off-target expression in these clinically relevant tissues, native GFP expression was assessed by immunofluorescence in the dorsal root ganglia (DRG) and livers harvested from these same animals (FIG. 11D). As any reporter expression in these tissues can be considered off-target, the overall positivity rate of neurons in DRG (defined by nuclear size and morphology) and cells in liver (DAPI-defined nuclei) were quantified and compared across conditions.

In the DRG, 84% and 17% of neurons were GFP+ in the CAG and \ENH control conditions, respectively. Enh98 demonstrated low off-target expression in the DRG (4.5%) comparable to the non-functional Enh57 construct (5.2%), but Enh119 failed to attain this same level of specificity with a positivity rate of 37.0%, suggesting potentially distinct mechanisms of transcriptional regulation between Enh98 and Enh 119. Both constructs demonstrate significantly reduced off target DRG expression relative to CAG.

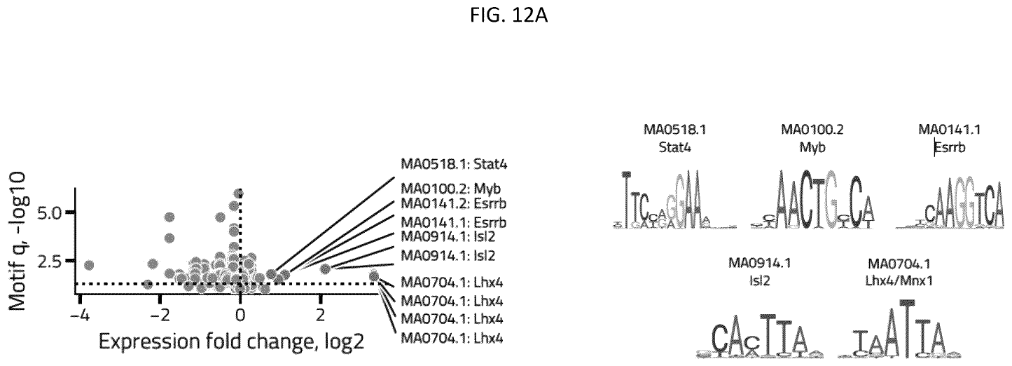

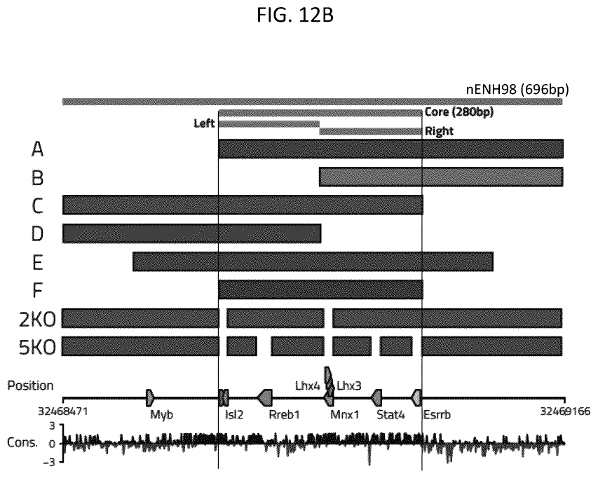

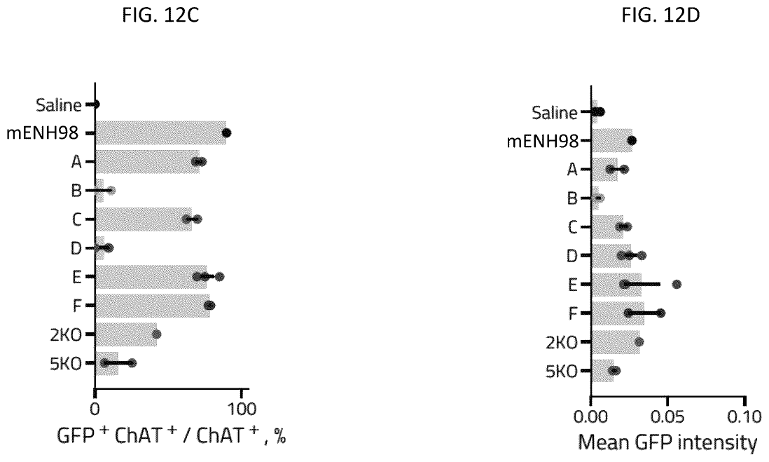

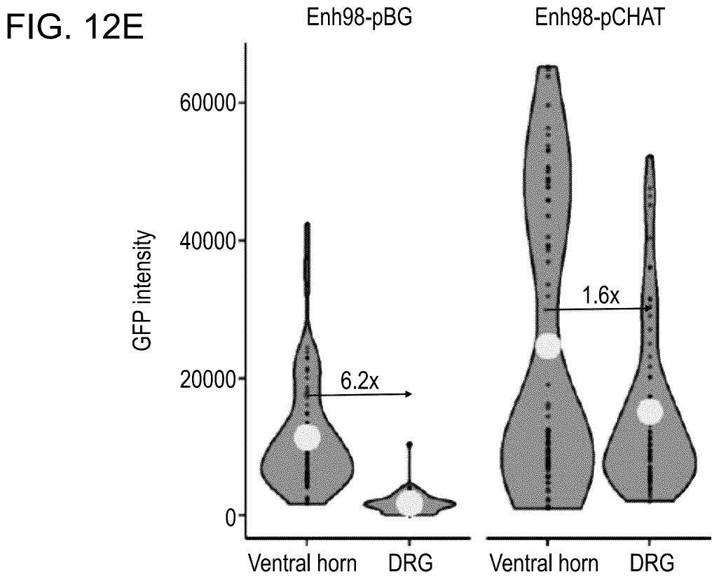

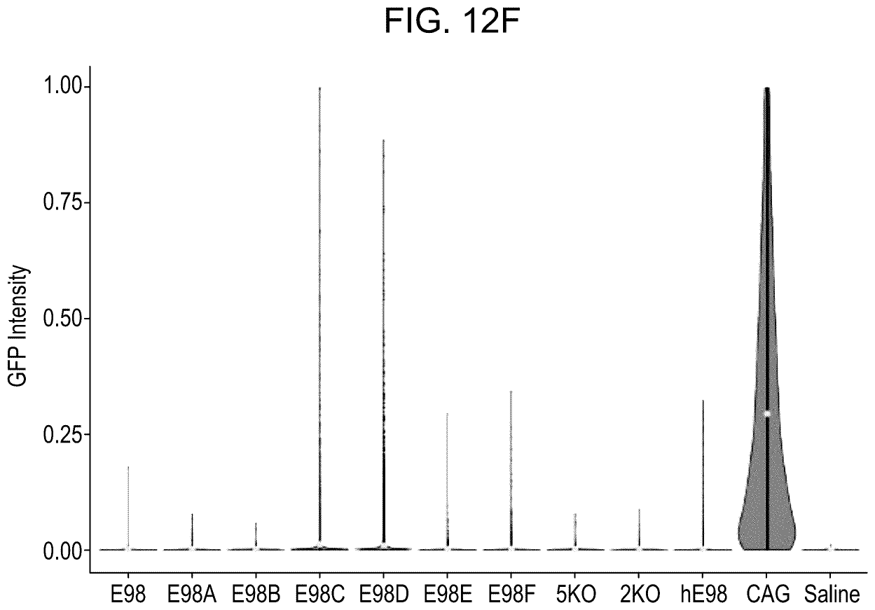

Mechanistic Investigation of Enh98 and Identification of Functional TF Binding Motifs Having confirmed Enh98 as the more motor neuron-selective of the hits from the screen, the key regions conferring this feature to the mouse Enh98 (mEnh98) sequence needed to be identified. All known transcription factor (TF) binding motifs in the JASPAR mouse database present within the full 696 bp sequence of mEnh98 were identified, and an adjusted p-value threshold of 0.05 was used to determine confidence in motif matching. To better distinguish between functional motifs and incidental sequences without transcription factor recruitment, motifs whose associated TFs had non-zero and significant enrichment of expression in the purified Chatpos population (q<. 05) relative to Chatneg were identified, yielding the JASPAR motifs MA0704.1 (Lhx4 and Mnx1), MA0914.1 (Is12), MA0141.1/2 (Esrrb), MA0100.2 (Myb), and MA0518.1 (Stat4) (FIG. 12A). Of note, these TFs all have been demonstrated to either be motor neuron defining during differentiation, or markers of motor neuron subtypes. However, none of them are solely expressed in motor neurons, and the combination of some of these factors play long understood roles in inhibitory interneuron development as well.

All TF binding sites identified this way lay within a core 280-bp region of mEnh98, leading to the hypothesis that this core region was sufficient and necessary for motor neuron-selective Enh98 activity. To test this hypothesis, nine truncated or internally deleted mEnh98 vector constructs were generated: A, B, C, D, E, F. 2KO, and 5KO (FIG. 12B and Table 2). Of particular note, the F construct corresponds to the core region. The 5KO construct comprises precise deletions of four of the five TF binding sites identified in the above core region (Is12, Lhx4/Mnx1/Lhx3, Stat4, Esrrb), as well as deletion of the Rrebl motif, which while barely failing to meet significance thresholds for positive motif identification (FIMO q=. 076 and RNA-seq q=. 078) is implicated as a motor neuron subclass-specific gene in some profiling studies. The 2KO construct lacks only the two binding sites of TFs most associated with motor neuron identity (Is12 and Mnx1).

The full length mEnh98 construct drove GFP expression in 80-90% of ChAT+neurons (FIG. 12C). By comparison, both 5′ and 3′ truncations (constructs D and B) lost GFP expression in almost all ChATpos neurons, demonstrating that both left and right core regions are simultaneously necessary for motor neuron expression. The 2KO Enh98 construct showed a loss in expression in a moderate fraction of ChATpos neurons expressing GFP while the 5KO Enh98 construct resulted in nearly all motor neurons losing reporter expression. These findings suggest that the transcription factor binding sites knocked out in the 2KO and 5KO constructs indeed play an important role in Enh98 function. Furthermore, the identity-defining Is12 and Lhx4/Mnx1/Lhx3 motifs alone do not confer specificity and are not required for motor neuron expression. Intriguingly, the broader and narrower core region constructs (E and F respectively) drove roughly similar patterns of expression as the full length mEnh98 with similarly low off-target expression, implying that the core region is not only necessary but sufficient to drive expression in most motor neurons.

Comparing the GFP intensity in ChAT+ and ChAT-neurons (FIG. 12D), Enh98 has about a 9.5-fold greater expression in the ChAT+neurons than in ChAT-neurons (p<2.2e-16). We see a loss of expression in ChAT+neurons and therefore a reduction in specificity for ChAT+vs. ChAT-neurons for the D, 2KO, and 5KO vector constructs. The core-containing constructs (A, C, E, F) roughly preserved expression strength of full-length Enh98: 9.6-fold (p<2.2e-16) and 25-fold (p<2.2e-16) greater expression in ChAT+neurons than in ChAT-neurons, respectively.

In the DRG, the truncated and mutated constructs retain a similar background-like level of expression to Enh98 (FIG. 12E). In comparison, the CAG construct had a 470-fold greater expression in the DRG (FIG. 12F). The fact that truncating or knocking out key sequences in Enh98 did not amplify expression in non-target tissues such as the DRG suggests that the primary mechanism of how Enh98 achieves motor neuron-specific expression is by selectively amplifying the expression in the motor neurons.

TABLE 2

| Description | |
|---|---|
| SEQ ID NO | Sequence |
| Construct A | AGCACTTAAGTGCAGGCTTTAGTTC |
| (SEQ ID NO: | CAATGACACTCAGGAGCCTCTGGAT |
| 72) | TCCAGCACTGGGGATGGGGGTGGGG |
| | TAGAACGTTCTCAGGCCTCACCAAC |
| | CCCTCCCCTGTGTGCTGCCTTTGGG |
| | AGAGTCCCAAGGCTTCAGCATTACT |
| | TAATTAATTAGGCCTCTACTGCTAC |
| | ATAGGCTCAGATTCAAAAGAACAGA |
| | GTGGCCCACGTCAGCCATTCCCGGA |
| | AAAGTCTGATGGCTGGAAGCCAGAG |
| | GACTATGTGTCTGCCTTGCTGCCCT |
| | TGGCCAGCCCATCCTGAATGCCCAG |
| | ACTCGGACAATGGAGTAGGTACAGA |
| | AGGGTAAAGACAGTGTCTTCTGTAC |
| | CAGTAAGTGGGCCCTGATCTGCTCT |
| | CTACAGCTTCCAGAGAAAGGGCCTG |
| | GCCAATGAGCGGCCTTTTGAGTAGC |
| | AGATACCTCACATGCATTCTGATAG |
| | AAAGCCTGGCCCCAGATCACTGTGA |
| | CTTT |
| Construct B | AGCATTACTTAATTAATTAGGCCTC |
| (SEQ ID NO: | TACTGCTACATAGGCTCAGATTCAA |
| 73) | AAGAACAGAGTGGCCCACGTCAGCC |
| | ATTCCCGGAAAAGTCTGATGGCTGG |
| | AAGCCAGAGGACTATGTGTCTGCCT |
| | TGCTGCCCTTGGCCAGCCCATCCTG |
| | AATGCCCAGACTCGGACAATGGAGT |
| | AGGTACAGAAGGGTAAAGACAGTGT |
| | CTTCTGTACCAGTAAGTGGGCCCTG |
| | ATCTGCTCTCTACAGCTTCCAGAGA |
| | AAGGGCCTGGCCAATGAGCGGCCTT |
| | TTGAGTAGCAGATACCTCACATGCA |
| | TTCTGATAGAAAGCCTGGCCCCAGA |
| | TCACTGTGACTTT |
| Construct C | GAGTCTGGAGAGAGGGTGGGAGCAG |
| (SEQ ID NO: | CCATTCTGCAGCAGTGCCTTCTTGG |
| 74) | GGTCATGGGTCTGTAGGTGCTGCTG |
| | TGGAGGGAGAGATCAGCCTATTCTG |
| | GCTTCATTTCTGAGCTGCAAACTGC |
| | CTGGGTGTCTGGAGAAGCAGGTTGG |
| | CGTGGTGGTTAGCAGTGCGTGGGCG |
| | GGGTTGCCCGCTCTTGATTTATGAT |
| | TTCTTTGTCTCTGTGGAAGCACTTA |
| | AGTGCAGGCTTTAGTTCCAATGACA |
| | CTCAGGAGCCTCTGGATTCCAGCAC |
| | TGGGGATGGGGGTGGGGTAGAACGT |
| | TCTCAGGCCTCACCAACCCCTCCCC |
| | TGTGTGCTGCCTTTGGGAGAGTCCC |
| | AAGGCTTCAGCATTACTTAATTAAT |
| | TAGGCCTCTACTGCTACATAGGCTC |
| | AGATTCAAAAGAACAGAGTGGCCCA |
| | CGTCAGCCATTCCCGGAAAAGTCTG |
| | ATGGCTGGAAGCCAGAGGACTATGT |
| | GTCTGCCTTGCTGCCCTTGGCCAGC |
| | C |
| Construct D | GAGTCTGGAGAGAGGGTGGGAGCAG |
| (SEQ ID NO: | CCATTCTGCAGCAGTGCCTTCTTGG |
| 75) | GGTCATGGGTCTGTAGGTGCTGCTG |
| | TGGAGGGAGAGATCAGCCTATTCTG |
| | GCTTCATTTCTGAGCTGCAAACTGC |
| | CTGGGTGTCTGGAGAAGCAGGTTGG |
| | CGTGGTGGTTAGCAGTGCGTGGGCG |
| | GGGTTGCCCGCTCTTGATTTATGAT |
| | TTCTTTGTCTCTGTGGAAGCACTTA |
| | AGTGCAGGCTTTAGTTCCAATGACA |
| | CTCAGGAGCCTCTGGATTCCAGCAC |
| | TGGGGATGGGGGTGGGGTAGAACGT |
| | TCTCAGGCCTCACCAACCCCTCCCC |
| | TGTGTGCTGCCTTTGGGAGAGTCCC |
| | AAGGCTTC |
| Construct E | GGCTTCATTTCTGAGCTGCAAACTG |
| (SEQ ID NO: | CCTGGGTGTCTGGAGAAGCAGGTTG |
| 76) | GCGTGGTGGTTAGCAGTGCGTGGGC |
| | GGGGTTGCCCGCTCTTGATTTATGA |
| | TTTCTTTGTCTCTGTGGAAGCACTT |
| | AAGTGCAGGCTTTAGTTCCAATGAC |
| | ACTCAGGAGCCTCTGGATTCCAGCA |
| | CTGGGGATGGGGGTGGGGTAGAACG |
| | TTCTCAGGCCTCACCAACCCCTCCC |
| | CTGTGTGCTGCCTTTGGGAGAGTCC |
| | CAAGGCTTCAGCATTACTTAATTAA |
| | TTAGGCCTCTACTGCTACATAGGCT |
| | CAGATTCAAAAGAACAGAGTGGCCC |
| | ACGTCAGCCATTCCCGGAAAAGTCT |
| | GATGGCTGGAAGCCAGAGGACTATG |
| | TGTCTGCCTTGCTGCCCTTGGCCAG |
| | CCCATCCTGAATGCCCAGACTCGGA |
| | CAATGGAGTAGGTACAGAAGGGTAA |
| | AGACAGTGTCTTCTGTACCAGTAAG |
| | TGGGCCCTGATCTGCTCTCTACAGC |
| Construct F | GCACTTAAGTGCAGGCTTTAGTTCC |
| (SEQ ID NO: | AATGACACTCAGGAGCCTCTGGATT |
| 77) | CCAGCACTGGGGATGGGGGTGGGGT |
| | AGAACGTTCTCAGGCCTCACCAACC |
| | CCTCCCCTGTGTGCTGCCTTTGGGA |
| | GAGTCCCAAGGCTTCAGCATTACTT |
| | AATTAATTAGGCCTCTACTGCTACA |
| | TAGGCTCAGATTCAAAAGAACAGAG |
| | TGGCCCACGTCAGCCATTCCCGGAA |
| | AAGTCTGATGGCTGGAAGCCAGAGG |
| | ACTATGTGTCTGCCTTGCTGCCCTT |
| | GGCCA |
| Construct 2KO | GAGTCTGGAGAGAGGGTGGGAGCAG |
| (SEQ ID NO: | CCATTCTGCAGCAGTGCCTTCTTGG |
| 78) | GGTCATGGGTCTGTAGGTGCTGCTG |
| | TGGAGGGAGAGATCAGCCTATTCTG |
| | GCTTCATTTCTGAGCTGCAAACTGC |
| | CTGGGTGTCTGGAGAAGCAGGTTGG |
| | CGTGGTGGTTAGCAGTGCGTGGGCG |
| | GGGTTGCCCGCTCTTGATTTATGAT |
| | TTCTTTGTCTCTGTGGAAAGGCTTT |
| | AGTTCCAATGACACTCAGGAGCCTC |
| | TGGATTCCAGCACTGGGGATGGGGG |
| | TGGGGTAGAACGTTCTCAGGCCTCA |
| | CCAACCCCTCCCCTGTGTGCTGCCT |
| | TTGGGAGAGTCCCAAGGCTTCAGCA |
| | TTGCCTCTACTGCTACATAGGCTCA |
| | GATTCAAAAGAACAGAGTGGCCCAC |
| | GTCAGCCATTCCCGGAAAAGTCTGA |
| | TGGCTGGAAGCCAGAGGACTATGTG |
| | TCTGCCTTGCTGCCCTTGGCCAGCC |
| | CATCCTGAATGCCCAGACTCGGACA |
| | ATGGAGTAGGTACAGAAGGGTAAAG |
| | ACAGTGTCTTCTGTACCAGTAAGTG |
| | GGCCCTGATCTGCTCTCTACAGCTT |
| | CCAGAGAAAGGGCCTGGCCAATGAG |
| | CGGCCTTTTGAGTAGCAGATACCTC |
| | ACATGCATTCTGATAGAAAGCCTGG |
| | CCCCAGATCACTGTGACTTT |
| Construct 5KO | GAGTCTGGAGAGAGGGTGGGAGCAG |
| (SEQ ID NO: | CCATTCTGCAGCAGTGCCTTCTTGG |
| 79) | GGTCATGGGTCTGTAGGTGCTGCTG |
| | TGGAGGGAGAGATCAGCCTATTCTG |
| | GCTTCATTTCTGAGCTGCAAACTGC |
| | CTGGGTGTCTGGAGAAGCAGGTTGG |
| | CGTGGTGGTTAGCAGTGCGTGGGCG |
| | GGGTTGCCCGCTCTTGATTTATGAT |
| | TTCTTTGTCTCTGTGGAAAGGCTTT |
| | AGTTCCAATGACACTCAGGAGCCTC |
| | TGGATTCCAGTAGAACGTTCTCAGG |
| | CCTCACCAACCCCTCCCCTGTGTGC |
| | TGCCTTTGGGAGAGTCCCAAGGCTT |
| | CAGCATTGCCTCTACTGCTACATAG |
| | GCTCAGATTCAAAAGAACAGAGTGG |
| | CCCACGTCAAGTCTGATGGCTGGAA |
| | GCCAGAGGACTATGTGTCTGCCTTG |
| | CGCCCATCCTGAATGCCCAGACTCG |
| | GACAATGGAGTAGGTACAGAAGGGT |
| | AAAGACAGTGTCTTCTGTACCAGTA |
| | AGTGGGCCCTGATCTGCTCTCTACA |
| | GCTTCCAGAGAAAGGGCCTGGCCAA |
| | TGAGCGGCCTTTTGAGTAGCAGATA |
| | CCTCACATGCATTCTGATAGAAAGC |
| | CTGGCCCCAGATCACTGTGACTTT |

### Sequences

### Exemplary Enhancer Sequences

| Description | |
|---|---|
| SEQ ID NO | Sequence |
| Enh98 human | CCAAAGGGATTTGGAGGCCATGCTT |
| (772 bp) | CCAACGAATGATTCATAGTTAGTGT |
| (SEQ ID NO: 1) | CAGGGAGCCAGAAAAAAAGCAAGTG |
| | AGCAAGGTCCTGTCCCTGGGAGCTG |
| | TAGAGAGGAGCCCTGGGGCCCACCC |
| | ACAAAGCAGCACCTGCAGTCTCTTT |
| | CCCTCTCGAAGCCCAGCTATGTTGT |
| | GCACAAAGCAAGTCTGGGCACCGAG |
| | GACAGGCTGGCCAAGGGCAGGCAGG |
| | CAGGCACGTAGTCCTCTGGCTTCCA |
| | GCCACCACACTCACAGGTTTCTGGG |
| | AAAGGCTGACTGGGGCCACTTTGTT |
| | CCTTTGAATCTGAGAATATATGACT |
| | GGGGAAGCCTAAATTAATTAAATGA |
| | TGCTGAGGCCCGCCTGAGCCGGTGC |
| | ACAGGGGATGGGTTATGGAGCCCTG |
| | AGCAAACTGCACCCCTAGCCCCCAG |
| | TGCTGGAATCCAGAGAGGCTCATGA |
| | GCTCGATTGGAACGAAGCCTGTGCT |
| | TAAGTGCTTCCAGAGAGACAAAGAA |
| | ATAATAAATCAGGAGCAGGTGCCCC |
| | ACCCACACACTGCCATCACCAACAC |
| | CAGCCTGCTTCTCCACAGAAATACA |
| | GTGGTTTCACCTCTCTGGAACCAGA |
| | TGTTTCAGGGAAGCAACAAATGGCA |
| | AAGCCCTGGAAATGACATGGCCCCA |
| | CAACCTTCTCAGAAATGAGGCCAGG |
| | CTGGGCTGGCACCTCCATCCACAGC |
| | AGCACCCCACCACCACAACCCACCC |
| | AAGACCTCCAAACACCCCCTAGACC |
| | TCACCCAGGCACTGGTGCAGCA |
| Enh98 human | TGCTGCACCAGTGCCTGGGTGAGGT |
| reverse | CTAGGGGGTGTTTGGAGGTCTTGGG |
| complement | TGGGTTGTGGTGGTGGGGTGCTGCT |
| (772 bp) | GTGGATGGAGGTGCCAGCCCAGCCT |
| (SEQ ID NO: 2) | GGCCTCATTTCTGAGAAGGTTGTGG |
| | GGCCATGTCATTTCCAGGGCTTTGC |
| | CATTTGTTGCTTCCCTGAAACATCT |
| | GGTTCCAGAGAGGTGAAACCACTGT |
| | ATTTCTGTGGAGAAGCAGGCTGGTG |
| | TTGGTGATGGCAGTGTGTGGGTGGG |
| | GCACCTGCTCCTGATTTATTATTTC |
| | TTTGTCTCTCTGGAAGCACTTAAGC |
| | ACAGGCTTCGTTCCAATCGAGCTCA |
| | TGAGCCTCTCTGGATTCCAGCACTG |
| | GGGGCTAGGGGTGCAGTTTGCTCAG |
| | GGCTCCATAACCCATCCCCTGTGCA |
| | CCGGCTCAGGCGGGCCTCAGCATCA |
| | TTTAATTAATTTAGGCTTCCCCAGT |
| | CATATATTCTCAGATTCAAAGGAAC |
| | AAAGTGGCCCCAGTCAGCCTTTCCC |
| | AGAAACCTGTGAGTGTGGTGGCTGG |
| | AAGCCAGAGGACTACGTGCCTGCCT |
| | GCCTGCCCTTGGCCAGCCTGTCCTC |
| | GGTGCCCAGACTTGCTTTGTGCACA |
| | ACATAGCTGGGCTTCGAGAGGGAAA |
| | GAGACTGCAGGTGCTGCTTTGTGGG |
| | TGGGCCCCAGGGCTCCTCTCTACAG |
| | CTCCCAGGGACAGGACCTTGCTCAC |
| | TTGCTTTTTTTCTGGCTCCCTGACA |
| | CTAACTATGAATCATTCGTTGGAAG |
| | CATGGCCTCCAAATCCCTTTGG |
| Enh98 human | GCTGTAGAGAGGAGCCCTGGGGCCC |
| (576 bp) | ACCCACAAAGCAGCACCTGCAGTCT |
| (SEQ ID NO: 3) | CTTTCCCTCTCGAAGCCCAGCTATG |
| | TTGTGCACAAAGCAAGTCTGGGCAC |
| | CGAGGACAGGCTGGCCAAGGGCAGG |
| | CAGGCAGGCACGTAGTCCTCTGGCT |
| | TCCAGCCACCACACTCACAGGTTTC |
| | TGGGAAAGGCTGACTGGGGCCACTT |
| | TGTTCCTTTGAATCTGAGAATATAT |
| | GACTGGGGAAGCCTAAATTAATTAA |
| | ATGATGCTGAGGCCCGCCTGAGCCG |
| | GTGCACAGGGGATGGGTTATGGAGC |
| | CCTGAGCAAACTGCACCCCTAGCCC |
| | CCAGTGCTGGAATCCAGAGAGGCTC |
| | ATGAGCTCGATTGGAACGAAGCCTG |
| | TGCTTAAGTGCTTCCAGAGAGACAA |
| | AGAAATAATAAATCAGGAGCAGGTG |
| | CCCCACCCACACACTGCCATCACCA |
| | ACACCAGCCTGCTTCTCCACAGAAA |
| | TACAGTGGTTTCACCTCTCTGGAAC |
| | CAGATGTTTCAGGGAAGCAACAAAT |
| | GGCAAAGCCCTGGAAATGACATGGC |
| | CCCACAACCTTCTCAGAAATGAGGC |
| | C |
| Enh98 human | GGCCTCATTTCTGAGAAGGTTGTGG |
| reverse | GGCCATGTCATTTCCAGGGCTTTGC |
| complement | CATTTGTTGCTTCCCTGAAACATCT |
| (576 bp) | GGTTCCAGAGAGGTGAAACCACTGT |
| (SEQ ID NO: 4) | ATTTCTGTGGAGAAGCAGGCTGGTG |
| | TTGGTGATGGCAGTGTGTGGGTGGG |
| | GCACCTGCTCCTGATTTATTATTTC |
| | TTTGTCTCTCTGGAAGCACTTAAGC |
| | ACAGGCTTCGTTCCAATCGAGCTCA |
| | TGAGCCTCTCTGGATTCCAGCACTG |
| | GGGGCTAGGGGTGCAGTTTGCTCAG |
| | GGCTCCATAACCCATCCCCTGTGCA |
| | CCGGCTCAGGCGGGCCTCAGCATCA |
| | TTTAATTAATTTAGGCTTCCCCAGT |
| | CATATATTCTCAGATTCAAAGGAAC |
| | AAAGTGGCCCCAGTCAGCCTTTCCC |
| | AGAAACCTGTGAGTGTGGTGGCTGG |
| | AAGCCAGAGGACTACGTGCCTGCCT |
| | GCCTGCCCTTGGCCAGCCTGTCCTC |
| | GGTGCCCAGACTTGCTTTGTGCACA |
| | ACATAGCTGGGCTTCGAGAGGGAAA |
| | GAGACTGCAGGTGCTGCTTTGTGGG |
| | TGGGCCCCAGGGCTCCTCTCTACAG |
| | C |
| Enh98 human | GCCAAGGGCAGGCAGGCAGGCACGT |
| core | AGTCCTCTGGCTTCCAGCCACCACA |
| (SEQ ID NO: 5) | CTCACAGGTTTCTGGGAAAGGCTGA |
| | CTGGGGCCACTTTGTTCCTTTGAAT |
| | CTGAGAATATATGACTGGGGAAGCC |
| | TAAATTAATTAAATGATGCTGAGGC |
| | CCGCCTGAGCCGGTGCACAGGGGAT |
| | GGGTTATGGAGCCCTGAGCAAACTG |
| | CACCCCTAGCCCCCAGTGCTGGAAT |
| | CCAGAGAGGCTCATGAGCTCGATTG |
| | GAACGAAGCCTGTGCTTAAGTGCTT |
| | CCAGAGAGACAAAGAAATAATAAAT |
| | CA |
| Enh98 human | TGATTTATTATTTCTTTGTCTCTCT |
| reverse | GGAAGCACTTAAGCACAGGCTTCGT |
| complement | TCCAATCGAGCTCATGAGCCTCTCT |
| core | GGATTCCAGCACTGGGGGCTAGGGG |
| (SEQ ID NO: 6) | TGCAGTTTGCTCAGGGCTCCATAAC |
| | CCATCCCCTGTGCACCGGCTCAGGC |
| | GGGCCTCAGCATCATTTAATTAATT |
| | TAGGCTTCCCCAGTCATATATTCTC |
| | AGATTCAAAGGAACAAAGTGGCCCC |
| | AGTCAGCCTTTCCCAGAAACCTGTG |
| | AGTGTGGTGGCTGGAAGCCAGAGGA |
| | CTACGTGCCTGCCTGCCTGCCCTTG |
| | GC |
| Enh98 mouse | ACCGTGGCTTAGTNTGATAAACCAA |
| (long) | AACCTGCTCCATTATGAATCAGTGC |
| (SEQ ID NO: 7) | TGTGGGGAGTGGGTAGAGAGTGTGA |
| | AGTTCTGGGGTGGGGGAGTCTGGAG |
| | AGAGGGTGGGAGCAGCCATTCTGCA |
| | GCAGTGCCTTCTTGGGGTCATGGGT |
| | CTGTAGGTGCTGCTGTGGAGGGAGA |
| | GATCAGCCTATTCTGGCTTCATTTC |
| | TGAGCTGCAAACTGCCTGGGTGTCT |
| | GGAGAAGCAGGTTGGCGTGGTGGTT |
| | AGCAGTGCGTGGGCGGGGTTGCCCG |
| | CTCTTGATTTATGATTTCTTTGTCT |
| | CTGTGGAAGCACTTAAGTGCAGGCT |
| | TTAGTTCCAATGACACTCAGGAGCC |
| | TCTGGATTCCAGCACTGGGGATGGG |
| | GGTGGGGTAGAACGTTCTCAGGCCT |
| | CACCAACCCCTCCCCTGTGTGCTGC |
| | CTTTGGGAGAGTCCCAAGGCTTCAG |
| | CATTACTTAATTAATTAGGCCTCTA |
| | CTGCTACATAGGCTCAGATTCAAAA |
| | GAACAGAGTGGCCCACGTCAGCCAT |
| | TCCCGGAAAAGTCTGATGGCTGGAA |
| | GCCAGAGGACTATGTGTCTGCCTTG |
| | CTGCCCTTGGCCAGCCCATCCTGAA |
| | TGCCCAGACTCGGACAATGGAGTAG |
| | GTACAGAAGGGTAAAGACAGTGTCT |
| | TCTGTACCAGTAAGTGGGCCCTGAT |
| | CTGCTCTCTACAGCTTCCAGAGAAA |
| | GGGCCTGGCCAATGAGCGGCCTTTT |
| | GAGTAGCAGATACCTCACATGCATT |
| | CTGATAGAAAGCCTGGCCCCAGATC |
| | ACTGTGACTTTAGCCCTCAGGTTTC |
| | TTTTGCACTTCAATTCAATGACTTC |
| | TTGAGGTTCATTTCCCTCTCCAAGA |
| | TTTGCCACAGACCAGTGGTTCTCAA |
| Enh98 mouse | TTGAGAACCACTGGTCTGTGGCAAA |
| reverse | TCTTGGAGAGGGAAATGAACCTCAA |
| complement | GAAGTCATTGAATTGAAGTGCAAAA |
| (long) | GAAACCTGAGGGCTAAAGTCACAGT |
| (SEQ ID NO: 8) | GATCTGGGGCCAGGCTTTCTATCAG |
| | AATGCATGTGAGGTATCTGCTACTC |
| | AAAAGGCCGCTCATTGGCCAGGCCC |
| | TTTCTCTGGAAGCTGTAGAGAGCAG |
| | ATCAGGGCCCACTTACTGGTACAGA |
| | AGACACTGTCTTTACCCTTCTGTAC |
| | CTACTCCATTGTCCGAGTCTGGGCA |
| | TTCAGGATGGGCTGGCCAAGGGCAG |
| | CAAGGCAGACACATAGTCCTCTGGC |
| | TTCCAGCCATCAGACTTTTCCGGGA |
| | ATGGCTGACGTGGGCCACTCTGTTC |
| | TTTTGAATCTGAGCCTATGTAGCAG |
| | TAGAGGCCTAATTAATTAAGTAATG |
| | CTGAAGCCTTGGGACTCTCCCAAAG |
| | GCAGCACACAGGGGAGGGGTTGGTG |
| | AGGCCTGAGAACGTTCTACCCCACC |
| | CCCATCCCCAGTGCTGGAATCCAGA |
| | GGCTCCTGAGTGTCATTGGAACTAA |
| | AGCCTGCACTTAAGTGCTTCCACAG |
| | AGACAAAGAAATCATAAATCAAGAG |
| | CGGGCAACCCCGCCCACGCACTGCT |
| | AACCACCACGCCAACCTGCTTCTCC |
| | AGACACCCAGGCAGTTTGCAGCTCA |
| | GAAATGAAGCCAGAATAGGCTGATC |
| | TCTCCCTCCACAGCAGCACCTACAG |
| | ACCCATGACCCCAAGAAGGCACTGC |
| | TGCAGAATGGCTGCTCCCACCCTCT |
| | CTCCAGACTCCCCCACCCCAGAACT |
| | TCACACTCTCTACCCACTCCCCACA |
| | GCACTGATTCATAATGGAGCAGGTT |
| | TTGGTTTATCANACTAAGCCACGGT |
| Enh98 mouse | GGCTTCATTTCTGAGCTGCAAACTG |
| (500 bp) | CCTGGGTGTCTGGAGAAGCAGGTTG |
| (SEQ ID NO: 9) | GCGTGGTGGTTAGCAGTGCGTGGGC |
| | GGGGTTGCCCGCTCTTGATTTATGA |
| | TTTCTTTGTCTCTGTGGAAGCACTT |
| | AAGTGCAGGCTTTAGTTCCAATGAC |
| | ACTCAGGAGCCTCTGGATTCCAGCA |
| | CTGGGGATGGGGGTGGGGTAGAACG |
| | TTCTCAGGCCTCACCAACCCCTCCC |
| | CTGTGTGCTGCCTTTGGGAGAGTCC |
| | CAAGGCTTCAGCATTACTTAATTAA |
| | TTAGGCCTCTACTGCTACATAGGCT |
| | CAGATTCAAAAGAACAGAGTGGCCC |
| | ACGTCAGCCATTCCCGGAAAAGTCT |
| | GATGGCTGGAAGCCAGAGGACTATG |
| | TGTCTGCCTTGCTGCCCTTGGCCAG |
| | CCCATCCTGAATGCCCAGACTCGGA |
| | CAATGGAGTAGGTACAGAAGGGTAA |
| | AGACAGTGTCTTCTGTACCAGTAAG |
| | TGGGCCCTGATCTGCTCTCTACAGC |
| Enh98 mouse | GCTGTAGAGAGCAGATCAGGGCCCA |
| reverse | CTTACTGGTACAGAAGACACTGTCT |
| complement | TTACCCTTCTGTACCTACTCCATTG |
| (500 bp) | TCCGAGTCTGGGCATTCAGGATGGG |
| (SEQ ID NO: 10) | CTGGCCAAGGGCAGCAAGGCAGACA |
| | CATAGTCCTCTGGCTTCCAGCCATC |
| | AGACTTTTCCGGGAATGGCTGACGT |
| | GGGCCACTCTGTTCTTTTGAATCTG |
| | AGCCTATGTAGCAGTAGAGGCCTAA |
| | TTAATTAAGTAATGCTGAAGCCTTG |
| | GGACTCTCCCAAAGGCAGCACACAG |
| | GGGAGGGGTTGGTGAGGCCTGAGAA |
| | CGTTCTACCCCACCCCCATCCCCAG |
| | TGCTGGAATCCAGAGGCTCCTGAGT |
| | GTCATTGGAACTAAAGCCTGCACTT |
| | AAGTGCTTCCACAGAGACAAAGAAA |
| | TCATAAATCAAGAGCGGGCAACCCC |
| | GCCCACGCACTGCTAACCACCACGC |
| | CAACCTGCTTCTCCAGACACCCAGG |
| | CAGTTTGCAGCTCAGAAATGAAGCC |
| Enh98 mouse | GCACTTAAGTGCAGGCTTTAGTTCC |
| core | AATGACACTCAGGAGCCTCTGGATT |
| (SEQ ID NO: 11) | CCAGCACTGGGGATGGGGGTGGGGT |
| | AGAACGTTCTCAGGCCTCACCAACC |
| | CCTCCCCTGTGTGCTGCCTTTGGGA |
| | GAGTCCCAAGGCTTCAGCATTACTT |
| | AATTAATTAGGCCTCTACTGCTACA |
| | TAGGCTCAGATTCAAAAGAACAGAG |
| | TGGCCCACGTCAGCCATTCCCGGAA |
| | AAGTCTGATGGCTGGAAGCCAGAGG |
| | ACTATGTGTCTGCCTTGCTGCCCTT |
| | GGCCA |
| Enh98 mouse | TGGCCAAGGGCAGCAAGGCAGACAC |
| reverse | ATAGTCCTCTGGCTTCCAGCCATCA |
| complement | GACTTTTCCGGGAATGGCTGACGTG |
| core | GGCCACTCTGTTCTTTTGAATCTGA |
| (SEQ ID NO: 12) | GCCTATGTAGCAGTAGAGGCCTAAT |
| | TAATTAAGTAATGCTGAAGCCTTGG |
| | GACTCTCCCAAAGGCAGCACACAGG |
| | GGAGGGGTTGGTGAGGCCTGAGAAC |
| | GTTCTACCCCACCCCCATCCCCAGT |
| | GCTGGAATCCAGAGGCTCCTGAGTG |
| | TCATTGGAACTAAAGCCTGCACTTA |
| | AGTGC |
| Mouse Enh57 | TTTCTTAATAACTGCTATTTTGAAA |
| (mEnh57) | TGTATCATTATCATAACTCCAGTGT |
| (SEQ ID NO: 13) | AGAAGTGGTGTCCAGATTTCTGCTA |
| | TGTTGCTAATTTTTGATATGAGACA |
| | TTCTTATTAGAGTTGAGGGAATGTG |
| | CTTGTATCACTTAGGTGCACACACC |
| | AGAAGCCAGTGCAGGCTCAAGGTGA |
| | ACACAGAGACTCGTGGTACCCCAAA |
| | TGGCTCTCTATCTGACTTCAGCTCT |
| | CTTCCACTTCTTCAACTAGAAATAT |
| | TGCTGAGGGCTTGTTAAACACACAA |
| | AAGCCATGGCTTTTGACCATCTTGC |
| | AAGCAAAAGAAACACCATTTTAAAC |
| | TCCTTTGAAAACGTTCTCTTCTTTC |
| | ACATTAAGAGGCTGCCACACGAACA |
| | GAACGTGCCATAAATAATGTGTGCT |
| | AACATTTTCCAAAAACTGGACATCA |
| | ATTAACGTTAATTTATGAGAACACT |
| | TCTTGAGAGGAGCACAGTTCAGACT |
| | CATAACTACTGAAAAGGCTCATTAA |
| | TAGAAATGTGTAGGGAGAGGGTTTT |
| | TTTCTTCTTCTAAAGGGAACATTAA |
| | AGTAAACACATATCATTGCAAGGAA |
| | GGCTCATGATTTATTGCAAACTCAG |
| | TGGAAAGGAGACTTTACGCTGTGTT |
| | TCCAGGGTGAATTTTGAGCAAAGGA |
| | ATCAAGCAAACAAAATGAAATGAGG |
| | ATATTCTCTTAGGAAAGGCATCCTG |
| | TGACAACCCAGACAAATGATAGCTA |
| | ATACTTATATAATAAGTACTACATA |
| | TCAGGTCAGGCACTATGCCAACATG |
| | ATCTTGTGTGTGTCTCACCAAGAAC |
| | ACTGCCAGGGAAATTTGTTTTGCTG |
| | CCATATACAAAGTTAAAAATCAAGC |
| | CCCC |
| Mouse Enh57 | GGGGGCTTGATTTTTAACTTTGTAT |
| (mEnh57) | ATGGCAGCAAAACAAATTTCCCTGG |
| sequence | CAGTGTTCTTGGTGAGACACACACA |
| reverse | AGATCATGTTGGCATAGTGCCTGAC |
| complement | CTGATATGTAGTACTTATTATATAA |
| (SEQ ID NO: 14) | GTATTAGCTATCATTTGTCTGGGTT |
| | GTCACAGGATGCCTTTCCTAAGAGA |
| | ATATCCTCATTTCATTTTGTTTGCT |
| | TGATTCCTTTGCTCAAAATTCACCC |
| | TGGAAACACAGCGTAAAGTCTCCTT |
| | TCCACTGAGTTTGCAATAAATCATG |
| | AGCCTTCCTTGCAATGATATGTGTT |
| | TACTTTAATGTTCCCTTTAGAAGAA |
| | GAAAAAAACCCTCTCCCTACACATT |
| | TCTATTAATGAGCCTTTTCAGTAGT |
| | TATGAGTCTGAACTGTGCTCCTCTC |
| | AAGAAGTGTTCTCATAAATTAACGT |
| | TAATTGATGTCCAGTTTTTGGAAAA |
| | TGTTAGCACACATTATTTATGGCAC |
| | GTTCTGTTCGTGTGGCAGCCTCTTA |
| | ATGTGAAAGAAGAGAACGTTTTCAA |
| | AGGAGTTTAAAATGGTGTTTCTTTT |
| | GCTTGCAAGATGGTCAAAAGCCATG |
| | GCTTTTGTGTGTTTAACAAGCCCTC |
| | AGCAATATTTCTAGTTGAAGAAGTG |
| | GAAGAGAGCTGAAGTCAGATAGAGA |
| | GCCATTTGGGGTACCACGAGTCTCT |
| | GTGTTCACCTTGAGCCTGCACTGGC |
| | TTCTGGTGTGTGCACCTAAGTGATA |
| | CAAGCACATTCCCTCAACTCTAATA |
| | AGAATGTCTCATATCAAAAATTAGC |
| | AACATAGCAGAAATCTGGACACCAC |
| | TTCTACACTGGAGTTATGATAATGA |
| | TACATTTCAAAATAGCAGTTATTAA |
| | GAAA |
| Enh119 mouse | TTGCTACCTACTAACACTTCATAAT |
| (SEQ ID NO: 60) | CTTACCAAGATAGGAAAAGGAACGG |
| | GACCTTATAATAGAATGGAACATAA |
| | TGACACACTCATCCCAGAGTCTCAC |
| | TCAGGATCTGCATTTGGGACAATCA |
| | AAGGTCCCCTGGCCCTTGTTCAGTC |
| | ACTTAATGGAGAAGACTCCAAAGAC |
| | AGAATGCCACTGGTGTCCTTCCAAT |
| | TATAGAATCATCTGATTAGAATTAC |
| | AGTAAATGCATAGCTCAGTTTGCAT |
| | TGTCCTGATGTGAACTATGAGGCCT |
| | CTCTCCTGGAGCATCTGAGGGTACT |
| | GTACTCTGGAAGTGTACCGCCACGT |
| | CACAGTAGGGTCCTTGTGCCAGGAC |
| | CAGCTTAGAAACGGGACAGAAACAA |
| | GTTAGGACACTCCATTTCTGTGGAC |
| | CTTAGAGCCCAAGGTACCAGAGCTA |
| | GATGGTTTGTTTTTTTTGGGTTTTG |
| | GGGTGTTTTTTTTTTTGTTTGTTTG |
| | TTTGTTTTTTTAGATTAATGCTTAG |
| | AAGAAAAACTGAAGCCTCACAAACT |
| | TGAGATAGTAGCATAGTTCAGACGT |
| | GTAGTAGGAAGGGTTGACTTTGGGA |
| | TAATTTTAGAATTAGTTATTCTAAG |
| | AGGTGGTCCATAGAACACAAGTGTG |
| | TAGCATCTCGGTCCATGATGAAACT |
| | GGTCCTATCTGGCTAT |
| Enh119 mouse | TAACACTTCATAATCTTACCAAGAT |
| chr16: | AGGAAAAGGAACGGGACCTTATAAT |
| 24210965-24211221 | AGAATGGAACATAATGACACACTCA |
| (SEQ ID NO: 61) | TCCCAGAGTCTCACTCAGGATCTGC |
| | ATTTGGGACAATCAAAGGTCCCCTG |
| | GCCCTTGTTCAGTCACTTAATGGAG |
| | AAGACTCCAAAGACAGAATGCCACT |
| | GGTGTCCTTCCAATTATAGAATCAT |
| | CTGATTAGAATTACAGTAAATGCAT |
| | AGCTCAGTTTGCATTGTCCTGATGT |
| | GAACTAT |
| Enh119 mouse | TAATGCTTAGAAGAAAAACTGAAGC |
| chr16: | CTCACAAACTTGAGATAGTAGCATA |
| 24211444-24211600 | GTTCAGACGTGTAGTAGGAAGGGTT |
| (SEQ ID NO: 62) | GACTTTGGGATAATTTTAGAATTAG |
| | TTATTCTAAGAGGTGGTCCATAGAA |
| | CACAAGTGTGTAGCATCTCGGTCCA |
| | TGATGAA |
| Enh119 mouse | ATAGCCAGATAGGACCAGTTTCATC |
| Reverse complement | ATGGACCGAGATGCTACACACTTGT |
| (SEQ ID NO: 63) | GTTCTATGGACCACCTCTTAGAATA |
| | ACTAATTCTAAAATTATCCCAAAGT |
| | CAACCCTTCCTACTACACGTCTGAA |
| | CTATGCTACTATCTCAAGTTTGTGA |
| | GGCTTCAGTTTTTCTTCTAAGCATT |
| | AATCTAAAAAAACAAACAAACAAAC |
| | AAAAAAAAAAACACCCCAAAACCCA |
| | AAAAAAACAAACCATCTAGCTCTGG |
| | TACCTTGGGCTCTAAGGTCCACAGA |
| | AATGGAGTGTCCTAACTTGTTTCTG |
| | TCCCGTTTCTAAGCTGGTCCTGGCA |
| | CAAGGACCCTACTGTGACGTGGCGG |
| | TACACTTCCAGAGTACAGTACCCTC |
| | AGATGCTCCAGGAGAGAGGCCTCAT |
| | AGTTCACATCAGGACAATGCAAACT |
| | GAGCTATGCATTTACTGTAATTCTA |
| | ATCAGATGATTCTATAATTGGAAGG |
| | ACACCAGTGGCATTCTGTCTTTGGA |
| | GTCTTCTCCATTAAGTGACTGAACA |
| | AGGGCCAGGGGACCTTTGATTGTCC |
| | CAAATGCAGATCCTGAGTGAGACTC |
| | TGGGATGAGTGTGTCATTATGTTCC |
| | ATTCTATTATAAGGTCCCGTTCCTT |
| | TTCCTATCTTGGTAAGATTATGAAG |
| | TGTTAGTAGGTAGCAA |
| Enh119 mouse | ATAGTTCACATCAGGACAATGCAAA |
| chr16: | CTGAGCTATGCATTTACTGTAATTC |
| 24210965-24211221 | TAATCAGATGATTCTATAATTGGAA |
| Reverse complement | GGACACCAGTGGCATTCTGTCTTTG |
| (SEQ ID NO: 64) | GAGTCTTCTCCATTAAGTGACTGAA |
| | CAAGGGCCAGGGGACCTTTGATTGT |
| | CCCAAATGCAGATCCTGAGTGAGAC |
| | TCTGGGATGAGTGTGTCATTATGTT |
| | CCATTCTATTATAAGGTCCCGTTCC |
| | TTTTCCTATCTTGGTAAGATTATGA |
| | AGTGTTA |
| Enh119 mouse | TTCATCATGGACCGAGATGCTACAC |
| chr16: | ACTTGTGTTCTATGGACCACCTCTT |
| 24211444-24211600 | AGAATAACTAATTCTAAAATTATCC |
| Reverse complement | CAAAGTCAACCCTTCCTACTACACG |
| (SEQ ID NO: 65) | TCTGAACTATGCTACTATCTCAAGT |
| | TTGTGAGGCTTCAGTTTTTCTTCTA |
| | AGCATTA |
| Enh119 human | GATTAAGAAATTCAGGTTATTTTTC |
| chr3: | TTATTACTTTAGTCAACAATTATCA |
| 187981138-187982252 | TATATGATTATAATCTAGACTTGGA |
| (SEQ ID NO: 66) | AATATTTACCTAAAATATTCAGTCA |
| | CTATATTCAAGCATACATACACACA |
| | CTCCCCACCACAAATACACACAAAC |
| | ACTTTGCTCATTTCATTTGTTTTTC |
| | ATTGTTAGGAGAGCAGTTGGTCAGA |
| | ATTTATTGAAAGTACGGGTGAAATG |
| | ACTGCTACACACATTTTATGATCTT |
| | ACCAAGAAAAAATTAAGAACTTGAT |
| | CCTGTTATAGAATGGAACATAGTAT |
| | CCAGATCTCAGAGTCTCTATCACGA |
| | TCTGCGTTTGGGACAAGTAAAGGTC |
| | CCCTGGCCCTTGTTCAATTGCTTAA |
| | TGGAAAAGACTCCAAAGACAGAATG |
| | CCACTGGTGTTCTTCCAATTATAGA |
| | ATCATCTGATTAGAATTACAGTAAA |
| | TGCATAGCTCAGTTTGCATTGTCCT |
| | GAGGTGAACCGCAAACCAAGCTGCT |
| | CTGGTTGGAGCATCGGAGGGTACTG |
| | AATGCTGAAAGCCCACTACCTCATC |
| | TCAGCGGGGCACTCATACAAGGGCT |
| | AACTTGGAAAGGGACAGATACCAGT |
| | TAGGATATTCCACTTCTGGGGACCC |
| | TGGAGCTCTGGGGGCCAGAGCTAGA |
| | TGGATTATTTAATTAATGTTTAGTA |
| | GAAATAGTCAAATAGCACACACTCT |
| | AGACATTAAGCCAATCCAGACCTTT |
| | GGACTGAATTGGAGGGAAGATTTGT |
| | CTTCGTGACTATTTTAGAATTAATT |
| | ATTCTAGTTTATTTCCAGCCTGTCA |
| | GCATTGAGTCTTGAGAGGTGGTCTG |
| | TAAAACACAAGTTTTTCCAATCATG |
| | GGGTTGTGTTGTGGTCCCATGGGTT |
| | TTCTTGCTCTGTCTGGCCATAGAAG |
| | AACAGATCAGGAATCCTACAGAAGA |
| | ATCCCAAATCCATTCCTCCCCTTCT |
| | ACTTATTTCAGTTACAGCTAGAGGG |
| | TTGGGACTCATTCGTGTGTTAGAAC |
| | CAAACCTGACTATTGTGTTATTATT |
| | GCTTCTAATTTAACTACCAGACTGT |
| | TAAACATTACTGCCCCAAGCTCAGC |
| | CAGGGGTGGGCACTGCACTTTGAAG |
| | CCACCAAGTCAATAG |
| Enh119 human | AACATAGTATCCAGATCTCAGAGTC |
| chr3: | TCTATCACGATCTGCGTTTGGGACA |
| 187981428-187981627 | AGTAAAGGTCCCCTGGCCCTTGTTC |
| (SEQ ID NO: 67) | AATTGCTTAATGGAAAAGACTCCAA |
| | AGACAGAATGCCACTGGTGTTCTTC |
| | CAATTATAGAATCATCTGATTAGAA |
| | TTACAGTAAATGCATAGCTCAGTTT |
| | GCATTGTCCTGAGGTGAACCGCAAA |
| Enh119 human | CTATTGTGTTATTATTGCTTCTAAT |
| chr3: | TTAACTACCAGACTGTTAAACATTA |
| 187982147-187982250 | CTGCCCCAAGCTCAGCCAGGGGTGG |
| (SEQ ID NO: 68) | GCACTGCACTTTGAAGCCACCAAGT |
| | CAAT |
| Enh119 human | CTATTGACTTGGTGGCTTCAAAGTG |
| chr3: | CAGTGCCCACCCCTGGCTGAGCTTG |
| 187981138-187982252 | GGGCAGTAATGTTTAACAGTCTGGT |
| Reverse complement | AGTTAAATTAGAAGCAATAATAACA |
| (SEQ ID NO: 69) | CAATAGTCAGGTTTGGTTCTAACAC |
| | ACGAATGAGTCCCAACCCTCTAGCT |
| | GTAACTGAAATAAGTAGAAGGGGAG |
| | GAATGGATTTGGGATTCTTCTGTAG |
| | GATTCCTGATCTGTTCTTCTATGGC |
| | CAGACAGAGCAAGAAAACCCATGGG |
| | ACCACAACACAACCCCATGATTGGA |
| | AAAACTTGTGTTTTACAGACCACCT |
| | CTCAAGACTCAATGCTGACAGGCTG |
| | GAAATAAACTAGAATAATTAATTCT |
| | AAAATAGTCACGAAGACAAATCTTC |
| | CCTCCAATTCAGTCCAAAGGTCTGG |
| | ATTGGCTTAATGTCTAGAGTGTGTG |
| | CTATTTGACTATTTCTACTAAACAT |
| | TAATTAAATAATCCATCTAGCTCTG |
| | GCCCCCAGAGCTCCAGGGTCCCCAG |
| | AAGTGGAATATCCTAACTGGTATCT |
| | GTCCCTTTCCAAGTTAGCCCTTGTA |
| | TGAGTGCCCCGCTGAGATGAGGTAG |
| | TGGGCTTTCAGCATTCAGTACCCTC |
| | CGATGCTCCAACCAGAGCAGCTTGG |
| | TTTGCGGTTCACCTCAGGACAATGC |
| | AAACTGAGCTATGCATTTACTGTAA |
| | TTCTAATCAGATGATTCTATAATTG |
| | GAAGAACACCAGTGGCATTCTGTCT |
| | TTGGAGTCTTTTCCATTAAGCAATT |
| | GAACAAGGGCCAGGGGACCTTTACT |
| | TGTCCCAAACGCAGATCGTGATAGA |
| | GACTCTGAGATCTGGATACTATGTT |
| | CCATTCTATAACAGGATCAAGTTCT |
| | TAATTTTTTCTTGGTAAGATCATAA |
| | AATGTGTGTAGCAGTCATTTCACCC |
| | GTACTTTCAATAAATTCTGACCAAC |
| | TGCTCTCCTAACAATGAAAAACAAA |
| | TGAAATGAGCAAAGTGTTTGTGTGT |
| | ATTTGTGGTGGGGAGTGTGTGTATG |
| | TATGCTTGAATATAGTGACTGAATA |
| | TTTTAGGTAAATATTTCCAAGTCTA |
| | GATTATAATCATATATGATAATTGT |
| | TGACTAAAGTAATAAGAAAAATAAC |
| | CTGAATTTCTTAATC |
| Enh119 human | TTTGCGGTTCACCTCAGGACAATGC |
| chr3: | AAACTGAGCTATGCATTTACTGTAA |
| 187981428-187981627 | TTCTAATCAGATGATTCTATAATTG |
| Reverse complement | GAAGAACACCAGTGGCATTCTGTCT |
| (SEQ ID NO: 70) | TTGGAGTCTTTTCCATTAAGCAATT |
| | GAACAAGGGCCAGGGGACCTTTACT |
| | TGTCCCAAACGCAGATCGTGATAGA |
| | GACTCTGAGATCTGGATACTATGTT |
| Enh119 human | ATTGACTTGGTGGCTTCAAAGTGCA |
| chr3: | GTGCCCACCCCTGGCTGAGCTTGGG |
| 187982147-187982250 | GCAGTAATGTTTAACAGTCTGGTAG |
| Reverse complement | TTAAATTAGAAGCAATAATAACACA |
| (SEQ ID NO: 71) | ATAG |

L-ITR
(SEQ ID NO: 50)
cctgcaggcagctgcgcgctcgctcgctcactgaggccgcccggg
caaagcccgggcgtcgggcgacctttggtcgcccggcctcagtga
gcgagcgagcgcgcagagagggagtggccaactccatcactaggg
gttcct
pBG-X₍₀₋₅₀₀₎-pBG intron
(SEQ ID NO: 22)
ctgggcataaaagtcagggcagagccatctattgcttacatttgc
ttct-X₍₀₋₅₀₀₎-gtaagtatcaaggttacaagacaggtttaag
gagaccaatagaaactgggcttgtcgagacagagaagactcttgc
gtttctgataggcacctattggtcttactgacatccactttgcct
ttctctccacag
pBG-X84-DBG intron
(SEQ ID NO: 58)
ctgggcataaaagtcagggcagagccatctattgcttacatttgc
ttct-X₍₈₄₎-gtaagtatcaaggttacaagacaggtttaaggag
accaatagaaactgggcttgtcgagacagagaagactcttgcgtt
tctgataggcacctattggtcttactgacatccactttgcctttc
tctccacag
pBG-linker-pBG intron
(SEQ ID NO: 59)
ctgggcataaaagtcagggcagagccatctattgcttacatttgc
ttctagcctgcaggtcgaggagcgcagccttccagaagcagagcg
cggcgccttaagctgcagaagttggtcgtgaggcactgggcaggt
aagtatcaaggttacaagacaggtttaaggagaccaatagaaact
gggcttgtcgagacagagaagactcttgcgtttctgataggcacc
tattggtcttactgacatccactttgcctttctctccacag
pCHAT
(SEQ ID NO: 23)
TCTCTTGTCCAATGGGGCTTGGAGCACCGAGGCCAGCGAAGCCAT
CGCGCTCCTTGCGGAGGTGAAGAGGACCCTGAGTCCCCACCTGCG
GCTCCCCTGTGTAGAGCCTGCATCTGTCTGTCCTTCCTTCCATTG
CTCCCAGTGCCAAACTTGGGCCGCTGCACCGCGGCGCCTCCGCCC
AAATCAATAAACTGTGTCTGTCCCAGGAGGCCGAGTCTCTTTACT
GGTGGGGGGTGCGTGGAGGCGCGCAGGGCCAGAGCAGAGGGGAGG
GTGAACTGGGTCTCCAAGTCCCAATCCAGACCTAAGCCAAACTAA
CACGTAGGCACCTGTAGCTGTTTTTCTACCTGGAAAAGGGGATAG
GAAGGAAGCAAACCCAACAAAGGCTGTCACCCACGGTCACCAAGG
AGCACCATGCTCCCCTCAGCCCAGGATAGACCCTCTTTTCCAGGC
CTAGCGCAGAGCCCGGGGATGCCGCCCGGGGGAGCCTGAGGACCC
GCTCCAGCTAGGCACGCCAGGCCCCGCCCTTTGAGGACACGCCCC
ACACCAGCCTCAGAGCTCTGAGGTGCCTGGGCTGAGCTTCCCTTC
AGACCAGAATCCCGCCCCGTTGAGGCTTTGAGAAAGGAGTAGGAG
CCGAGCATTCCGGCAGAGGAAGAAAAACGGCCC
eGFP
(SEQ ID NO: 51)
atggtgagcaagggcgaggagctgttcaccggggtggtgcccatc
ctggtcgagctggacggcgacgtaaacggccacaagttcagcgtg
tccggcgagggcgagggcgatgccacctacggcaagctgaccctg
aagttcatctgcaccaccggcaagctgcccgtgccctggcccacc
ctcgtgaccaccctgacctacggcgtgcagtgcttcagccgctac
cccgaccacatgaagcagcacgacttcttcaagtccgccatgccc
gaaggctacgtccaggagcgcaccatcttcttcaaggacgacggc
aactacaagacccgcgccgaggtgaagttcgagggcgacaccctg
gtgaaccgcatcgagctgaagggcatcgacttcaaggaggacggc
aacatcctggggcacaagctggagtacaactacaacagccacaac
gtctatatcatggccgacaagcagaagaacggcatcaaggtgaac
ttcaagatccgccacaacatcgaggacggcagcgtgcagctcgcc
gaccactaccagcagaacacccccatcggcgacggccccgtgctg
ctgcccgacaaccactacctgagcacccagtccgccctgagcaaa
gaccccaacgagaagcgcgatcacatggtcctgctggagttcgtg
accgccgccgggatcactctcggcatggacgagctgtacaagtaa
WPRE
(SEQ ID NO: 52)
aatcaacctctggattacaaaatttgtgaaagattgactggtatt
cttaactatgttgctccttttacgctatgtggatacgctgcttta
atgcctttgtatcatgctattgcttcccgtatggctttcattttc
tcctccttgtataaatcctggttgctgtctctttatgaggagttg
tggcccgttgtcaggcaacgtggcgtggtgtgcactgtgtttgct
gacgcaacccccactggttggggcattgccaccacctgtcagctc
ctttccgggactttcgctttccccctccctattgccacggcggaa
ctcatcgccgcctgccttgcccgctgctggacaggggctcggctg
ttgggcactgacaattccgtggtgttgtcggggaaatcatcgtcc
tttccttggctgctcgcctatgttgccacctggattctgcgcggg
acgtccttctgctacgtcccttcggccctcaatccagcggacctt
ccttcccgcggcctgctgccggctctgcggcctcttccgcgtctt
cgccttcgccctcagacgagtcggatctccctttgggccgcctcc
ccgc
SV40pA
(SEQ ID NO: 53)
aacttgtttattgcagcttataatggttacaaataaagcaatagc
atcacaaatttcacaaataaagcatttttttcactgc
R-ITR
(SEQ ID NO: 54)
aggaacccctagtgatggagttggccactccctctctgcgcgctc
gctcgctcactgaggccgggcgaccaaaggtcgcccgacgcccgg
gctttgcccgggcggcctcagtgagcgagcgagcgcgcagctgcc
tgcagg
Enh98(mouse)-pBG-GFP vector
(c0108_ssAAV.Enh098.pBg.NLS*.eGFP.WPRE.SV40pA)
(SEQ ID NO: 46)
tgagtattcaacatttccgtgtcgcccttattcccttttttgcgg
cattttgccttcctgtttttgctcacccagaaacgctggtgaaag
taaaagatgctgaagatcagttgggtgcacgagtgggttacatcg
aactggatctcaacagcggtaagatccttgagagttttcgccccg
aagaacgttttccaatgatgagcacttttaaagttctgctatgtg
gcgcggtattatcccgtattgacgccgggcaagagcaactcggtc
gccgcatacactattctcagaatgacttggttgagtactcaccag
tcacagaaaagcatcttacggatggcatgacagtaagagaattat
gcagtgctgccataaccatgagtgataacactgcggccaacttac
ttctgacaacgatcggaggaccgaaggagctaaccgcttttttgc
acaacatgggggatcatgtaactcgccttgatcgttgggaaccgg
agctgaatgaagccataccaaacgacgagcgtgacaccacgatgc
ctgtagcaatggcaacaacgttgcgcaaactattaactggcgaac
tacttactctagcttcccggcaacaattaatagactggatggagg
cggataaagttgcaggaccacttctgcgctcggcccttccggctg
gctggtttattgctgataaatctggagccggtgagcgtgggtctc
gcggtatcattgcagcactggggccagatggtaagccctcccgta
tcgtagttatctacacgacggggagtcaggcaactatggatgaac
gaaatagacagatcgctgagataggtgcctcactgattaagcatt
ggtaactgtcagaccaagtttactcatatatactttagattgatt
taaaacttcatttttaatttaaaaggatctaggtgaagatccttt
ttgataatctcatgaccaaaatcccttaacgtgagttttcgttcc
actgagcgtcagaccccgtagaaaagatcaaaggatcttcttgag
atcctttttttctgcgcgtaatctgctgcttgcaaacaaaaaaac
caccgctaccagcggtggtttgtttgccggatcaagagctaccaa
ctctttttccgaaggtaactggcttcagcagagcgcagataccaa
atactgtccttctagtgtagccgtagttaggccaccacttcaaga
actctgtagcaccgcctacatacctcgctctgctaatcctgttac
cagtggctgctgccagtggcgataagtcgtgtcttaccgggttgg
actcaagacgatagttaccggataaggcgcagcggtcgggctgaa
cggggggttcgtgcacacagcccagcttggagcgaacgacctaca
ccgaactgagatacctacagcgtgagctatgagaaagcgccacgc
ttcccgaagggagaaaggcggacaggtatccggtaagcggcaggg
tcggaacaggagagcgcacgagggagcttccagggggaaacgcct
ggtatctttatagtcctgtcgggtttcgccacctctgacttgagc
gtcgatttttgtgatgctcgtcaggggggcggagcctatggaaaa
acgccagcaacgcggcctttttacggttcctggccttttgctggc
cttttgctcacatgtcctgcaggcagctgcgcgctcgctcgctca
ctgaggccgcccgggcaaagcccgggcgtcgggcgacctttggtc
gcccggcctcagtgagcgagcgagcgcgcagagagggagtggcca
actccatcactaggggttcctgcggccgcacgcgtttaatACCGT
GGCTTAGTNTGATAAACCAAAACCTGCTCCATTATGAATCAGTGC
TGTGGGGAGTGGGTAGAGAGTGTGAAGTTCTGGGGTGGGGGAGTC
TGGAGAGAGGGTGGGAGCAGCCATTCTGCAGCAGTGCCTTCTTGG
GGTCATGGGTCTGTAGGTGCTGCTGTGGAGGGAGAGATCAGCCTA
TTCTGGCTTCATTTCTGAGCTGCAAACTGCCTGGGTGTCTGGAGA
AGCAGGTTGGCGTGGTGGTTAGCAGTGCGTGGGCGGGGTTGCCCG
CTCTTGATTTATGATTTCTTTGTCTCTGTGGAAGCACTTAAGTGC
AGGCTTTAGTTCCAATGACACTCAGGAGCCTCTGGATTCCAGCAC
TGGGGATGGGGGTGGGGTAGAACGTTCTCAGGCCTCACCAACCCC
TCCCCTGTGTGCTGCCTTTGGGAGAGTCCCAAGGCTTCAGCATTA
CTTAATTAATTAGGCCTCTACTGCTACATAGGCTCAGATTCAAAA
GAACAGAGTGGCCCACGTCAGCCATTCCCGGAAAAGTCTGATGGC
TGGAAGCCAGAGGACTATGTGTCTGCCTTGCTGCCCTTGGCCAGC
CCATCCTGAATGCCCAGACTCGGACAATGGAGTAGGTACAGAAGG
GTAAAGACAGTGTCTTCTGTACCAGTAAGTGGGCCCTGATCTGCT
CTCTACAGCTTCCAGAGAAAGGGCCTGGCCAATGAGCGGCCTTTT
GAGTAGCAGATACCTCACATGCATTCTGATAGAAAGCCTGGCCCC
AGATCACTGTGACTTTAGCCCTCAGGTTTCTTTTGCACTTCAATT
CAATGACTTCTTGAGGTTCATTTCCCTCTCCAAGATTTGCCACAG
ACCAGTGGTTCTCAAgtcgacagatctaattcctgcagcccgggc
tgggcataaaagtcagggcagagccatctattgcttacatttgct
tctagcctgcaggtcgaggagcgcagccttccagaagcagagcgc
ggcgccttaagctgcagaagttggtcgtgaggcactgggcaggta
agtatcaaggttacaagacaggtttaaggagaccaatagaaactg
ggcttgtcgagacagagaagactcttgcgtttctgataggcacct
attggtcttactgacatccactttgcctttctctccacaggtgtc
cactcccaGTTCAATTACAGCTCTTAAGAAGAATTCccaaagaaa
aagcggaaagtgctagtAGCCACCatggtgagcaagggcgaggag
ctgttcaccggggtggtgcccatcctggtcgagctggacggcgac
gtaaacggccacaagttcagcgtgtccggcgagggcgagggcgat
gccacctacggcaagctgaccctgaagttcatctgcaccaccggc
aagctgcccgtgccctggcccaccctcgtgaccaccctgacctac
ggcgtgcagtgcttcagccgctaccccgaccacatgaagcagcac
gacttcttcaagtccgccatgcccgaaggctacgtccaggagcgc
accatcttcttcaaggacgacggcaactacaagacccgcgccgag
gtgaagttcgagggcgacaccctggtgaaccgcatcgagctgaag
ggcatcgacttcaaggaggacggcaacatcctggggcacaagctg
gagtacaactacaacagccacaacgtctatatcatggccgacaag
cagaagaacggcatcaaggtgaacttcaagatccgccacaacatc
gaggacggcagcgtgcagctcgccgaccactaccagcagaacacc
cccatcggcgacggccccgtgctgctgcccgacaaccactacctg
agcacccagtccgccctgagcaaagaccccaacgagaagcgcgat
cacatggtcctgctggagttcgtgaccgccgccgggatcactctc
ggcatggacgagctgtacaagtaaaagcttatcgataatcaacct
ctggattacaaaatttgtgaaagattgactggtattcttaactat
gttgctccttttacgctatgtggatacgctgctttaatgcctttg
tatcatgctattgcttcccgtatggctttcattttctcctccttg
tataaatcctggttgctgtctctttatgaggagttgtggcccgtt
gtcaggcaacgtggcgtggtgtgcactgtgtttgctgacgcaacc
cccactggttggggcattgccaccacctgtcagctcctttccggg
actttcgctttccccctccctattgccacggcggaactcatcgcc
gcctgccttgcccgctgctggacaggggctcggctgttgggcact
gacaattccgtggtgttgtcggggaaatcatcgtcctttccttgg
ctgctcgcctatgttgccacctggattctgcgcgggacgtccttc
tgctacgtcccttcggccctcaatccagcggaccttccttcccgg
gcctgctgccggctctgcggcctcttccgcgtcttcgccttcgcc
ctcagacgagtcggatctccctttgggccgcctccccgcatcgat
accgagcgctgctcgagaGCGATCGCtgtgatagcggccatcaag
ctggccgcgactctagatcataatcagccataccacatttgtaga
ggttttacttgctttaaaaaacctcccacacctccccctgaacct
gaaacataaaatgaatgcaattgttgttgttaacttgtttattgc
agcttataatggttacaaataaagcaatagcatcacaaatttcac
aaataaagcatttttttcactgcattctagttgtggtttgtccaa
actcatcaatgtatcagcttatcgataccgcatgcacgtgcggac
cgagcggccgcaggaacccctagtgatggagttggccactccctc
tctgcgcgctcgctcgctcactgaggccgggcgaccaaaggtcgc
ccgacgcccgggctttgcccgggggcctcagtgagcgagcgagcg
cgcagctgcctgcaggggcgcctgatgcggtattttctccttacg
catctgtgcggtatttcacaccgcatacgtcaaagcaaccatagt
acgcgccctgtagcggcgcattaagcgcggcgggtgtggtggtta
cgcgcagcgtgaccgctacacttgccagcgccctagcgcccgctc
ctttcgctttcttcccttcctttctcgccacgttcgccggctttc
cccgtcaagctctaaatcgggggctccctttagggttccgattta
gtgctttacggcacctcgaccccaaaaaacttgatttgggtgatg
gttcacgtagtgggccatcgccctgatagacggtttttcgccctt
tgacgttggagtccacgttctttaatagtggactcttgttccaaa
ctggaacaacactcaaccctatctcgggctattcttttgatttat
aagggattttgccgatttcggcctattggttaaaaaatgagctga
tttaacaaaaatttaacgcgaattttaacaaaatattaacgttta
caattttatggtgcactctcagtacaatctgctctgatgccgcat
agttaagccagccccgacacccgccaacacccgctgacgcgccct
gacgggcttgtctgctcccggcatccgcttacagacaagctgtga
ccgtctccgggagctgcatgtgtcagaggttttcaccgtcatcac
cgaaacgcgcgagacgaaagggcctcgtgatacgcctatttttat
aggttaatgtcatgataataatggtttcttagacgtcaggtggca
cttttcggggaaatgtgcgcggaacccctatttgtttatttttct
aaatacattcaaatatgtatccgctcatgagacaataaccctgat
aaatgcttcaataatattgaaaaaggaagagta
Enh57(mouse)-pBG-GFP vector
(c0106_ssAAV.Enh057.pBg.NLS*.eGFP.WPRE.SV40pA)
(SEQ ID NO: 47)
tgagtattcaacatttccgtgtcgcccttattcccttttttgcgg
cattttgccttcctgtttttgctcacccagaaacgctggtgaaag
taaaagatgctgaagatcagttgggtgcacgagtgggttacatcg
aactggatctcaacagcggtaagatccttgagagttttcgccccg
aagaacgttttccaatgatgagcacttttaaagttctgctatgtg
gcgcggtattatcccgtattgacgccgggcaagagcaactcggtc
gccgcatacactattctcagaatgacttggttgagtactcaccag
tcacagaaaagcatcttacggatggcatgacagtaagagaattat
gcagtgctgccataaccatgagtgataacactgcggccaacttac
ttctgacaacgatcggaggaccgaaggagctaaccgcttttttgc
acaacatgggggatcatgtaactcgccttgatcgttgggaaccgg
agctgaatgaagccataccaaacgacgagcgtgacaccacgatgc
ctgtagcaatggcaacaacgttgcgcaaactattaactggcgaac
tacttactctagcttcccggcaacaattaatagactggatggagg
cggataaagttgcaggaccacttctgcgctcggcccttccggctg
gctggtttattgctgataaatctggagccggtgagcgtgggtctc
gcggtatcattgcagcactggggccagatggtaagccctcccgta
tcgtagttatctacacgacggggagtcaggcaactatggatgaac
gaaatagacagatcgctgagataggtgcctcactgattaagcatt
ggtaactgtcagaccaagtttactcatatatactttagattgatt
taaaacttcatttttaatttaaaaggatctaggtgaagatccttt
ttgataatctcatgaccaaaatcccttaacgtgagttttcgttcc
actgagcgtcagaccccgtagaaaagatcaaaggatcttcttgag
atcctttttttctgcgcgtaatctgctgcttgcaaacaaaaaaac
caccgctaccagcggtggtttgtttgccggatcaagagctaccaa
ctctttttccgaaggtaactggcttcagcagagcgcagataccaa
atactgtccttctagtgtagccgtagttaggccaccacttcaaga
actctgtagcaccgcctacatacctcgctctgctaatcctgttac
cagtggctgctgccagtggcgataagtcgtgtcttaccgggttgg
actcaagacgatagttaccggataaggcgcagcggtcgggctgaa
cggggggttcgtgcacacagcccagcttggagcgaacgacctaca
ccgaactgagatacctacagcgtgagctatgagaaagcgccacgc
ttcccgaagggagaaaggcggacaggtatccggtaagcggcaggg
tcggaacaggagagcgcacgagggagcttccagggggaaacgcct
ggtatctttatagtcctgtcgggtttcgccacctctgacttgagc
gtcgatttttgtgatgctcgtcaggggggcggagcctatggaaaa
acgccagcaacgcggcctttttacggttcctggccttttgctggc
cttttgctcacatgtcctgcaggcagctgcgcgctcgctcgctca
ctgaggccgcccgggcaaagcccgggcgtcgggcgacctttggtc
gcccggcctcagtgagcgagcgagcgcgcagagagggagtggcca
actccatcactaggggttcctgcggccgcacgcgtttaatTTTCT
TAATAACTGCTATTTTGAAATGTATCATTATCATAACTCCAGTGT
AGAAGTGGTGTCCAGATTTCTGCTATGTTGCTAATTTTTGATATG
AGACATTCTTATTAGAGTTGAGGGAATGTGCTTGTATCACTTAGG
TGCACACACCAGAAGCCAGTGCAGGCTCAAGGTGAACACAGAGAC
TCGTGGTACCCCAAATGGCTCTCTATCTGACTTCAGCTCTCTTCC
ACTTCTTCAACTAGAAATATTGCTGAGGGCTTGTTAAACACACAA
AAGCCATGGCTTTTGACCATCTTGCAAGCAAAAGAAACACCATTT
TAAACTCCTTTGAAAACGTTCTCTTCTTTCACATTAAGAGGCTGC
CACACGAACAGAACGTGCCATAAATAATGTGTGCTAACATTTTCC
AAAAACTGGACATCAATTAACGTTAATTTATGAGAACACTTCTTG
AGAGGAGCACAGTTCAGACTCATAACTACTGAAAAGGCTCATTAA
TAGAAATGTGTAGGGAGAGGGTTTTTTTCTTCTTCTAAAGGGAAC
ATTAAAGTAAACACATATCATTGCAAGGAAGGCTCATGATTTATT
GCAAACTCAGTGGAAAGGAGACTTTACGCTGTGTTTCCAGGGTGA
ATTTTGAGCAAAGGAATCAAGCAAACAAAATGAAATGAGGATATT
CTCTTAGGAAAGGCATCCTGTGACAACCCAGACAAATGATAGCTA
ATACTTATATAATAAGTACTACATATCAGGTCAGGCACTATGCCA
ACATGATCTTGTGTGTGTCTCACCAAGAACACTGCCAGGGAAATT
TGTTTTGCTGCCATATACAAAGTTAAAAATCAAGCCCCCgtcgac
agatctaattcctgcagcccgggctgggcataaaagtcagggcag
agccatctattgcttacatttgcttctagcctgcaggtcgaggag
cgcagccttccagaagcagagcgcggcgccttaagctgcagaagt
tggtcgtgaggcactgggcaggtaagtatcaaggttacaagacag
gtttaaggagaccaatagaaactgggcttgtcgagacagagaaga
ctcttgcgtttctgataggcacctattggtcttactgacatccac
tttgcctttctctccacaggtgtccactcccaGTTCAATTACAGC
TCTTAAGAAGAATTCccaaagaaaaagcggaaagtgctagtAGCC
ACCatggtgagcaagggcgaggagctgttcaccggggtggtgccc
atcctggtcgagctggacggcgacgtaaacggccacaagttcagc
gtgtccggcgagggcgagggcgatgccacctacggcaagctgacc
ctgaagttcatctgcaccaccggcaagctgcccgtgccctggccc
accctcgtgaccaccctgacctacggcgtgcagtgcttcagccgc
taccccgaccacatgaagcagcacgacttcttcaagtccgccatg
cccgaaggctacgtccaggagcgcaccatcttcttcaaggacgac
ggcaactacaagacccgcgccgaggtgaagttcgagggcgacacc
ctggtgaaccgcatcgagctgaagggcatcgacttcaaggaggac
ggcaacatcctggggcacaagctggagtacaactacaacagccac
aacgtctatatcatggccgacaagcagaagaacggcatcaaggtg
aacttcaagatccgccacaacatcgaggacggcagcgtgcagctc
gccgaccactaccagcagaacacccccatcggcgacggccccgtg
ctgctgcccgacaaccactacctgagcacccagtccgccctgagc
aaagaccccaacgagaagcgcgatcacatggtcctgctggagttc
gtgaccgccgccgggatcactctcggcatggacgagctgtacaag
taaaagcttatcgataatcaacctctggattacaaaatttgtgaa
agattgactggtattcttaactatgttgctccttttacgctatgt
ggatacgctgctttaatgcctttgtatcatgctattgcttcccgt
atggctttcattttctcctccttgtataaatcctggttgctgtct
ctttatgaggagttgtggcccgttgtcaggcaacgtggcgtggtg
tgcactgtgtttgctgacgcaacccccactggttggggcattgcc
accacctgtcagctcctttccgggactttcgctttccccctccct
attgccacggcggaactcatcgccgcctgccttgcccgctgctgg
acaggggctcggctgttgggcactgacaattccgtggtgttgtcg
gggaaatcatcgtcctttccttggctgctcgcctatgttgccacc
tggattctgcgcgggacgtccttctgctacgtcccttcggccctc
aatccagcggaccttccttcccgcggcctgctgccggctctgcgg
cctcttccgcgtcttcgccttcgccctcagacgagtcggatctcc
ctttgggccgcctccccgcatcgataccgagcgctgctcgagaGC
GATCGCtgtgatagcggccatcaagctggccgcgactctagatca
taatcagccataccacatttgtagaggttttacttgctttaaaaa
acctcccacacctccccctgaacctgaaacataaaatgaatgcaa
ttgttgttgttaacttgtttattgcagcttataatggttacaaat
aaagcaatagcatcacaaatttcacaaataaagcatttttttcac
tgcattctagttgtggtttgtccaaactcatcaatgtatcagctt
atcgataccgcatgcacgtgcggaccgagcggccgcaggaacccc
tagtgatggagttggccactccctctctgcgcgctcgctcgctca
ctgaggccgggcgaccaaaggtcgcccgacgcccgggctttgccc
gggcggcctcagtgagcgagcgagcgcgcagctgcctgcaggggc
gcctgatgcggtattttctccttacgcatctgtgcggtatttcac
accgcatacgtcaaagcaaccatagtacgcgccctgtagcggcgc
attaagcgcggcgggtgtggtggttacgcgcagcgtgaccgctac
acttgccagcgccctagcgcccgctcctttcgctttcttcccttc
ctttctcgccacgttcgccggctttccccgtcaagctctaaatcg
ggggctccctttagggttccgatttagtgctttacggcacctcga
ccccaaaaaacttgatttgggtgatggttcacgtagtgggccatc
gccctgatagacggtttttcgccctttgacgttggagtccacgtt
ctttaatagtggactcttgttccaaactggaacaacactcaaccc
tatctcgggctattcttttgatttataagggattttgccgatttc
ggcctattggttaaaaaatgagctgatttaacaaaaatttaacgc
gaattttaacaaaatattaacgtttacaattttatggtgcactct
cagtacaatctgctctgatgccgcatagttaagccagccccgaca
cccgccaacacccgctgacgcgccctgacgggcttgtctgctccc
ggcatccgcttacagacaagctgtgaccgtctccgggagctgcat
gtgtcagaggttttcaccgtcatcaccgaaacgcgcgagacgaaa
gggcctcgtgatacgcctatttttataggttaatgtcatgataat
aatggtttcttagacgtcaggtggcacttttcggggaaatgtgcg
cggaacccctatttgtttatttttctaaatacattcaaatatgta
tccgctcatgagacaataaccctgataaatgcttcaataatattg
aaaaaggaagagta
Enh98(mouse)-pChAT-GFP vector
(c0104_ssAAV.Enh098.pCHAT.NLS*.eGFP.WPRE.SV40pA)
(SEQ ID NO: 48)
tgagtattcaacatttccgtgtcgcccttattcccttttttgcgg
cattttgccttcctgtttttgctcacccagaaacgctggtgaaag
taaaagatgctgaagatcagttgggtgcacgagtgggttacatcg
aactggatctcaacagcggtaagatccttgagagttttcgccccg
aagaacgttttccaatgatgagcacttttaaagttctgctatgtg
gcgcggtattatcccgtattgacgccgggcaagagcaactcggtc
gccgcatacactattctcagaatgacttggttgagtactcaccag
tcacagaaaagcatcttacggatggcatgacagtaagagaattat
gcagtgctgccataaccatgagtgataacactgcggccaacttac
ttctgacaacgatcggaggaccgaaggagctaaccgcttttttgc
acaacatgggggatcatgtaactcgccttgatcgttgggaaccgg
agctgaatgaagccataccaaacgacgagcgtgacaccacgatgc
ctgtagcaatggcaacaacgttgcgcaaactattaactggcgaac
tacttactctagcttcccggcaacaattaatagactggatggagg
cggataaagttgcaggaccacttctgcgctcggcccttccggctg
gctggtttattgctgataaatctggagccggtgagcgtgggtctc
gcggtatcattgcagcactggggccagatggtaagccctcccgta
tcgtagttatctacacgacggggagtcaggcaactatggatgaac
gaaatagacagatcgctgagataggtgcctcactgattaagcatt
ggtaactgtcagaccaagtttactcatatatactttagattgatt
taaaacttcatttttaatttaaaaggatctaggtgaagatccttt
ttgataatctcatgaccaaaatcccttaacgtgagttttcgttcc
actgagcgtcagaccccgtagaaaagatcaaaggatcttcttgag
atcctttttttctgcgcgtaatctgctgcttgcaaacaaaaaaac
caccgctaccagcggtggtttgtttgccggatcaagagctaccaa
ctctttttccgaaggtaactggcttcagcagagcgcagataccaa
atactgtccttctagtgtagccgtagttaggccaccacttcaaga
actctgtagcaccgcctacatacctcgctctgctaatcctgttac
cagtggctgctgccagtggcgataagtcgtgtcttaccgggttgg
actcaagacgatagttaccggataaggcgcagcggtcgggctgaa
cggggggttcgtgcacacagcccagcttggagcgaacgacctaca
ccgaactgagatacctacagcgtgagctatgagaaagcgccacgc
ttcccgaagggagaaaggcggacaggtatccggtaagcggcaggg
tcggaacaggagagcgcacgagggagcttccagggggaaacgcct
ggtatctttatagtcctgtcgggtttcgccacctctgacttgagc
gtcgatttttgtgatgctcgtcaggggggcggagcctatggaaaa
acgccagcaacgcggcctttttacggttcctggccttttgctggc
cttttgctcacatgtcctgcaggcagctgcgcgctcgctcgctca
ctgaggccgcccgggcaaagcccgggcgtcgggcgacctttggtc
gcccggcctcagtgagcgagcgagcgcgcagagagggagtggcca
actccatcactaggggttcctgcggccgcacgcgtttaatACCGT
GGCTTAGTNTGATAAACCAAAACCTGCTCCATTATGAATCAGTGC
TGTGGGGAGTGGGTAGAGAGTGTGAAGTTCTGGGGTGGGGGAGTC
TGGAGAGAGGGTGGGAGCAGCCATTCTGCAGCAGTGCCTTCTTGG
GGTCATGGGTCTGTAGGTGCTGCTGTGGAGGGAGAGATCAGCCTA
TTCTGGCTTCATTTCTGAGCTGCAAACTGCCTGGGTGTCTGGAGA
AGCAGGTTGGCGTGGTGGTTAGCAGTGCGTGGGCGGGGTTGCCCG
CTCTTGATTTATGATTTCTTTGTCTCTGTGGAAGCACTTAAGTGC
AGGCTTTAGTTCCAATGACACTCAGGAGCCTCTGGATTCCAGCAC
TGGGGATGGGGGTGGGGTAGAACGTTCTCAGGCCTCACCAACCCC
TCCCCTGTGTGCTGCCTTTGGGAGAGTCCCAAGGCTTCAGCATTA
CTTAATTAATTAGGCCTCTACTGCTACATAGGCTCAGATTCAAAA
GAACAGAGTGGCCCACGTCAGCCATTCCCGGAAAAGTCTGATGGC
TGGAAGCCAGAGGACTATGTGTCTGCCTTGCTGCCCTTGGCCAGC
CCATCCTGAATGCCCAGACTCGGACAATGGAGTAGGTACAGAAGG
GTAAAGACAGTGTCTTCTGTACCAGTAAGTGGGCCCTGATCTGCT
CTCTACAGCTTCCAGAGAAAGGGCCTGGCCAATGAGCGGCCTTTT
GAGTAGCAGATACCTCACATGCATTCTGATAGAAAGCCTGGCCCC
AGATCACTGTGACTTTAGCCCTCAGGTTTCTTTTGCACTTCAATT
CAATGACTTCTTGAGGTTCATTTCCCTCTCCAAGATTTGCCACAG
ACCAGTGGTTCTCAAgtcgacagatctTCTCTTGTCCAATGGGGC
TTGGAGCACCGAGGCCAGCGAAGCCATCGCGCTCCTTGCGGAGGT
GAAGAGGACCCTGAGTCCCCACCTGCGGCTCCCCTGTGTAGAGCC
TGCATCTGTCTGTCCTTCCTTCCATTGCTCCCAGTGCCAAACTTG
GGCCGCTGCACCGCGGCGCCTCCGCCCAAATCAATAAACTGTGTC
TGTCCCAGGAGGCCGAGTCTCTTTACTGGTGGGGGGTGCGTGGAG
GCGCGCAGGGCCAGAGCAGAGGGGAGGGTGAACTGGGTCTCCAAG
TCCCAATCCAGACCTAAGCCAAACTAACACGTAGGCACCTGTAGC
TGTTTTTCTACCTGGAAAAGGGGATAGGAAGGAAGCAAACCCAAC
AAAGGCTGTCACCCACGGTCACCAAGGAGCACCATGCTCCCCTCA
GCCCAGGATAGACCCTCTTTTCCAGGCCTAGCGCAGAGCCCGGGG
ATGCCGCCCGGGGGAGCCTGAGGACCCGCTCCAGCTAGGCACGCC
AGGCCCCGCCCTTTGAGGACACGCCCCACACCAGCCTCAGAGCTC
TGAGGTGCCTGGGCTGAGCTTCCCTTCAGACCAGAATCCCGCCCC
GTTGAGGCTTTGAGAAAGGAGTAGGAGCCGAGCATTCCGGCAGAG
GAAGAAAAACGGCCCGAATTCccaaagaaaaagcggaaagtgcta
gtAGCCACCatggtgagcaagggcgaggagctgttcaccggggtg
gtgcccatcctggtcgagctggacggcgacgtaaacggccacaag
ttcagcgtgtccggcgagggcgagggcgatgccacctacggcaag
ctgaccctgaagttcatctgcaccaccggcaagctgcccgtgccc
tggcccaccctcgtgaccaccctgacctacggcgtgcagtgcttc
agccgctaccccgaccacatgaagcagcacgacttcttcaagtcc
gccatgcccgaaggctacgtccaggagcgcaccatcttcttcaag
gacgacggcaactacaagacccgcgccgaggtgaagttcgagggc
gacaccctggtgaaccgcatcgagctgaagggcatcgacttcaag
gaggacggcaacatcctggggcacaagctggagtacaactacaac
agccacaacgtctatatcatggccgacaagcagaagaacggcatc
aaggtgaacttcaagatccgccacaacatcgaggacggcagcgtg
cagctcgccgaccactaccagcagaacacccccatcggcgacggc
cccgtgctgctgcccgacaaccactacctgagcacccagtccgcc
ctgagcaaagaccccaacgagaagcgcgatcacatggtcctgctg
gagttcgtgaccgccgccgggatcactctcggcatggacgagctg
tacaagtaaaagcttatcgataatcaacctctggattacaaaatt
tgtgaaagattgactggtattcttaactatgttgctccttttacg
ctatgtggatacgctgctttaatgcctttgtatcatgctattgct
tcccgtatggctttcattttctcctccttgtataaatcctggttg
ctgtctctttatgaggagttgtggcccgttgtcaggcaacgtggc
gtggtgtgcactgtgtttgctgacgcaacccccactggttggggc
attgccaccacctgtcagctcctttccgggactttcgctttcccc
ctccctattgccacggcggaactcatcgccgcctgccttgcccgc
tgctggacaggggctcggctgttgggcactgacaattccgtggtg
ttgtcggggaaatcatcgtcctttccttggctgctcgcctatgtt
gccacctggattctgcgcgggacgtccttctgctacgtcccttcg
gccctcaatccagcggaccttccttcccgcggcctgctgccggct
ctgcggcctcttccgcgtcttcgccttcgccctcagacgagtcgg
atctccctttgggccgcctccccgcatcgataccgagcgctgctc
gagaGCGATCGCtgtgatagcggccatcaagctggccgcgactct
agatcataatcagccataccacatttgtagaggttttacttgctt
taaaaaacctcccacacctccccctgaacctgaaacataaaatga
atgcaattgttgttgttaacttgtttattgcagcttataatggtt
acaaataaagcaatagcatcacaaatttcacaaataaagcatttt
tttcactgcattctagttgtggtttgtccaaactcatcaatgtat
cagcttatcgataccgcatgcacgtgcggaccgagcggccgcagg
aacccctagtgatggagttggccactccctctctgcgcgctcgct
cgctcactgaggccgggcgaccaaaggtcgcccgacgcccgggct
ttgcccgggcggcctcagtgagcgagcgagcgcgcagctgcctgc
aggggcgcctgatgcggtattttctccttacgcatctgtgcggta
tttcacaccgcatacgtcaaagcaaccatagtacgcgccctgtag
cggcgcattaagcgcggcgggtgtggtggttacgcgcagcgtgac
cgctacacttgccagcgccctagcgcccgctcctttcgctttctt
cccttcctttctcgccacgttcgccggctttccccgtcaagctct
aaatcgggggctccctttagggttccgatttagtgctttacggca
cctcgaccccaaaaaacttgatttgggtgatggttcacgtagtgg
gccatcgccctgatagacggtttttcgccctttgacgttggagtc
cacgttctttaatagtggactcttgttccaaactggaacaacact
caaccctatctcgggctattcttttgatttataagggattttgcc
gatttcggcctattggttaaaaaatgagctgatttaacaaaaatt
taacgcgaattttaacaaaatattaacgtttacaattttatggtg
cactctcagtacaatctgctctgatgccgcatagttaagccagcc
ccgacacccgccaacacccgctgacgcgccctgacgggcttgtct
gctcccggcatccgcttacagacaagctgtgaccgtctccgggag
ctgcatgtgtcagaggttttcaccgtcatcaccgaaacgcgcgag
acgaaagggcctcgtgatacgcctatttttataggttaatgtcat
gataataatggtttcttagacgtcaggtggcacttttcggggaaa
tgtgcgcggaacccctatttgtttatttttctaaatacattcaaa
tatgtatccgctcatgagacaataaccctgataaatgcttcaata
atattgaaaaaggaagagta
Enh57(mouse)-pChAT-GFP vector
(c0102_ssAAV.Enh057.pCHAT.NLS*.eGFP.WPRE.SV40pA)
(SEQ ID NO: 49)
tgagtattcaacatttccgtgtcgcccttattcccttttttgcgg
cattttgccttcctgtttttgctcacccagaaacgctggtgaaag
taaaagatgctgaagatcagttgggtgcacgagtgggttacatcg
aactggatctcaacagcggtaagatccttgagagttttcgccccg
aagaacgttttccaatgatgagcacttttaaagttctgctatgtg
gcgcggtattatcccgtattgacgccgggcaagagcaactcggtc
gccgcatacactattctcagaatgacttggttgagtactcaccag
tcacagaaaagcatcttacggatggcatgacagtaagagaattat
gcagtgctgccataaccatgagtgataacactgcggccaacttac
ttctgacaacgatcggaggaccgaaggagctaaccgcttttttgc
acaacatgggggatcatgtaactcgccttgatcgttgggaaccgg
agctgaatgaagccataccaaacgacgagcgtgacaccacgatgc
ctgtagcaatggcaacaacgttgcgcaaactattaactggcgaac
tacttactctagcttcccggcaacaattaatagactggatggagg
cggataaagttgcaggaccacttctgcgctcggcccttccggctg
gctggtttattgctgataaatctggagccggtgagcgtgggtctc
gcggtatcattgcagcactggggccagatggtaagccctcccgta
tcgtagttatctacacgacggggagtcaggcaactatggatgaac
gaaatagacagatcgctgagataggtgcctcactgattaagcatt
ggtaactgtcagaccaagtttactcatatatactttagattgatt
taaaacttcatttttaatttaaaaggatctaggtgaagatccttt
ttgataatctcatgaccaaaatcccttaacgtgagttttcgttcc
actgagcgtcagaccccgtagaaaagatcaaaggatcttcttgag
atcctttttttctgcgcgtaatctgctgcttgcaaacaaaaaaac
caccgctaccagcggtggtttgtttgccggatcaagagctaccaa
ctctttttccgaaggtaactggcttcagcagagcgcagataccaa
atactgtccttctagtgtagccgtagttaggccaccacttcaaga
actctgtagcaccgcctacatacctcgctctgctaatcctgttac
cagtggctgctgccagtggcgataagtcgtgtcttaccgggttgg
actcaagacgatagttaccggataaggcgcagcggtcgggctgaa
cggggggttcgtgcacacagcccagcttggagcgaacgacctaca
ccgaactgagatacctacagcgtgagctatgagaaagcgccacgc
ttcccgaagggagaaaggcggacaggtatccggtaagcggcaggg
tcggaacaggagagcgcacgagggagcttccagggggaaacgcct
ggtatctttatagtcctgtcgggtttcgccacctctgacttgagc
gtcgatttttgtgatgctcgtcaggggggcggagcctatggaaaa
acgccagcaacgcggcctttttacggttcctggccttttgctggc
cttttgctcacatgtcctgcaggcagctgcgcgctcgctcgctca
ctgaggccgcccgggcaaagcccgggcgtcgggcgacctttggtc
gcccggcctcagtgagcgagcgagcgcgcagagagggagtggcca
actccatcactaggggttcctgcggccgcacgcgtttaatTTTCT
TAATAACTGCTATTTTGAAATGTATCATTATCATAACTCCAGTGT
AGAAGTGGTGTCCAGATTTCTGCTATGTTGCTAATTTTTGATATG
AGACATTCTTATTAGAGTTGAGGGAATGTGCTTGTATCACTTAGG
TGCACACACCAGAAGCCAGTGCAGGCTCAAGGTGAACACAGAGAC
TCGTGGTACCCCAAATGGCTCTCTATCTGACTTCAGCTCTCTTCC
ACTTCTTCAACTAGAAATATTGCTGAGGGCTTGTTAAACACACAA
AAGCCATGGCTTTTGACCATCTTGCAAGCAAAAGAAACACCATTT
TAAACTCCTTTGAAAACGTTCTCTTCTTTCACATTAAGAGGCTGC
CACACGAACAGAACGTGCCATAAATAATGTGTGCTAACATTTTCC
AAAAACTGGACATCAATTAACGTTAATTTATGAGAACACTTCTTG
AGAGGAGCACAGTTCAGACTCATAACTACTGAAAAGGCTCATTAA
TAGAAATGTGTAGGGAGAGGGTTTTTTTCTTCTTCTAAAGGGAAC
ATTAAAGTAAACACATATCATTGCAAGGAAGGCTCATGATTTATT
GCAAACTCAGTGGAAAGGAGACTTTACGCTGTGTTTCCAGGGTGA
ATTTTGAGCAAAGGAATCAAGCAAACAAAATGAAATGAGGATATT
CTCTTAGGAAAGGCATCCTGTGACAACCCAGACAAATGATAGCTA
ATACTTATATAATAAGTACTACATATCAGGTCAGGCACTATGCCA
ACATGATCTTGTGTGTGTCTCACCAAGAACACTGCCAGGGAAATT
TGTTTTGCTGCCATATACAAAGTTAAAAATCAAGCCCCCgtcgac
agatctTCTCTTGTCCAATGGGGCTTGGAGCACCGAGGCCAGCGA
AGCCATCGCGCTCCTTGCGGAGGTGAAGAGGACCCTGAGTCCCCA
CCTGCGGCTCCCCTGTGTAGAGCCTGCATCTGTCTGTCCTTCCTT
CCATTGCTCCCAGTGCCAAACTTGGGCCGCTGCACCGCGGCGCCT
CCGCCCAAATCAATAAACTGTGTCTGTCCCAGGAGGCCGAGTCTC
TTTACTGGTGGGGGGTGCGTGGAGGCGCGCAGGGCCAGAGCAGAG
GGGAGGGTGAACTGGGTCTCCAAGTCCCAATCCAGACCTAAGCCA
AACTAACACGTAGGCACCTGTAGCTGTTTTTCTACCTGGAAAAGG
GGATAGGAAGGAAGCAAACCCAACAAAGGCTGTCACCCACGGTCA
CCAAGGAGCACCATGCTCCCCTCAGCCCAGGATAGACCCTCTTTT
CCAGGCCTAGCGCAGAGCCCGGGGATGCCGCCCGGGGGAGCCTGA
GGACCCGCTCCAGCTAGGCACGCCAGGCCCCGCCCTTTGAGGACA
CGCCCCACACCAGCCTCAGAGCTCTGAGGTGCCTGGGCTGAGCTT
CCCTTCAGACCAGAATCCCGCCCCGTTGAGGCTTTGAGAAAGGAG
TAGGAGCCGAGCATTCCGGCAGAGGAAGAAAAACGGCCCGAATTC
ccaaagaaaaagcggaaagtgctagtAGCCACCatggtgagcaag
ggcgaggagctgttcaccggggtggtgcccatcctggtcgagctg
gacggcgacgtaaacggccacaagttcagcgtgtccggcgagggc
gagggcgatgccacctacggcaagctgaccctgaagttcatctgc
accaccggcaagctgcccgtgccctggcccaccctcgtgaccacc
ctgacctacggcgtgcagtgcttcagccgctaccccgaccacatg
aagcagcacgacttcttcaagtccgccatgcccgaaggctacgtc
caggagcgcaccatcttcttcaaggacgacggcaactacaagacc
cgcgccgaggtgaagttcgagggcgacaccctggtgaaccgcatc
gagctgaagggcatcgacttcaaggaggacggcaacatcctgggg
cacaagctggagtacaactacaacagccacaacgtctatatcatg
gccgacaagcagaagaacggcatcaaggtgaacttcaagatccgc
cacaacatcgaggacggcagcgtgcagctcgccgaccactaccag
cagaacacccccatcggcgacggccccgtgctgctgcccgacaac
cactacctgagcacccagtccgccctgagcaaagaccccaacgag
aagcgcgatcacatggtcctgctggagttcgtgaccgccgccggg
atcactctcggcatggacgagctgtacaagtaaaagcttatcgat
aatcaacctctggattacaaaatttgtgaaagattgactggtatt
cttaactatgttgctccttttacgctatgtggatacgctgcttta
atgcctttgtatcatgctattgcttcccgtatggctttcattttc
tcctccttgtataaatcctggttgctgtctctttatgaggagttg
tggcccgttgtcaggcaacgtggcgtggtgtgcactgtgtttgct
gacgcaacccccactggttggggcattgccaccacctgtcagctc
ctttccgggactttcgctttccccctccctattgccacggcggaa
ctcatcgccgcctgccttgcccgctgctggacaggggctcggctg
ttgggcactgacaattccgtggtgttgtcggggaaatcatcgtcc
tttccttggctgctcgcctatgttgccacctggattctgcgcggg
acgtccttctgctacgtcccttcggccctcaatccagcggacctt
ccttcccgcggcctgctgccggctctgcggcctcttccgcgtctt
cgccttcgccctcagacgagtcggatctccctttgggccgcctcc
ccgcatcgataccgagcgctgctcgagaGCGATCGCtgtgatagc
ggccatcaagctggccgcgactctagatcataatcagccatacca
catttgtagaggttttacttgctttaaaaaacctcccacacctcc
ccctgaacctgaaacataaaatgaatgcaattgttgttgttaact
tgtttattgcagcttataatggttacaaataaagcaatagcatca
caaatttcacaaataaagcatttttttcactgcattctagttgtg
gtttgtccaaactcatcaatgtatcagcttatcgataccgcatgc
acgtgcggaccgagcggccgcaggaacccctagtgatggagttgg
ccactccctctctgcgcgctcgctcgctcactgaggccgggcgac
caaaggtcgcccgacgcccgggctttgcccgggcggcctcagtga
gcgagcgagcgcgcagctgcctgcaggggcgcctgatgcggtatt
ttctccttacgcatctgtgcggtatttcacaccgcatacgtcaaa
gcaaccatagtacgcgccctgtagcggcgcattaagcgcggcggg
tgtggtggttacgcgcagcgtgaccgctacacttgccagcgccct
agcgcccgctcctttcgctttcttcccttcctttctcgccacgtt
cgccggctttccccgtcaagctctaaatcgggggctccctttagg
gttccgatttagtgctttacggcacctcgaccccaaaaaacttga
tttgggtgatggttcacgtagtgggccatcgccctgatagacggt
ttttcgccctttgacgttggagtccacgttctttaatagtggact
cttgttccaaactggaacaacactcaaccctatctcgggctattc
ttttgatttataagggattttgccgatttcggcctattggttaaa
aaatgagctgatttaacaaaaatttaacgcgaattttaacaaaat
attaacgtttacaattttatggtgcactctcagtacaatctgctc
tgatgccgcatagttaagccagccccgacacccgccaacacccgc
tgacgcgccctgacgggcttgtctgctcccggcatccgcttacag
acaagctgtgaccgtctccgggagctgcatgtgtcagaggttttc
accgtcatcaccgaaacgcgcgagacgaaagggcctcgtgatacg
cctatttttataggttaatgtcatgataataatggtttcttagac
gtcaggtggcacttttcggggaaatgtgcgcggaacccctatttg
tttatttttctaaatacattcaaatatgtatccgctcatgagaca
ataaccctgataaatgcttcaataatattgaaaaaggaagagta

### REFERENCES

Alkaslasi, M. R., Piccus, Z. E., Hareendran, S., Silberberg, H., Chen, L., Zhang, Y., Petros, T. J., & Le Pichon, C. E. (2021). Single nucleus RNA-sequencing defines unexpected diversity of cholinergic neuron types in the adult mouse spinal cord. Nature Communications, 12(1), 2471.
Armbruster, N., Lattanzi, A., Jeavons, M., Van Wittenberghe, L., Gjata, B., Marais, T., Martin, S., Vignaud, A., Voit, T., Mavilio, F., Barkats, M., & Buj-Bello, A. (2016). Efficacy and biodistribution analysis of intracerebroventricular administration of an optimized scAAV9-SMN1 vector in a mouse model of spinal muscular atrophy. Molecular Therapy-Methods & Clinical Development, 3, 16060.
Buenrostro, J. D., Wu, B., Chang, H. Y., & Greenleaf, W. J. (2015). ATAC-seq: A method for assaying chromatin accessibility genome-wide. Current Protocols in Molecular Biology/Edited by Frederick M. Ausubel . . . [et Al.], 10⁹(1), 21.29.1-21.29.9.
Chan, K. Y., Jang, M. J., Yoo, B. B., Greenbaum, A., Ravi, N., Wu, W.-L., Sánchez-Guardado, L., Lois, C., Mazmanian, S. K., Deverman, B. E., & Gradinaru, V. (2017). Engineered AAVs for efficient noninvasive gene delivery to the central and peripheral nervous systems. Nature Neuroscience, 20(8), 1172-1179.
Hrvatin, S., Tzeng, C. P., Nagy, M. A., Stroud, H., Koutsioumpa, C., Wilcox, O. F., Assad, E. G., Green, J., Harvey, C. D., Griffith, E. C., & Greenberg, M. E. (2019). A scalable platform for the development of cell-type-specific viral drivers, eLife, 8, https://doi.org/10.7554/eLife.48089
Love, M. I., Huber, W., & Anders, S. (2014). Moderated estimation of fold change and dispersion for RNA-seq data with DESeq2. Genome Biology, 15(12), 550.
Mo, A., Mukamel, E. A., Davis, F. P., Luo, C., Henry, G. L., Picard, S., Urich, M. A., Nery, J. R., Sejnowski, T. J., Lister, R., Eddy, S. R., Ecker, J. R., & Nathans, J. (2015). Epigenomic Signatures of Neuronal Diversity in the Mammalian Brain. Neuron, 86(6), 1369-1384.
Patel, T., Hammelman, J., Closser, M., Gifford, D. K., & Wichterle, H. (2021). General and cell-type-specific aspects of the motor neuron maturation transcriptional program. In bioRxiv (p. 2021.03.05.434185), https://doi.org/10.1101/2021.03.05.434185
Rhee, H. S., Closser, M., Guo, Y., Bashkirova, E. V., Tan, G. C., Gifford, D. K., & Wichterle, H. (2016). Expression of Terminal Effector Genes in Mammalian Neurons Is Maintained by a Dynamic Relay of Transient Enhancers. Neuron, 92(6), 1252-1265.
Rossi, J., Balthasar, N., Olson, D., Scott, M., Berglund, E., Lee, C. E., Choi, M. J., Lauzon, D., Lowell, B. B., & Elmquist, J. K. (2011). Melanocortin-4 receptors expressed by cholinergic neurons regulate energy balance and glucose homeostasis. Cell Metabolism, 13(2), 195-204.
Sathyamurthy, A., Johnson, K. R., Matson, K. J. E., Dobrott, C. I., Li, L., Ryba, A. R., Bergman, T. B., Kelly, M. C., Kelley, M. W., & Levine, A. J. (2018). Massively Parallel Single Nucleus Transcriptional Profiling Defines Spinal Cord Neurons and Their Activity during Behavior. Cell Reports, 22(8), 2216-2225.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. A nucleic acid comprising a regulatory element sequence that has at least 85% identity to SEQ ID NOs: 1-14 or 60-71 or a fragment thereof.

2. The nucleic acid of claim 1,
(a) wherein the regulatory element sequence has at least 90%, 95%, 98%, 99% or 100% identity to SEQ ID NOs: 1-14 or 60-71, and/or
(b) wherein the sequence comprises at least one modification relative to SEQ ID NO: 1-14 or 60-71; and/or
(c) wherein the nucleic acid comprises at least one additional regulatory element sequence; optionally,
(1) wherein the nucleic acid comprises at least two, three, four, five or six additional regulatory element sequences; and/or
(2) wherein the at least one additional regulatory element sequence has at least 85% identity to SEQ ID NO: 1-14 and 60-71; and/or
(3) wherein the at least one additional regulatory element sequence has at least 90%, 95%, 98%, 99% or 100% identity to SEQ ID NOs: 1-14 or 60-71; and/or
(4) wherein the at least one additional regulatory sequence comprises at least one modification relative to SEQ ID NO: 1-14 or 60-71, optionally a substitute modification; and/or
(d) wherein the nucleic acid comprises two or more identical copies of a regulatory element sequence selected from the group consisting of SEQ ID NO: 1-14 or 60-71, optionally, wherein the nucleic acid comprises two, three, four, five or six identical copies of the regulatory element sequence; and/or
(e) wherein the nucleic acid comprises at least two or more versions of a regulatory element sequence having at least 85%, 90%, 95%, 98%, 99% or 100% identity to SEQ ID NO: 1-14 or 60-71, wherein the first version of the regulatory element sequence differs from the second version of the regulatory element sequence; and/or
(f) wherein the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides; and/or
(g) wherein the regulatory element comprises one or more transcription factor binding sites; optionally, wherein the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA) or a combination thereof.

3-11. (canceled)

12. The nucleic acid of claim 1,
(a) further comprising a promoter; optionally
(1) wherein the promoter is a promoter from a gene expressed in motor neurons, optionally wherein the gene expressed in motor neurons is selected from the group consisting of a Dnajc22, Sycp1, Slit3, Hrasls5, Otop3, Conb3, Nlrp3, Hormad1, Chat, Anxa4, Tnfsf4, Myo3b, Cdh15, Nr2e1, 1117f, Apela, Gnb3, Pappa, Tmprss15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, II22ra1, Galp, Meil, Aox1, Prph, Slc25a54, Cdhr1, Tgm6, Ppm1j, Esrp1, Gem, Is11, Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd, Terb2, Ins15, Card11, Platr7, Miat, Slc5a7, Iqcg, Topaz1, Tex14, Slc5a10, Map4k1, Calcb, Got111, Slc46a2, Nanos 2, Plekhg4, Themis3, Cpa3, Aknad1, Cdcp2, Uts2, Slc44a4, Popdc3, Tbata, Pkp1, Dysf, Pkp2, Sds, Nipsnap3a, Apo17e, Tex22, Mapk 11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNCIH1, PLEKHG5, SIGMAR1, DNAJB2, SMAX3, TPII, ATLI, SPAST, NIPA1, KIAA1096, KIF5A, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS VapBC, ANG, TARDBP, FIG4, OPTN, ATXN2, VCP, CHMP2B, PFN1, ERBB4, HNRNPA2B1, MATR3, TUBA4A, ANXA1I, NEK1, DAO, NEFH, SOSTM1, CYLD, CHCHD10, UBQLN2, HEXA, MFN2, RAB7A, NEFL, GDAP1, LRSAM1, MORC2, SOD1, and C9orf72; and/or
(2) wherein the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx 1 promoter, Lhx3 promoter, Lhx4 promoter, and variants thereof; optionally
(i) wherein the promoter is pBG (optionally comprising SEQ ID NO: 55); optionally further comprising a pBG intron (optionally comprising SEQ ID NO: 56), optionally wherein there is a linker between the pBG and pBG intron of zero to 500 nucleotides.

13. The nucleic acid of claim 1,
(a) further comprising a heterologous gene; optionally
(1) wherein the heterologous gene is naturally expressed in a neuron; optionally, wherein the neuron is selected from the group consisting of a motor neuron, a sensory neuron, and an interneuron; and/or
(2) wherein the heterologous gene is selectively expressed in a motor neuron; optionally, wherein the heterologous gene is selectively expressed in a motor neuron, but is not expressed in dorsal root ganglia; and/or
(3) wherein the heterologous gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7BI (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2BI (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), and a combination thereof; optionally wherein the heterologous gene is SMN1; optionally, SMN1 gene comprises the sequence set forth in SEQ ID NO: 25-28; and/or
(4) wherein the heterologous gene is an inhibitory nucleic acid; optionally,
(i) wherein the inhibitory nucleic acid is a microRNA (miRNA), artificial microRNA (amiRNA), or short hairpin RNA (shRNA), optionally wherein the inhibitory nucleic acid is a microRNA, wherein the microRNA binds to mRNA of a target gene; optionally wherein the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic I Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7BI (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member IA), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), SOD1 (superoxide dismutase 1), C9orf72 (C9orf72-SMCR8 Complex Subunit), or a combination thereof; optionally,
wherein the target gene is SOD1, optionally SOD1 gene comprises the sequence set forth in SEQ ID NO: 33; or
wherein the target gene is C9orf72, optionally wherein the C9orf72 gene comprises the sequence set forth in SEQ ID NO: 35, 36, or 37; and/or
(b) wherein the regulatory element comprises SEQ ID NO: 1-14 or 60-71.

14-37. (canceled)

38. The nucleic acid of claim 1, wherein the nucleic acid is associated with an adeno-associated virus comprising a capsid that crosses the blood brain barrier and/or the blood spinal cord barrier, optionally wherein the adeno-associated virus comprising a capsid is selected from the group consisting of AAV1, AAV2, AAV3, AAV4, AAV5, AAV6, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, and AAV-PHP.eB.

39. (canceled)

40. A vector comprising the nucleic acid of claim 1, optionally wherein the vector is a viral vector, such as a recombinant adeno-associated viral (AAV) vector.

41. (canceled)

42. (canceled)

43. A recombinant adeno-associated viral (rAAV) vector comprising a regulatory element sequence that has at least 85% identity to SEQ ID NOs: 1-14 or 60-71 or a fragment thereof.

44. The rAAV vector of claim 43,
(a) wherein the regulatory element sequence has at least 90%, 95%, 98%, 99%, or 100% identity to SEQ ID NO: 1-14 or 60-71; and/or
(b) wherein the sequence comprises at least one modification relative to SEQ ID NO: 1-14 or 60-71, optionally a substitute modification; and/or
(c) wherein the nucleic acid comprises at least one additional regulatory element sequence; optionally,
(1) wherein the nucleic acid comprises at least two, three, four, five or six additional regulatory element sequences; and/or
(2) wherein the at least one additional regulatory element sequence has at least 85% identity to SEQ ID NO: 1-14 and 60-71; and/or
(3) wherein the at least one additional regulatory element sequence has at least 90%, 95%, 98%, 99% or 100% identity to SEQ ID NOs: 1-14 or 60-71; and/or
(4) wherein the at least one additional regulatory sequence comprises at least one modification relative to SEQ ID NO: 1-14 or 60-71, optionally a substitute modification; and/or
(d) wherein the nucleic acid comprises two or more identical copies of a regulatory element sequence selected from the group consisting of SEQ ID NO: 1-14 or 60-71, optionally, wherein the nucleic acid comprises two, three, four, five or six identical copies of the regulatory element sequence; and/or
(e) wherein the nucleic acid comprises at least two or more versions of a regulatory element sequence having at least 85%, 90%, 95%, 98%, 99% or 100% identity to SEQ ID NO: 1-14 or 60-71, wherein the first version of the regulatory element sequence differs from the second version of the regulatory element sequence; and/or
(f) wherein the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides; and/or
(g) wherein the regulatory element comprises one or more transcription factor binding sites; optionally, wherein the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA) or a combination thereof; and/or
(h) wherein the rAAV vector is replication-competent.

45-53. (canceled)

54. The rAAV vector of claim 43,
(a) further comprising a heterologous gene; optionally
(1) wherein the heterologous gene is naturally expressed in a neuron; optionally, wherein the neuron is selected from the group consisting of a motor neuron, a sensory neuron, and an interneuron; and/or
(2) wherein the heterologous gene is selectively expressed in a motor neuron; optionally, wherein the heterologous gene is selectively expressed in a motor neuron, but is not expressed in dorsal root ganglia; and/or
(3) wherein the heterologous gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7BI (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2BI (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), and a combination thereof; optionally wherein the heterologous gene is SMN1; optionally, SMN1 gene comprises the sequence set forth in SEQ ID NO: 25-28; and/or
(4) wherein the heterologous gene is an inhibitory nucleic acid; optionally,
(i) wherein the inhibitory nucleic acid is a microRNA (miRNA), artificial microRNA (amiRNA), or short hairpin RNA (shRNA), optionally wherein the inhibitory nucleic acid is a microRNA, wherein the microRNA binds to mRNA of a target gene; optionally wherein the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member IA), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2BI (Heterogeneous Nuclear Ribonucleoprotein A2/B1), MATR3 (Matrin 3), TUBA4A (Tubulin Alpha 4a), ANXA1l (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), SOD1 (superoxide dismutase 1), C9orf72 (C9orf72-SMCR8 Complex Subunit), or a combination thereof; optionally,
wherein the target gene is SOD1, optionally SOD1 gene comprises the sequence set forth in SEQ ID NO: 33; or
wherein the target gene is C9orf72, optionally wherein the C9orf72 gene comprises the sequence set forth in SEQ ID NO: 35, 36, or 37; and/or
(b) wherein the regulatory element comprises SEQ ID NO: 1-14 or 60-71.

55-74. (canceled)

75. The rAAV of claim 43,
(a) further comprising a promoter; optionally
(1) wherein the promoter is a promoter from a gene expressed in motor neurons, optionally wherein the gene expressed in motor neurons is selected from the group consisting of a Dnajc22, Sycp1, Slit3, Hrasls5, Otop3, Ccnb3, Nlrp3, Hormad1, Chat, Anxa4, Tnfsf4, Myo3b, Cdh15, Nr2e1, 1117f, Apela, Gnb3, Pappa, Tmprss15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, 1122ra1, Galp, Meil, Aox1, Prph, Slc25a54, Cdhr1, Tgm6, Ppm1j, Esrp1, Gem, Is11, Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd, Terb2, Ins15, Card11, Platr7, Miat, Slc5a7, Iqcg, Topaz1, Tex14, Slc5a10, Map4k1, Calcb, Got111, Slc46a2, Nanos2, Plekhg4, Themis3, Cpa3, Aknad1, Cdcp2, Uts2, Slc44a4, Popdc3, Tbata, Pkp1, Dysf, Pkp2, Sds, Nipsnap3a, Apo17e, Tex22, Mapk 11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, Nlrp12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNCIH1, PLEKHG5, SIGMAR1, DNAJB2, SMAX3, TPII, ATLI, SPAST, NIPA1, KIAA1096, KIF5A, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP, CHMP2B, PFN1, ERBB4, HNRNPA2B1, MATR3, TUBA4A, ANXA1I, NEK1, DAO, NEFH, SOSTM1, CYLD, CHCHD10, UBOLN2, HEXA, MFN2, RAB7A, NEFL, GDAP1, LRSAM1, MORC2, SOD1, and C9orf72; and/or
(2) wherein the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx 1 promoter, Lhx3 promoter, Lhx4 promoter, and variants thereof; optionally
(i) wherein the promoter is pBG (optionally comprising SEQ ID NO: 55); optionally further comprising a pBG intron (optionally comprising SEQ ID NO: 56), optionally wherein there is a linker between the pBG and pBG intron of zero to 500 nucleotides.

76-79. (canceled)

80. A transgenic cell comprising the nucleic acid of claim 1; optionally
(a) wherein the transgenic cell is a neuron;
(b) wherein the transgenic cell is selected from the group consisting of a motor neuron, a sensory neuron, and an interneuron; and/or
(c) wherein the transgenic cell is murine, human, or non-human primate.

81-84. (canceled)

85. A composition comprising the nucleic acid of claim 1; and a pharmaceutically acceptable excipient.

86. (canceled)

87. A method for selectively expressing a heterologous gene within a population of neural cells in vivo or in vitro, the method comprising providing a pharmaceutical composition comprising a nucleic acid of claim 1 and a pharmaceutically acceptable excipient in a therapeutically effective dosage to a sample or a subject comprising the population of neural cells thereby selectively expressing the gene within the population of neural cells; optionally,
(a) wherein the pharmaceutical composition comprises a lipid nanoparticle;
(b) wherein the providing comprises administering to a living subject, optionally, wherein the living subject is a human, non-human primate, or a mouse; and/or
(c) wherein the administering to the living subject is through injection; optionally, wherein the injection comprises intracerebroventricular (ICV) or intravenous (IV) injection, optionally into the cisterna magna (ICM).

88-94. (canceled)

95. A method for the treatment of a motor neuron disease or disorder in a subject in need thereof, the method comprising administering a recombinant adeno-associated virus (rAAV) comprising a regulatory element sequence that has at least 85% identity to SEQ ID NOs: 1-14 or 60-71 or a fragment thereof to the subject, wherein one or more symptoms associated with the motor neuron disease or disorder are inhibited or prevented.

96. The method of claim 95,
(a) wherein the regulatory element sequence has at least 90%, 95%, 98%, 99%, or 100% identity to SEQ ID NO: 1-14 or 60-71;
(b) wherein the sequence comprises at least one modification relative to SEQ ID NO: 1-14 or 60-71, optionally a substitute modification; and/or
(c) wherein the nucleic acid comprises at least one additional regulatory element sequence; optionally,
(1) wherein the nucleic acid comprises at least two, three, four, five or six additional regulatory element sequences; and/or
(2) wherein the at least one additional regulatory element sequence has at least 85% identity to SEQ ID NO: 1-14 and 60-71; and/or
(3) wherein the at least one additional regulatory element sequence has at least 90%, 95%, 98%, 99% or 100% identity to SEQ ID NOs: 1-14 or 60-71; and/or
(4) wherein the at least one additional regulatory sequence comprises at least one modification relative to SEQ ID NO: 1-14 or 60-71, optionally a substitute modification; and/or
(d) wherein the nucleic acid comprises two or more identical copies of a regulatory element sequence selected from the group consisting of SEQ ID NO: 1-14 or 60-71, optionally, wherein the nucleic acid comprises two, three, four, five or six identical copies of the regulatory element sequence; and/or
(e) wherein the nucleic acid comprises at least two or more versions of a regulatory element sequence having at least 85%, 90%, 95%, 98%, 99% or 100% identity to SEQ ID NO: 1-14 or 60-71, wherein the first version of the regulatory element sequence differs from the second version of the regulatory element sequence; and/or
(f) wherein the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides; and/or
(g) wherein the regulatory element comprises one or more transcription factor binding sites; optionally, wherein the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrrb (SEQ ID NO: 21), a binding site for Myb (AACTGCCA) or a combination thereof.

97-105. (canceled)

106. The method of claim 95,
(a) further comprising a heterologous gene; optionally
(1) wherein the heterologous gene is naturally expressed in a neuron; optionally, wherein the neuron is selected from the group consisting of a motor neuron, a sensory neuron, and an interneuron; and/or
(2) wherein the heterologous gene is selectively expressed in a motor neuron; optionally, wherein the heterologous gene is selectively expressed in a motor neuron, but is not expressed in dorsal root ganglia; and/or
(3) wherein the heterologous gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7BI (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member 1A), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA1l (Annexin A11), NEKI (NIMA Related Kinase 1), DAO (D-Amino Acid Oxidase), NEFH (Neurofilament Heavy Chain), SOSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), superoxide dismutase 1 (SOD1), C9orf72 (C9orf72-SMCR8 Complex Subunit), and a combination thereof; optionally wherein the heterologous gene is SMN1; optionally, SMN1 gene comprises the sequence set forth in SEQ ID NO: 25-28; and/or
(4) wherein the heterologous gene is an inhibitory nucleic acid; optionally,
(i) wherein the inhibitory nucleic acid is a microRNA (miRNA), artificial microRNA (amiRNA), or short hairpin RNA (shRNA), optionally wherein the inhibitory nucleic acid is a microRNA, wherein the microRNA binds to mRNA of a target gene; optionally wherein the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATL1 (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7BI (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member IA), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2BI (Heterogeneous Nuclear Ribonucleoprotein A2/B1), MATR3 (Matrin 3), TUBA4A (Tubulin Alpha 4a), ANXA11 (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), SOD1 (superoxide dismutase 1), C9orf72 (C9orf72-SMCR8 Complex Subunit), or a combination thereof; optionally,
wherein the target gene is SOD1, optionally SOD1 gene comprises the sequence set forth in SEQ ID NO: 33; or
wherein the target gene is C9orf72, optionally wherein the C9orf72 gene comprises the sequence set forth in SEQ ID NO: 35, 36, or 37; and/or
(b) wherein the regulatory element comprises SEQ ID NO: 1-14 or 60-71.

107-110. (canceled)

111. The method of claim 95,
(a) further comprising a promoter; optionally
(1) wherein the promoter is a promoter from a gene expressed in motor neurons, optionally wherein the gene expressed in motor neurons is selected from the group consisting of a Dnajc22, Sycp1, Slit3, Hrasls5, Otop3, Conb3, Nlrp3, Hormad1, Chat, Anxa4, Tnfsf4, Myo3b, Cdh15, Nr2e1, 1117f, Apela, Gnb3, Pappa, Tmprss 15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, 1122ra1, Galp, Meil, Aox1, Prph, Slc25a54, Cdhr1, Tgm6, Ppm1j, Esrp1, Gem, Is11, Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd, Terb2, Ins15, Card11, Platr7, Miat, Slc5a7, Iqcg, Topaz1, Tex14, Slc5a10, Map4k1, Calcb, Got111, Slc46a2, Nanos 2, Plekhg4, Themis3, Cpa3, Aknad1, Cdcp2, Uts2, Slc44a4, Popdc3, Tbata, Pkp1, Dysf, Pkp2, Sds, Nipsnap3a, Apo17e, Tex22, Mapk 11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, N1rp 12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNCIH1, PLEKHG5, SIGMAR1, DNAJB2, SMAX3, TPII, ATLI, SPAST, NIPA1, KIAA1096, KIF5A, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS, VapBC, ANG, TARDBP, FIG. 4, OPTN, ATXN2, VCP, CHMP2B, PFN1, ERBB4, HNRNPA2B1, MATR3, TUBA4A, ANXA1I, NEK1, DAO, NEFH, SOSTM1, CYLD, CHCHD10, UBOLN2, HEXA, MFN2, RAB7A, NEFL, GDAP1, LRSAM1, MORC2, SOD1, and C9orf72; and/or
(2) wherein the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx 1 promoter, Lhx3 promoter, Lhx4 promoter, and variants thereof; optionally
(i) wherein the promoter is pBG (optionally comprising SEQ ID NO: 55); optionally further comprising a pBG intron (optionally comprising SEQ ID NO: 56), optionally wherein there is a linker between the pBG and pBG intron of zero to 500 nucleotides.

112-129. (canceled)

130. The method of claim 95,
(a) wherein the motor neuron disease or disorder is Spinal-bulbar muscular atrophy (SBMA), Spinal muscular atrophy (SMA) (e.g., non-5q SMA, Spinal muscular atrophy with congenital bone fractures, Autosomal dominant spinal muscular atrophy, and lower extremity-predominant 2 (SMALED2)), Distal hereditary motor neuropathy (dHMN) (e.g., autosomal dominant, autosomal recessive, type 1, 2, 4, 5, 6, 7, 7b), Triosephosphate isomerase deficiency (TIP), Hereditary spastic paraplegia (HSP) (also known as familial spastic paraparesis (FSP))(e.g., autosomal dominant or autosomal recessive or X-linked), amyotrophic lateral sclerosis (ALS), ALS with frontotemporal dementia (FTD), Adrenomyeloneuropathy (AMN), Allgrove syndrome (also known as triple A (3A) syndrome), Friedreich ataxia (FA), Spinocerebellar ataxia (SCA), Pontocerebellar hypoplasias (PCH), Neurodegeneration with brain iron accumulation (NBIA), mitochondrial complex I deficiency nuclear type 21 (MC1DN21), GM2 gangliosidosis (also known as Tay-Sachs disease or HexA deficiency), Charcot-Marie-Tooth disease (CMT), or a combination thereof; and/or
(b) wherein the one or more symptoms associated with the motor neuron disease or disorder are muscle weakness and decreased muscle tone, limited mobility, breathing problems, problems eating and swallowing, delayed gross motor skills, congenital hemolytic anemia, progressive neuromuscular dysfunction, spontaneous tongue movements, behavioral/cognitive symptoms, cerebellar degeneration, or scoliosis.

131. (canceled)

132. A method of silencing the expression of a target gene in a motor neuron, the method comprising contacting a recombinant adeno-associated virus (rAAV) comprising a nucleic acid comprising a regulatory element sequence that has at least 85% identity to SEQ ID NOs: 1-14 or 60-71 or a fragment thereof to the motor neuron.

133. The method of claim 132,
(a) wherein the regulatory element sequence has at least 90%, 95%, 98%, 99%, or 100% identity to SEQ ID NOs: 1-14 or 60-71;
(b) wherein the sequence comprises at least one modification relative to SEQ ID NOs: 1-14 or 60-71; and/or
(c) wherein the nucleic acid comprises at least one additional regulatory element sequence; optionally
(1) wherein the nucleic acid comprises at least two, three, four, five or six additional regulatory element sequences; and/or
(2) wherein the at least one additional regulatory element sequence has at least 85% identity to SEQ ID NO: 1-14 and 60-71; and/or
(3) wherein the at least one additional regulatory element sequence has at least 90%, 95%, 98%, 99%, or 100% identity to SEQ ID NOs: 1-14 or 60-71; and/or
(4) wherein the at least one additional regulatory sequence comprises at least one modification relative to SEQ ID NO: 1-14 or 60-71, optionally a substitute modification; and/or
(d) wherein the nucleic acid comprises two, three, four, five or six identical copies of a regulatory element sequence selected from the group consisting of SEQ ID NO: 1-14 or 60-71; and/or
(e) wherein the nucleic acid comprises at least two or more versions of a regulatory element sequence having at least 85%, 90%, 95%, 98%, 99%, or 100% identity to SEQ ID NO: 1-14 or 60-71, wherein the first version of the regulatory element sequence differs from the second version of the regulatory element sequence; and/or
(f) wherein the nucleic acid further comprising a heterologous gene; and/or
(g) wherein the regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides; and/or
(h) wherein the regulatory element comprises one or more transcription factor binding sites; optionally
(1) wherein the one or more transcription factor binding sites is selected from the group consisting of a binding site for Lhx3 (TTAATTAG), a binding site for Lhx4 (SEQ ID NO: 16), a binding site for Mnx1 (TTAATTAA), a binding site for Is12 (GCACTTAA), a binding site for RREB1 (SEQ ID NO: 19), a binding site for STAT4 (SEQ ID NO: 20), a binding site for Esrb (SEQ ID NO: 21), a binding site for Myb (AACTGCCA), or a combination thereof; and/or
(i) wherein the rAAV further comprises a promoter; optionally
(1) wherein the promoter is a promoter from a gene expressed in motor neurons, optionally wherein the gene expressed in motor neurons is selected from the group consisting of a Dnajc22, Sycp1, Slit3, Hrasls5, Otop3, Conb3, Nlrp3, Hormad1, Chat, Anxa4, Tnfsf4, Myo3b, Cdh15, Nr2e1, 1117f, Apela, Gnb3, Pappa, Tmprss 15, Crp, Nxpe5, Tex21, Ttc24, Ttc231, Ahnak2, Vipr2, Gstt4, Aox3, Plac811, Grin3b, Adam2, Co15a1, Clca3a1, Serpinb7, Edn2, Mgarp, Atp12a, Lhx4, Pip5k11, Slc25a48, Tfcp211, Clec18a, Spint2, 1122ra1, Galp, Meil, Aox1, Prph, Slc25a54, Cdhr1, Tgm6, Ppm1j, Esrp1, Gem, Is11, Itpr3, Sec16b, Pde6b, Haol, Oaslf, I121r, Ropn1, Pax6os1, Ctf2, Abcb5, Fcr15, Rxfp1, Cfhr1, Co16a4, Grid2ip, Myo15, Uts2b, Slc15a1, Rgs11, Spag6, Msh5, Tc2n, Trim31, Nanog, Mett14-ps1, Hpd, Terb2, Ins15, Card11, Platr7, Miat, Slc5a7, Iqcg, Topaz1, Tex14, Slc5a10, Map4k1, Calcb, Got111, Slc46a2, Nanos2, Plekhg4, Themis3, Cpa3, Aknad1, Cdcp2, Uts2, Slc44a4, Popdc3, Thata, Pkp1, Dysf, Pkp2, Sds, Nipsnap3a, Apo17e, Tex22, Mapk11, Fndc3c1, Axdnd1, Olah, Arhgap9, Cd4, Cpne6, Etnk2, Slc38a8, Sapcd1, Esp11, Glyat, Htr2a, Slc10a4, Retn, Abcb11, Fam71f1, Is12, Lcp1, Usp50, Echdc2, Ankrd60, N1rp 12, Noslap, Mnx1, Lhx3, Lhx4, SMN1, AR, BICD2, TRIP4, HSPB1, HSPB8, HSPB3, FBXO38, REEP1, BSCL2, GARS1, SLC5A7, TRPV4, ATP7A, IGHMBP2, DCTN1, DYNCIH1, PLEKHG5, SIGMAR1, DNAJB2, SMAX3, TPII, ATLI, SPAST, NIPA1, KIAA1096, KIF5A, RTN2, Hsp60, SPG37, SPG41, SLC33A1, REEP2, CPTIC, UBAP1, ALDH18A1, SPG11, CYP7B1, SPG7, ZFYVE26, SPG20, SPG21(ACP33), GJC2, SPG24, DDHD1, KIFIA, AP5Z1, FARS2, L1CAM, PLP1, ALS2, WDR7, TBK1, ABCD1, ALADIN, FXN, NOP56, ANO10, EXOSC3, C19orf12, NUBPL, FUS VapBC, ANG, TARDBP, FIG. 4, OPTN, ATXN2, VCP, CHMP2B, PFN1, ERBB4, HNRNPA2B1, MATR3, TUBA4A, ANXA1I, NEK1, DAO, NEFH, SOSTM1, CYLD, CHCHD10, UBQLN2, HEXA, MFN2, RAB7A, NEFL, GDAP1, LRSAM1, MORC2, SOD1, and C9orf72; optionally wherein the promoter is selected from the group consisting of beta globin promoter (pBG) (optionally comprising SEQ ID NO: 55), a choline acetyltransferase promoter (pChAT) (optionally comprising SEQ ID NO: 23), CAG promoter (pCAG) (optionally comprising SEQ ID NO: 24 or 57), minimal CMV promoter, human synapsin promoter, chicken beta actin, PGK promoter, Efla promoter, ubiquitin promoter, a TATA-box containing promoter, Slc5a7 promoter, Is11 promoter, Mnx1 promoter, Lhx3 promoter, Lhx4 promoter, and variants thereof; optionally
(1) wherein the promoter is pBG (optionally comprising SEQ ID NO: 55); optionally
(i) further comprising a pBG intron (optionally comprising SEQ ID NO: 56), optionally wherein there is a linker between the pBG and pBG intron of zero to 500 nucleotides.

134-151. (canceled)

152. The method of claim 132,
(a) wherein the heterologous gene is an inhibitory nucleic acid; optionally
(1) wherein the inhibitory nucleic acid is a microRNA (miRNA), artificial microRNA (amiRNA), or short hairpin RNA (shRNA), optionally wherein the inhibitory nucleic acid is a microRNA, wherein the microRNA binds to mRNA of a target gene, optionally wherein the target gene is selected from the group consisting of survival of motor neuron 1 (SMN1), AR (androgen receptor), BICD2 (BICD Cargo Adaptor 2), TRIP4 (Thyroid Hormone Receptor Interactor 4), HSPB1 (Heat Shock Protein Family B (Small) Member 1), HSPB8 (Heat Shock Protein Family B (Small) Member 8), HSPB3 (Heat Shock Protein Family B (Small) Member 3), FBXO38 (F-Box Protein 38), REEP1 (Receptor Accessory Protein 1), BSCL2 (BSCL2 Lipid Droplet Biogenesis Associated, Seipin), GARS1 (Glycyl-TRNA Synthetase 1), SLC5A7 (Solute Carrier Family 5 Member 7), TRPV4 (Transient Receptor Potential Cation Channel Subfamily V Member 4), ATP7A (ATPase Copper Transporting Alpha), IGHMBP2 (Immunoglobulin Mu DNA Binding Protein 2, SETX (Senataxin), DCTN1 (Dynactin Subunit 1), DYNC1H1 (Dynein Cytoplasmic 1 Heavy Chain 1), PLEKHG5 (Pleckstrin Homology And RhoGEF Domain Containing G5), SIGMAR1 (Sigma Non-Opioid Intracellular Receptor 1), DNAJB2 (DnaJ Heat Shock Protein Family (Hsp40) Member B2), SMAX3 (spinal muscular atrophy-3), TPII (Triosephosphate Isomerase 1), ATLI (Atlastin GTPase 1), SPAST (Spastin), NIPA1 (Non-imprinted in Prader-Willi/Angelman syndrome region protein 1), KIAA1096, KIF5A (Kinesin Family Member 5A), RTN2 (Reticulon 2), Heat Shock Protein Family D (Hsp60), SPG37 (Spastic Paraplegia 37), SPG41 (Spastic Paraplegia 41), SLC33A1 (Solute Carrier Family 33 Member 1), REEP2 (Receptor Accessory Protein 2), CPTIC (Carnitine Palmitoyltransferase 1C), UBAP1 (Ubiquitin Associated Protein 1), ALDH18A1 (Aldehyde Dehydrogenase 18 Family Member A1), SPG11 (SPG11 Vesicle Trafficking Associated, Spatacsin), CYP7B1 (Cytochrome P450 Family 7 Subfamily B Member 1), SPG7 (SPG7 Matrix AAA Peptidase Subunit, Paraplegin), ZFYVE26 (Zinc Finger FYVE-Type Containing 26), SPG20 (Spastic paraplegia 20, autosomal recessive), SPG21(ACP33) (Spastic paraplegia 21, autosomal recessive), GJC2 (Gap Junction Protein Gamma 2), SPG24 (Spastic Paraplegia 24 (Autosomal Recessive)), DDHD1 (DDHD Domain Containing 1), KIFIA (Kinesin Family Member IA), AP5Z1 (Adaptor Related Protein Complex 5 Subunit Zeta 1), FARS2 (Phenylalanyl-TRNA Synthetase 2, Mitochondrial), L1CAM (L1 Cell Adhesion Molecule), PLP1 (Proteolipid Protein 1), ALS2 (Alsin Rho Guanine Nucleotide Exchange Factor ALS2), WDR7 (WD Repeat Domain 7), TBK1 (TANK-binding kinase 1), ABCD1 (ATP Binding Cassette Subfamily D Member 1), ALADIN (ALacrima Achalasia aDrenal Insufficiency Neurologic disorder), FXN (Frataxin), NOP56 (NOP56 Ribonucleoprotein), ANO10 (Anoctamin 10), EXOSC3 (Exosome Component 3), C19orf12 (Chromosome 19 Open Reading Frame 12), NUBPL (Nucleotide Binding Protein Like), FUS (FUS RNA Binding Protein), VapBC (virulence associated proteins B and C), ANG (Angiogenin), TARDBP (TAR DNA Binding Protein), FIG4 (FIG4 Phosphoinositide 5-Phosphatase), OPTN (Optineurin), ATXN2 (Ataxin 2), VCP (Valosin Containing Protein), CHMP2B (Charged Multivesicular Body Protein 2B), PFN1 (Profilin 1), ERBB4 (Erb-B2 Receptor Tyrosine Kinase 4), HNRNPA2B1 (Heterogeneous Nuclear Ribonucleoprotein A2/B1), MATR3 (Matrin 3), TUBA4A (Tubulin Alpha 4a), ANXA1l (Annexin A11), NEKI (NIMA Related Kinase 1), DAO (D-Amino Acid Oxidase), NEFH (Neurofilament Heavy Chain), SQSTM1 (Sequestosome 1), CYLD (CYLD Lysine 63 Deubiquitinase), CHCHD10 (Coiled-Coil-Helix-Coiled-Coil-Helix Domain Containing 10), UBQLN2 (Ubiquilin 2), HEXA (Hexosaminidase Subunit Alpha), MFN2 (Mitofusin 2), RAB7A (RAB7A, Member RAS Oncogene Family), NEFL (Neurofilament light chain polypeptide), GDAP1 (Ganglioside Induced Differentiation Associated Protein 1), LRSAM1 (Leucine Rich Repeat And Sterile Alpha Motif Containing 1), MORC2 (MORC Family CW-Type Zinc Finger 2), SOD1 (superoxide dismutase 1), C9orf72 (C9orf72-SMCR8 Complex Subunit), or a combination thereof; and/or
(b) wherein the neuron is from a subject, optionally
(1) wherein the subject is mammalian, optionally wherein the subject is human; and/or
(2) wherein the subject has been diagnosed or is suspected of having a motor neuron disease or disorder, optionally wherein the motor neuron disease or disorder is Spinal-bulbar muscular atrophy (SBMA), Spinal muscular atrophy (SMA) (e.g., non-5q SMA, Spinal muscular atrophy with congenital bone fractures, Autosomal dominant spinal muscular atrophy, and lower extremity-predominant 2 (SMALED2)), Distal hereditary motor neuropathy (dHMN) (e.g., autosomal dominant, autosomal recessive, type 1, 2, 4, 5, 6, 7, 7b), Triosephosphate isomerase deficiency (TIP), Hereditary spastic paraplegia (HSP) (also known as familial spastic paraparesis (FSP))(e.g., autosomal dominant or autosomal recessive or X-linked), amyotrophic lateral sclerosis (ALS), ALS with frontotemporal dementia (FTD), Adrenomyeloneuropathy (AMN), Allgrove syndrome (also known as triple A (3A) syndrome), Friedreich ataxia (FA), Spinocerebellar ataxia (SCA), Pontocerebellar hypoplasias (PCH), Neurodegeneration with brain iron accumulation (NBIA), mitochondrial complex I deficiency nuclear type 21 (MC1DN21), GM2 gangliosidosis (also known as Tay-Sachs disease or HexA deficiency), Charcot-Marie-Tooth disease (CMT), or a combination thereof.

153-172. (canceled)


SEQUENCE LISTING (79 sequences)

SEQ ID NO: 1 - DNA, 772 bp, Homo sapiens
  source 1..772: mol_type = genomic DNA
ccaaagggatttggaggccatgcttccaacgaatgattcatagttagtgtcagggagccagaaaaaaagcaagtgagcaaggtcctgtccctgggagctgtagagaggagccctggggcccacccacaaagcagcacctgcagtctctttccctctcgaagcccagctatgttgtgcacaaagcaagtctgggcaccgaggacaggctggccaagggcaggcaggcaggcacgtagtcctctggcttccagccaccacactcacaggtttctgggaaaggctgactggggccactttgttcctttgaatctgagaatatatgactggggaagcctaaattaattaaatgatgctgaggcccgcctgagccggtgcacaggggatgggttatggagccctgagcaaactgcacccctagcccccagtgctggaatccagagaggctcatgagctcgattggaacgaagcctgtgcttaagtgcttccagagagacaaagaaataataaatcaggagcaggtgccccacccacacactgccatcaccaacaccagcctgcttctccacagaaatacagtggtttcacctctctggaaccagatgtttcagggaagcaacaaatggcaaagccctggaaatgacatggccccacaaccttctcagaaatgaggccaggctgggctggcacctccatccacagcagcaccccaccaccacaacccacccaagacctccaaacaccccctagacctcacccaggcactggtgcagca

SEQ ID NO: 2 - DNA, 772 bp, Homo sapiens
  source 1..772: mol_type = genomic DNA
tgctgcaccagtgcctgggtgaggtctagggggtgtttggaggtcttgggtgggttgtggtggtggggtgctgctgtggatggaggtgccagcccagcctggcctcatttctgagaaggttgtggggccatgtcatttccagggctttgccatttgttgcttccctgaaacatctggttccagagaggtgaaaccactgtatttctgtggagaagcaggctggtgttggtgatggcagtgtgtgggtggggcacctgctcctgatttattatttctttgtctctctggaagcacttaagcacaggcttcgttccaatcgagctcatgagcctctctggattccagcactgggggctaggggtgcagtttgctcagggctccataacccatcccctgtgcaccggctcaggcgggcctcagcatcatttaattaatttaggcttccccagtcatatattctcagattcaaaggaacaaagtggccccagtcagcctttcccagaaacctgtgagtgtggtggctggaagccagaggactacgtgcctgcctgcctgcccttggccagcctgtcctcggtgcccagacttgctttgtgcacaacatagctgggcttcgagagggaaagagactgcaggtgctgctttgtgggtgggccccagggctcctctctacagctcccagggacaggaccttgctcacttgctttttttctggctccctgacactaactatgaatcattcgttggaagcatggcctccaaatccctttgg

SEQ ID NO: 3 - DNA, 576 bp, Homo sapiens
  source 1..576: mol_type = genomic DNA
gctgtagagaggagccctggggcccacccacaaagcagcacctgcagtctctttccctctcgaagcccagctatgttgtgcacaaagcaagtctgggcaccgaggacaggctggccaagggcaggcaggcaggcacgtagtcctctggcttccagccaccacactcacaggtttctgggaaaggctgactggggccactttgttcctttgaatctgagaatatatgactggggaagcctaaattaattaaatgatgctgaggcccgcctgagccggtgcacaggggatgggttatggagccctgagcaaactgcacccctagcccccagtgctggaatccagagaggctcatgagctcgattggaacgaagcctgtgcttaagtgcttccagagagacaaagaaataataaatcaggagcaggtgccccacccacacactgccatcaccaacaccagcctgcttctccacagaaatacagtggtttcacctctctggaaccagatgtttcagggaagcaacaaatggcaaagccctggaaatgacatggccccacaaccttctcagaaatgaggcc

SEQ ID NO: 4 - DNA, 576 bp, Homo sapiens
  source 1..576: mol_type = genomic DNA
ggcctcatttctgagaaggttgtggggccatgtcatttccagggctttgccatttgttgcttccctgaaacatctggttccagagaggtgaaaccactgtatttctgtggagaagcaggctggtgttggtgatggcagtgtgtgggtggggcacctgctcctgatttattatttctttgtctctctggaagcacttaagcacaggcttcgttccaatcgagctcatgagcctctctggattccagcactgggggctaggggtgcagtttgctcagggctccataacccatcccctgtgcaccggctcaggcgggcctcagcatcatttaattaatttaggcttccccagtcatatattctcagattcaaaggaacaaagtggccccagtcagcctttcccagaaacctgtgagtgtggtggctggaagccagaggactacgtgcctgcctgcctgcccttggccagcctgtcctcggtgcccagacttgctttgtgcacaacatagctgggcttcgagagggaaagagactgcaggtgctgctttgtgggtgggccccagggctcctctctacagc

SEQ ID NO: 5 - DNA, 302 bp, Homo sapiens
  source 1..302: mol_type = genomic DNA
gccaagggcaggcaggcaggcacgtagtcctctggcttccagccaccacactcacaggtttctgggaaaggctgactggggccactttgttcctttgaatctgagaatatatgactggggaagcctaaattaattaaatgatgctgaggcccgcctgagccggtgcacaggggatgggttatggagccctgagcaaactgcacccctagcccccagtgctggaatccagagaggctcatgagctcgattggaacgaagcctgtgcttaagtgcttccagagagacaaagaaataataaatca

SEQ ID NO: 6 - DNA, 302 bp, Homo sapiens
  source 1..302: mol_type = genomic DNA
tgatttattatttctttgtctctctggaagcacttaagcacaggcttcgttccaatcgagctcatgagcctctctggattccagcactgggggctaggggtgcagtttgctcagggctccataacccatcccctgtgcaccggctcaggcgggcctcagcatcatttaattaatttaggcttccccagtcatatattctcagattcaaaggaacaaagtggccccagtcagcctttcccagaaacctgtgagtgtggtggctggaagccagaggactacgtgcctgcctgcctgcccttggc

SEQ ID NO: 7 - DNA, 875 bp, Mus sp.
  source 1..875: mol_type = genomic DNA
  misc_difference 14: note = c, g, t, or a
accgtggcttagtntgataaaccaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaa

SEQ ID NO: 8 - DNA, 875 bp, Mus sp.
  source 1..875: mol_type = genomic DNA
  misc_difference 862: note = c, g, t, or a
ttgagaaccactggtctgtggcaaatcttggagagggaaatgaacctcaagaagtcattgaattgaagtgcaaaagaaacctgagggctaaagtcacagtgatctggggccaggctttctatcagaatgcatgtgaggtatctgctactcaaaaggccgctcattggccaggccctttctctggaagctgtagagagcagatcagggcccacttactggtacagaagacactgtctttacccttctgtacctactccattgtccgagtctgggcattcaggatgggctggccaagggcagcaaggcagacacatagtcctctggcttccagccatcagacttttccgggaatggctgacgtgggccactctgttcttttgaatctgagcctatgtagcagtagaggcctaattaattaagtaatgctgaagccttgggactctcccaaaggcagcacacaggggaggggttggtgaggcctgagaacgttctaccccacccccatccccagtgctggaatccagaggctcctgagtgtcattggaactaaagcctgcacttaagtgcttccacagagacaaagaaatcataaatcaagagcgggcaaccccgcccacgcactgctaaccaccacgccaacctgcttctccagacacccaggcagtttgcagctcagaaatgaagccagaataggctgatctctccctccacagcagcacctacagacccatgaccccaagaaggcactgctgcagaatggctgctcccaccctctctccagactcccccaccccagaacttcacactctctacccactccccacagcactgattcataatggagcaggttttggtttatcanactaagccacggt

SEQ ID NO: 9 - DNA, 500 bp, Mus sp.
  source 1..500: mol_type = genomic DNA
ggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagc

SEQ ID NO: 10 - DNA, 500 bp, Mus sp.
  source 1..500: mol_type = genomic DNA
gctgtagagagcagatcagggcccacttactggtacagaagacactgtctttacccttctgtacctactccattgtccgagtctgggcattcaggatgggctggccaagggcagcaaggcagacacatagtcctctggcttccagccatcagacttttccgggaatggctgacgtgggccactctgttcttttgaatctgagcctatgtagcagtagaggcctaattaattaagtaatgctgaagccttgggactctcccaaaggcagcacacaggggaggggttggtgaggcctgagaacgttctaccccacccccatccccagtgctggaatccagaggctcctgagtgtcattggaactaaagcctgcacttaagtgcttccacagagacaaagaaatcataaatcaagagcgggcaaccccgcccacgcactgctaaccaccacgccaacctgcttctccagacacccaggcagtttgcagctcagaaatgaagcc

SEQ ID NO: 11 - DNA, 280 bp, Mus sp.
  source 1..280: mol_type = genomic DNA
gcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggcca

SEQ ID NO: 12 - DNA, 280 bp, Mus sp.
  source 1..280: mol_type = genomic DNA
tggccaagggcagcaaggcagacacatagtcctctggcttccagccatcagacttttccgggaatggctgacgtgggccactctgttcttttgaatctgagcctatgtagcagtagaggcctaattaattaagtaatgctgaagccttgggactctcccaaaggcagcacacaggggaggggttggtgaggcctgagaacgttctaccccacccccatccccagtgctggaatccagaggctcctgagtgtcattggaactaaagcctgcacttaagtgc

SEQ ID NO: 13 - DNA, 854 bp, Mus sp.
  source 1..854: mol_type = genomic DNA
tttcttaataactgctattttgaaatgtatcattatcataactccagtgtagaagtggtgtccagatttctgctatgttgctaatttttgatatgagacattcttattagagttgagggaatgtgcttgtatcacttaggtgcacacaccagaagccagtgcaggctcaaggtgaacacagagactcgtggtaccccaaatggctctctatctgacttcagctctcttccacttcttcaactagaaatattgctgagggcttgttaaacacacaaaagccatggcttttgaccatcttgcaagcaaaagaaacaccattttaaactcctttgaaaacgttctcttctttcacattaagaggctgccacacgaacagaacgtgccataaataatgtgtgctaacattttccaaaaactggacatcaattaacgttaatttatgagaacacttcttgagaggagcacagttcagactcataactactgaaaaggctcattaatagaaatgtgtagggagagggtttttttcttcttctaaagggaacattaaagtaaacacatatcattgcaaggaaggctcatgatttattgcaaactcagtggaaaggagactttacgctgtgtttccagggtgaattttgagcaaaggaatcaagcaaacaaaatgaaatgaggatattctcttaggaaaggcatcctgtgacaacccagacaaatgatagctaatacttatataataagtactacatatcaggtcaggcactatgccaacatgatcttgtgtgtgtctcaccaagaacactgccagggaaatttgttttgctgccatatacaaagttaaaaatcaagccccc

SEQ ID NO: 14 - DNA, 854 bp, Mus sp.
  source 1..854: mol_type = genomic DNA
gggggcttgatttttaactttgtatatggcagcaaaacaaatttccctggcagtgttcttggtgagacacacacaagatcatgttggcatagtgcctgacctgatatgtagtacttattatataagtattagctatcatttgtctgggttgtcacaggatgcctttcctaagagaatatcctcatttcattttgtttgcttgattcctttgctcaaaattcaccctggaaacacagcgtaaagtctcctttccactgagtttgcaataaatcatgagccttccttgcaatgatatgtgtttactttaatgttccctttagaagaagaaaaaaaccctctccctacacatttctattaatgagccttttcagtagttatgagtctgaactgtgctcctctcaagaagtgttctcataaattaacgttaattgatgtccagtttttggaaaatgttagcacacattatttatggcacgttctgttcgtgtggcagcctcttaatgtgaaagaagagaacgttttcaaaggagtttaaaatggtgtttcttttgcttgcaagatggtcaaaagccatggcttttgtgtgtttaacaagccctcagcaatatttctagttgaagaagtggaagagagctgaagtcagatagagagccatttggggtaccacgagtctctgtgttcaccttgagcctgcactggcttctggtgtgtgcacctaagtgatacaagcacattccctcaactctaataagaatgtctcatatcaaaaattagcaacatagcagaaatctggacaccacttctacactggagttatgataatgatacatttcaaaatagcagttattaagaaa

SEQ ID NO: 15 - skipped

SEQ ID NO: 16 - DNA, 13 bp, Mus sp.
  source 1..13: mol_type = genomic DNA
taattaattaagt

SEQ ID NO: 17-18 - skipped (2 entries)

SEQ ID NO: 19 - DNA, 20 bp, Mus sp.
  source 1..20: mol_type = genomic DNA
gcactggggatgggggtggg

SEQ ID NO: 20 - DNA, 14 bp, Mus sp.
  source 1..14: mol_type = genomic DNA
tttccgggaatggc

SEQ ID NO: 21 - DNA, 12 bp, Homo sapiens
  source 1..12: mol_type = genomic DNA
tggccaagggca

SEQ ID NO: 22 - DNA, 682 bp, synthetic construct
  source 1..682: mol_type = other DNA
  misc_difference 50: note = c, g, t, or a
  misc_difference 51: note = c, g, t, or a
  misc_difference 52: note = c, g, t, or a
  misc_difference 53: note = c, g, t, or a
  misc_difference 54: note = c, g, t, or a
  misc_difference 55: note = c, g, t, or a
  misc_difference 56: note = c, g, t, or a
  misc_difference 57: note = c, g, t, or a
  misc_difference 58: note = c, g, t, or a
  misc_difference 59: note = c, g, t, or a
  misc_difference 60: note = c, g, t, or a
  misc_difference 61: note = c, g, t, or a
  misc_difference 62: note = c, g, t, or a
  misc_difference 63: note = c, g, t, or a
  misc_difference 64: note = c, g, t, or a
  misc_difference 65: note = c, g, t, or a
  misc_difference 66: note = c, g, t, or a
  misc_difference 67: note = c, g, t, or a
  misc_difference 68: note = c, g, t, or a
  misc_difference 69: note = c, g, t, or a
  misc_difference 70: note = c, g, t, or a
  misc_difference 71: note = c, g, t, or a
  misc_difference 72: note = c, g, t, or a
  misc_difference 73: note = c, g, t, or a
  misc_difference 74: note = c, g, t, or a
  misc_difference 75: note = c, g, t, or a
  misc_difference 76: note = c, g, t, or a
  misc_difference 77: note = c, g, t, or a
  misc_difference 78: note = c, g, t, or a
  misc_difference 79: note = c, g, t, or a
  misc_difference 80: note = c, g, t, or a
  misc_difference 81: note = c, g, t, or a
  misc_difference 82: note = c, g, t, or a
  misc_difference 83: note = c, g, t, or a
  misc_difference 84: note = c, g, t, or a
  misc_difference 85: note = c, g, t, or a
  misc_difference 86: note = c, g, t, or a
  misc_difference 87: note = c, g, t, or a
  misc_difference 88: note = c, g, t, or a
  misc_difference 89: note = c, g, t, or a
  misc_difference 90: note = c, g, t, or a
  misc_difference 91: note = c, g, t, or a
  misc_difference 92: note = c, g, t, or a
  misc_difference 93: note = c, g, t, or a
  misc_difference 94: note = c, g, t, or a
  misc_difference 95: note = c, g, t, or a
  misc_difference 96: note = c, g, t, or a
  misc_difference 97: note = c, g, t, or a
  misc_difference 98: note = c, g, t, or a
  misc_difference 99: note = c, g, t, or a
  misc_difference 100: note = c, g, t, or a
  misc_difference 101: note = c, g, t, or a
  misc_difference 102: note = c, g, t, or a
  misc_difference 103: note = c, g, t, or a
  misc_difference 104: note = c, g, t, or a
  misc_difference 105: note = c, g, t, or a
  misc_difference 106: note = c, g, t, or a
  misc_difference 107: note = c, g, t, or a
  misc_difference 108: note = c, g, t, or a
  misc_difference 109: note = c, g, t, or a
  misc_difference 110: note = c, g, t, or a
  misc_difference 111: note = c, g, t, or a
  misc_difference 112: note = c, g, t, or a
  misc_difference 113: note = c, g, t, or a
  misc_difference 114: note = c, g, t, or a
  misc_difference 115: note = c, g, t, or a
  misc_difference 116: note = c, g, t, or a
  misc_difference 117: note = c, g, t, or a
  misc_difference 118: note = c, g, t, or a
  misc_difference 119: note = c, g, t, or a
  misc_difference 120: note = c, g, t, or a
  misc_difference 121: note = c, g, t, or a
  misc_difference 122: note = c, g, t, or a
  misc_difference 123: note = c, g, t, or a
  misc_difference 124: note = c, g, t, or a
  misc_difference 125: note = c, g, t, or a
  misc_difference 126: note = c, g, t, or a
  misc_difference 127: note = c, g, t, or a
  misc_difference 128: note = c, g, t, or a
  misc_difference 129: note = c, g, t, or a
  misc_difference 130: note = c, g, t, or a
  misc_difference 131: note = c, g, t, or a
  misc_difference 132: note = c, g, t, or a
  misc_difference 133: note = c, g, t, or a
  misc_difference 134: note = c, g, t, or a
  misc_difference 135: note = c, g, t, or a
  misc_difference 136: note = c, g, t, or a
  misc_difference 137: note = c, g, t, or a
  misc_difference 138: note = c, g, t, or a
  misc_difference 139: note = c, g, t, or a
  misc_difference 140: note = c, g, t, or a
  misc_difference 141: note = c, g, t, or a
  misc_difference 142: note = c, g, t, or a
  misc_difference 143: note = c, g, t, or a
  misc_difference 144: note = c, g, t, or a
  misc_difference 145: note = c, g, t, or a
  misc_difference 146: note = c, g, t, or a
  misc_difference 147: note = c, g, t, or a
  misc_difference 148: note = c, g, t, or a
  misc_difference 149: note = c, g, t, or a
  misc_difference 150: note = c, g, t, or a
  misc_difference 151: note = c, g, t, or a
  misc_difference 152: note = c, g, t, or a
  misc_difference 153: note = c, g, t, or a
  misc_difference 154: note = c, g, t, or a
  misc_difference 155: note = c, g, t, or a
  misc_difference 156: note = c, g, t, or a
  misc_difference 157: note = c, g, t, or a
  misc_difference 158: note = c, g, t, or a
  misc_difference 159: note = c, g, t, or a
  misc_difference 160: note = c, g, t, or a
  misc_difference 161: note = c, g, t, or a
  misc_difference 162: note = c, g, t, or a
  misc_difference 163: note = c, g, t, or a
  misc_difference 164: note = c, g, t, or a
  misc_difference 165: note = c, g, t, or a
  misc_difference 166: note = c, g, t, or a
  misc_difference 167: note = c, g, t, or a
  misc_difference 168: note = c, g, t, or a
  misc_difference 169: note = c, g, t, or a
  misc_difference 170: note = c, g, t, or a
  misc_difference 171: note = c, g, t, or a
  misc_difference 172: note = c, g, t, or a
  misc_difference 173: note = c, g, t, or a
  misc_difference 174: note = c, g, t, or a
  misc_difference 175: note = c, g, t, or a
  misc_difference 176: note = c, g, t, or a
  misc_difference 177: note = c, g, t, or a
  misc_difference 178: note = c, g, t, or a
  misc_difference 179: note = c, g, t, or a
  misc_difference 180: note = c, g, t, or a
  misc_difference 181: note = c, g, t, or a
  misc_difference 182: note = c, g, t, or a
  misc_difference 183: note = c, g, t, or a
  misc_difference 184: note = c, g, t, or a
  misc_difference 185: note = c, g, t, or a
  misc_difference 186: note = c, g, t, or a
  misc_difference 187: note = c, g, t, or a
  misc_difference 188: note = c, g, t, or a
  misc_difference 189: note = c, g, t, or a
  misc_difference 190: note = c, g, t, or a
  misc_difference 191: note = c, g, t, or a
  misc_difference 192: note = c, g, t, or a
  misc_difference 193: note = c, g, t, or a
  misc_difference 194: note = c, g, t, or a
  misc_difference 195: note = c, g, t, or a
  misc_difference 196: note = c, g, t, or a
  misc_difference 197: note = c, g, t, or a
  misc_difference 198: note = c, g, t, or a
  misc_difference 199: note = c, g, t, or a
  misc_difference 200: note = c, g, t, or a
  misc_difference 201: note = c, g, t, or a
  misc_difference 202: note = c, g, t, or a
  misc_difference 203: note = c, g, t, or a
  misc_difference 204: note = c, g, t, or a
  misc_difference 205: note = c, g, t, or a
  misc_difference 206: note = c, g, t, or a
  misc_difference 207: note = c, g, t, or a
  misc_difference 208: note = c, g, t, or a
  misc_difference 209: note = c, g, t, or a
  misc_difference 210: note = c, g, t, or a
  misc_difference 211: note = c, g, t, or a
  misc_difference 212: note = c, g, t, or a
  misc_difference 213: note = c, g, t, or a
  misc_difference 214: note = c, g, t, or a
  misc_difference 215: note = c, g, t, or a
  misc_difference 216: note = c, g, t, or a
  misc_difference 217: note = c, g, t, or a
  misc_difference 218: note = c, g, t, or a
  misc_difference 219: note = c, g, t, or a
  misc_difference 220: note = c, g, t, or a
  misc_difference 221: note = c, g, t, or a
  misc_difference 222: note = c, g, t, or a
  misc_difference 223: note = c, g, t, or a
  misc_difference 224: note = c, g, t, or a
  misc_difference 225: note = c, g, t, or a
  misc_difference 226: note = c, g, t, or a
  misc_difference 227: note = c, g, t, or a
  misc_difference 228: note = c, g, t, or a
  misc_difference 229: note = c, g, t, or a
  misc_difference 230: note = c, g, t, or a
  misc_difference 231: note = c, g, t, or a
  misc_difference 232: note = c, g, t, or a
  misc_difference 233: note = c, g, t, or a
  misc_difference 234: note = c, g, t, or a
  misc_difference 235: note = c, g, t, or a
  misc_difference 236: note = c, g, t, or a
  misc_difference 237: note = c, g, t, or a
  misc_difference 238: note = c, g, t, or a
  misc_difference 239: note = c, g, t, or a
  misc_difference 240: note = c, g, t, or a
  misc_difference 241: note = c, g, t, or a
  misc_difference 242: note = c, g, t, or a
  misc_difference 243: note = c, g, t, or a
  misc_difference 244: note = c, g, t, or a
  misc_difference 245: note = c, g, t, or a
  misc_difference 246: note = c, g, t, or a
  misc_difference 247: note = c, g, t, or a
  misc_difference 248: note = c, g, t, or a
  misc_difference 249: note = c, g, t, or a
  misc_difference 250: note = c, g, t, or a
  misc_difference 251: note = c, g, t, or a
  misc_difference 252: note = c, g, t, or a
  misc_difference 253: note = c, g, t, or a
  misc_difference 254: note = c, g, t, or a
  misc_difference 255: note = c, g, t, or a
  misc_difference 256: note = c, g, t, or a
  misc_difference 257: note = c, g, t, or a
  misc_difference 258: note = c, g, t, or a
  misc_difference 259: note = c, g, t, or a
  misc_difference 260: note = c, g, t, or a
  misc_difference 261: note = c, g, t, or a
  misc_difference 262: note = c, g, t, or a
  misc_difference 263: note = c, g, t, or a
  misc_difference 264: note = c, g, t, or a
  misc_difference 265: note = c, g, t, or a
  misc_difference 266: note = c, g, t, or a
  misc_difference 267: note = c, g, t, or a
  misc_difference 268: note = c, g, t, or a
  misc_difference 269: note = c, g, t, or a
  misc_difference 270: note = c, g, t, or a
  misc_difference 271: note = c, g, t, or a
  misc_difference 272: note = c, g, t, or a
  misc_difference 273: note = c, g, t, or a
  misc_difference 274: note = c, g, t, or a
  misc_difference 275: note = c, g, t, or a
  misc_difference 276: note = c, g, t, or a
  misc_difference 277: note = c, g, t, or a
  misc_difference 278: note = c, g, t, or a
  misc_difference 279: note = c, g, t, or a
  misc_difference 280: note = c, g, t, or a
  misc_difference 281: note = c, g, t, or a
  misc_difference 282: note = c, g, t, or a
  misc_difference 283: note = c, g, t, or a
  misc_difference 284: note = c, g, t, or a
  misc_difference 285: note = c, g, t, or a
  misc_difference 286: note = c, g, t, or a
  misc_difference 287: note = c, g, t, or a
  misc_difference 288: note = c, g, t, or a
  misc_difference 289: note = c, g, t, or a
  misc_difference 290: note = c, g, t, or a
  misc_difference 291: note = c, g, t, or a
  misc_difference 292: note = c, g, t, or a
  misc_difference 293: note = c, g, t, or a
  misc_difference 294: note = c, g, t, or a
  misc_difference 295: note = c, g, t, or a
  misc_difference 296: note = c, g, t, or a
  misc_difference 297: note = c, g, t, or a
  misc_difference 298: note = c, g, t, or a
  misc_difference 299: note = c, g, t, or a
  misc_difference 300: note = c, g, t, or a
  misc_difference 301: note = c, g, t, or a
  misc_difference 302: note = c, g, t, or a
  misc_difference 303: note = c, g, t, or a
  misc_difference 304: note = c, g, t, or a
  misc_difference 305: note = c, g, t, or a
  misc_difference 306: note = c, g, t, or a
  misc_difference 307: note = c, g, t, or a
  misc_difference 308: note = c, g, t, or a
  misc_difference 309: note = c, g, t, or a
  misc_difference 310: note = c, g, t, or a
  misc_difference 311: note = c, g, t, or a
  misc_difference 312: note = c, g, t, or a
  misc_difference 313: note = c, g, t, or a
  misc_difference 314: note = c, g, t, or a
  misc_difference 315: note = c, g, t, or a
  misc_difference 316: note = c, g, t, or a
  misc_difference 317: note = c, g, t, or a
  misc_difference 318: note = c, g, t, or a
  misc_difference 319: note = c, g, t, or a
  misc_difference 320: note = c, g, t, or a
  misc_difference 321: note = c, g, t, or a
  misc_difference 322: note = c, g, t, or a
  misc_difference 323: note = c, g, t, or a
  misc_difference 324: note = c, g, t, or a
  misc_difference 325: note = c, g, t, or a
  misc_difference 326: note = c, g, t, or a
  misc_difference 327: note = c, g, t, or a
  misc_difference 328: note = c, g, t, or a
  misc_difference 329: note = c, g, t, or a
  misc_difference 330: note = c, g, t, or a
  misc_difference 331: note = c, g, t, or a
  misc_difference 332: note = c, g, t, or a
  misc_difference 333: note = c, g, t, or a
  misc_difference 334: note = c, g, t, or a
  misc_difference 335: note = c, g, t, or a
  misc_difference 336: note = c, g, t, or a
  misc_difference 337: note = c, g, t, or a
  misc_difference 338: note = c, g, t, or a
  misc_difference 339: note = c, g, t, or a
  misc_difference 340: note = c, g, t, or a
  misc_difference 341: note = c, g, t, or a
  misc_difference 342: note = c, g, t, or a
  misc_difference 343: note = c, g, t, or a
  misc_difference 344: note = c, g, t, or a
  misc_difference 345: note = c, g, t, or a
  misc_difference 346: note = c, g, t, or a
  misc_difference 347: note = c, g, t, or a
  misc_difference 348: note = c, g, t, or a
  misc_difference 349: note = c, g, t, or a
  misc_difference 350: note = c, g, t, or a
  misc_difference 351: note = c, g, t, or a
  misc_difference 352: note = c, g, t, or a
  misc_difference 353: note = c, g, t, or a
  misc_difference 354: note = c, g, t, or a
  misc_difference 355: note = c, g, t, or a
  misc_difference 356: note = c, g, t, or a
  misc_difference 357: note = c, g, t, or a
  misc_difference 358: note = c, g, t, or a
  misc_difference 359: note = c, g, t, or a
  misc_difference 360: note = c, g, t, or a
  misc_difference 361: note = c, g, t, or a
  misc_difference 362: note = c, g, t, or a
  misc_difference 363: note = c, g, t, or a
  misc_difference 364: note = c, g, t, or a
  misc_difference 365: note = c, g, t, or a
  misc_difference 366: note = c, g, t, or a
  misc_difference 367: note = c, g, t, or a
  misc_difference 368: note = c, g, t, or a
  misc_difference 369: note = c, g, t, or a
  misc_difference 370: note = c, g, t, or a
  misc_difference 371: note = c, g, t, or a
  misc_difference 372: note = c, g, t, or a
  misc_difference 373: note = c, g, t, or a
  misc_difference 374: note = c, g, t, or a
  misc_difference 375: note = c, g, t, or a
  misc_difference 376: note = c, g, t, or a
  misc_difference 377: note = c, g, t, or a
  misc_difference 378: note = c, g, t, or a
  misc_difference 379: note = c, g, t, or a
  misc_difference 380: note = c, g, t, or a
  misc_difference 381: note = c, g, t, or a
  misc_difference 382: note = c, g, t, or a
  misc_difference 383: note = c, g, t, or a
  misc_difference 384: note = c, g, t, or a
  misc_difference 385: note = c, g, t, or a
  misc_difference 386: note = c, g, t, or a
  misc_difference 387: note = c, g, t, or a
  misc_difference 388: note = c, g, t, or a
  misc_difference 389: note = c, g, t, or a
  misc_difference 390: note = c, g, t, or a
  misc_difference 391: note = c, g, t, or a
  misc_difference 392: note = c, g, t, or a
  misc_difference 393: note = c, g, t, or a
  misc_difference 394: note = c, g, t, or a
  misc_difference 395: note = c, g, t, or a
  misc_difference 396: note = c, g, t, or a
  misc_difference 397: note = c, g, t, or a
  misc_difference 398: note = c, g, t, or a
  misc_difference 399: note = c, g, t, or a
  misc_difference 400: note = c, g, t, or a
  misc_difference 401: note = c, g, t, or a
  misc_difference 402: note = c, g, t, or a
  misc_difference 403: note = c, g, t, or a
  misc_difference 404: note = c, g, t, or a
  misc_difference 405: note = c, g, t, or a
  misc_difference 406: note = c, g, t, or a
  misc_difference 407: note = c, g, t, or a
  misc_difference 408: note = c, g, t, or a
  misc_difference 409: note = c, g, t, or a
  misc_difference 410: note = c, g, t, or a
  misc_difference 411: note = c, g, t, or a
  misc_difference 412: note = c, g, t, or a
  misc_difference 413: note = c, g, t, or a
  misc_difference 414: note = c, g, t, or a
  misc_difference 415: note = c, g, t, or a
  misc_difference 416: note = c, g, t, or a
  misc_difference 417: note = c, g, t, or a
  misc_difference 418: note = c, g, t, or a
  misc_difference 419: note = c, g, t, or a
  misc_difference 420: note = c, g, t, or a
  misc_difference 421: note = c, g, t, or a
  misc_difference 422: note = c, g, t, or a
  misc_difference 423: note = c, g, t, or a
  misc_difference 424: note = c, g, t, or a
  misc_difference 425: note = c, g, t, or a
  misc_difference 426: note = c, g, t, or a
  misc_difference 427: note = c, g, t, or a
  misc_difference 428: note = c, g, t, or a
  misc_difference 429: note = c, g, t, or a
  misc_difference 430: note = c, g, t, or a
  misc_difference 431: note = c, g, t, or a
  misc_difference 432: note = c, g, t, or a
  misc_difference 433: note = c, g, t, or a
  misc_difference 434: note = c, g, t, or a
  misc_difference 435: note = c, g, t, or a
  misc_difference 436: note = c, g, t, or a
  misc_difference 437: note = c, g, t, or a
  misc_difference 438: note = c, g, t, or a
  misc_difference 439: note = c, g, t, or a
  misc_difference 440: note = c, g, t, or a
  misc_difference 441: note = c, g, t, or a
  misc_difference 442: note = c, g, t, or a
  misc_difference 443: note = c, g, t, or a
  misc_difference 444: note = c, g, t, or a
  misc_difference 445: note = c, g, t, or a
  misc_difference 446: note = c, g, t, or a
  misc_difference 447: note = c, g, t, or a
  misc_difference 448: note = c, g, t, or a
  misc_difference 449: note = c, g, t, or a
  misc_difference 450: note = c, g, t, or a
  misc_difference 451: note = c, g, t, or a
  misc_difference 452: note = c, g, t, or a
  misc_difference 453: note = c, g, t, or a
  misc_difference 454: note = c, g, t, or a
  misc_difference 455: note = c, g, t, or a
  misc_difference 456: note = c, g, t, or a
  misc_difference 457: note = c, g, t, or a
  misc_difference 458: note = c, g, t, or a
  misc_difference 459: note = c, g, t, or a
  misc_difference 460: note = c, g, t, or a
  misc_difference 461: note = c, g, t, or a
  misc_difference 462: note = c, g, t, or a
  misc_difference 463: note = c, g, t, or a
  misc_difference 464: note = c, g, t, or a
  misc_difference 465: note = c, g, t, or a
  misc_difference 466: note = c, g, t, or a
  misc_difference 467: note = c, g, t, or a
  misc_difference 468: note = c, g, t, or a
  misc_difference 469: note = c, g, t, or a
  misc_difference 470: note = c, g, t, or a
  misc_difference 471: note = c, g, t, or a
  misc_difference 472: note = c, g, t, or a
  misc_difference 473: note = c, g, t, or a
  misc_difference 474: note = c, g, t, or a
  misc_difference 475: note = c, g, t, or a
  misc_difference 476: note = c, g, t, or a
  misc_difference 477: note = c, g, t, or a
  misc_difference 478: note = c, g, t, or a
  misc_difference 479: note = c, g, t, or a
  misc_difference 480: note = c, g, t, or a
  misc_difference 481: note = c, g, t, or a
  misc_difference 482: note = c, g, t, or a
  misc_difference 483: note = c, g, t, or a
  misc_difference 484: note = c, g, t, or a
  misc_difference 485: note = c, g, t, or a
  misc_difference 486: note = c, g, t, or a
  misc_difference 487: note = c, g, t, or a
  misc_difference 488: note = c, g, t, or a
  misc_difference 489: note = c, g, t, or a
  misc_difference 490: note = c, g, t, or a
  misc_difference 491: note = c, g, t, or a
  misc_difference 492: note = c, g, t, or a
  misc_difference 493: note = c, g, t, or a
  misc_difference 494: note = c, g, t, or a
  misc_difference 495: note = c, g, t, or a
  misc_difference 496: note = c, g, t, or a
  misc_difference 497: note = c, g, t, or a
  misc_difference 498: note = c, g, t, or a
  misc_difference 499: note = c, g, t, or a
  misc_difference 500: note = c, g, t, or a
  misc_difference 501: note = c, g, t, or a
  misc_difference 502: note = c, g, t, or a
  misc_difference 503: note = c, g, t, or a
  misc_difference 504: note = c, g, t, or a
  misc_difference 505: note = c, g, t, or a
  misc_difference 506: note = c, g, t, or a
  misc_difference 507: note = c, g, t, or a
  misc_difference 508: note = c, g, t, or a
  misc_difference 509: note = c, g, t, or a
  misc_difference 510: note = c, g, t, or a
  misc_difference 511: note = c, g, t, or a
  misc_difference 512: note = c, g, t, or a
  misc_difference 513: note = c, g, t, or a
  misc_difference 514: note = c, g, t, or a
  misc_difference 515: note = c, g, t, or a
  misc_difference 516: note = c, g, t, or a
  misc_difference 517: note = c, g, t, or a
  misc_difference 518: note = c, g, t, or a
  misc_difference 519: note = c, g, t, or a
  misc_difference 520: note = c, g, t, or a
  misc_difference 521: note = c, g, t, or a
  misc_difference 522: note = c, g, t, or a
  misc_difference 523: note = c, g, t, or a
  misc_difference 524: note = c, g, t, or a
  misc_difference 525: note = c, g, t, or a
  misc_difference 526: note = c, g, t, or a
  misc_difference 527: note = c, g, t, or a
  misc_difference 528: note = c, g, t, or a
  misc_difference 529: note = c, g, t, or a
  misc_difference 530: note = c, g, t, or a
  misc_difference 531: note = c, g, t, or a
  misc_difference 532: note = c, g, t, or a
  misc_difference 533: note = c, g, t, or a
  misc_difference 534: note = c, g, t, or a
  misc_difference 535: note = c, g, t, or a
  misc_difference 536: note = c, g, t, or a
  misc_difference 537: note = c, g, t, or a
  misc_difference 538: note = c, g, t, or a
  misc_difference 539: note = c, g, t, or a
  misc_difference 540: note = c, g, t, or a
  misc_difference 541: note = c, g, t, or a
  misc_difference 542: note = c, g, t, or a
  misc_difference 543: note = c, g, t, or a
  misc_difference 544: note = c, g, t, or a
  misc_difference 545: note = c, g, t, or a
  misc_difference 546: note = c, g, t, or a
  misc_difference 547: note = c, g, t, or a
  misc_difference 548: note = c, g, t, or a
  misc_difference 549: note = c, g, t, or a
  misc_difference 50..549: note = This region may encompass 0-500 nucleotides
ctgggcataaaagtcagggcagagccatctattgcttacatttgcttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 23 - DNA, 663 bp, Homo sapiens
  source 1..663: mol_type = genomic DNA
tctcttgtccaatggggcttggagcaccgaggccagcgaagccatcgcgctccttgcggaggtgaagaggaccctgagtccccacctgcggctcccctgtgtagagcctgcatctgtctgtccttccttccattgctcccagtgccaaacttgggccgctgcaccgcggcgcctccgcccaaatcaataaactgtgtctgtcccaggaggccgagtctctttactggtggggggtgcgtggaggcgcgcagggccagagcagaggggagggtgaactgggtctccaagtcccaatccagacctaagccaaactaacacgtaggcacctgtagctgtttttctacctggaaaaggggataggaaggaagcaaacccaacaaaggctgtcacccacggtcaccaaggagcaccatgctcccctcagcccaggatagaccctcttttccaggcctagcgcagagcccggggatgccgcccgggggagcctgaggacccgctccagctaggcacgccaggccccgccctttgaggacacgccccacaccagcctcagagctctgaggtgcctgggctgagcttcccttcagaccagaatcccgccccgttgaggctttgagaaaggagtaggagccgagcattccggcagaggaagaaaaacggccc

SEQ ID NO: 24 - DNA, 584 bp, Homo sapiens
  source 1..584: mol_type = genomic DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 25 - DNA, 1428 bp, Homo sapiens
  source 1..1428: mol_type = genomic DNA
gcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaatta

SEQ ID NO: 26 - DNA, 1538 bp, Homo sapiens
  source 1..1538: mol_type = genomic DNA
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaattaaaaaaa

SEQ ID NO: 27 - DNA, 1484 bp, Homo sapiens
  source 1..1484: mol_type = genomic DNA
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagataattcccccaccacctcccatatgtccagattctcttgatgatgctgatgctttgggaagtatgttaatttcatggtacatgagtggctatcatactggctattatatggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaattaaaaaaa

SEQ ID NO: 28 - DNA, 1482 bp, Homo sapiens
  source 1..1482: mol_type = genomic DNA
gcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaatta

SEQ ID NO: 29 - protein, 282 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMEMLA

SEQ ID NO: 30 - protein, 262 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKIIPPPPPICPDSLDDADALGSMLISWYMSGYHTGYYMGFRQNQKEGRCSHSLN

SEQ ID NO: 31 - protein, 250 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKIIPPPPPICPDSLDDADALGSMLISWYMSGYHTGYYMEMLA

SEQ ID NO: 32 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 33 - DNA, 895 bp, Homo sapiens
  source 1..895: mol_type = genomic DNA
gcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 34 - protein, 154 aa, Homo sapiens [sequence omitted]

SEQ ID NO: 35 - DNA, 3338 bp, Homo sapiens
  source 1..3338: mol_type = genomic DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 36 - DNA, 3231 bp, Homo sapiens
  source 1..3231: mol_type = genomic DNA
ggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 37 - DNA, 1950 bp, Homo sapiens
  source 1..1950: mol_type = genomic DNA
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttata

SEQ ID NO: 38 - protein, 481 aa, Homo sapiens
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLNAISSHLQTCGCSVVVGSSAEKVNKIVRTLCLFLTPAERKCSRLCEAESSFKYESGLFVQGLLKDSTGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFQLKPGLSLRSTFLAQFLLVLHRKALTLIKYIEDDTQKGKKPFKSLRNLKIDLDLTAEGDLNIIMALAEKIKPGLHSFIFGRPFYTSVQERDVLMTF

SEQ ID NO: 39 - protein, 481 aa, Homo sapiens; identical to SEQ ID NO: 38

SEQ ID NO: 40 - protein, 222 aa, Homo sapiens
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLK

SEQ ID NO: 41 - protein, 9 aa, synthetic construct
DGTLAVPFK

SEQ ID NO: 42 - protein, 7 aa, synthetic construct
NRGTEWD

SEQ ID NO: 43 - protein, 7 aa, synthetic construct
QAVRTSL

SEQ ID NO: 44 - protein, 7 aa, synthetic construct
TLAVPFK

SEQ ID NO: 45 - protein, 7 aa, synthetic construct
DSPAHPS

SEQ ID NO: 46 - DNA, 5795 bp, synthetic construct
  source 1..5795: mol_type = other DNA
  misc_difference 1854: note = c, g, t, or a
tgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaataccgtggcttagtntgataaaccaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaagtcgacagatctaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaagaagaattcccaaagaaaaagcggaaagtgctagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagcgatcgctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagta

SEQ ID NO: 47 - DNA, 5774 bp, synthetic construct
  source 1..5774: mol_type = other DNA
tgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattttcttaataactgctattttgaaatgtatcattatcataactccagtgtagaagtggtgtccagatttctgctatgttgctaatttttgatatgagacattcttattagagttgagggaatgtgcttgtatcacttaggtgcacacaccagaagccagtgcaggctcaaggtgaacacagagactcgtggtaccccaaatggctctctatctgacttcagctctcttccacttcttcaactagaaatattgctgagggcttgttaaacacacaaaagccatggcttttgaccatcttgcaagcaaaagaaacaccattttaaactcctttgaaaacgttctcttctttcacattaagaggctgccacacgaacagaacgtgccataaataatgtgtgctaacattttccaaaaactggacatcaattaacgttaatttatgagaacacttcttgagaggagcacagttcagactcataactactgaaaaggctcattaatagaaatgtgtagggagagggtttttttcttcttctaaagggaacattaaagtaaacacatatcattgcaaggaaggctcatgatttattgcaaactcagtggaaaggagactttacgctgtgtttccagggtgaattttgagcaaaggaatcaagcaaacaaaatgaaatgaggatattctcttaggaaaggcatcctgtgacaacccagacaaatgatagctaatacttatataataagtactacatatcaggtcaggcactatgccaacatgatcttgtgtgtgtctcaccaagaacactgccagggaaatttgttttgctgccatatacaaagttaaaaatcaagcccccgtcgacagatctaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaagaagaattcccaaagaaaaagcggaaagtgctagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagcgatcgctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagta

SEQ ID NO: 48 - DNA, 6140 bp, synthetic construct
  source 1..6140: mol_type = other DNA
  misc_difference 1854: note = c, g, t, or a
tgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaataccgtggcttagtntgataaaccaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaagtcgacagatcttctcttgtccaatggggcttggagcaccgaggccagcgaagccatcgcgctccttgcggaggtgaagaggaccctgagtccccacctgcggctcccctgtgtagagcctgcatctgtctgtccttccttccattgctcccagtgccaaacttgggccgctgcaccgcggcgcctccgcccaaatcaataaactgtgtctgtcccaggaggccgagtctctttactggtggggggtgcgtggaggcgcgcagggccagagcagaggggagggtgaactgggtctccaagtcccaatccagacctaagccaaactaacacgtaggcacctgtagctgtttttctacctggaaaaggggataggaaggaagcaaacccaacaaaggctgtcacccacggtcaccaaggagcaccatgctcccctcagcccaggatagaccctcttttccaggcctagcgcagagcccggggatgccgcccgggggagcctgaggacccgctccagctaggcacgccaggccccgccctttgaggacacgccccacaccagcctcagagctctgaggtgcctgggctgagcttcccttcagaccagaatcccgccccgttgaggctttgagaaaggagtaggagccgagcattccggcagaggaagaaaaacggcccgaattcccaaagaaaaagcggaaagtgctagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagcgatcgctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagta

SEQ ID NO: 49 - DNA, 6119 bp, synthetic construct
  source 1..6119: mol_type = other DNA
tgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattttcttaataactgctattttgaaatgtatcattatcataactccagtgtagaagtggtgtccagatttctgctatgttgctaatttttgatatgagacattcttattagagttgagggaatgtgcttgtatcacttaggtgcacacaccagaagccagtgcaggctcaaggtgaacacagagactcgtggtaccccaaatggctctctatctgacttcagctctcttccacttcttcaactagaaatattgctgagggcttgttaaacacacaaaagccatggcttttgaccatcttgcaagcaaaagaaacaccattttaaactcctttgaaaacgttctcttctttcacattaagaggctgccacacgaacagaacgtgccataaataatgtgtgctaacattttccaaaaactggacatcaattaacgttaatttatgagaacacttcttgagaggagcacagttcagactcataactactgaaaaggctcattaatagaaatgtgtagggagagggtttttttcttcttctaaagggaacattaaagtaaacacatatcattgcaaggaaggctcatgatttattgcaaactcagtggaaaggagactttacgctgtgtttccagggtgaattttgagcaaaggaatcaagcaaacaaaatgaaatgaggatattctcttaggaaaggcatcctgtgacaacccagacaaatgatagctaatacttatataataagtactacatatcaggtcaggcactatgccaacatgatcttgtgtgtgtctcaccaagaacactgccagggaaatttgttttgctgccatatacaaagttaaaaatcaagcccccgtcgacagatcttctcttgtccaatggggcttggagcaccgaggccagcgaagccatcgcgctccttgcggaggtgaagaggaccctgagtccccacctgcggctcccctgtgtagagcctgcatctgtctgtccttccttccattgctcccagtgccaaacttgggccgctgcaccgcggcgcctccgcccaaatcaataaactgtgtctgtcccaggaggccgagtctctttactggtggggggtgcgtggaggcgcgcagggccagagcagaggggagggtgaactgggtctccaagtcccaatccagacctaagccaaactaacacgtaggcacctgtagctgtttttctacctggaaaaggggataggaaggaagcaaacccaacaaaggctgtcacccacggtcaccaaggagcaccatgctcccctcagcccaggatagaccctcttttccaggcctagcgcagagcccggggatgccgcccgggggagcctgaggacccgctccagctaggcacgccaggccccgccctttgaggacacgccccacaccagcctcagagctctgaggtgcctgggctgagcttcccttcagaccagaatcccgccccgttgaggctttgagaaaggagtaggagccgagcattccggcagaggaagaaaaacggcccgaattcccaaagaaaaagcggaaagtgctagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagcgatcgctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagta

SEQ ID NO: 50 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 51 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 52 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 53 - DNA, 82 bp, synthetic construct
  source 1..82: mol_type = other DNA
aacttgtttattgcagcttataatggttacaaataaagcaatagcatcacaaatttcacaaataaagcatttttttcactgc

SEQ ID NO: 54 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 55 - DNA, 49 bp, synthetic construct
  source 1..49: mol_type = other DNA
ctgggcataaaagtcagggcagagccatctattgcttacatttgcttct

SEQ ID NO: 56 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 57 - DNA, 1600 bp, synthetic construct
  source 1..1600: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 58 - DNA, 266 bp, synthetic construct
  source 1..266: mol_type = other DNA
  misc_difference 50: note = c, g, t, or a
  misc_difference 51: note = c, g, t, or a
  misc_difference 52: note = c, g, t, or a
  misc_difference 53: note = c, g, t, or a
  misc_difference 54: note = c, g, t, or a
  misc_difference 55: note = c, g, t, or a
  misc_difference 56: note = c, g, t, or a
  misc_difference 57: note = c, g, t, or a
  misc_difference 58: note = c, g, t, or a
  misc_difference 59: note = c, g, t, or a
  misc_difference 60: note = c, g, t, or a
  misc_difference 61: note = c, g, t, or a
  misc_difference 62: note = c, g, t, or a
  misc_difference 63: note = c, g, t, or a
  misc_difference 64: note = c, g, t, or a
  misc_difference 65: note = c, g, t, or a
  misc_difference 66: note = c, g, t, or a
  misc_difference 67: note = c, g, t, or a
  misc_difference 68: note = c, g, t, or a
  misc_difference 69: note = c, g, t, or a
  misc_difference 70: note = c, g, t, or a
  misc_difference 71: note = c, g, t, or a
  misc_difference 72: note = c, g, t, or a
  misc_difference 73: note = c, g, t, or a
  misc_difference 74: note = c, g, t, or a
  misc_difference 75: note = c, g, t, or a
  misc_difference 76: note = c, g, t, or a
  misc_difference 77: note = c, g, t, or a
  misc_difference 78: note = c, g, t, or a
  misc_difference 79: note = c, g, t, or a
  misc_difference 80: note = c, g, t, or a
  misc_difference 81: note = c, g, t, or a
  misc_difference 82: note = c, g, t, or a
  misc_difference 83: note = c, g, t, or a
  misc_difference 84: note = c, g, t, or a
  misc_difference 85: note = c, g, t, or a
  misc_difference 86: note = c, g, t, or a
  misc_difference 87: note = c, g, t, or a
  misc_difference 88: note = c, g, t, or a
  misc_difference 89: note = c, g, t, or a
  misc_difference 90: note = c, g, t, or a
  misc_difference 91: note = c, g, t, or a
  misc_difference 92: note = c, g, t, or a
  misc_difference 93: note = c, g, t, or a
  misc_difference 94: note = c, g, t, or a
  misc_difference 95: note = c, g, t, or a
  misc_difference 96: note = c, g, t, or a
  misc_difference 97: note = c, g, t, or a
  misc_difference 98: note = c, g, t, or a
  misc_difference 99: note = c, g, t, or a
  misc_difference 100: note = c, g, t, or a
  misc_difference 101: note = c, g, t, or a
  misc_difference 102: note = c, g, t, or a
  misc_difference 103: note = c, g, t, or a
  misc_difference 104: note = c, g, t, or a
  misc_difference 105: note = c, g, t, or a
  misc_difference 106: note = c, g, t, or a
  misc_difference 107: note = c, g, t, or a
  misc_difference 108: note = c, g, t, or a
  misc_difference 109: note = c, g, t, or a
  misc_difference 110: note = c, g, t, or a
  misc_difference 111: note = c, g, t, or a
  misc_difference 112: note = c, g, t, or a
  misc_difference 113: note = c, g, t, or a
  misc_difference 114: note = c, g, t, or a
  misc_difference 115: note = c, g, t, or a
  misc_difference 116: note = c, g, t, or a
  misc_difference 117: note = c, g, t, or a
  misc_difference 118: note = c, g, t, or a
  misc_difference 119: note = c, g, t, or a
  misc_difference 120: note = c, g, t, or a
  misc_difference 121: note = c, g, t, or a
  misc_difference 122: note = c, g, t, or a
  misc_difference 123: note = c, g, t, or a
  misc_difference 124: note = c, g, t, or a
  misc_difference 125: note = c, g, t, or a
  misc_difference 126: note = c, g, t, or a
  misc_difference 127: note = c, g, t, or a
  misc_difference 128: note = c, g, t, or a
  misc_difference 129: note = c, g, t, or a
  misc_difference 130: note = c, g, t, or a
  misc_difference 131: note = c, g, t, or a
  misc_difference 132: note = c, g, t, or a
  misc_difference 133: note = c, g, t, or a
ctgggcataaaagtcagggcagagccatctattgcttacatttgcttctnnnnnnnnnnnnnnnnnnnnnnnnnnnnnnnnnnnnnnnnnnnnnnnnnnnnnnnnnnnnnnnnnnnnnnnnnnnnnnnnnnnngtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 59 - DNA, 266 bp, synthetic construct
  source 1..266: mol_type = other DNA
ctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 60 - DNA, 666 bp, Mus sp.
  source 1..666: mol_type = genomic DNA
ttgctacctactaacacttcataatcttaccaagataggaaaaggaacgggaccttataatagaatggaacataatgacacactcatcccagagtctcactcaggatctgcatttgggacaatcaaaggtcccctggcccttgttcagtcacttaatggagaagactccaaagacagaatgccactggtgtccttccaattatagaatcatctgattagaattacagtaaatgcatagctcagtttgcattgtcctgatgtgaactatgaggcctctctcctggagcatctgagggtactgtactctggaagtgtaccgccacgtcacagtagggtccttgtgccaggaccagcttagaaacgggacagaaacaagttaggacactccatttctgtggaccttagagcccaaggtaccagagctagatggtttgttttttttgggttttggggtgtttttttttttgtttgtttgtttgtttttttagattaatgcttagaagaaaaactgaagcctcacaaacttgagatagtagcatagttcagacgtgtagtaggaagggttgactttgggataattttagaattagttattctaagaggtggtccatagaacacaagtgtgtagcatctcggtccatgatgaaactggtcctatctggctat

SEQ ID NO: 61 - DNA, 257 bp, Mus sp.
  source 1..257: mol_type = genomic DNA
taacacttcataatcttaccaagataggaaaaggaacgggaccttataatagaatggaacataatgacacactcatcccagagtctcactcaggatctgcatttgggacaatcaaaggtcccctggcccttgttcagtcacttaatggagaagactccaaagacagaatgccactggtgtccttccaattatagaatcatctgattagaattacagtaaatgcatagctcagtttgcattgtcctgatgtgaactat

SEQ ID NO: 62 - DNA, 157 bp, Mus sp.
  source 1..157: mol_type = genomic DNA
taatgcttagaagaaaaactgaagcctcacaaacttgagatagtagcatagttcagacgtgtagtaggaagggttgactttgggataattttagaattagttattctaagaggtggtccatagaacacaagtgtgtagcatctcggtccatgatgaa

SEQ ID NO: 63 - DNA, 666 bp, Mus sp.
  source 1..666: mol_type = genomic DNA
atagccagataggaccagtttcatcatggaccgagatgctacacacttgtgttctatggaccacctcttagaataactaattctaaaattatcccaaagtcaacccttcctactacacgtctgaactatgctactatctcaagtttgtgaggcttcagtttttcttctaagcattaatctaaaaaaacaaacaaacaaacaaaaaaaaaaacaccccaaaacccaaaaaaaacaaaccatctagctctggtaccttgggctctaaggtccacagaaatggagtgtcctaacttgtttctgtcccgtttctaagctggtcctggcacaaggaccctactgtgacgtggcggtacacttccagagtacagtaccctcagatgctccaggagagaggcctcatagttcacatcaggacaatgcaaactgagctatgcatttactgtaattctaatcagatgattctataattggaaggacaccagtggcattctgtctttggagtcttctccattaagtgactgaacaagggccaggggacctttgattgtcccaaatgcagatcctgagtgagactctgggatgagtgtgtcattatgttccattctattataaggtcccgttccttttcctatcttggtaagattatgaagtgttagtaggtagcaa

SEQ ID NO: 64 - DNA, 257 bp, Mus sp.
  source 1..257: mol_type = genomic DNA
atagttcacatcaggacaatgcaaactgagctatgcatttactgtaattctaatcagatgattctataattggaaggacaccagtggcattctgtctttggagtcttctccattaagtgactgaacaagggccaggggacctttgattgtcccaaatgcagatcctgagtgagactctgggatgagtgtgtcattatgttccattctattataaggtcccgttccttttcctatcttggtaagattatgaagtgtta

SEQ ID NO: 65 - DNA, 157 bp, Mus sp.
  source 1..157: mol_type = genomic DNA
ttcatcatggaccgagatgctacacacttgtgttctatggaccacctcttagaataactaattctaaaattatcccaaagtcaacccttcctactacacgtctgaactatgctactatctcaagtttgtgaggcttcagtttttcttctaagcatta

SEQ ID NO: 66 - DNA, 1115 bp, Homo sapiens
  source 1..1115: mol_type = genomic DNA
gattaagaaattcaggttatttttcttattactttagtcaacaattatcatatatgattataatctagacttggaaatatttacctaaaatattcagtcactatattcaagcatacatacacacactccccaccacaaatacacacaaacactttgctcatttcatttgtttttcattgttaggagagcagttggtcagaatttattgaaagtacgggtgaaatgactgctacacacattttatgatcttaccaagaaaaaattaagaacttgatcctgttatagaatggaacatagtatccagatctcagagtctctatcacgatctgcgtttgggacaagtaaaggtcccctggcccttgttcaattgcttaatggaaaagactccaaagacagaatgccactggtgttcttccaattatagaatcatctgattagaattacagtaaatgcatagctcagtttgcattgtcctgaggtgaaccgcaaaccaagctgctctggttggagcatcggagggtactgaatgctgaaagcccactacctcatctcagcggggcactcatacaagggctaacttggaaagggacagataccagttaggatattccacttctggggaccctggagctctgggggccagagctagatggattatttaattaatgtttagtagaaatagtcaaatagcacacactctagacattaagccaatccagacctttggactgaattggagggaagatttgtcttcgtgactattttagaattaattattctagtttatttccagcctgtcagcattgagtcttgagaggtggtctgtaaaacacaagtttttccaatcatggggttgtgttgtggtcccatgggttttcttgctctgtctggccatagaagaacagatcaggaatcctacagaagaatcccaaatccattcctccccttctacttatttcagttacagctagagggttgggactcattcgtgtgttagaaccaaacctgactattgtgttattattgcttctaatttaactaccagactgttaaacattactgccccaagctcagccaggggtgggcactgcactttgaagccaccaagtcaatag

SEQ ID NO: 67 - DNA, 200 bp, Homo sapiens
  source 1..200: mol_type = genomic DNA
aacatagtatccagatctcagagtctctatcacgatctgcgtttgggacaagtaaaggtcccctggcccttgttcaattgcttaatggaaaagactccaaagacagaatgccactggtgttcttccaattatagaatcatctgattagaattacagtaaatgcatagctcagtttgcattgtcctgaggtgaaccgcaaa

SEQ ID NO: 68 - DNA, 104 bp, Homo sapiens
  source 1..104: mol_type = genomic DNA
ctattgtgttattattgcttctaatttaactaccagactgttaaacattactgccccaagctcagccaggggtgggcactgcactttgaagccaccaagtcaat

SEQ ID NO: 69 - DNA, 1115 bp, Homo sapiens
  source 1..1115: mol_type = genomic DNA
ctattgacttggtggcttcaaagtgcagtgcccacccctggctgagcttggggcagtaatgtttaacagtctggtagttaaattagaagcaataataacacaatagtcaggtttggttctaacacacgaatgagtcccaaccctctagctgtaactgaaataagtagaaggggaggaatggatttgggattcttctgtaggattcctgatctgttcttctatggccagacagagcaagaaaacccatgggaccacaacacaaccccatgattggaaaaacttgtgttttacagaccacctctcaagactcaatgctgacaggctggaaataaactagaataattaattctaaaatagtcacgaagacaaatcttccctccaattcagtccaaaggtctggattggcttaatgtctagagtgtgtgctatttgactatttctactaaacattaattaaataatccatctagctctggcccccagagctccagggtccccagaagtggaatatcctaactggtatctgtccctttccaagttagcccttgtatgagtgccccgctgagatgaggtagtgggctttcagcattcagtaccctccgatgctccaaccagagcagcttggtttgcggttcacctcaggacaatgcaaactgagctatgcatttactgtaattctaatcagatgattctataattggaagaacaccagtggcattctgtctttggagtcttttccattaagcaattgaacaagggccaggggacctttacttgtcccaaacgcagatcgtgatagagactctgagatctggatactatgttccattctataacaggatcaagttcttaattttttcttggtaagatcataaaatgtgtgtagcagtcatttcacccgtactttcaataaattctgaccaactgctctcctaacaatgaaaaacaaatgaaatgagcaaagtgtttgtgtgtatttgtggtggggagtgtgtgtatgtatgcttgaatatagtgactgaatattttaggtaaatatttccaagtctagattataatcatatatgataattgttgactaaagtaataagaaaaataacctgaatttcttaatc

SEQ ID NO: 70 - DNA, 200 bp, Homo sapiens
  source 1..200: mol_type = genomic DNA
tttgcggttcacctcaggacaatgcaaactgagctatgcatttactgtaattctaatcagatgattctataattggaagaacaccagtggcattctgtctttggagtcttttccattaagcaattgaacaagggccaggggacctttacttgtcccaaacgcagatcgtgatagagactctgagatctggatactatgtt

SEQ ID NO: 71 - DNA, 104 bp, Homo sapiens
  source 1..104: mol_type = genomic DNA
attgacttggtggcttcaaagtgcagtgcccacccctggctgagcttggggcagtaatgtttaacagtctggtagttaaattagaagcaataataacacaatag

SEQ ID NO: 72 - DNA, 479 bp, synthetic construct
  source 1..479: mol_type = other DNA
agcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgacttt

SEQ ID NO: 73 - DNA, 338 bp, synthetic construct
  source 1..338: mol_type = other DNA
agcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgacttt

SEQ ID NO: 74 - DNA, 501 bp, synthetic construct
  source 1..501: mol_type = other DNA
gagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcc

SEQ ID NO: 75 - DNA, 358 bp, synthetic construct
  source 1..358: mol_type = other DNA
gagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttc

SEQ ID NO: 76 - DNA, 500 bp, synthetic construct; identical to SEQ ID NO: 9
  source 1..500: mol_type = other DNA

SEQ ID NO: 77 - DNA, 280 bp, synthetic construct; identical to SEQ ID NO: 11
  source 1..280: mol_type = other DNA

SEQ ID NO: 78 - DNA, 670 bp, synthetic construct
  source 1..670: mol_type = other DNA
gagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaaaggctttagttccaatgacactcaggagcctctggattccagcactggggatgggggtggggtagaacgttctcaggcctcaccaacccctcccctgtgtgctgcctttgggagagtcccaaggcttcagcattgcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgacttt

SEQ ID NO: 79 - DNA, 624 bp, synthetic construct
  source 1..624: mol_type = other DNA
gagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaaaggctttagttccaatgacactcaggagcctctggattccagtagaacgttctcaggcctcaccaacccctcccctgtgtgctgcctttgggagagtcccaaggcttcagcattgcctctactgctacataggctcagattcaaaagaacagagtggcccacgtcaagtctgatggctggaagccagaggactatgtgtctgccttgcgcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgacttt